(19) United States
(12) Patent Application Publication
(10) Pub. No.: US 2026/0327233 A1
(43) Pub. Date: Oct. 22, 2026

(54) Heteroaryl Compounds as Ligand Directed Degraders of IRAK4

(71) Applicant: Celgene Corporation, Princeton, NJ (US)

(72) Inventors: Geraint Davies, Arlington, MA (US); Paul E. Gormisky, Belmont, MA (US); Timothy Gordon Rasmusson, Sudbury, MA (US); Guobin Miao, Lexington, MA (US)

(73) Assignee: Celgene Corporation, Princeton, NJ (US)

(21) Appl. No.: 19/481,292

(22) PCT Filed: May 9, 2024
(86) PCT No.: PCT/US2024/028518

§ 371 Date: Nov. 4, 2025

Related U.S. Application Data
(60) Provisional application No. 63/465,604, filed on May 11, 2023.

(51) Int. Cl. C07D 487/04; A61K 31/519

(52) U.S. Cl. CPC C07D 487/04; A61K 31/519

(57) ABSTRACT

Provided herein are compounds and compositions thereof for modulating IRAK4. In some embodiments, the compounds and compositions are provided for treatment of inflammatory or autoimmune diseases.

### CROSS-REFERENCE TO RELATED APPLICATIONS

This application claims priority to U.S. Provisional Application No. 63/465,604, filed on May 11, 2023, which is incorporated herein by reference in its entirety for any purpose.

### FIELD

The present disclosure relates generally to compounds, compositions, and methods for their preparation and use of the compounds and compositions for treating inflammatory or autoimmune diseases.

### BACKGROUND

The recruitment of immune cells to sites of injury involves the concerted interactions of a large number of soluble mediators. Several cytokines appear to play key roles in these processes, including interleukin-1 (IL-1). IL-1 produces proinflammatory responses and contributes to the tissue degeneration observed in chronic inflammatory conditions. IL-1 has also been implicated in the process of bone resorption and adipose tissue regulation. Thus, IL-1 plays a key role in a large number of pathological conditions including rheumatoid arthritis, inflammatory bowel disease, multiple sclerosis, diabetes, obesity, cancer, and sepsis.

IL-1 treatment of cells induces the formation of a complex consisting of the two IL-1 receptor chains, IL-1R1 and IL-1RAcP, and the resulting heterodimer recruits an adaptor molecule designated as MyD88, which binds to IL-1 receptor associated kinase (IRAK) (Wesche et al., J. Biol. Chem. 1999, 274, 19403-19410; O'Neill et al., J. Leukoc. Biol. 1998, 63, 650-657; Auron, Cytokine Growth Factor Rev. 1998, 9:221-237; and O'Neill, Biochem. Soc. Trans. 2000, 28, 557-563). Four members of the IRAK family have been identified: IRAK1, IRAK2, IRAK3, and IRAK4. These proteins are characterized by a typical N-terminal death domain that mediates interaction with MyD88-family adaptor proteins and a centrally located kinase domain. Of the four members in the mammalian TRAK family, IRAK-4 is considered to be the “master IRAK.” IRAK-4 is a serine/threonine kinase that plays an essential role in signal transduction by Toll/IL-1 receptors (TIRs). Under overexpression conditions, all TRAKs can mediate the activation of nuclear factor-kappa B and stress-induced mitogen activated protein kinase (MAPK)-signaling cascades. Studies have shown that IRAK4 kinase activity is essential for cytokine production, activation of MAPKs, and induction of NF-kappa B regulated genes in response to TLR ligands (Koziczak-Holbro M. et al., J Biol. Chem. 2007, 282, 13552-13560). Given the central role of IRAK4 in Toll-like/IL-1R signaling and immunological protection, compounds that modulate the function of IRAK4 may be useful in treating inflammatory, cell proliferative, and immune-related conditions and diseases associated with IRAK-mediated signal transduction such as rheumatoid arthritis, inflammatory bowel disease, multiple sclerosis, diabetes, obesity, allergic disease, psoriasis, asthma, graft rejection, cancer and sepsis.

Protein degradation is a highly regulated and essential process that maintains cellular homeostasis. Selective identification and removal of damaged, misfolded, or excess proteins is achieved through the ubiquitin-proteasome pathway (UPP). The UPP is central to the regulation of almost all cellular processes. Ubiquitination of the protein is accomplished by an E3 ubiquitin ligase that binds to a protein and adds ubiquitin molecules to the protein, thus marking the protein for proteasome degradation.

Harnessing the UPP for therapeutic use has received significant interest (Zhou et al., Mol. Cell 2000, 6, 751-756). One promising therapy uses proteolysis targeting chimeras, commonly referred to as PROTACs, to effect removal of unwanted proteins by protein degradation (Scheepstra et al., Comp. Struct. Biotech. J. 2019, 17, 160-176). PROTACS are ligand directed degraders that bring together an E3 ligase and a target protein that is to be degraded. These bivalent molecules usually consist of an E3 ligase ligand connected through a linker moiety to small molecule that binds to the target protein. A PROTAC positions the E3 ligase at the appropriate distance and orientation to the target protein, allowing the latter to be ubiquitinated. The ubiquitinated target protein is subsequently recognized by the proteasome, where it is degraded.

Accordingly, in one aspect, provided herein are compounds that target IRAK4 for degradation.

### SUMMARY

Described herein, in certain embodiments, are compounds and compositions thereof for degrading IRAK4. In various embodiments, the compounds and compositions thereof may be used in treatment of inflammatory or autoimmune diseases.

The present embodiments can be understood more fully by reference to the detailed description and examples, which are intended to exemplify non-limiting embodiments.

Embodiment 1 is a compound of Formula (I):

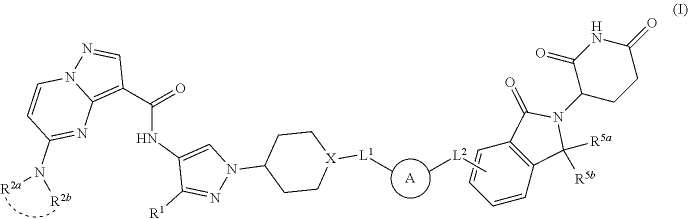

or a pharmaceutically acceptable salt thereof, wherein:
R¹ is C₁-C₆ haloalkyl, C₁-C₆ alkyl, or —CN;
R^{2a} is H or C₁-C₆ alkyl;
R^{2b} is C₅-C₆ cycloalkyl optionally substituted with 1-5 R³ groups;
or the dashed line between R^{2a} and R^{2b} represents a ring structure where R^{2a} and R^{2b} are taken together with the nitrogen atom to which they are attached to form a 6-membered heterocyclyl optionally containing one additional heteroatom selected from N and O, wherein the heterocyclyl is optionally substituted by 1-5 R³ groups;
each R³ is independently —NH₂, —OH, halo, C₁-C₆ alkyl, or C₁-C₆ haloalkyl;
X is CH or N;
L¹ is —C(O)N(H)—, —N(H)C(O)—, —C(O)—, —(C₁-C₆ alkylene)N(R⁴)—, or C₁-C₆ alkylene;
R⁴ is H or C₁-C₆ alkyl;
R^{5a} and R^{5b} are each H or are taken together to form an oxo group;
Ring A is

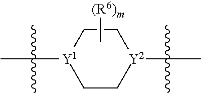

or an 8- to 10-membered spiro heterocyclylene containing 1-3 nitrogen atoms, wherein the heterocyclylene is substituted by m R⁶ groups;
Y¹ and Y² are independently CH or N;
each R⁶ is independently halo, C₁-C₆ alkyl, or C₁-C₆ haloalkyl;
m is 0-5;
L² is a bond or —N(R⁷)—; and
R⁷ is H or C₁-C₆ alkyl.

Embodiment 2 is the compound of embodiment 1, or a pharmaceutically acceptable salt thereof, wherein:
R¹ is C₁-C₃ haloalkyl, C₁-C₃ alkyl, or —CN.

Embodiment 3 is the compound of embodiment 2, or a pharmaceutically acceptable salt thereof, wherein:
R¹ is —CHF₂, —CF₃, —CH₃, or —CN.

Embodiment 4 is the compound of any one of embodiments 1-3, or a pharmaceutically acceptable salt thereof, wherein:
R^{2a} is H or C₁-C₃ alkyl; and
R^{2b} is cyclohexyl optionally substituted with 1-3 R³ groups.

Embodiment 5 is the compound of any one of embodiments 1-3, or a pharmaceutically acceptable salt thereof, wherein:
R^{2a} and R^{2b} are taken together with the nitrogen atom to which they are attached to form a 6-membered heterocyclyl optionally containing one additional heteroatom selected from N and O,
wherein the heterocyclyl is optionally substituted by 1-2 R³ groups.

Embodiment 6 is the compound of any one of embodiments 1-5, or a pharmaceutically acceptable salt thereof, wherein:
each R³ is independently —NH₂, —OH, halo, C₁-C₃ alkyl, or C₁-C₃ haloalkyl.

Embodiment 7 is the compound of embodiment 6, or a pharmaceutically acceptable salt thereof, wherein:
each R³ is independently —NH₂, —OH, F, or —CH₃.

Embodiment 8 is the compound of any one of embodiments 1-7, or a pharmaceutically acceptable salt thereof, wherein:

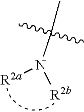

is

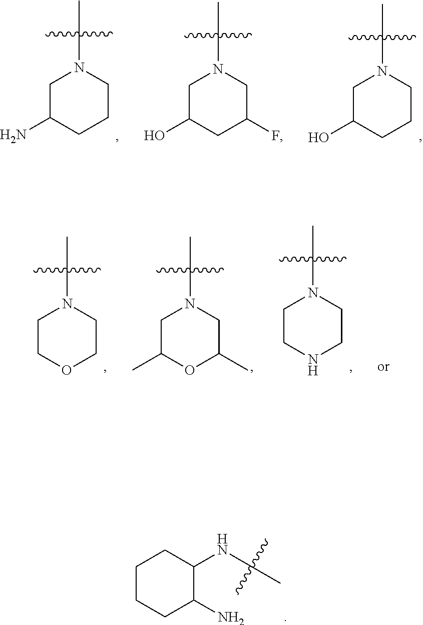

Embodiment 9 is the compound of any one of embodiments 1-8, or a pharmaceutically acceptable salt thereof, wherein:
L¹ is —C(O)N(H)—, —N(H)C(O)—, —C(O)—, —(C₁-C₃ alkylene)N(R⁴)—, or C₁-C₃ alkylene; and
R⁴ is H or C₁-C₃ alkyl.

Embodiment 10 is the compound of embodiment 9, or a pharmaceutically acceptable salt thereof, wherein:
L¹ is —C(O)N(H)—, —N(H)C(O)—, —C(O)—, —CH₂N(CH₃)—, —CH₂—, or —CH₂CH₂—.

Embodiment 11 is the compound of any one of embodiments 1-10, or a pharmaceutically acceptable salt thereof, wherein:
Ring A is

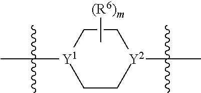

or a 10-membered spiro heterocyclylene containing 2 nitrogen atoms, wherein the spiro heterocyclylene is substituted by m R⁶ groups.

Embodiment 12 is the compound of any one of embodiments 1-11, or a pharmaceutically acceptable salt thereof, wherein:
each R⁶ is independently halo, C₁-C₃ alkyl, or C₁-C₃ haloalkyl.

Embodiment 13 is the compound of any one of embodiments 1-12, or a pharmaceutically acceptable salt thereof, wherein:
m is 0, 1, or 2.

Embodiment 14 is the compound of any one of embodiments 1-13, or a pharmaceutically acceptable salt thereof, wherein:
Ring A is

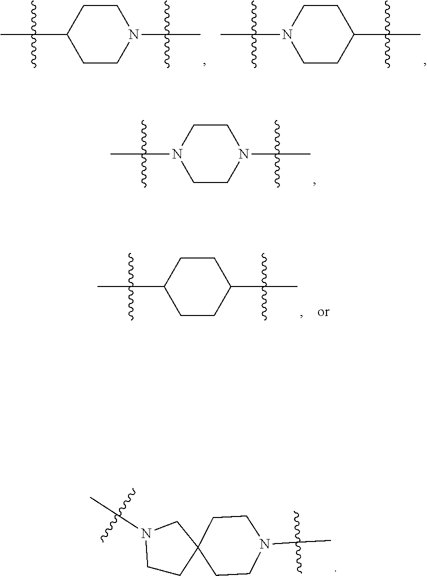 [SMILES: *C1CCN(*)CC1 ; *C1CCN(*)CC1 ; *N1CCN(*)CC1 ; *C1CCC(*)CC1 ; *N1CCC2(CC1)CCN(*)C2]

Embodiment 15 is the compound of any one of embodiments 1-14, or a pharmaceutically acceptable salt thereof, wherein:
(i) L² is a bond; or
(ii) L² is —N(R⁷)—; and
R⁷ is H or C₁-C₃ alkyl.

Embodiment 16 is the compound of any one of embodiments 1-15, or a pharmaceutically acceptable salt thereof, wherein:

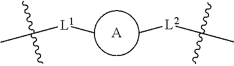

is

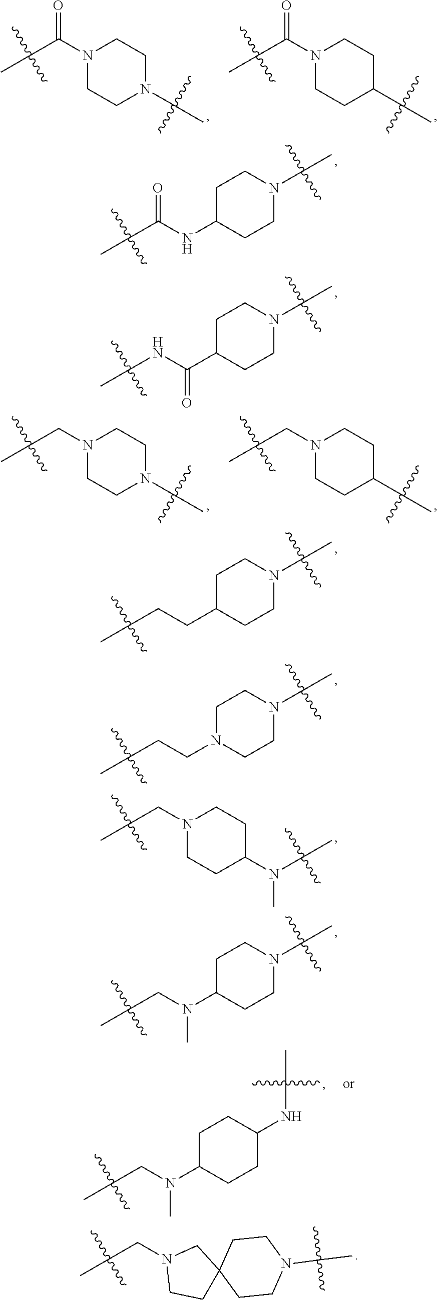

Embodiment 17 is the compound of any one of embodiments 1-16, or a pharmaceutically acceptable salt thereof, wherein the compound is of Formula (IIa), (IIb), (IIIa), or (IIIb):

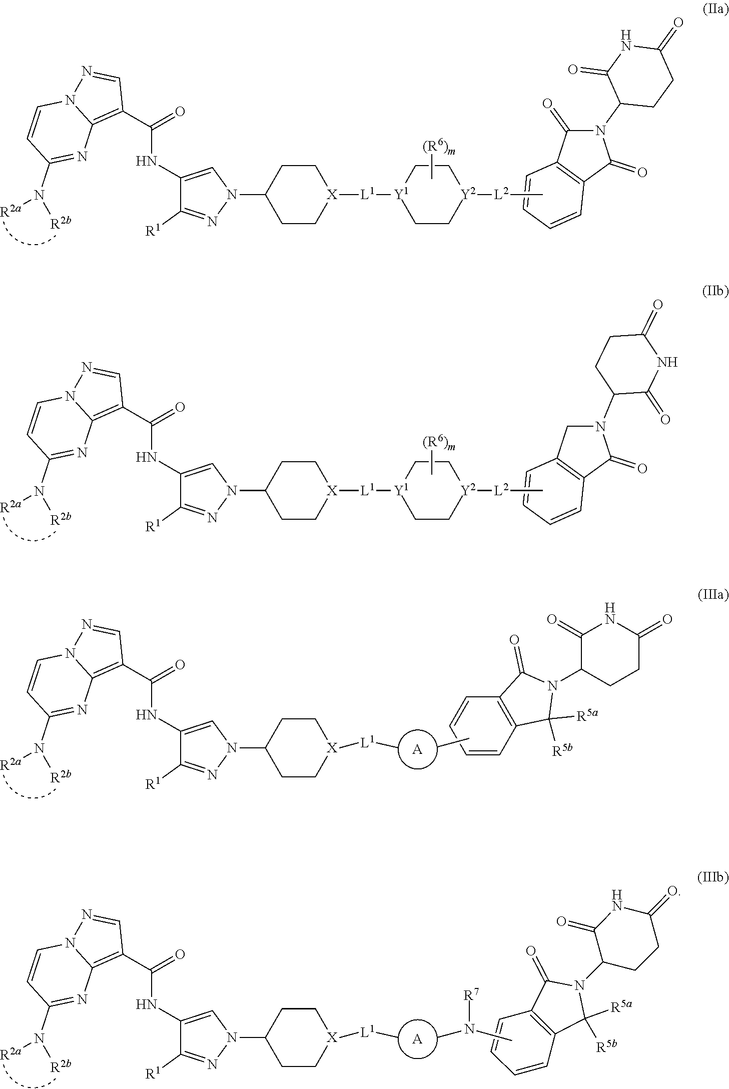

Embodiment 18 is a compound selected from the compounds of Table 1 or a pharmaceutically acceptable salt thereof.

Embodiment 19 is a pharmaceutical composition comprising the compound of any one of embodiments 1-18, or a pharmaceutically acceptable salt thereof, and a pharmaceutically acceptable excipient.

Embodiment 20 is a method of (i) modulating interleukin-1 (IL1) receptor-associated kinase 4 (IRAK4) comprising contacting IRAK4 with an effective amount of the compound of any one of embodiments 1-18, or a pharmaceutically acceptable salt thereof, or the pharmaceutical composition of embodiment 19; or (ii) treating an inflammatory or autoimmune disease in a subject in need thereof, comprising administering to the subject an effective amount of the compound of any one of embodiments 1-18, or a pharmaceutically acceptable salt thereof, or the pharmaceutical composition of embodiment 19, optionally wherein the inflammatory or autoimmune disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean ±20%, ±10%, ±5%, or ±1% of the indicated range, value, or structure, unless otherwise indicated.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, tert-butyl, -isopentyl, -neopentyl, tertpentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, CH₂C≡C(CH₃) and CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy; oxo (═O); amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₈ cycloalkyl), whereas in other embodiments the number of ring carbon atoms ranges from 3 to 5 (C₃-C₅ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1methylcyclopropyl, 2methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturated cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms (C₆-C₁₄aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “halogen” or “halo” is fluorine, chlorine, bromine or iodine.

“Haloalkyl” refers to an alkyl radical, as defined above, that is substituted by one or more halo radicals, as defined above, e.g., trifluoromethyl, difluoromethyl, trichloromethyl, 2,2,2trifluoroethyl, 1,2difluoroethyl, 3bromo2fluoropropyl, 1,2dibromoethyl, and the like. In some embodiments, the haloalkyl group has one to six carbon atoms and is substituted by one or more halo radicals (C₁-C₆ haloalkyl), or the haloalkyl group has one to three carbon atoms and is substituted by one or more halo radicals (C₁-C₃ haloalkyl). The halo radicals may be all the same or the halo radicals may be different. Unless specifically stated otherwise, a haloalkyl group is optionally substituted.

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1Hpyrrolo[2,3b]pyridyl), indazolyl, benzimidazolyl (e.g., 1Hbenzo[d]imidazolyl), imidazopyridyl (e.g., azabenzimidazolyl or 1Himidazo[4,5b]pyridyl), pyrazolopyridyl, triazolopyridyl, benzotriazolyl (e.g., 1Hbenzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4dihydroisoquinolin-1(2H)-onyl), tetrahydroquinolinyl, quinoxalinyl, and quinazolinyl groups. A heteroaryl group can be substituted or unsubstituted.

A “heterocyclyl” is a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4dioxaspiro[4.5]decanyl, homopiperazinyl, quinuclidyl, or tetrahydropyrimidin-2(1H)-one. Representative substituted heterocyclyl groups may be monosubstituted or substituted more than once, such as, but not limited to, pyridyl or morpholinyl groups, which are 2-, 3-, 4-, 5-, or 6substituted, or disubstituted with various substituents such as those listed below.

An “oxo” refers to the chemical group ═O.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Certain commonly used alternative chemical names may be used. For example, a divalent group such as a divalent “alkyl” group, a divalent “phenyl” group, a divalent “heteroaryl” group, a divalent “heterocyclyl” group etc., may also be referred to as an “alkylene” group, a “phenylene” group, a “heteroarylene” group, or a “heterocyclylene” group, respectively.

Embodiments of the disclosure are meant to encompass pharmaceutically acceptable salts, tautomers, isotopologues, and stereoisomers of the compounds provided herein, such as the compounds of Formula (I).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (Nmethyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and ptoluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science andPractice ofPharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGrawHill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuj a, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

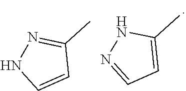

As readily understood by one skilled in the art, a wide variety of functional groups and other structures may exhibit tautomerism and all tautomers of compounds of Formula (I) are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (3H), iodine-125 (¹²⁵), sulfur35 (³⁵S), or carbon-14 (¹⁴C), or may be isotopically enriched, such as with deuterium (2H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a neurodegenerative disease, as described herein, or a symptom thereof.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is a neurodegenerative disease, as described herein, or symptoms thereof.

The term “effective amount” in connection with a compound disclosed herein means an amount capable of treating or preventing a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an S1P5 mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein is a compound of Formula (I):

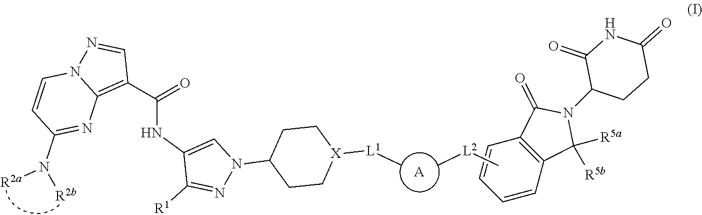

or a pharmaceutically acceptable salt thereof, wherein:
R¹ is C₁-C₆ haloalkyl, C₁-C₆ alkyl, or —CN;
R^{2a} is H or C₁-C₆ alkyl;
R^{2b} is C₅-C₆ cycloalkyl optionally substituted with 1-5 R³ groups;
or the dashed line between R^{2a} and R^{2b} represents a ring structure where R^{2a} and R^{2b} are taken together with the nitrogen atom to which they are attached to form a 6-membered heterocyclyl optionally containing one additional heteroatom selected from N and O, wherein the heterocyclyl is optionally substituted by 1-5 R³ groups;
each R³ is independently —NH₂, —OH, halo, C₁-C₆ alkyl, or C₁-C₆ haloalkyl;
X is CH or N;
L¹ is —C(O)N(H)—, —N(H)C(O)—, —C(O)—, —(C₁-C₆ alkylene)N(R⁴)—, or C₁-C₆ alkylene;
R⁴ is H or C₁-C₆ alkyl;
R^{5a} and R^{5b} are each H or are taken together to form an oxo group;
Ring A is

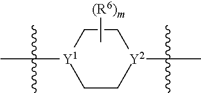

or an 8- to 10-membered spiro heterocyclylene containing 1-3 nitrogen atoms, wherein the heterocyclylene is substituted by m R⁶ groups;
Y¹ and Y² are independently CH or N;
each R⁶ is independently halo, C₁-C₆ alkyl, or C₁-C₆ haloalkyl;
m is 0-5;
L² is a bond or —N(R⁷)—; and
R⁷ is H or C₁-C₆ alkyl.

In some embodiments, R¹ is C₁-C₆ haloalkyl, C₁-C₆ alkyl, or —CN. In some embodiments, R¹ is C₁-C₃ haloalkyl, C₁-C₃ alkyl, or —CN. In some embodiments, R¹ is —CHF₂, —CF₃, —CH₃, or —CN.

In some embodiments, R¹ is C₁-C₆ haloalkyl. In some embodiments, R¹ is C₁-C₆ haloalkyl containing 1-13 halogen atoms. In some embodiments, R¹ is C₁-C₃ haloalkyl. In some embodiments, R¹ is C₁-C₃ haloalkyl containing 1-7 halogen atoms. In some embodiments, R¹ is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R¹ is —CF₃. In some embodiments, R¹ is —CHF₂.

In some embodiments, R¹ is C₁-C₆ alkyl. In some embodiments, R¹ is C₁-C₃ alkyl. In some embodiments, R¹ is methyl, ethyl, n-propyl, or isopropyl. In some embodiments, R¹ is methyl. In some embodiments, R¹ is ethyl. In some embodiments, R¹ is n-propyl. In some embodiments, R¹ is isopropyl.

In some embodiments, R¹ is —CN.

In some embodiments, R^{2a} is H or C₁-C₆ alkyl. In some embodiments, R^{2a} is H or C₁-C₃ alkyl. In some embodiments, R^{2a} is H or —CH₃.

In some embodiments, R^{2a} is H.

In some embodiments, R^{2a} is C₁-C₆ alkyl. In some embodiments, R^{2a} is C₁-C₃ alkyl. In some embodiments, R^{2a} is methyl, ethyl, n-propyl, or isopropyl. In some embodiments, R^{2a} is methyl. In some embodiments, R^{2a} is ethyl. In some embodiments, R^{2a} is n-propyl. In some embodiments, R^{2a} is isopropyl.

In some embodiments, R^{2b} is C₅-C₆ cycloalkyl optionally substituted with 1-5 R³ groups. In some embodiments, R^{2b} is cyclohexyl optionally substituted with 1-3 R³ groups. In some embodiments, R^{2b} is cyclohexyl optionally substituted with 1-2 R³ groups. In some embodiments, R^{2b} is cyclohexyl optionally substituted with 1 R³ group.

In some embodiments, R^{2b} is C₅-C₆ cycloalkyl optionally substituted with 1-5 R³ groups. In some embodiments, R^{2b} is cyclopentyl optionally substituted with 1-5 R³ groups. In some embodiments, R^{2b} is cyclohexyl optionally substituted with 1-5 R³ groups. In some embodiments, the cycloalkyl is optionally substituted with 1-5 R³ groups. In some embodiments, the cycloalkyl is optionally substituted with 1-3 R³ groups. In some embodiments, the cycloalkyl is optionally substituted with 1 or 2 R³ groups. In some embodiments, the cycloalkyl is optionally substituted with one R³ group. In some embodiments, the cycloalkyl is unsubstituted.

In some embodiments, R^{2a} and R^{2b} are taken together with the nitrogen atom to which they are attached to form a 6-membered heterocyclyl optionally containing one additional heteroatom selected from N and O and optionally substituted by 1-5 R³ groups. In some embodiments, R^{2a} and R^{2b} are taken together with the nitrogen atom to which they are attached to form a 6-membered heterocyclyl optionally containing one additional heteroatom selected from N and O and optionally substituted by 1-5 R³ groups. In some embodiments, R^{2a} and R^{2b}are taken together with the nitrogen atom to which they are attached to form a 6-membered heterocyclyl optionally containing one additional heteroatom selected from N and O and optionally substituted by 1-2 R³ groups.

In some embodiments, R^{2a} and R^{2b} are taken together with the nitrogen atom to which they are attached to form a 6-membered heterocyclyl containing one additional heteroatom selected from N and O and optionally substituted by 1-5 R³ groups. In some embodiments, R^{2a} and R^{2b} are taken together with the nitrogen atom to which they are attached to form a 6-membered heterocyclyl containing one additional nitrogen atom and optionally substituted by 1-5 R³ groups. In some embodiments, R^{2a} and R^{2b} are taken together with the nitrogen atom to which they are attached to form a 6-membered heterocyclyl containing one oxygen atom and optionally substituted by 1-5 R³ groups. In some embodiments, the heterocyclyl is substituted by 1-5 R³ groups. In some embodiments, the heterocyclyl is substituted by 1-3 R³ groups. In some embodiments, the heterocyclyl is substituted by 1 or 2 R³ groups. In some embodiments, the heterocyclyl is substituted by one R³ group. In some embodiments, the heterocyclyl is unsubstituted. In some embodiments, the heterocyclyl is piperidinyl, morpholinyl, or piperazinyl.

In some embodiments, each R³ is independently —NH₂, —OH, halo, C₁-C₆ alkyl, or C₁-C₆ haloalkyl. In some embodiments, each R³ is independently —NH₂, —OH, halo, C₁-C₃ alkyl, or C₁-C₃ haloalkyl. In some embodiments, each R³ is independently —NH₂, —OH, F, C₁, —CH₃, or —CF₃. In some embodiments, each R³ is independently —NH₂, —OH, F, or —CH₃.

In some embodiments, R³ is —NH₂.

In some embodiments, R³ is —OH.

In some embodiments, R³ is halo. In some embodiments, R³ is C₁, F, or Br. In some embodiments, R³ is C₁. In some embodiments, R³ is F. In some embodiments, R³ is Br.

In some embodiments, R³ is C₁-C₆ alkyl. In some embodiments, R³ is C₁-C₃ alkyl. In some embodiments, R³ is methyl, ethyl, n-propyl, or isopropyl. In some embodiments, R³ is methyl. In some embodiments, R³ is ethyl. In some embodiments, R³ is n-propyl. In some embodiments, R³ is isopropyl.

In some embodiments, R³ is C₁-C₆ haloalkyl. In some embodiments, R³ is C₁-C₆ haloalkyl containing 1-13 halogen atoms. In some embodiments, R³ is C₁-C₃ haloalkyl. In some embodiments, R³ is C₁-C₃ haloalkyl containing 1-7 halogen atoms. In some embodiments, R³ is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R³ is —CF₃. In some embodiments, R³ is —CHF₂.

In some embodiments,

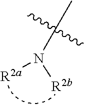

is:

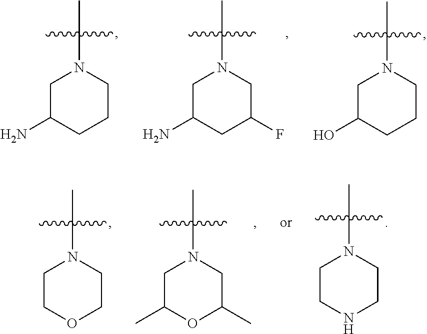

In some embodiments,

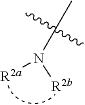

is

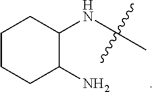 [SMILES: *NC1CCCCC1N]

In some embodiments, X is CH or N. In some embodiments, X is CH. In some embodiments, X is N.

In some embodiments, L¹ is —C(O)N(H)—, —N(H)C(O)—, —C(O)—, —(C₁-C₆ alkylene)N(R⁴)—, or C₁-C₆ alkylene. In some embodiments, L¹ is —C(O)N(H)—, —N(H)C(O)—, —C(O)—, —(C₁-C₃ alkylene)N(R⁴)—, or C₁-C₃ alkylene; wherein R⁴ is H or C₁-C₃ alkyl. In some embodiments, L¹ is —C(O)N(H)—, —N(H)C(O)—, —C(O)—, —CH₂N(CH₃)—, —CH₂—, or —CH₂CH₂—.

In some embodiments, L¹ is —C(O)N(H)—. In some embodiments, L¹ is —N(H)C(O)—. In some embodiments, L¹ is —C(O)—.

In some embodiments, L¹ is —(C₁-C₆ alkylene)N(R⁴)—. In some embodiments, L¹ is —(C₁-C₃ alkylene)N(R⁴)—. In some embodiments, L¹ is —CH₂N(R⁴)—, —CH₂CH₂N(R⁴)—, or —CH₂CH₂CH₂N(R⁴)—. In any of these variations, R⁴ is H or C₁-C₆ alkyl. In some embodiments, R⁴ is H or C₁-C₃ alkyl. In some embodiments, R⁴ is H. In some embodiments, R⁴ is C₁-C₆ alkyl. In some embodiments, R⁴ is C₁-C₃ alkyl. In some embodiments, R⁴ is methyl, ethyl, n-propyl, or isopropyl. In some embodiments, R⁴ is methyl. In some embodiments, R⁴ is ethyl. In some embodiments, R⁴ is n-propyl. In some embodiments, R⁴ is isopropyl. In some embodiments, L¹ is —CH₂N(H)—, —CH₂CH₂N(H)—, or —CH₂CH₂CH₂N(H)—. In some embodiments, L¹ is —CH₂N(CH₃)—, —CH₂CH₂N(CH₃)—, or —CH₂CH₂CH₂N(CH₃)—.

In some embodiments, L¹ is C₁-C₆ alkylene. In some embodiments, L¹ is C₁-C₃ alkylene. In some embodiments, L¹ is —CH₂—, —CH₂CH₂—, or —CH₂CH₂CH₂—.

In some embodiments, R^{5a} and R^{5b} are each H or are taken together to form an oxo group. In some embodiments, R^{5a} and R^{5b} are each H. In some embodiments, R^{5a} and R^{5b} are taken together to form an oxo group.

In some embodiments, Ring A is

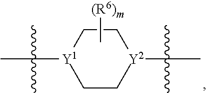

wherein Y¹ and Y² are independently CH or N, or an 8- to 10-membered spiro heterocyclylene containing 1-3 nitrogen atoms, wherein the heterocyclylene is substituted by m R⁶ groups.

In some embodiments, Ring A is

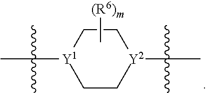

In some embodiments, Y¹ is N and Y² is CH. In some embodiments, Y¹ is CH and Y² is N. In some embodiments, Y¹ and Y² are each N. In some embodiments, Y¹ and Y² are each CH.

In some embodiments, Ring A is an 8- to 10-membered spiro heterocyclylene containing 1-3 nitrogen atoms and is substituted by m R⁶ groups. In some embodiments, Ring A is an 8-membered spiro heterocyclylene containing 1-3 nitrogen atoms and is substituted by m R⁶ groups. In some embodiments, Ring A is a 9-membered spiro heterocyclylene containing 1-3 nitrogen atoms and is substituted by m R⁶ groups. In some embodiments, Ring A is a 10-membered spiro heterocyclylene containing 1-3 nitrogen atoms and is substituted by m R⁶ groups. In some embodiments, the spiro heterocyclylene contains 1 nitrogen atom. In some embodiments, the spiro heterocyclylene contains 2 nitrogen atoms. In some embodiments, the spiro heterocyclylene contains 3 nitrogen atoms. In some embodiments, Ring A is a 10-membered spiro heterocyclylene containing 2 nitrogen atoms and is substituted by m R⁶ groups.

In some embodiments, each R⁶ is independently halo, C₁-C₆ alkyl, or C₁-C₆ haloalkyl. In some embodiments, each R⁶ is independently halo, C₁-C₃ alkyl, or C₁-C₃ haloalkyl. In some embodiments, each R⁶ is independently C₁, —CH₃, or —CF₃.

In some embodiments, R⁶ is halo. In some embodiments, R⁶ is C₁, F, or Br. In some embodiments, R⁶ is C₁. In some embodiments, R⁶ is F. In some embodiments, R⁶ is Br.

In some embodiments, R⁶ is C₁-C₆ alkyl. In some embodiments, R⁶ is C₁-C₃ alkyl. In some embodiments, R⁶ is methyl, ethyl, n-propyl, or isopropyl. In some embodiments, R⁶ is methyl. In some embodiments, R⁶ is ethyl. In some embodiments, R⁶ is n-propyl. In some embodiments, R⁶ is isopropyl.

In some embodiments, R⁶ is C₁-C₆ haloalkyl. In some embodiments, R⁶ is C₁-C₆ haloalkyl containing 1-13 halogen atoms. In some embodiments, R⁶ is C₁-C₃ haloalkyl. In some embodiments, R⁶ is C₁-C₃ haloalkyl containing 1-7 halogen atoms. In some embodiments, R⁶ is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R⁶ is —CF₃. In some embodiments, R⁶ is —CHF₂.

In some embodiments, m is 0-5. In some embodiments, m is 0. In some embodiments, m is 1 or 2. In some embodiments, m is 1. In some embodiments, m is 2. In some embodiments, m is 3. In some embodiments, m is 4. In some embodiments, m is 5.

In some embodiments, Ring A is:

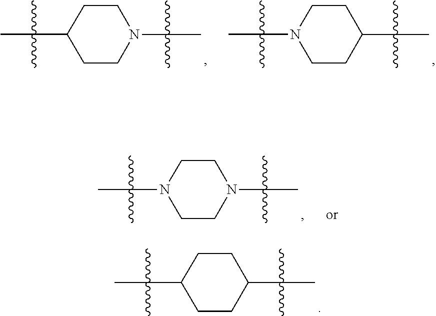

In some embodiments, Ring A is

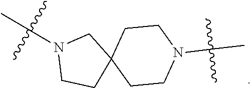 [SMILES: *N1CCC2(CC1)CCN(*)C2]

In some embodiments, L² is a bond or —N(R⁷)—. In some embodiments, L² is a bond. In some embodiments, L² is —N(R⁷)—, wherein R⁷ is H or C₁-C₃ alkyl. In some embodiments, L² is —N(R⁷)—, wherein R⁷ is H or —CH₃.

In some embodiments, L² is a bond.

In some embodiments, L² is —N(R⁷)—. In some embodiments, R⁷ is H or C₁-C₆ alkyl. In some embodiments, R⁷ is H. In some embodiments, R⁷ is C₁-C₆ alkyl. In some embodiments, R⁷ is C₁-C₃ alkyl. In some embodiments, R⁷ is methyl, ethyl, n-propyl, or isopropyl. In some embodiments, R⁷ is methyl. In some embodiments, R⁷ is ethyl. In some embodiments, R⁷ is n-propyl. In some embodiments, R⁷ is isopropyl. In some embodiments, L² is —N(H)—. In some embodiments, L² is —N(CH₃)—. In some embodiments, L² is —N(CH₂CH₃)—.

In some embodiments,

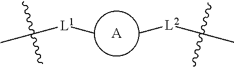

is:

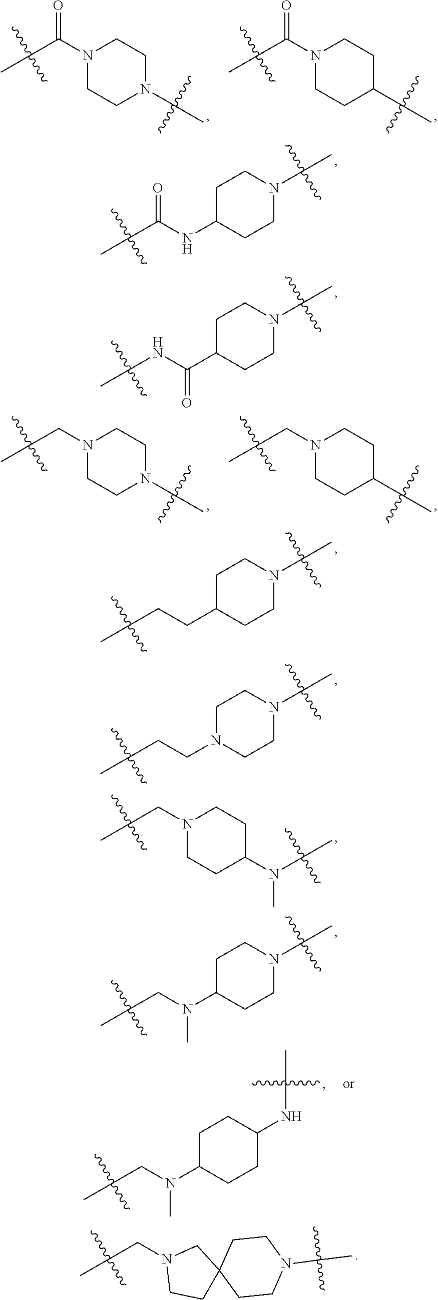

In some embodiments, the compound of Formula (I) is a compound of Formula (IIA) or (IIB):

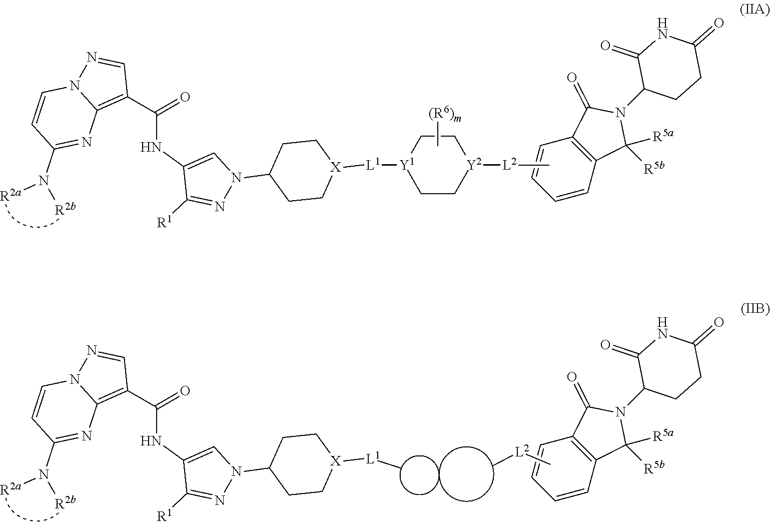

wherein

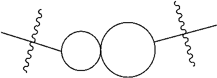

is 8- to 10-membered spiro heterocyclylene containing 1-3 nitrogen atoms, wherein the heterocyclylene is substituted by m R⁶groups; wherein R¹, R^{2a}, R^{2b}, R^{5a}, R^{5b}, R⁶, m, X, L¹, L², Y¹, and Y² are as described for Formula (I).

In some embodiments, the compound of Formula (I) is a compound of Formula (IIa) or (IIb):

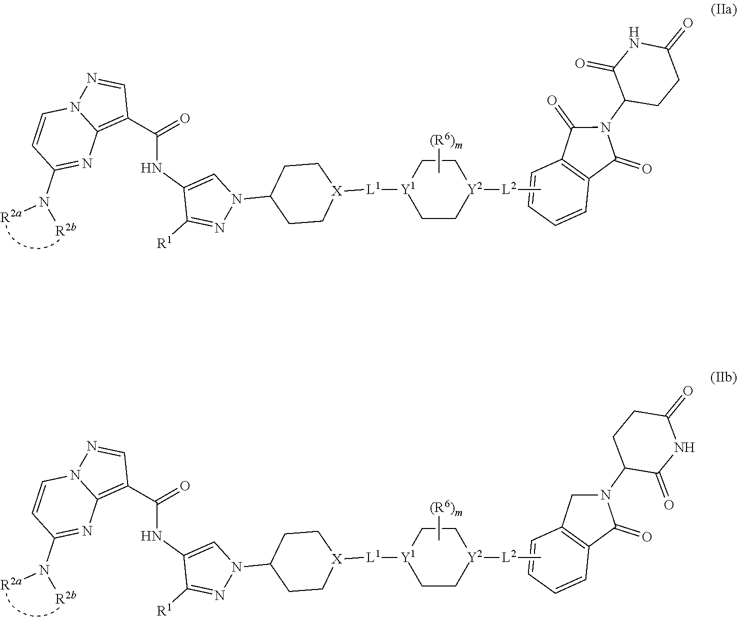

wherein R¹, R^{2a}, R^{2b}, R⁶, m, X, L¹, L², Y¹, and Y² are as described for Formula (I).

In some embodiments, the compound of Formula (I) is a compound of Formula (IIc) or (IId):

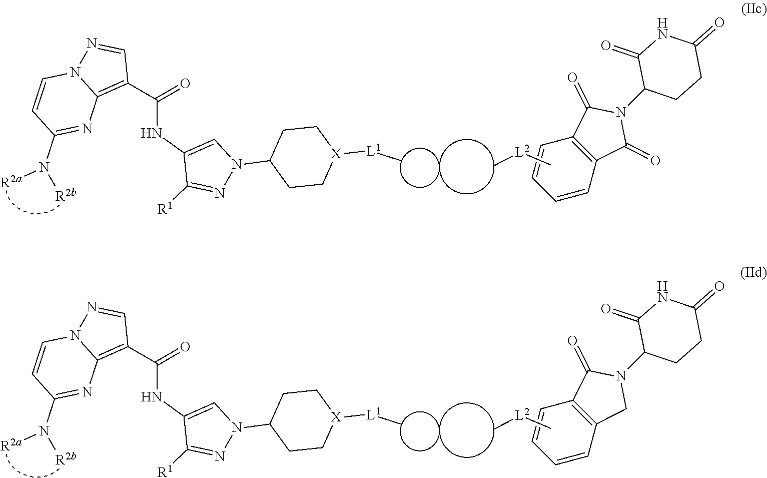

wherein

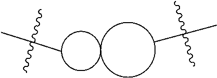

is 8- to 10-membered spiro heterocyclylene containing 1-3 nitrogen atoms, wherein the heterocyclylene is substituted by m R⁶ groups; and wherein R¹, R^{2a}, R^{2b}, X, L¹, and L² are as described for Formula (I).

In some embodiments, the compound of Formula (I) is a compound of Formula (IIIa) or (IIIb):

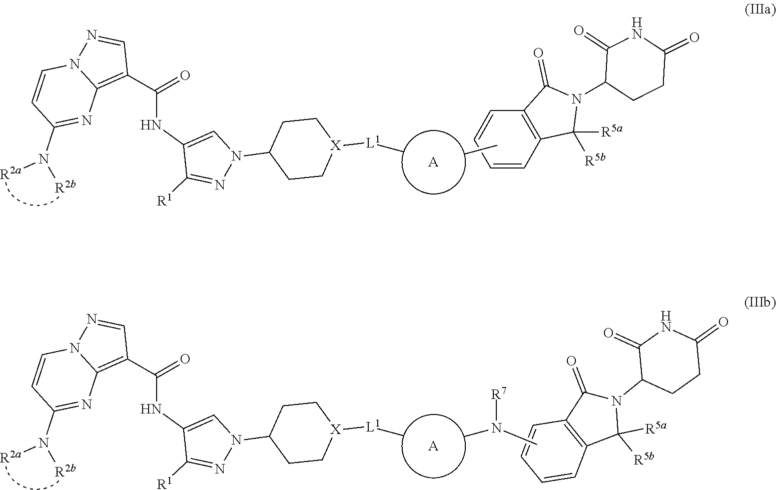

wherein R¹, R^{2a}, R^{2b}, R^{5a}, R^{5b}, R⁷, X, L¹, and Ring A are as described for Formula (I).

In some embodiments, the compound of Formula (I) is a compound of Formula (IVa), (IVb), (IVc), (IVd), or (IVe):

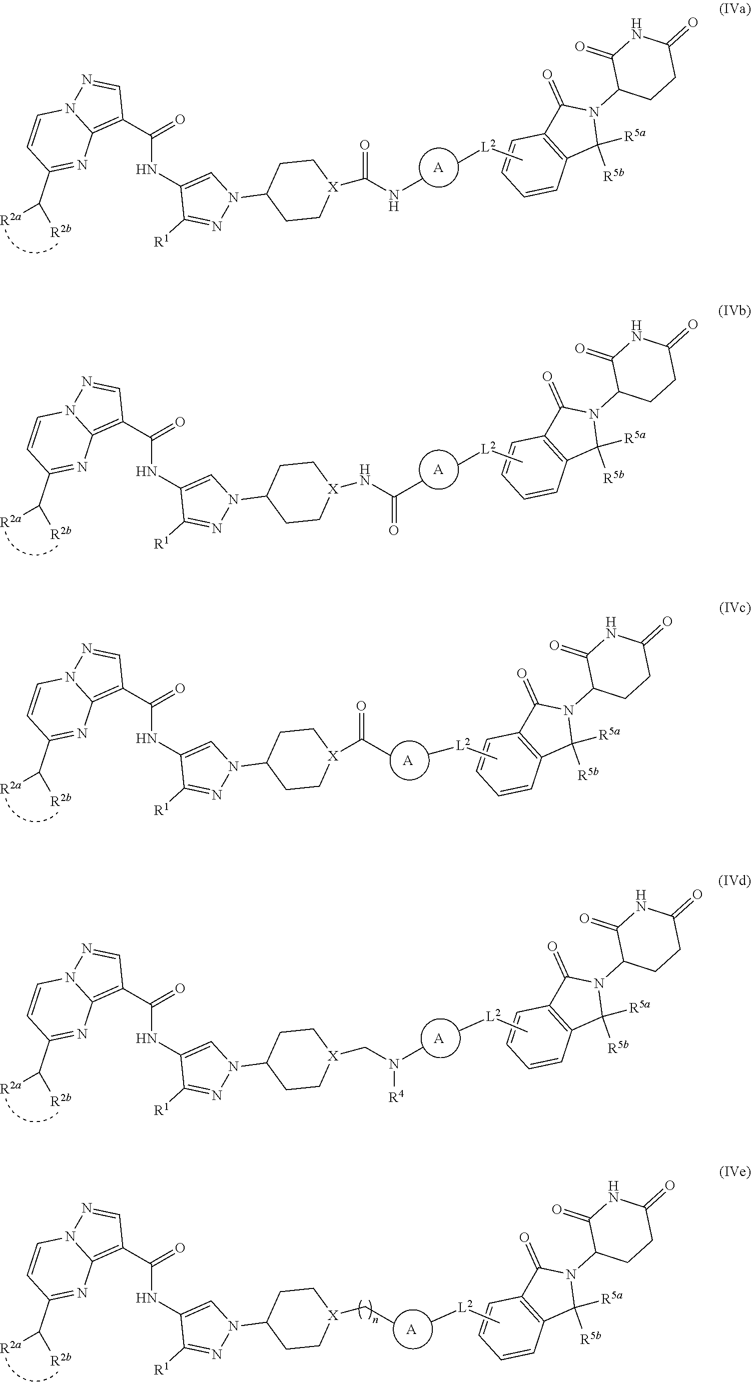

wherein n is an integer 1-6; and wherein R¹, R^{2a}, R^{2b}, R⁴, R^{5a}, R^{5b}, X, Ring A, and L² are as described for Formula (I).

In some embodiments, the compound of Formula (I) is a compound of Formula (Va) or (Vb):

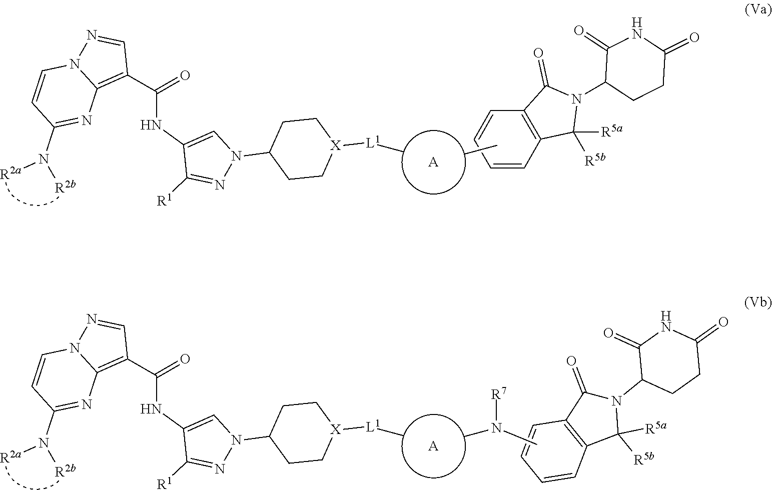

wherein R1, R^{2a}, R^{2b}, R^{5a}, R^{5b}, R⁷, X, L¹, and Ring A are as described for Formula (I).

In some embodiments, the compound of Formula (I) is a compound of Formula (VIa) or (VIb):

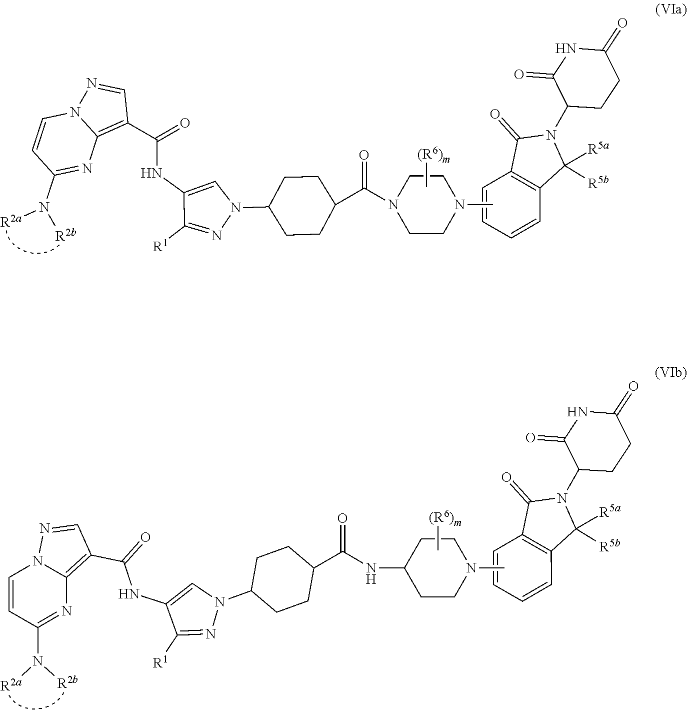

wherein R1, R^{2a}, R^{2b}, R^{5a}, R^{5b}, R⁶, m, are as described for Formula (I).

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to Ring A of Formula (I) may be combined with every description, variation, embodiment, or aspect of R1, R^{2a}, R^{2b}, R³, R⁴, R^{5a}, R^{5b}, R⁶, R⁷, X, L¹, L², Y¹, Y², and m the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (IIA), (IIB), (Ila), (IIb), (IIc), (IId), (IIIa), (IIIb), (IVa), (IVb), (IVc), (IVd), (IVe), (Va), (Vb), (VIa), and (VIb) are equally described, the same as if each and every description, variation, embodiment, or aspect were separately and individually listed for all formulae.

In some embodiments, provided is a compound selected from the compounds in Table 1 or a pharmaceutically acceptable salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Compound | |
|---|---|
| No. | Structure |
| P-1 | 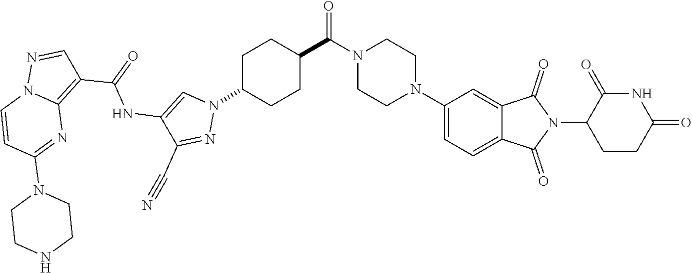 |
| P-2 | 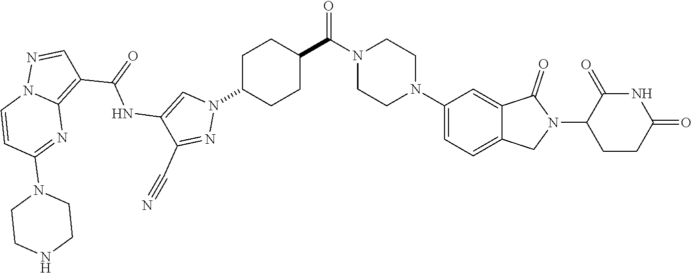 |
| P-3 | 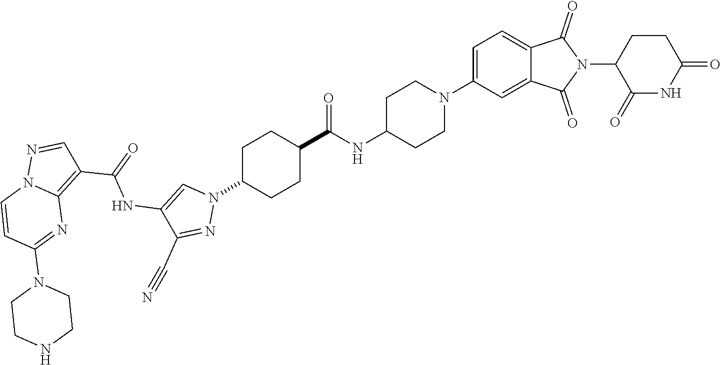 |
| P-4 | 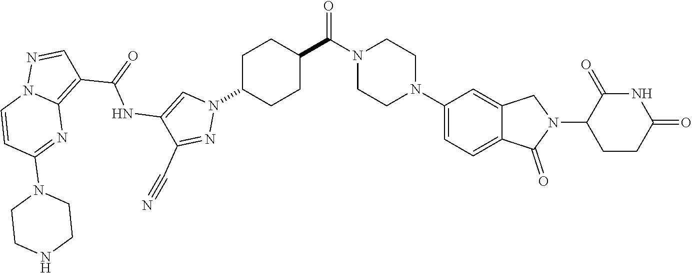 |
| P-5 | 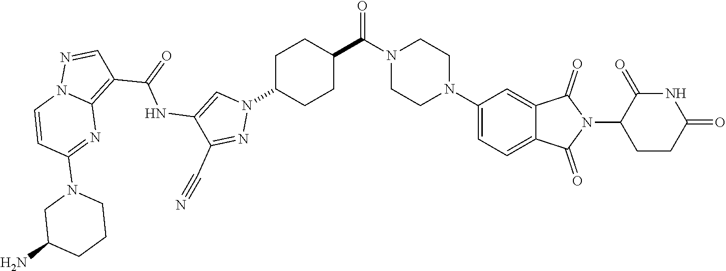 |
| P-6 | 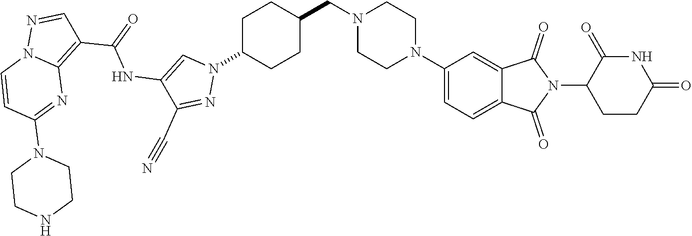 |
| P-7 | 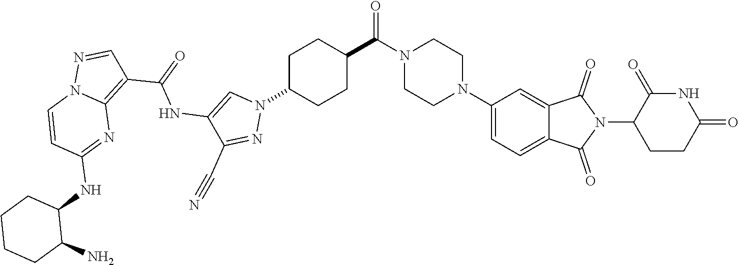 |
| P-8 | 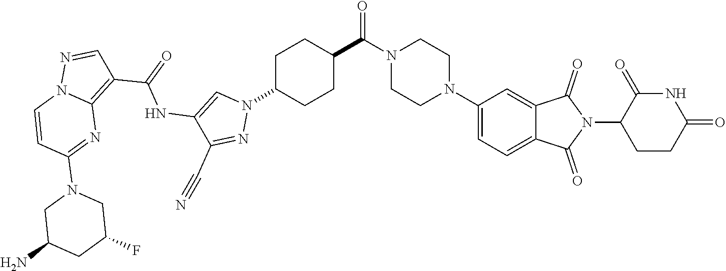 |
| P-9 | 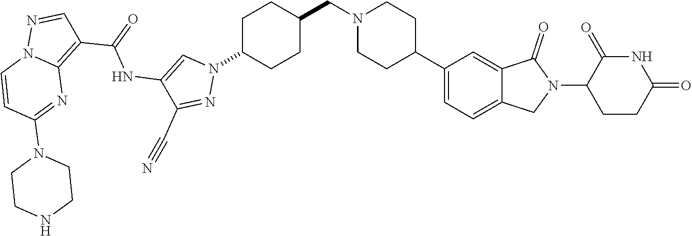 |
| P-10 | 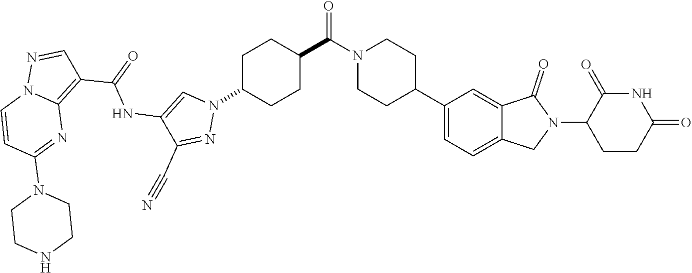 |
| P-11 | 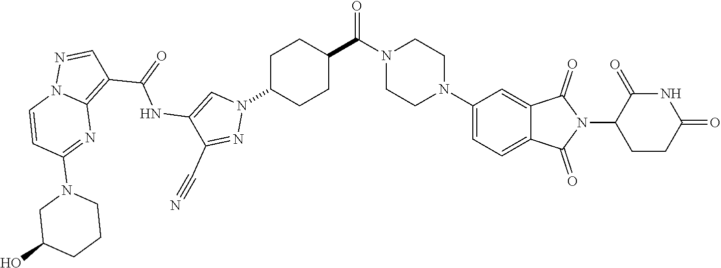 |
| P-12 | 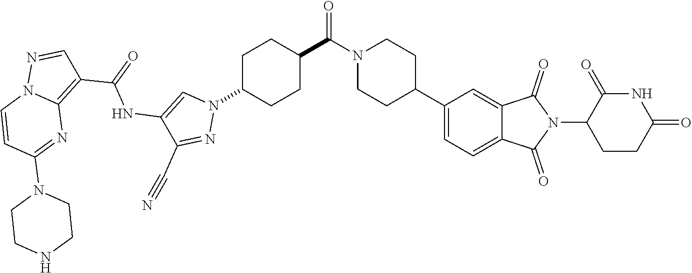 |
| P-13 | 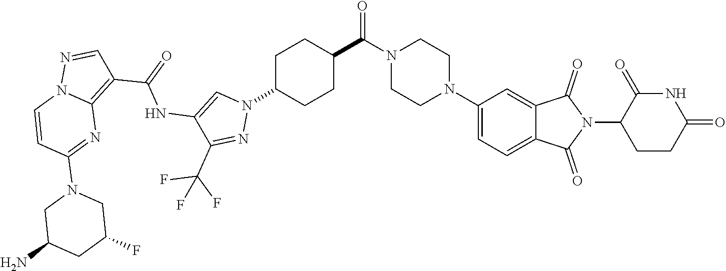 |
| P-14 | 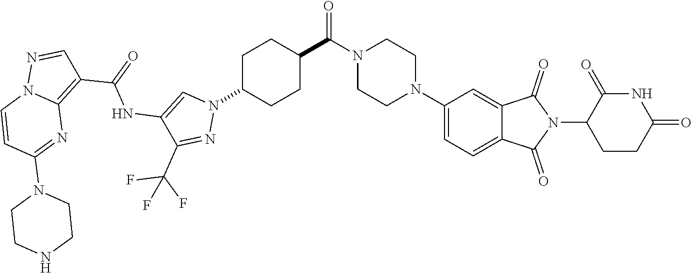 |
| P-15 | 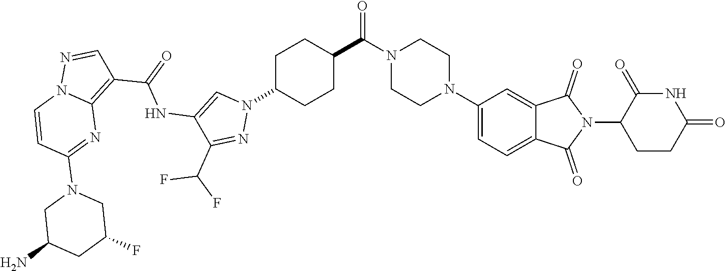 |
| P-16 | 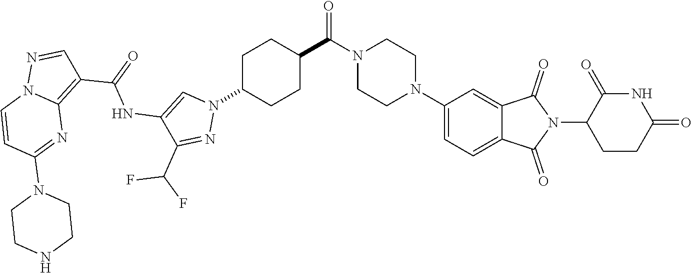 |
| P-17 | 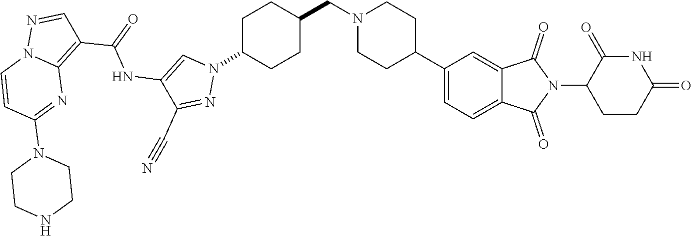 |
| P-18 | 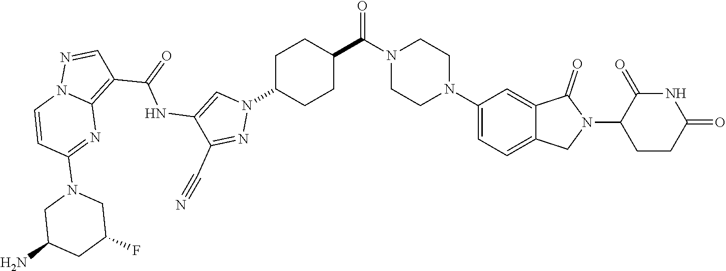 |
| P-19 | 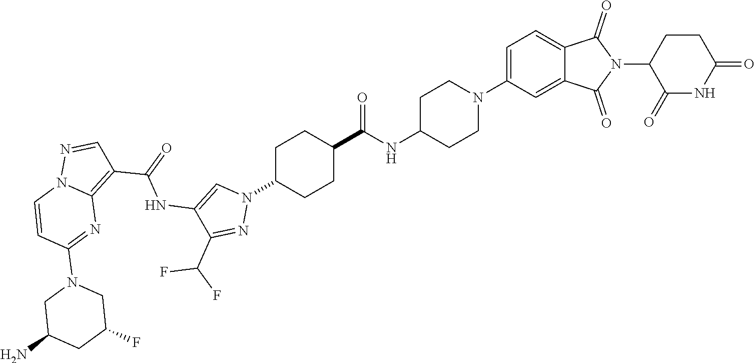 [SMILES: N[C@@H]1C[C@@H](F)CN(c2ccn3ncc(C(=O)Nc4cn([C@H]5CC[C@H](C(=O)NC6CCN(c7ccc8c(c7)C(=O)N(C7CCC(=O)NC7=O)C8=O)CC6)CC5)nc4C(F)F)c3n2)C1] |
| P-20 | 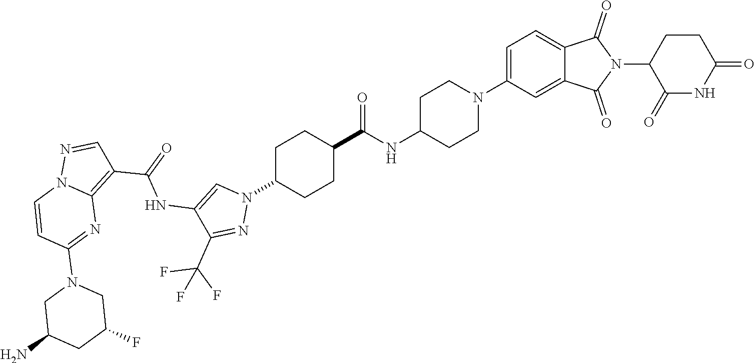 [SMILES: N[C@@H]1C[C@@H](F)CN(c2ccn3ncc(C(=O)Nc4cn([C@H]5CC[C@H](C(=O)NC6CCN(c7ccc8c(c7)C(=O)N(C7CCC(=O)NC7=O)C8=O)CC6)CC5)nc4C(F)(F)F)c3n2)C1] |
| P-21 | 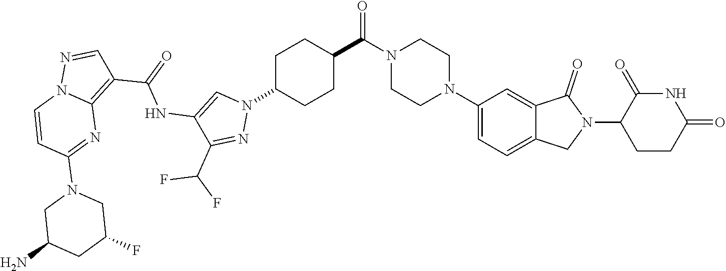 |
| P-22 | 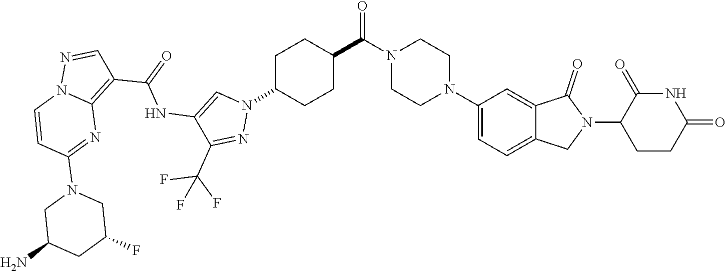 |
| P-23 | 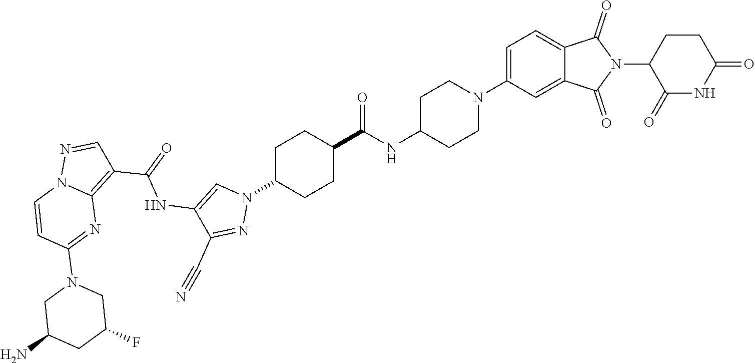 [SMILES: N#Cc1nn([C@H]2CC[C@H](C(=O)NC3CCN(c4ccc5c(c4)C(=O)N(C4CCC(=O)NC4=O)C5=O)CC3)CC2)cc1NC(=O)c1cnn2ccc(N3C[C@H](N)C[C@@H](F)C3)nc12] |
| P-24 | 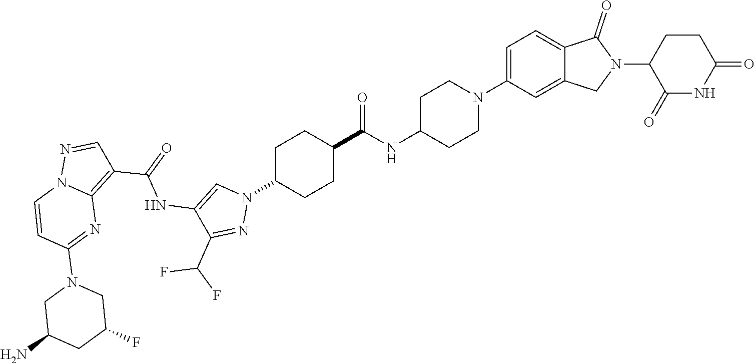 [SMILES: N[C@@H]1C[C@@H](F)CN(c2ccn3ncc(C(=O)Nc4cn([C@H]5CC[C@H](C(=O)NC6CCN(c7ccc8c(c7)CN(C7CCC(=O)NC7=O)C8=O)CC6)CC5)nc4C(F)F)c3n2)C1] |
| P-25 | 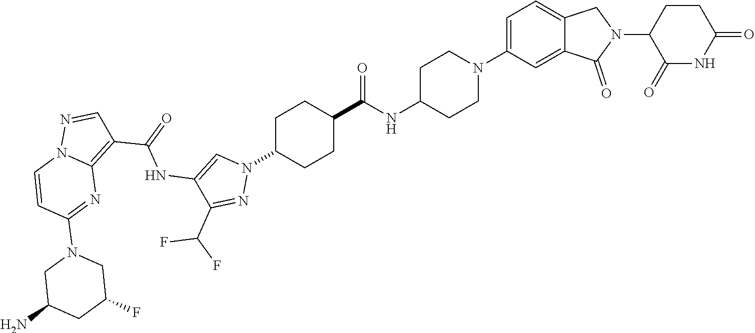 [SMILES: N[C@@H]1C[C@@H](F)CN(c2ccn3ncc(C(=O)Nc4cn([C@H]5CC[C@H](C(=O)NC6CCN(c7ccc8c(c7)C(=O)N(C7CCC(=O)NC7=O)C8)CC6)CC5)nc4C(F)F)c3n2)C1] |
| P-26 | 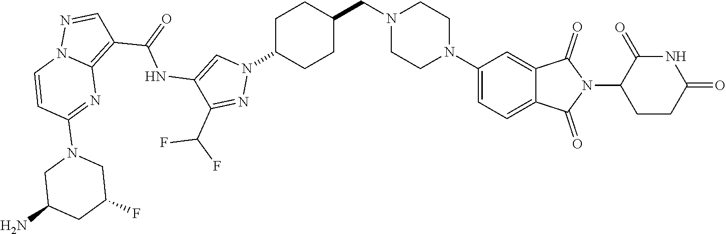 |
| P-27 | 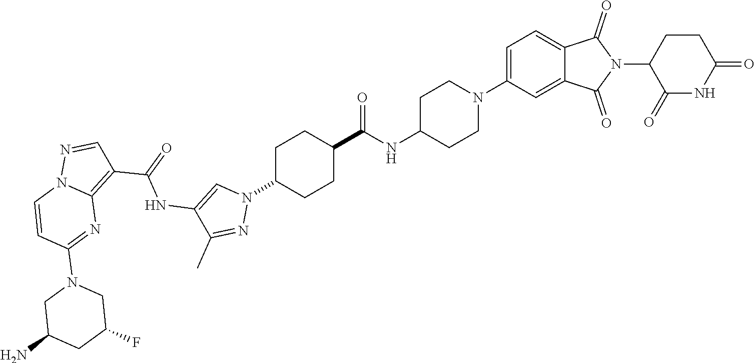 |
| P-28 | 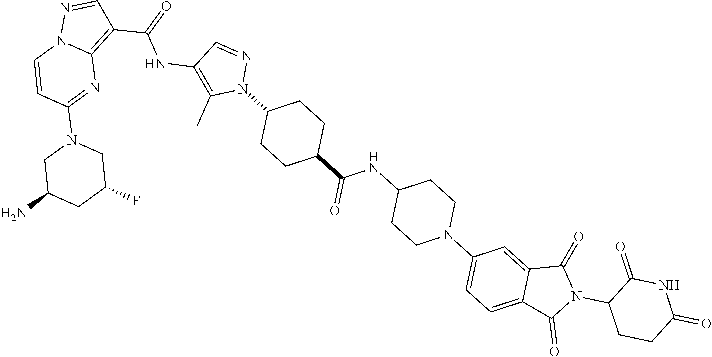 |
| P-29 | 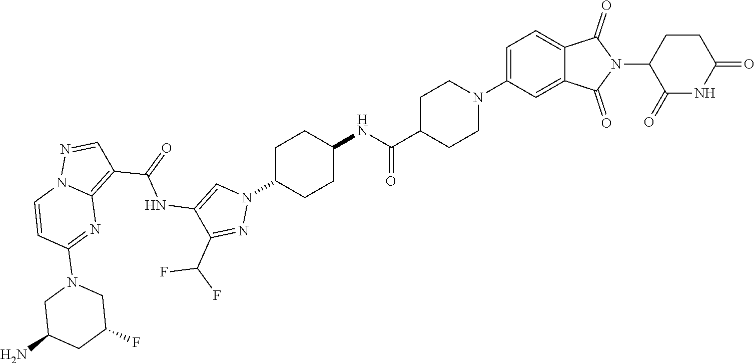 [SMILES: N[C@@H]1C[C@@H](F)CN(c2ccn3ncc(C(=O)Nc4cn([C@H]5CC[C@H](NC(=O)C6CCN(c7ccc8c(c7)C(=O)N(C7CCC(=O)NC7=O)C8=O)CC6)CC5)nc4C(F)F)c3n2)C1] |
| P-30 | 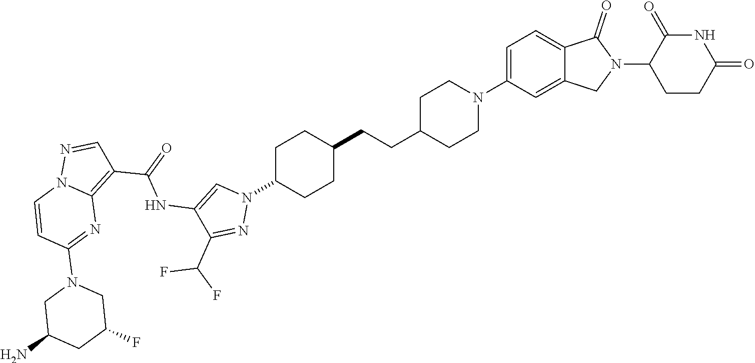 [SMILES: N[C@@H]1C[C@@H](F)CN(c2ccn3ncc(C(=O)Nc4cn([C@H]5CC[C@H](CCC6CCN(c7ccc8c(c7)CN(C7CCC(=O)NC7=O)C8=O)CC6)CC5)nc4C(F)F)c3n2)C1] |
| P-31 | 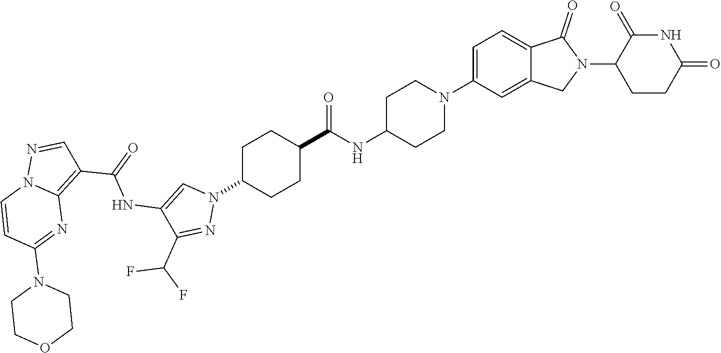 [SMILES: O=C1CCC(N2Cc3cc(N4CCC(NC(=O)[C@H]5CC[C@H](n6cc(NC(=O)c7cnn8ccc(N9CCOCC9)nc78)c(C(F)F)n6)CC5)CC4)ccc3C2=O)C(=O)N1] |
| P-32 | 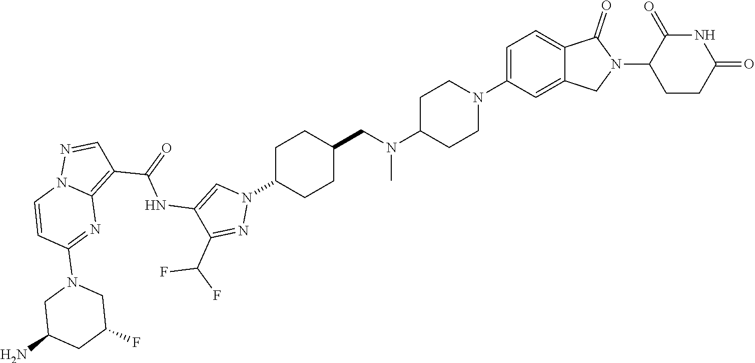 [SMILES: CN(C[C@H]1CC[C@H](n2cc(NC(=O)c3cnn4ccc(N5C[C@H](N)C[C@@H](F)C5)nc34)c(C(F)F)n2)CC1)C1CCN(c2ccc3c(c2)CN(C2CCC(=O)NC2=O)C3=O)CC1] |
| P-33 | 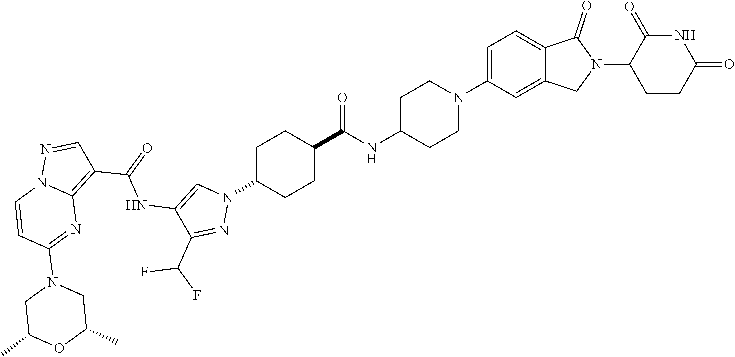 [SMILES: C[C@@H]1CN(c2ccn3ncc(C(=O)Nc4cn([C@H]5CC[C@H](C(=O)NC6CCN(c7ccc8c(c7)CN(C7CCC(=O)NC7=O)C8=O)CC6)CC5)nc4C(F)F)c3n2)C[C@H](C)O1] |
| P-34 | 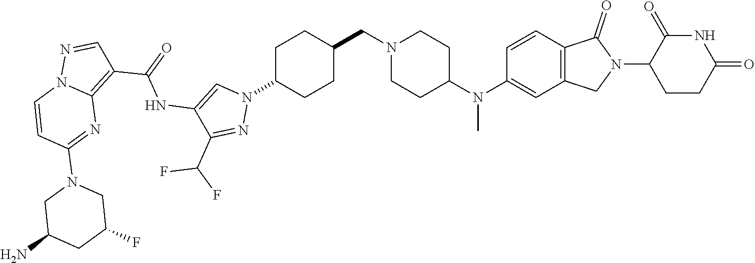 |
| P-35 | 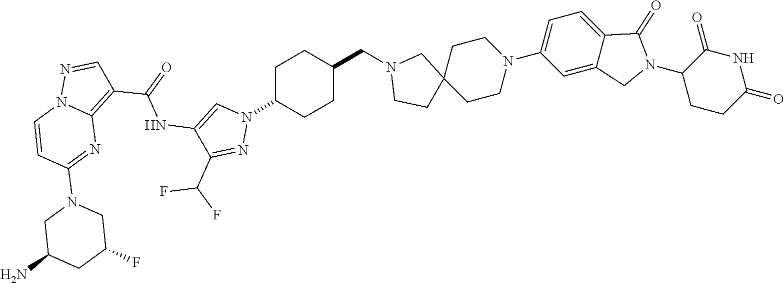 |
| P-36 | 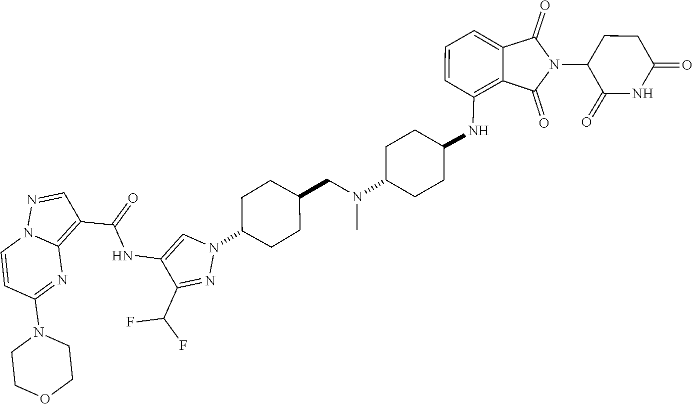 |
| P-37 | 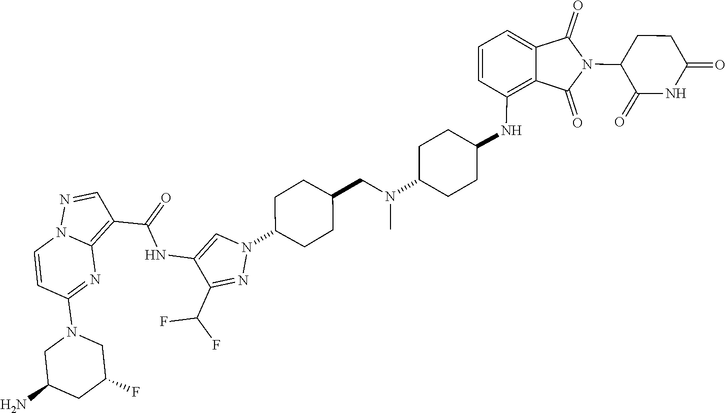 |
| P-38 | 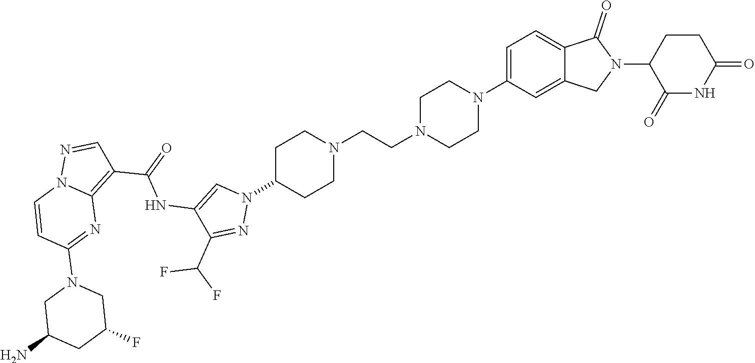 |

or a pharmaceutically acceptable salt thereof.

It is understood that in the present description, combinations of substituents and/or variables of the depicted formulae are permissible only if such contributions result in stable compounds.

Furthermore, all compounds of Formula (I) that exist in free base or acid form can be converted to their pharmaceutically acceptable salts by treatment with the appropriate inorganic or organic base or acid by methods known to one skilled in the art. Salts of the compounds of Formula (I) can be converted to their free base or acid form by standard techniques.

### Methods of Synthesis

The compounds described herein can be made using conventional organic syntheses and commercially available starting materials, or the methods provided herein. By way of example and not limitation, compounds of Formula (I) can be prepared as outlined in Schemes 1 and 2, as well as in the examples set forth herein. It should be noted that one skilled in the art would know how to modify the procedures set forth in the illustrative schemes and examples to arrive at the desired products.

Compounds of Formula A can be prepared as outlined in Scheme 1. Coupling of intermediate a-1 with intermediate a-2 under basic conditions, such as in the presence of DIPEA, forms intermediate a-3, which is then deprotected under basic conditions to form intermediate a-4. Subsequent coupling of intermediate a-4 with intermediate a-5 using TCFH/NMI affords intermediate a-6, which is then deprotected under basic conditions to yield intermediate a-7, followed by coupling with intermediate a-8 (for example, using HATU) affords compounds of Formula A

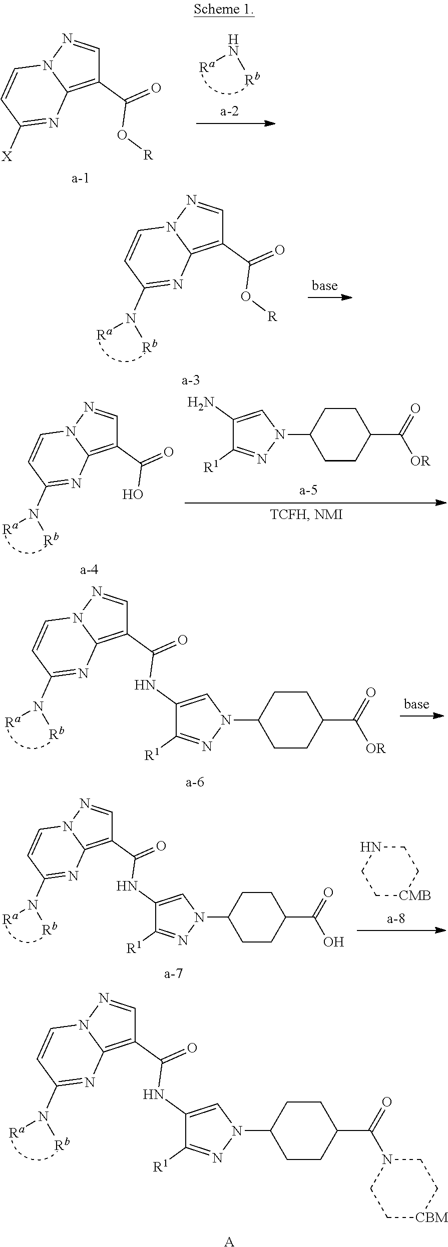

wherein X is halo such as chloro; each R is independently alkyl such as methyl or ethyl; R¹, R^{a}, and R^{b} are as described for Formula (I); and

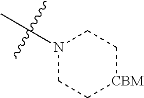

represents the portion of Formula (I) corresponding to

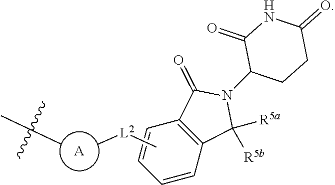

Compounds of Formula B can be prepared as outlined in Scheme 2. Intermediate a-5 is reduced (for example, using NaBH₄) to intermediate b-1, which is then coupled with intermediate a-4 using TCFH/NMI to yield intermediate b-2. Subsequent oxidation of b-2 followed by reductive amination with a-8 (for example, using IBX and NaBH(OAc)₃) affords compounds of Formula B.

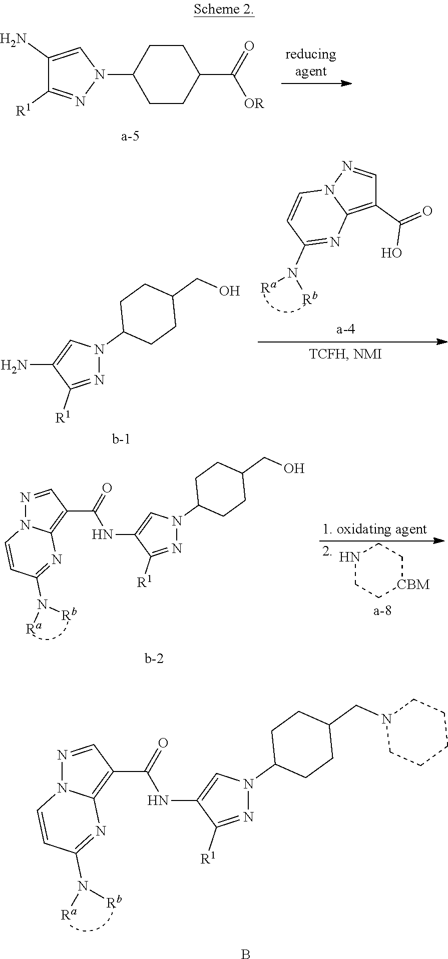

wherein R is alkyl such as methyl or ethyl; R¹, R^{a}, and R^{b} are as described for Formula (I); and

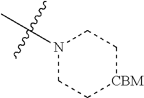

represents the portion of Formula (I) corresponding to

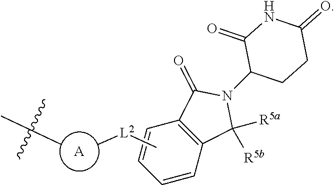

### Methods of Use

Embodiments of the present disclosure provide a method for modulating IRAK4 in a subject in need thereof, the method comprising administering to the subject an effective amount of a compound of Formula (I). Modulation (e.g., inhibition or activation) of IRAK4 can be assessed and demonstrated by a wide variety of ways known in the art. Kits and commercially available assays can be utilized for determining whether and to what degree IRAK4 has been modulated (e.g., inhibited or activated).

In one aspect, provided herein is a method of modulating IRAK4 comprising contacting IRAK4 with an effective amount of a compound of Formula (I) or any embodiment or variation thereof. In some embodiments, the compound of Formula (I) inhibits IRAK4. In other embodiments, the compound of Formula (I) activates IRAK4. In some embodiments, the compound of Formula (I) is an agonist of IRAK4. In some embodiments, the compound of Formula (I) is an antagonist of IRAK4.

In some embodiments, provided herein is a method for targeting IRAK4 for degradation comprising contacting IRAK4 with an effective amount of a compound of Formula (I) or any embodiment or variation thereof.

In some embodiments, a compound of Formula (I) modulates the activity of IRAK4 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) modulates the activity of IRAK4 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

Also provided in certain embodiments of the present disclosure is a method for degrading IRAK4 in a subject in need thereof, the method comprising administering to the subject an effective amount of a compound of Formula (I). Degradation of RAK4 can be assessed and demonstrated by a wide variety of ways known in the art. Kits and commercially available assays, including cell-based assays, can be utilized for determining whether and to what degree IRAK4 has been degraded.

In one aspect, provided herein is a method of degrading IRAK4 comprising contacting IRAK4 with an effective amount of a compound of Formula (I) or any embodiment or variation thereof. In some embodiments, the compound of Formula (I) partially degrades IRAK3. In some embodiments, the compound of Formula (I) fully degrades IRAK4.

In some embodiments, a compound of Formula (I) degrades IRAK4 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) degrades IRAK4 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In another aspect, provided herein is a method for treating an inflammatory or autoimmune disease in a subject in need thereof, comprising administering to the subject an effective amount of a compound of Formula (I). In some embodiments, provided herein is a method for treating an inflammatory disease in a subject in need thereof, comprising administering to the subject an effective amount of a compound of Formula (I). In some embodiments, provided herein is a method for treating an autoimmune disease in a subject in need thereof, comprising administering to the subject an effective amount of a compound of Formula (I). In some embodiments, provided herein is a method for preventing an inflammatory or autoimmune disease in a subject in need thereof, comprising administering to the subject an effective amount of a compound of Formula (I). In some embodiments, provided herein is a method for preventing an inflammatory disease in a subject in need thereof, comprising administering to the subject an effective amount of a compound of Formula (I). In some embodiments, provided herein is a method for preventing an autoimmune disease in a subject in need thereof, comprising administering to the subject an effective amount of a compound of Formula (I). Non-limiting examples of an inflammatory or autoimmune disease include atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

In some embodiments, administering a compound of Formula (I) to a subject that is predisposed to an inflammatory or autoimmune disease prevents the subject from developing any symptoms of the inflammatory or autoimmune disease. In some embodiments, administering a compound of Formula (I) to a subject that does not yet display symptoms of an inflammatory or autoimmune disease prevents the subject from developing any symptoms of the inflammatory or autoimmune disease. In some embodiments, administering a compound of Formula (I) to a subject in need thereof diminishes the extent of the inflammatory or autoimmune disease in the subject. In some embodiments, administering a compound of Formula (I) to a subject in need thereof stabilizes the inflammatory or autoimmune disease (prevents or delays the worsening of the inflammatory or autoimmune disease). In some embodiments, administering a compound of Formula (I) to a subject in need thereof delays the occurrence or recurrence of the inflammatory or autoimmune disease. In some embodiments, administering a compound of Formula (I) to a subject in need thereof slows the progression of the inflammatory or autoimmune disease. In some embodiments, administering a compound of Formula (I) to a subject in need thereof provides a partial remission of the inflammatory or autoimmune disease. In some embodiments, administering a compound of Formula (I) to a subject in need thereof provides a total remission of the inflammatory or autoimmune disease. In some embodiments, administering a compound of Formula (I) to a subject in need thereof decreases the dose of one or more other medications required to treat the inflammatory or autoimmune disease. In some embodiments, administering a compound of Formula (I) to a subject in need thereof enhances the effect of another medication used to treat the inflammatory or autoimmune disease. In some embodiments, administering a compound of Formula (I) to a subject in need thereof delays the progression of the inflammatory or autoimmune disease. In some embodiments, administering a compound of Formula (I) to a subject in need thereof increases the quality of life of the subject having an inflammatory or autoimmune disease. In some embodiments, administering a compound of Formula (I) to a subject in need thereof prolongs survival of a subject having an inflammatory or autoimmune disease.

In one aspect, provided herein is method of preventing a subject that is predisposed to an inflammatory or autoimmune disease from developing any symptoms of the inflammatory or autoimmune disease, the method comprising administering a compound of Formula (I) to the subject. In some embodiments, provided herein is a method of preventing a subject that does not yet display symptoms of an inflammatory or autoimmune disease from developing any symptoms of the inflammatory or autoimmune disease, the method comprising administering a compound of Formula (I) to the subject.

In some aspects, provided herein is a method of diminishing the extent of an inflammatory or autoimmune disease in a subject, the method comprising administering a compound of Formula (I) to the subject. In some embodiments, provided herein is a method of stabilizing an inflammatory or autoimmune disease in a subject, the method comprising administering a compound of Formula (I) to the subject. In some embodiments, the method prevents the worsening of the inflammatory or autoimmune disease. In some embodiments, the method delays the worsening of the inflammatory or autoimmune disease.

In another aspect, provided herein is a method of delaying the occurrence or recurrence of an inflammatory or autoimmune disease in a subject, the method comprising administering a compound of Formula (I) to the subject.

In some embodiments, provided herein is a method of slowing the progression of an inflammatory or autoimmune disease in a subject, the method comprising administering a compound of Formula (I) to the subject. In some embodiments, the method provides a partial remission of the inflammatory or autoimmune disease. In some embodiments, the method provides a total remission of the inflammatory or autoimmune disease.

In further aspects, provided herein is a method of decreasing the dose of one or more other medications required to treat an inflammatory or autoimmune disease in a subject, the method comprising administering a compound of Formula (I) to the subject. In some embodiments, provided herein is a method of enhancing the effect of another medication used to treat an inflammatory or autoimmune disease in a subject, the method comprising administering a compound of Formula (I) to the subject.

Also provided here is a method of delaying the progression of an inflammatory or autoimmune disease in a subject, the method comprising administering a compound of Formula (I) to the subject. In some embodiments, the method increases the quality of life of the subject having an inflammatory or autoimmune disease. In some embodiments, the method prolongs survival of the subject having an inflammatory or autoimmune disease.

In another aspect, provided herein is a method for treating inflammatory or autoimmune symptoms caused by a disease in a subject in need thereof, comprising administering to the subject an effective amount of a compound of Formula (I). In some embodiments, provided herein is a method for preventing inflammatory or autoimmune symptoms caused by a disease in a subject in need thereof, comprising administering to the subject an effective amount of a compound of Formula (I). In some embodiments, administering a compound of Formula (I) to a subject that is predisposed to a disease which causes inflammatory or autoimmune symptoms prevents the subject from developing any inflammatory or autoimmune symptoms. In some embodiments, administering a compound of Formula (I) to a subject that does not yet display inflammatory or autoimmune symptoms of a disease which causes inflammatory or autoimmune symptoms prevents the subject from developing any inflammatory or autoimmune symptoms. In some embodiments, administering a compound of Formula (I) to a subject in need thereof diminishes the extent of the inflammatory or autoimmune symptoms caused by the disease in the subject. In some embodiments, administering a compound of Formula (I) to a subject in need thereof stabilizes the inflammatory or autoimmune symptoms of the disease (prevents or delays the worsening of the inflammatory or autoimmune symptoms). In some embodiments, administering a compound of Formula (I) to a subject in need thereof delays the occurrence or recurrence of the inflammatory or autoimmune symptoms caused by the disease. In some embodiments, administering a compound of Formula (I) to a subject in need thereof slows the progression of the inflammatory or autoimmune symptoms caused by the disease. In some embodiments, administering a compound of Formula (I) to a subject in need thereof provides a partial remission of the disease which causes inflammatory or autoimmune symptoms. In some embodiments, administering a compound of Formula (I) to a subject in need thereof provides a total remission of the disease which causes inflammatory or autoimmune symptoms. In some embodiments, administering a compound of Formula (I) to a subject in need thereof decreases the dose of one or more other medications required to treat the disease which causes inflammatory or autoimmune symptoms. In some embodiments, administering a compound of Formula (I) to a subject in need thereof enhances the effect of another medication used to treat the inflammatory or autoimmune symptoms of the disease. In some embodiments, administering a compound of Formula (I) to a subject in need thereof delays the progression of the disease which causes inflammatory or autoimmune symptoms. In some embodiments, administering a compound of Formula (I) to a subject in need thereof increases the quality of life of the subject having a disease which causes inflammatory or autoimmune symptoms. In some embodiments, administering a compound of Formula (I) to a subject in need thereof prolongs survival of a subject having a disease which causes inflammatory or autoimmune symptoms. In some embodiments, the disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

In some embodiments, compounds of Formula (I) are useful for treating a disorder selected from atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, and familial cold autoinflammatory syndrome.

### Pharmaceutical Compositions and Routes of Administration

The compounds provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds disclosed herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of Formula (I) in the pharmaceutical composition may be at a level that will exercise the desired effect; for example, about 0.005 mg/kg of a subject's body weight to about 10 mg/kg of a subject's body weight in unit dosage for both oral and parenteral administration.

The dose of a compound of Formula (I) to be administered to a subject is rather widely variable and can be subject to the judgment of a health-care practitioner. In general, the compounds disclosed herein can be administered one to four times a day in a dose of about 0.001 mg/kg of a subject's body weight to about 10 mg/kg of a subject's body weight, but the above dosage may be properly varied depending on the age, body weight and medical condition of the subject and the type of administration. In any given case, the amount of the compound of Formula (I) administered may depend on such factors as the solubility of the active component, the formulation used and the route of administration.

A compound of Formula (I) can be administered orally for reasons of convenience. In one embodiment, when administered orally, a compound of Formula (I) is administered with a meal and water. In another embodiment, the compound of Formula (I) is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend inpart upon the site of the medical condition.

In one embodiment, provided herein are capsules containing a compound of Formula (I) without an additional carrier, excipient or vehicle.

In another embodiment, provided herein are compositions comprising an effective amount of a compound of Formula (I) and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of Formula (I) with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of Formula (I) as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of Formula (I) can be delayed or prolonged by proper formulation. For example, a slowly soluble pellet of the compound of Formula (I) can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of Formula (I) in oily or emulsified vehicles that allow it to disperse slowly in the serum.

### EXEMPLARY EMBODIMENTS

The present disclosure is further described by the following embodiments.

Embodiment P1. A compound of Formula (I):

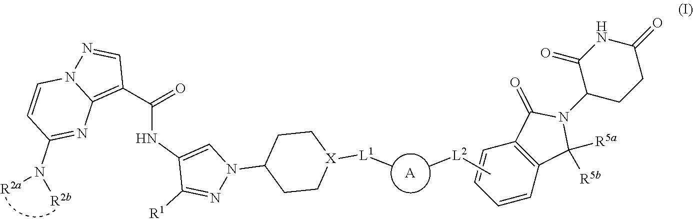

or a pharmaceutically acceptable salt thereof, wherein:
R¹ is C₁-C₆ haloalkyl, C₁-C₆ alkyl, or —CN;
R^{2a} is H or C₁-C₆ alkyl;
R^{2b} is C₅-C₆ cycloalkyl optionally substituted with 1-5 R³ groups;
or the dashed line between R^{2a} and R^{2b} represents a ring structure where R^{2a} and R^{2b} are taken together with the nitrogen atom to which they are attached to form a 6-membered heterocyclyl optionally containing one additional heteroatom selected from N and O, wherein the heterocyclyl is optionally substituted by 1-5 R³ groups;
each R³ is independently —NH₂, —OH, halo, C₁-C₆ alkyl, or C₁-C₆ haloalkyl;
X is CH or N;
L¹ is —C(O)N(H)—, —N(H)C(O)—, —C(O)—, —(C₁-C₆ alkylene)N(R⁴)—, or C₁-C₆ alkylene;
R⁴ is H or C₁-C₆ alkyl;
R^{5a} and R^{5b} are each H or are taken together to form an oxo group;
Ring A is

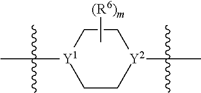

or an 8- to 10-membered spiro heterocyclylene containing 1-3 nitrogen atoms, wherein the heterocyclylene is substituted by m R⁶ groups;
Y¹ and Y² are independently CH or N;
each R⁶ is independently halo, C₁-C₆ alkyl, or C₁-C₆ haloalkyl;
m is 0-5;
L² is a bond or —N(R⁷)—; and
R⁷ is H or C₁-C₆ alkyl.

Embodiment P2. The compound of embodiment P1, or a pharmaceutically acceptable salt thereof, wherein:
R¹ is C₁-C₃ haloalkyl, C₁-C₃ alkyl, or —CN.

Embodiment P3. The compound of embodiment P2, or a pharmaceutically acceptable salt thereof, wherein:
R¹ is —CHF₂, —CF₃, —CH₃, or —CN.

Embodiment P4. The compound of any one of embodiments P1-P3, or a pharmaceutically acceptable salt thereof, wherein:
R^{2a} is H or C₁-C₃ alkyl; and
R^{2b} is cyclohexyl optionally substituted with 1-3 R³ groups.

Embodiment P5. The compound of embodiment P4, or a pharmaceutically acceptable salt thereof, wherein:
R^{2a} is H or —CH₃; and
R^{2b} is cyclohexyl optionally substituted with 1-2 R³ groups.

Embodiment P6. The compound of embodiment P5, or a pharmaceutically acceptable salt thereof, wherein:
R^{2a} is H; and
R^{2b} is cyclohexyl optionally substituted with 1 R³ group.

Embodiment P7. The compound of any one of embodiments P1-P3, or a pharmaceutically acceptable salt thereof, wherein:
R^{2a} and R^{2b} are taken together with the nitrogen atom to which they are attached to form a 6-membered heterocyclyl optionally containing one additional heteroatom selected from N and O, wherein the heterocyclyl is optionally substituted by 1-5 R³ groups.

Embodiment P8. The compound of embodiment P7, or a pharmaceutically acceptable salt thereof, wherein:
R^{2a} and R^{2b} are taken together with the nitrogen atom to which they are attached to form a 6-membered heterocyclyl optionally containing one additional heteroatom selected from N and O, wherein the heterocyclyl is optionally substituted by 1-2 R³ groups.

Embodiment P9. The compound of any one of embodiments P1-P8, or a pharmaceutically acceptable salt thereof, wherein:
each R³ is independently —NH₂, —OH, halo, C₁-C₃ alkyl, or C₁-C₃ haloalkyl.

Embodiment P10. The compound of embodiment P9, or a pharmaceutically acceptable salt thereof, wherein:
each R³ is independently —NH₂, —OH, F, C₁, —CH₃, or —CF₃.

Embodiment P11. The compound of embodiment P10, or a pharmaceutically acceptable salt thereof, wherein:
each R³ is independently —NH₂, —OH, F, or —CH₃.

Embodiment P12. The compound of any one of embodiments P1-P3 and P7-P11, or a pharmaceutically acceptable salt thereof, wherein:

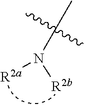

is

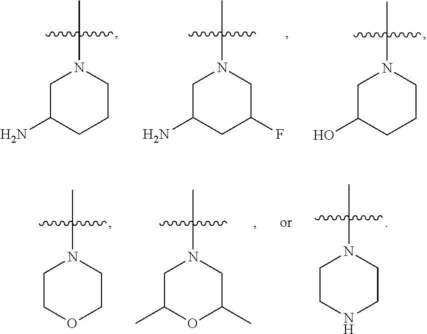

Embodiment P13. The compound of any one of P1-P6 and P9-P11, or a pharmaceutically acceptable salt thereof, wherein:

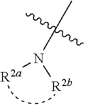

is

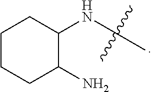 [SMILES: *NC1CCCCC1N]

Embodiment P14. The compound of any one of embodiments P1-P13, or a pharmaceutically acceptable salt thereof, wherein:
X is CH.

Embodiment P15. The compound of any one of embodiments P1-P13, or a pharmaceutically acceptable salt thereof, wherein:
X is N.

Embodiment P16. The compound of any one of embodiments P1-P15, or a pharmaceutically acceptable salt thereof, wherein:
L¹ is —C(O)N(H)—, —N(H)C(O)—, —C(O)—, —(C₁-C₃ alkylene)N(R⁴)—, or C₁-C₃ alkylene; and
R⁴ is H or C₁-C₃ alkyl.

Embodiment P17. The compound of embodiment P16, or a pharmaceutically acceptable salt thereof, wherein:
L¹ is —C(O)N(H)—, —N(H)C(O)—, —C(O)—, —CH₂N(CH₃)—, —CH₂—, or —CH₂CH₂—.

Embodiment P18. The compound of any one of embodiments P1-P17, or a pharmaceutically acceptable salt thereof, wherein:
R^{5a} and R^{5b} are each H.

Embodiment P19. The compound of any one of embodiments P1-P17, or a pharmaceutically acceptable salt thereof, wherein:
R^{5a} and R^{5b} are taken together to form an oxo group.

Embodiment P20. The compound of any one of embodiments P1-P19, or a pharmaceutically acceptable salt thereof, wherein:
Ring A is

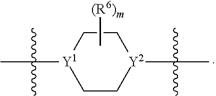

Embodiment P21. The compound of embodiment P20, or a pharmaceutically acceptable salt thereof, wherein:
Y¹ is N; and
Y² is CH.

Embodiment P22. The compound of embodiment P20, or a pharmaceutically acceptable salt thereof, wherein:
Y¹ is CH; and
Y² is N.

Embodiment P23. The compound of embodiment P20, or a pharmaceutically acceptable salt thereof, wherein:
Y¹ and Y² are each N.

Embodiment P24. The compound of embodiment P20, or a pharmaceutically acceptable salt thereof, wherein:
Y¹ and Y² are each CH.

Embodiment P25. The compound of any one of embodiments P1-P19, or a pharmaceutically acceptable salt thereof, wherein:
Ring A is an 8- to 10-membered spiro heterocyclylene containing 2 nitrogen atoms, wherein the heterocyclylene is substituted by m R⁶ groups.

Embodiment P26. The compound of embodiment P25, or a pharmaceutically acceptable salt thereof, wherein:
Ring A is a 10-membered spiro heterocyclylene containing 2 nitrogen atoms, wherein the heterocyclylene is substituted by m R⁶ groups.

Embodiment P27. The compound of any one of embodiments P1-P26, or a pharmaceutically acceptable salt thereof, wherein:
each R⁶ is independently halo, C₁-C₃ alkyl, or C₁-C₃ haloalkyl.

Embodiment P28. The compound of embodiment P27, or a pharmaceutically acceptable salt thereof, wherein:
each R⁶ is independently C₁, —CH₃, or —CF₃.

Embodiment P29. The compound of any one of embodiments P1-P26, or a pharmaceutically acceptable salt thereof, wherein:
m is 0.

Embodiment P30. The compound of any one of embodiments P1-P28, or a pharmaceutically acceptable salt thereof, wherein:
m is 1 or 2.

Embodiment P31. The compound of any one of embodiments P1-P24 and P29, or a pharmaceutically acceptable salt thereof, wherein:
Ring A is

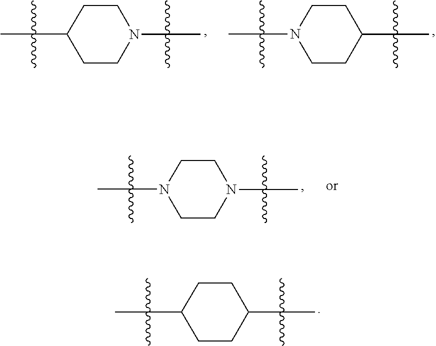 [SMILES: *C1CCN(*)CC1 ; *C1CCN(*)CC1 ; *N1CCN(*)CC1 ; *C1CCC(*)CC1]

Embodiment P32. The compound of any one of embodiments P1-P19, P25, P26, and P29, or a pharmaceutically acceptable salt thereof, wherein:
Ring A is

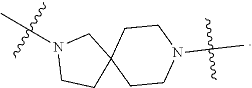 [SMILES: *N1CCC2(CC1)CCN(*)C2]

Embodiment P33. The compound of any one of embodiments P1-P32, or a pharmaceutically acceptable salt thereof, wherein:
L² is a bond.

Embodiment P34. The compound of any one of embodiments P1-P32, or a pharmaceutically acceptable salt thereof, wherein:
L² is —N(R⁷)—; and
R⁷ is H or C₁-C₃ alkyl.

Embodiment P35. The compound of embodiment P34, or a pharmaceutically acceptable salt thereof, wherein:
L² is —N(R⁷)—; and
R⁷ is H or —CH₃.

Embodiment P36. The compound of any one of embodiments P1-P35, or a pharmaceutically acceptable salt thereof, wherein:

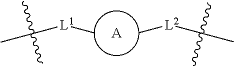

is

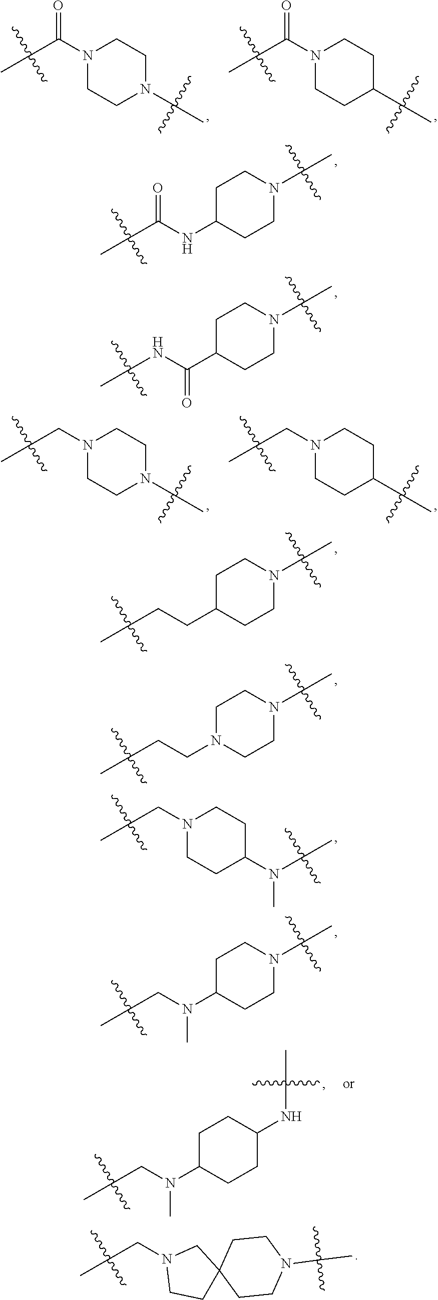

Embodiment P37. The compound of any one of embodiments P1-P24, P27-P31, and P33-P35, or a pharmaceutically acceptable salt thereof, wherein the compound is of Formula (IIa) or (IIb):

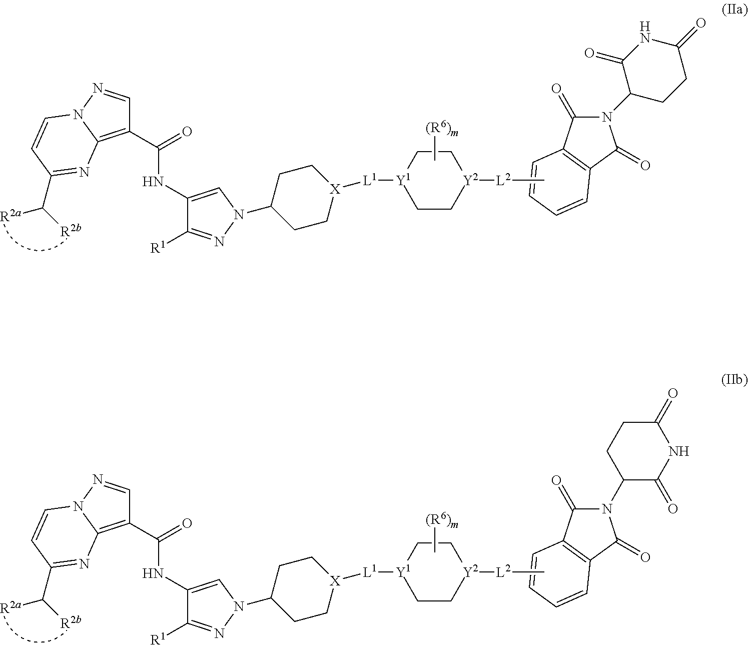

Embodiment P38. The compound of any one of embodiments P1-P36, or a pharmaceutically acceptable salt thereof, wherein the compound is of Formula (IIIa) or (IIIb):

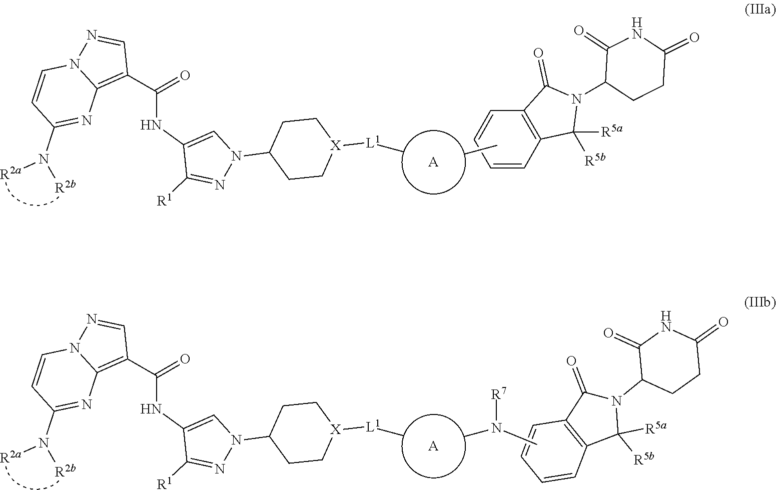

Embodiment P39. A compound selected from the compounds of Table 1 or a pharmaceutically acceptable salt thereof.

Embodiment P40. A pharmaceutical composition comprising the compound of any one of embodiments P1-P39, or a pharmaceutically acceptable salt thereof, and a pharmaceutically acceptable excipient.

Embodiment P41. A method of modulating interleukin-1 (IL1) receptor-associated kinase 4 (IRAK4) comprising contacting IRAK4 with an effective amount of the compound of any one of embodiments P1-P39, or a pharmaceutically acceptable salt thereof, or the pharmaceutical composition of embodiment P40.

Embodiment P42. A method of treating an inflammatory or autoimmune disease in a subject in need thereof, comprising administering to the subject an effective amount of the compound of any one of embodiments P1-P39, or a pharmaceutically acceptable salt thereof, or the pharmaceutical composition of embodiment P40.

Embodiment P43. The method of embodiment P42, wherein the inflammatory or autoimmune disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

The following abbreviations may be relevant for the application.

### Abbreviations

ACN or MeCN: acetonitrile
aq: aqueous
BCA assay: Bicinchoninic acid assay
n-Bu₄OAc: tetrabutylacetate
CBM: Cereblon Binding Moiety
CV: column volume
DABCO: 1,4-diazabicyclo[2.2.2]octane
DCE: dichloroethane
DCM: dichloromethane
DIBAL-H: diisobutylaluminium hydride
DIPEA: N,N-diisopropylethylamine
DMA: dimethylacetamide
DMF: dimethylformamide
DMPAO: 2-((2,6dimethylphenyl)amino)₂-oxoacetic acid
DMPU: N,N′-dimethylpropyleneurea
DMSO: dimethylsulfoxide
DP: desired product
equiv. or eq.: equivalents
ESI: electrospray ionization
Et₃N: triethylamine
EtOAc: ethyl acetate
FA: formic acid
FBS: fetal bovine serum
FC: flash chromatography
h: hour(s)
HATU: 1-[bis(dimethylamino)methylene]-1H-1,2,3-triazolo[4,5-b]pyridinium 3-oxide hexafluorophosphate
HPLC: high-performance liquid chromatography
IBX: 2-iodoxybenzoic acid
Ir(ppy)₂(dtbbpy)PF₆: 4,4′-bis(1,1-dimethylethyl)-2,2′-bipyridine-N1,N1′]bis[2-(2-pyridinyl-N)phenyl-C]iridium(III) hexafluorophosphate
i-PrOH: isopropanol
LCMS: liquid chromatography mass spectrometry
MeOH: methanol
MsCl: mesyl chloride
MSD: mass selective detector
MTBE: methyl tert-butyl ether
NaBH(OAc)₃: sodium triacetoxyborohydride
Ni-dtbbpy-Br₂: [4,4′-bis(1,1-dimethylethyl)-2,2′-bipyridine]nickel(II) dichloride
NMI: N-methyl imidazole
Pd-PEPPSI-IPent: dichloro[1,3-bis(2,6-di-3-pentylphenyl)imidazol-2-ylidene](3-chloropyridyl)palladium(II)
pdt: product
PhMe: toluene
Rf: retardation factor
rt: room temperature
r.t.: retention time
sat.: saturated
SM: starting material
TCFH: chloro-N,N,N′,N′-tetramethylformamidinium hexafluorophosphate
TFA: trifluoroacetic acid
THF: tetrahydrofuran
THP: tetrahydropyran
TLC: thin layer chromatography

### SYNTHETIC EXAMPLES

### LCMS Methods

### LCMS Method 1

Column: Luna C18 (2) 50×3 mm, 3 um. Temperature: 45° C., Flow: 1.5 mL/min, run time: 2.5 min. Mobile phase conditions: Initial 95% H₂O 0.1% FA/5% MeCN 0.1% FA, linear gradient to 95% MeCN 0.1% FA over 1.3 min then hold for 1.2 minute at 95% MeCN 0.1% FA. MSD: ESI Positive.

### LCMS Method 2

Column: Kinetex Polar C18 2.6 um, 50×3.0 mm. Temperature: 45° C., Flow: 1.2 mL/min, Run time: 3 min. Mobile phase conditions: Initial 95% H₂O+0.1% FA/5% MeCN+0.1% FA then linear gradient to 95% MeCN+0.1% FA for 1.5 min then hold for 1.5 min at 95% MeCN+0.1% FA. MSD: Positive.

### LCMS Method 3

Column: Kinetex Polar C18 2.6 um, 50×3.0 mm. Temperature: 40° C., Flow: 1.2 mL/min, Run time: 6 min. Mobile phase conditions: Initial 95% H₂O+0.1% FA/5% MeCN+0.1% FA then linear gradient to 95% MeCN+0.1% FA for 3.5 min then hold for 2.5 min at 95% MeCN+0.1% FA. MSD: Positive.

### LCMS Method 4

Column: Luna C18 (2) 50×3 mm, 3 um. Temperature: 45° C., Flow: 1.5 mL/min, run time: 3.5 min. Mobile phase conditions: Initial 95% H₂O 0.1% FA/5% MeCN 0.1% FA, linear gradient to 95% MeCN 0.1% FA over 1.3 min then hold for 2.2 minute at 95% MeCN 0.1% FA. MSD: ESI Positive.

### LCMS Method 5

Column: SunFire C18 75×4.6 mm, 3.5 um. Temperature: 45° C., Flow: 1.5 mL/min, run time: 6 min. Mobile phase conditions: Initial 95% H₂O+0.1% FA/5% MeCN+0.1% FA then linear gradient to 95% MeCN+0.1% FA for 4 min then hold for 2 min at 95% MeCN+0.1% FA. MSD: Positive.

### LCMS Method 6

Column: C18 4.6×100 mm, Initial Gradient at 95% H₂O+0.1% FA/5% MeCN+0.1% FA 10 min run with 1.5 min equilibration, Gradient 0 to 8 min at 95% H₂O to 0% and hold for 2 minute.

### Synthesis of Intermediates

Reference to a particular intermediate compound by number, such as 1 or 2, is specific to the example in which it is described. As such, multiple examples may refer to the same intermediate compound number, such as 1 or 2, but the chemical structure of the compound will be different across the different examples.

### Example I-1. Synthesis of Common Intermediate T-1

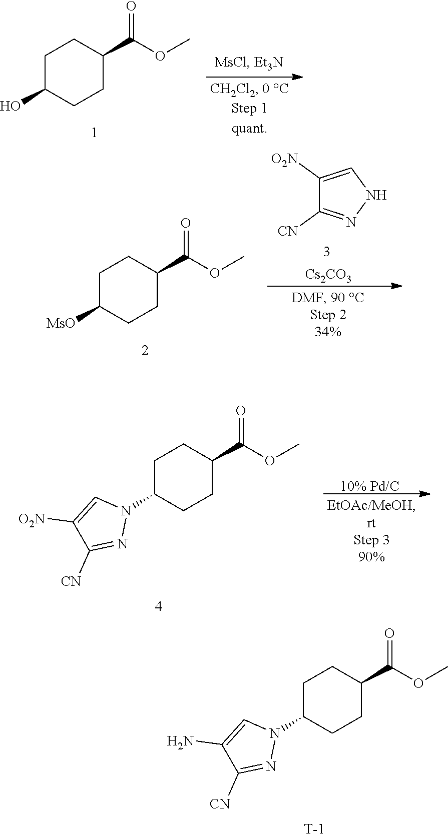

Step 1. Preparation of Methyl 4-methylsulfonyloxycyclohexanecarboxylate (2). To a solution of methyl 4-hydroxycyclohexanecarboxylate 1 (3.0 g, 18.96 mmol) and Et₃N (3.96 mL, 28.45 mmol) in CH₂Cl₂ (60 mL) at 0° C. was added methanesulfonyl chloride (1.91 mL, 24.65 mmol) dropwise. The reaction was stirred for 65 min at 0° C. The reaction was followed by TLC (6/4: EtOAc/Heptane), Rf of the desired compound 2=0.30, co-spotted with 1 (Rf=0.25) until complete conversion. Water was added and the aqueous phase was extracted with 3×30 mL of CH₂Cl₂. The organic phases were dried over Na₂SO₄ and the solvent was evaporated to dryness to give 2 as a light yellow oil, 4.45 g, quantitative yield. No NMR was taken.

LCMS method 1: 99.9% purity at 215 nm, [M−Ms+H]+=141.2; [M+Na]+=259.2.

Step 2. Preparation of Methyl 4-(3-cyano-4-nitro-pyrazol-1-yl)cyclohexanecarboxylate (4). To a solution of methyl 4-methylsulfonyloxycyclohexanecarboxylate 2 (2.05 g, 8.69 mmol) and 4-nitro-1H-pyrazole-3-carbonitrile 3 (1.0 g, 7.24 mmol) in dry DMF (1.5 mL) at rt was added cesium carbonate. (3.54 g, 10.86 mmol). The resulting mixture was stirred overnight at 90° C. LCMS showed 50% conversion (r.t=1.70 min). Water was added to the mixture and the aqueous phase was extracted with EtOAc (3×). The organic phase was washed once with brine and dried over sodium sulfate. The solvent was evaporated to give 4 as a light yellow solid, 694 mg, yield=34% with 97.7% purity by LCMS.

LCMS method 1: 97.7% purity at 215 nm, [M+H]+=279.2.

¹H NMR (400 MHz, DMSO-d6) δ ppm 1.47-1.59 (m, 2H), 1.69-1.73 (m, 1H), 1.84 (qd, J=12.5, 3.3 Hz, 2H), 2.03-2.15 (m, 4H), 2.37-2.47 (m, 1H), 3.61-3.63 (m, 3H), 4.42 (tt, J=11.9, 3.9 Hz, 1H), 9.21 (s, 1H).

Step 3. Preparation of Methyl 4-(4-amino-3-cyano-pyrazol-1-yl)cyclohexanecarboxylate (T-1). Nitrogen was bubbled for 5 minutes through a solution of methyl 4-(3-cyano-4-nitro-pyrazol-1-yl)cyclohexanecarboxylate 4 (750.0 mg, 2.7 mmol) in a mixture of ethyl acetate (20.2 mL) and methanol (6.7 mL). 10% Pd/C (573.67 mg, 0.54 mmol) was then added and nitrogen was bubbled through the solution for another 5 minutes. Hydrogen was then bubbled through the solution for 5 minutes and the resulting mixture was stirred under hydrogen atmosphere (1 atm) for 5 h. LCMS showed complete conversion. The reaction mixture was filtered on Celite, washed with EtOAc. The filtrate was concentrated under reduced pressure. The crude mixture was then purified by reverse phase flash column chromatography on C18 RediSep Rf Gold (liquid deposit with DMSO, eluted with MeOH/0.1% HCOOH 5/95% for 4 CV, then 12 CV to 60% MeOH). Fractions were concentrated to dryness to give T-1 as an off-white solid, m=603 mg, 90.1% yield.

LCMS method 1: 99.9% purity at 215 nm, [M+H]+=249.4, [M+Na]=271.2.

¹H NMR (400 MHz, DMSO-d6) δ ppm 1.44-1.56 (m, 2H), 1.66-1.80 (m, 2H), 2.00 (br d, J=11.5 Hz, 4H), 2.33-2.45 (m, 1H), 3.61 (s, 3H), 4.12 (tt, J=11.8, 3.2 Hz, 1H), 4.74 (s, 2H), 7.24 (s, 1H).

### Example I-2. Synthesis of Common Intermediate T-2

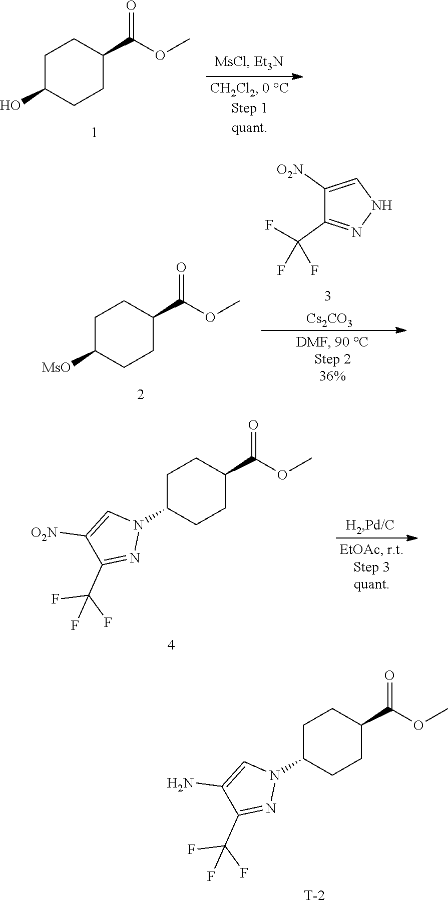

Step 1. Preparation of Methyl 4-methylsulfonyloxycyclohexanecarboxylate (2). To a solution of methyl 4-hydroxycyclohexanecarboxylate (1) (5.0 g, 31.61 mmol, 1.0 equiv.) and Et₃N (6.61 mL, 47.41 mmol. 1.5 equiv.) in CH₂Cl₂ (158 mL, 0.2 M) at 0° C. was added methanesulfonyl chloride (3.18 mL, 41.09 mmol, 1.3 equiv.) dropwise. The resulting mixture was stirred at 0° C. for 30 minutes. TLC (KMnO₄): SM: Rf=0.25, product: Rf=0.30 (60% EtOAc/heptanes). Water was added and the aqueous phase was extracted with CH₂Cl₂ (3×). The combined organics were washed with aqueous 1 M HCl (1×), dried over Na₂SO₄, filtered, then concentrated to dryness under reduced pressure to afford pure 2 (7.47 g, quantitative yield) without any further purification.

LCMS Method 1: 99.9% purity at 215 nm; [M−MsOH+H]=141.2 m/z, [M+Na]+=259.2 m/z.

¹H NMR (400 MHz, CDCl₃) δ ppm 1.67-1.82 (m, 4H), 1.87-1.97 (m, 2H), 1.97-2.07 (m, 2H), 2.36-2.45 (m, 1H), 3.01 (s, 3H), 3.68 (s, 3H), 4.88-4.94 (m, 1H).

Step 2. Preparation of Methyl (1r,4r)-4-(4-nitro-3-(trifluoromethyl)-1H-pyrazol-1-yl)cyclohexane-1-carboxylate (4). To a solution of 4-nitro-3-(trifluoromethyl)-1H-pyrazole 3 (1.0 g, 5.52 mmol, 1.0 eq.) and methyl 4-methylsulfonyloxycyclohexanecarboxylate 2 (1.3 g, 5.52 mmol, 2.0 eq.) in dry DMF (18.41 mL, 0.3 M) was added Cs₂CO₃ (3.6 g, 11.05 mmol, 2.0 eq.). After stirring at 90° C. overnight, LCMS showed complete conversion into 4. The reaction was quenched with water and the aqueous layer was extracted three times with EtOAc. The combined organic layers were washed two times with water, one time with brine, dried over Na₂SO₄, filtered and concentrated under reduced pression. The crude product was purified by flash column chromatography (80 g SiO₂ eluting with 0 to 35% MTBE in Heptane over 15 CVs, then 35% MTBE in Heptane over 3 CVs. The desired product came out 30% MTBE in Heptane, Rf=0.30 on TLC eluting 60% MTBE in Heptane, revealing UV+KMnO₄. The fractions were combined and concentrated to dryness to give 4 (671 mg, 36% yield) as a light yellow solid.

LCMS method 1: retention time: 1.819 min, 99.9% purity at 215 nm, [M+H]+=322.2.

¹H NMR (400 MHz, DMSO-d6) δ ppm 1.52 (qd, J=13.0, 3.0 Hz, 2H), 1.83 (br dd, J=12.5, 3.1 Hz, 2H), 2.00-2.17 (m, 4H), 2.42 (tt, J=12.2, 3.5 Hz, 1H), 3.61 (s, 3H), 4.39 (tt, J=11.9, 3.8 Hz, 1H), 9.19 (s, 1H).

¹⁹F NMR (377 MHz, DMSO-d6) δ ppm-61.45 (s, 3 F).

Step 3. Preparation of Methyl 4-[4-amino-3-(trifluoromethyl)pyrazol-1-yl]cyclohexanecarboxylate (T-2). To a solution of methyl 4-[4-nitro-3-(trifluoromethyl)pyrazol-1-yl]cyclohexanecarboxylate 4 (671 mg, 2.09 mmol, 1.0 eq.) in ethyl acetate (13.93 mL, 0.15 M) was added 10% Pd/C (444 mg, 0.42 mmol, 0.2 eq.). Nitrogen was bubbled through the solution for 10 min and the balloon was replaced by hydrogen. Hydrogen was bubbled through the solution for 5 minutes and the resulting mixture was stirred under a hydrogen atmosphere (1 atm). After stirring for 5 hours at room temperature, LCMS showed complete conversion into product. The reaction mixture was filtered through a pad of celite and washed with EtOAc. The filtrate was concentrated under reduced pressure to give T-2 (608 mg, quantitative yield) as a pink solid. The crude product was used in the next step without further purification.

LCMS method 1: retention time: 1.562 min, 99.9% purity at 215 nm, [M+H]+=292.2.

¹H NMR (400 MHz, DMSO-d6) δ ppm 1.41-1.60 (m, 2H), 1.64-1.81 (m, 2H), 1.91-2.06 (m, 4H), 2.32-2.45 (m, 1H), 3.60 (s, 3H), 4.00-4.13 (m, 1H), 4.21 (s, 2H), 7.22 (s, 1H).

¹⁹F NMR (377 MHz, DMSO-d6) δ ppm-58.85 (s, 3 F).

### Example I-3. Synthesis of Common Intermediate T-3

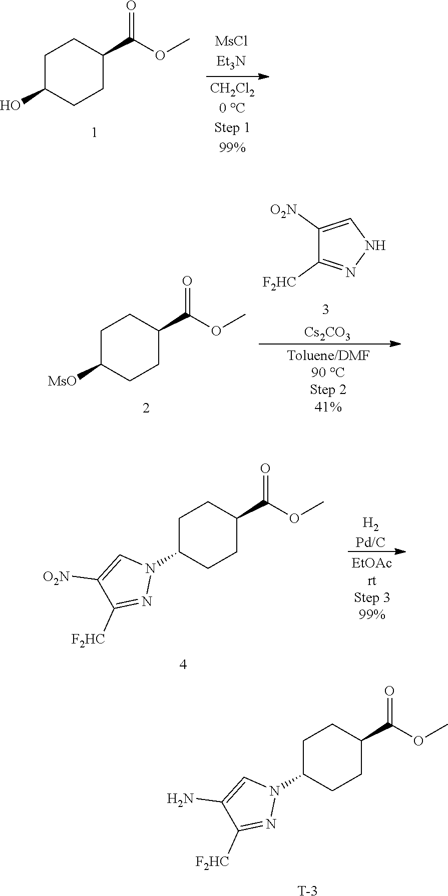

Step 1. Preparation of Methyl 4-methylsulfonyloxycyclohexanecarboxylate (2). Under nitrogen, a solution of methyl 4-hydroxycyclohexanecarboxylate 1 (5.0 g, 31.61 mmol, 1.0 eq.) in CH₂Cl₂ (105 mL, 0.3 M) was cooled to 0° C., then methanesulfonyl chloride (2.69 mL, 34.77 mmol, 1.1 eq.) and triethylamine (5.28 mL, 37.93 mmol, 1.2 eq.) were added, the latter dropwise. After stirring 2 h at 0° C., TLC (CH₂Cl₂/MeOH 5.5:0.5, KMnO₄ stain) showed full conversion. The reaction was quenched by addition of water, then phases were separated and the aqueous phase was extracted 3 times with CH₂Cl₂. The combined organic phases were washed twice with brine, dried over magnesium sulfate, filtered and concentrated to give 2 (7.45 g, 99% yield) as a yellow oil.

¹H NMR (400 MHz, CDCl₃) δ ppm 1.67-1.84 (m, 4H), 1.87-1.99 (m, 2H), 2.00-2.09 (m, 2H), 2.36-2.46 (m, 1H), 3.02 (s, 3H), 3.69 (s, 3H), 4.87-4.96 (m, 1H).

Step 2. Preparation of Methyl 4-[3-(difluoromethyl)-4-nitro-pyrazol-1-yl]cyclohexanecarboxylate (4). Under nitrogen, in a flame-dried round-bottom flask, a solution of 2 (1.16 g, 4.91 mmol, 1.0 eq.), 3 (0.80 g, 4.91 mmol, 1.0 eq.) and DMF (12.3 mL, 0.4 M) was stirred at room temperature for 5 minutes before Cs₂CO₃ (3.20 g, 9.81 mmol, 2.0 eq.) was added. The resulting mixture was stirred at 90° C. for 16 h. Incomplete conversion of 3 was observed by LCMS (method 1), then a second portion of 2 (1.16 g, 4.91 mmol, 1.0 eq.) was added and stirring at 90° C. was resumed for 48 h. A 90% conversion of 3 was observed by LCMS. The reaction was quenched by addition of water. Ethyl acetate was added and phases were separated. The aqueous phase was extracted 3 times with ethyl acetate, then the combined organic phases were washed once with water and once with brine, dried over magnesium sulfate and concentrated. The residue was taken up in MTBE and water, then phases were separated. The organic phase was washed 5 times with water, then once with brine, dried over magnesium sulfate, filtered and concentrated to give an orange oil. The residue was then purified by normal phase flash chromatography (80 g silica column, elution: 0 to 30% MTBE/heptanes over 10 CV). Fractions were combined and concentrated to give impure 4 (883 mg). The residue was then purified by reverse phase flash chromatography (100 g C18 RediSep Rf Gold column, liquid deposit (DMSO), elution: 5% MeOH/0.10% HCOOH over 4 CV, then 5% to 100% MeOH/0.1% HCOOH over 15 CV). Fractions were combined and concentrated to give 4 (618 mg, 41% yield) as a white solid.

LCMS method 1: retention time: 1.745 min, 99.9% purity at 215 nm, [M+H]+=304.2.

¹H NMR (400 MHz, DMSO-d6) δ ppm 1.45-1.60 (m, 2H), 1.76-1.89 (m, 2H), 2.00-2.15 (m, 4H), 2.36-2.47 (m, 1H), 3.61 (s, 3H), 4.28-4.40 (m, 1H), 7.14-7.47 (m, 1H), 9.05 (s, 1H).

¹⁹F NMR (377 MHz, DMSO-d6) δ ppm-117.40 (s, 2 F).

Step 3. Preparation of Methyl 4-[4-amino-3-(difluoromethyl)pyrazol-1-yl]cyclohexanecarboxylate (T-3). Under nitrogen, a solution of 4 (625 mg, 2.06 mmol, 1.0 eq.) and ethyl acetate (20.6 mL, 0.1 M) was degassed at room temperature by sparging with nitrogen for 15 minutes. Pd/C (438 mg, 10% w/w, 0.41 mmol, 0.2 eq.) was added and sparging was resumed for 15 minutes. The mixture is sparged with H₂ for 15 minutes, then the needle was kept just over the surface of the solvent and the mixture was stirred at room temperature for 16 h. Complete conversion of 4 was observed by LCMS (method 1). The mixture was filtered over celite, then the filter cake was washed thoroughly with ethyl acetate and the resulting solution was concentrated to give T-3 (563 mg, 99% yield) as a light orange solid.

LCMS method 1: retention time: 1.297 min, 99.9% purity at 215 nm, [M+H]+=274.2.

¹H NMR (400 MHz, CDCl₃) δ ppm 1.53-1.80 (m, 5H), 2.12-2.42 (m, 9H), 3.93-4.03 (m, 1H), 6.54-6.84 (m, 1H), 7.07 (s, 1H).

¹⁹F NMR (377 MHz, CDCl₃) δ ppm-112.22 (s, 2 F).

### Synthesis of Cereblon Binding Moieties (CBMs)

### Example I-4. Synthesis of Common Intermediate C-1

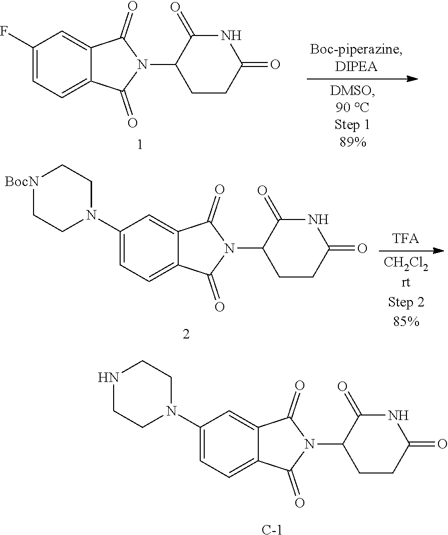

Step 1. Preparation of tert-Butyl 4-[2-(2,6-dioxo-3-piperidyl)-1,3-dioxo-isoindolin-5-yl]piperazine-1-carboxylate (2). A sealed tube was charged with 2-(2,6-dioxo-3-piperidyl)-5-fluoro-isoindoline-1,3-dione 1 (2.0 g, 7.24 mmol), tert-butyl piperazine-1-carboxylate (1.4 g, 7.52 mmol), DIPEA (3.87 mL, 22.2 mmol) and DMSO (15 mL). The tube was sealed and heated at 90° C. for 72 h. The progress of the reaction was followed by HPLC. Upon completion, the reaction mixture was directly purified by reverse phase chromatography C18 RediSep Rf Gold with 5-95% MeOH/0.1% formic acid in H₂O as eluent. The pure fractions were combined and concentrated under reduced pressure to afford 2 (2.86 g, 89% yield) as a yellow solid.

LCMS method 1: 99.9% purity at 215 nm, [M-tBu+H]+=387.2 m/z, [M+Na]+=466.2 m/z.

¹H NMR (400 MHz, DMSO-d6) δ ppm 1.42 (s, 9H), 1.99-2.09 (m, 1H), 2.52-2.64 (m, 2H), 2.82-2.95 (m, 1H), 3.47 (m, 8H), 5.07 (dd, J=13.0, 5.4 Hz, 1H), 7.24 (dd, J=8.6, 2.2 Hz, 1H), 7.35 (d, J=2.0 Hz, 1H), 7.70 (d, J=8.3 Hz, 1H), 11.08 (s, 1H).

Step 2. Preparation of 2-(2,6-dioxo-3-piperidyl)-5-piperazin-1-yl-isoindoline-1,3-dione trifluoroacetic acid salt (C-1). TFA (10.55 mL, 129.5 mmol) was added to a solution of tert-butyl 4-[2-(2,6-dioxo-3-piperidyl)-1,3-dioxo-isoindolin-5-yl]piperazine-1-carboxylate 2 (2.87 g, 6.48 mmol) in CH₂Cl₂ (30 mL) and the mixture was stirred at room temperature for 2 h. Upon completion by HPLC, the volatiles were removed under vacuum and the residue co-evaporated with MeCN (3×) and MTBE (2×). The residue was purified by C18 RediSep Rf Gold reverse phase chromatography with 5-20% MeCN/0.05% TFA in H₂O as eluent. The pure fractions were concentrated under reduced pressure to afford C-1 (2.52 g, 85% yield) as a yellow solid as a TFA salt.

LCMS method 1: 99.9% purity at 215 nm, [M+H]+=343.2 m/z.

¹H NMR (400 MHz, DMSO-d6) δ ppm 1.95-2.10 (m, 1H), 2.52-2.64 (m, 2H), 2.82-2.97 (m, 1H), 3.18-3.31 (m, 4H), 3.62-3.73 (m, 4H), 5.09 (dd, J=12.8, 5.5 Hz, 1H), 7.33 (dd, J=8.8, 2.2 Hz, 1H), 7.46 (d, J=2.0 Hz, 1H), 7.76 (d, J=8.3 Hz, 1H), 11.09 (s, 1H).

### Example I-5. Synthesis of Common Intermediate C-2

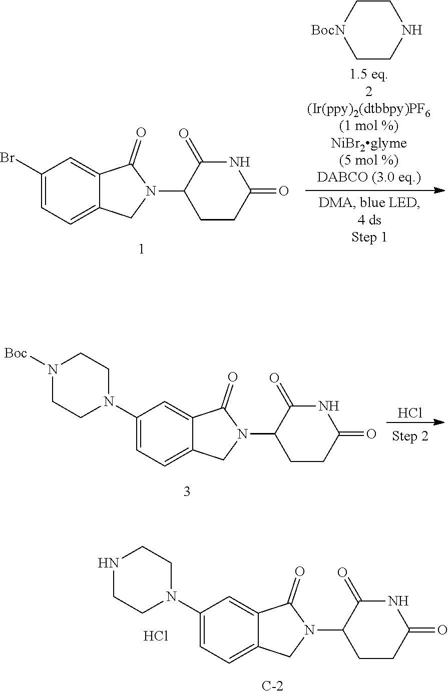

Step 1. Preparation of tert-butyl 4-[2-(2,6-dioxo-3-piperidyl)-3-oxo-isoindolin-5-yl]piperazine-1-carboxylate (3). To an oven-dried 40 ml vial equipped with a magnetic stir bar under ambient atmosphere, 3-(6-bromo-1-oxo-isoindolin-2-yl)piperidine-2,6-dione (1) (500 mg, 1.55 mmol), tert-butyl piperazine-1-carboxylate (2) (432 mg, 2.32 mmol), DABCO (521 mg, 4.64 mmol) and Ir(ppy)₂(dtbbpy)PF₆ (14.2 mg, 15.47 umol) were added, followed by DMA (10 mL). Then dibromonickel; 1,2-dimethoxyethane (23.9 mg, 77.4 umol) was added as a solution in DMA (0.5 mL) under N₂. The reaction was placed 6 cm in front of one 30 W blue LED at 25° C. for 96 h. LCMS showed the major peak was the expected product. The combined 8 batches were added into water (200 mL) dropwise, and the formed solid was filtered and concentrated. The residue was purified by prep-HPLC(column: Phenomenex luna c18 250 mm×100 mm×10 um; mobile phase: [water(0.1% TFA)-ACN]; B %: 16%-46%, 25 min) to give tert-butyl 4-[2-(2,6-dioxo-3-piperidyl)-3-oxo-isoindolin-5-yl]piperazine-1-carboxylate (3) (2.5 g, 47.1% yield) as a gray solid.

¹H NMR: 400 MHz, DMSO-d6 δ:10.97 (s, 1H), 7.45 (d, J=8.8 Hz, 1H), 7.30-7.27 (m, 1H), 7.20 (d, J=2.0 Hz, 1H), 5.13-5.08 (m, 1H), 4.30 (dd, J=16.8 Hz, 51.6 Hz, 2H), 3.48 (d, J=4.8 Hz, 4H), 3.16 (d, J=5.2 Hz, 4H), 2.92-2.68 (m, 1H), 2.62-2.51 (m, 1H), 2.41-2.37 (m, 1H), 2.01-1.99 (m, 1H), 1.45 (s, 9H).

Step 2. 3-(1-oxo-6-piperazin-1-yl-isoindolin-2-yl)piperidine-2,6-dione (C-2). Tert-butyl 4-[2-(2,6-dioxo-3-piperidyl)-3-oxo-isoindolin-5-yl]piperazine-1-carboxylate 3 (5 g, 11.7 mmol) was added to HCl (12 N, 15 mL) at 0° C. The reaction mixture was stirred at 20° C. for 1 h. LCMS showed the major peak was the expected product. The mixture was diluted with MeCN (500 mL) at 0-10° C., the formed solid was filtered and the cake was dried to give product 3-(1-oxo-6-piperazin-1-yl-isoindolin-2-yl)piperidine-2,6-dione C-2 (4.3 g, HCl salt, yield 100%) as a gray solid.

¹H NMR: 400 MHz, DMSO-d6 δ: 10.98 (s, 1H), 9.34 (s, 2H), 7.49 (d, J=5.2 Hz, 1H), 7.33-7.27 (m, 2H), 5.13-5.09 (m, 1H), 4.30 (dd, J=17.2 Hz, J=58.2 Hz, 2H), 3.46 (d, J=4.8 Hz, 4H), 3.23 (d, J=4.8 Hz, 1H), 2.93-2.78 (m, 1H), 2.62-2.51 (m, 1H), 2.48-2.37 (m, 1H), 2.01-1.98 (m, 1H).

### Example I-6. Synthesis of Common Intermediate C-3

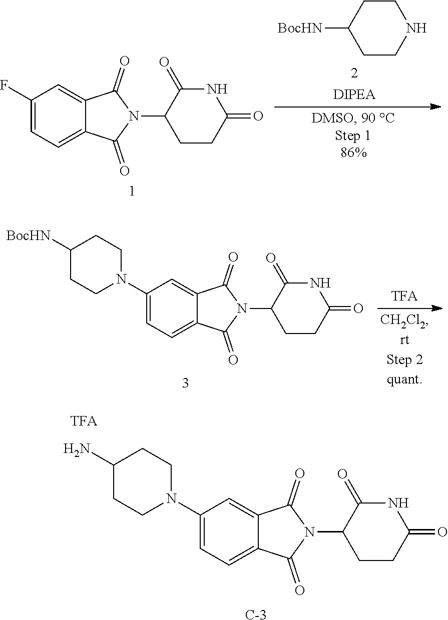

Step 1. Preparation of tert-Butyl N-[1-[2-(2,6-dioxo-3-piperidyl)-1,3-dioxo-isoindolin-5-yl]-4-piperidyl]carbamate (3). To a solution of 2-(2,6-dioxo-3-piperidyl)-5-fluoro-isoindoline-1,3-dione 1 (200.0 mg, 0.72 mmol) and tert-butyl N-(4-piperidyl)carbamate 2 (188.52 mg, 0.94 mmol) in dry DMSO (3.62 mL) was added DIPEA (252.23 uL, 1.45 mmol). The resulting mixture was stirred at 90° C. overnight. LCMS showed complete conversion. The reaction mixture was diluted with water and the aqueous phase was extracted with EtOAc (4×). The organic layers were washed with H₂O (5×), dried over Na₂SO₄ and concentrated to dryness. The residue was then purified by reverse phase flash chromatography (30 g C18 RediSep Rf Gold column, liquid deposit (DMSO), elution: 5% MeCN/0.1% HCOOH over 5 CV, then 5 to 100% MeCN/0.1% HCOOH over 20 CV). The desired fraction was concentrated to dryness to give 3 as a yellow solid (283 mg, 86% yield).

LCMS method 1: 99.9% purity at 215 nm, [M−2HCOOH+H]+=457.2.

¹H NMR (400 MHz, DMSO-d6) δ ppm 1.39 (s, 9H), 1.40-1.45 (m, 2H), 1.80 (br d, J=11.2 Hz, 2H), 1.97-2.05 (m, 1H), 2.53-2.63 (m, 2H), 2.83-2.95 (m, 1H), 3.05 (br t, J=11.9 Hz, 2H), 3.49-3.60 (m, 1H), 3.98 (br d, J=13.4 Hz, 2H), 5.06 (dd, J=13.0, 5.4 Hz, 1H), 6.87 (br d, J=7.6 Hz, 1H), 7.31 (d, J=1.7 Hz, 1H), 7.65 (d, J=8.6 Hz, 1H), 8.14 (s, 1H), 11.07 (s, 1H).

Step 2. Preparation of 5-(4-Amino-1-piperidyl)-2-(2,6-dioxo-3-piperidyl)isoindoline-1,3-dione trifluoroacetic acid salt (C-3). To a solution of tert-butyl N-[1-[2-(2,6-dioxo-3-piperidyl)-1,3-dioxo-isoindolin-5-yl]-4-piperidyl]carbamate 3 (283.0 mg, 0.62 mmol) in CH₂Cl₂ (6.19 mL) was added TFA (721.44 uL, 9.3 mmol). The resulting mixture was stirred at room temperature overnight. LCMS showed full conversion. The solvents were evaporated under high pressure. The residue was co-evaporated 2× with MeCN/Toluene and 2× MeCN, then dried under high vacuum to give C-3 as a yellow solid (291 mg, quant. yield). The crude product was used without purification in the next reaction.

LCMS method 1: 99.9% purity at 215 nm, [M−TFA+H]=357.2.

### Example I-7. Synthesis of Common Intermediate C-4

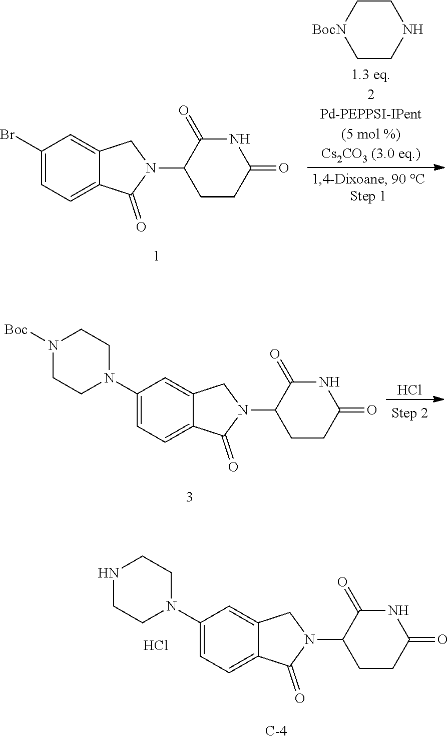

Step 1. Preparation of tert-butyl 4-[2-(2,6-dioxo-3-piperidyl)-1-oxo-isoindolin-5-yl]piperazine-1-carboxylate (3). To a sealed tube was added tert-butyl piperazine-1-carboxylate 2 (74.93 mg, 0.4000 mmol) and 3-(5-bromo-1-oxo-isoindolin-2-yl)piperidine-2,6-dione 1 (100. mg, 0.3100 mmol) in 1,4-dioxane (3.0946 mL). The solution was degassed, and then Cs₂CO₃ (302.49 mg, 0.9300 mmol) and Pd-PEPPSI-IPent (12.25 mg, 0.0200 mmol) were added. The tube was sealed and stirred at 90° C. overnight. After 20 h, reaction showed full conversion and was passed over celite and washed with DCM. The filtrate was concentrated in vacuo. The residue was purified by silica gel column chromatography (40 g, DCM/MeOH, 0% to 5% in 15 CV, pdt exit at 4% MeOH). The pure fractions were combined, volatiles evaporated, and dried under high vacuum to give compound 3 (82 mg, 0.1912 mmol, 61.779% yield) as a white solid.

Step 2. Preparation of 3-(1-oxo-5-piperazin-1-yl-isoindolin-2-yl)piperidine-2,6-dione (C-4). In a vial, tert-butyl 4-[2-(2,6-dioxo-3-piperidyl)-1-oxo-isoindolin-5-yl]piperazine-1-carboxylate 3 (200 mg, 0.4700 mmol) was suspended in HCl (2 mL, 8 mmol). Ethyl acetate (2 mL) was added to the mixture and stirring was continued for 30 minutes. After 30 minutes, HPLC showed not much conversion. Methanol (1 mL) was added to the reaction and stirring was continued. HPLC after 30 minutes showed incomplete conversion. The mixture was sonicated and stirred in a hot water bath (50° C.) for 30 minutes. HPLC showed complete conversion. The mixture was concentrated to dryness and co-evaporated with MeCN (3×) to afford the desired product C-4 as an off-white solid (180 mg, 0.4687 mmol, 99% yield).

### Example I-8. Synthesis of Common Intermediate C-5

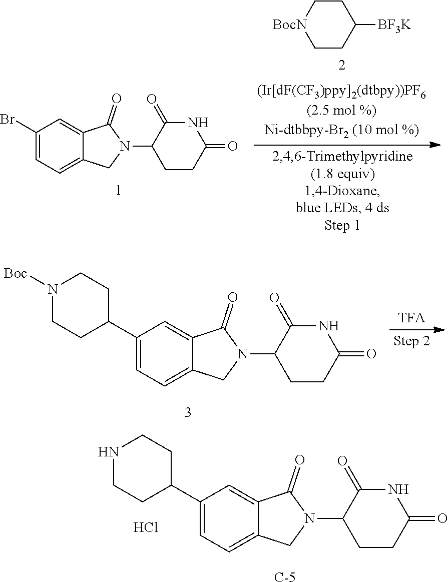

Step 1. Preparation of tert-butyl 4-[2-(2,6-dioxo-3-piperidyl)-3-oxo-isoindolin-5-yl]piperidine-1-carboxylatee (3). To a solution of 3-(6-bromo-1-oxo-isoindolin-2-yl)piperidine-2,6-dione 1 (250. mg, 0.7700 mmol), potassium (1-tert-butoxycarbonyl-4-piperidyl)-trifluoro-boranuide 2 (595.02 uL, 2.32 mmol) and 2,4,6-trimethylpyridine (0.18 mL, 1.39 mmol) in 1,4-dioxane (2 mL) in a Schlenk flask was added a solution of (Ir[dF(CF₃)ppy]₂(dtbpy))PF₆ (2.17 mg, 0.002 mmol) in 1,4-dioxane (1 mL). A solution of Ni-dtbbpy-Br₂ (37.67 mg, 0.0800 mmol) in 1,4-dioxane (1 mL), which had been sonicated for 1 minute, was then added. The reaction mixture was degassed 3× using the freeze-pump-thaw method. The flask was then placed under a dry nitrogen atmosphere and sealed with a parafilm. The reaction mixture was stirred under blue LED irradiation for 4 days. The solution was evaporated until it was dry. To the crude mixture was added DMSO. The crude mixture was purified by column chromatography C18 (5 to 100% MeCN in H₂O (0.1% formic acid), 5% MeCN to 100% in 15 CV) and product 3 was combined and concentrated to dryness.

LCMS method 1: 99.9% purity at 215 nm, [M+H]+=428.1.

Step 2. Preparation of 2-(2,6-dioxopiperidin-3-yl)-5-(piperidin-4-yl)isoindoline-1,3-dione (C-5). To a solution of tert-butyl 4-[2-(2,6-dioxo-3-piperidyl)-3-oxo-isoindolin-5-yl]piperidine-1-carboxylate 3 (46. mg, 0.1100 mmol) in DCM (1.4035 mL) was added TFA (0.18 mL, 2.15 mmol). The reaction was stirred at room temperature for 2 h, at which point full conversion was observed by LCMS. The crude reaction mixture was evaporated with MeCN/PhMe (4×) to afford product C-5 (46 mg, 0.1042 mmol, 97% yield) that was used without purification in the next step.

LCMS method 1: 99.9% purity at 215 nm, [M+H]+=328.2.

### Example I-9. Synthesis of Common Intermediate C-6

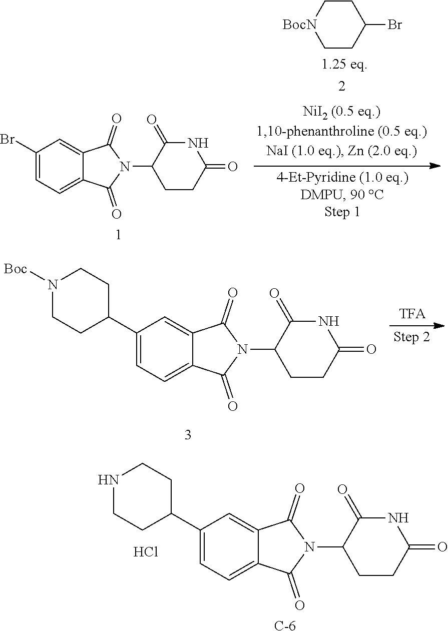

Step 1. Preparation of tert-butyl 4-[2-(2,6-dioxo-3-piperidyl)-3-oxo-isoindolin-5-yl]piperidine-1-carboxylate (3). In a reaction vial was introduced tert-butyl 4-bromopiperidine-1-carboxylate 2 (48.97 mg, 0.1900 mmol), 5-bromo-2-(2,6-dioxo-3-piperidyl)isoindoline-1,3-dione 1 (50 mg, 0.1500 mmol), nickel(II) iodide (23.17 mg, 0.0700 mmol), 1,10-phenanthroline (13.36 mg, 0.0700 mmol) and sodium iodide (22.25 mg, 0.1500 mmol), then DMPU (0.65 mL, 0.1500 mmol), followed by zinc (19.39 mg, 0.3000 mmol) and finally 4-ethylpyridine (16.87 uL, 0.1500 mmol). The mixture was stirred at 90° C. overnight. Upon completion, the mixture was purified by reverse phase chromatography using a gradient of acetonitrile and 0.1% FA water (5% to 100% over 10 CV). The pure fractions were combined and evaporated under reduced pressure to give 15.7 mg of compound 2 as a light yellow solid.

LCMS method 1: purity: 99.9% [M+Na]+: 464.2; [M-t-Bu]+: 386.2.

Step 2. Preparation of 2-(2,6-dioxo-3-piperidyl)-5-(4-piperidyl)isoindoline-1,3-dione (C-6). To a solution of tert-butyl 4-[2-(2,6-dioxo-3-piperidyl)-1,3-dioxo-isoindolin-5-yl]piperidine-1-carboxylate 3 (75 mg, 0.1700 mmol) in DCM (1.5856 mL) was added TFA (0.43 mL, 1.7 mmol). The mixture was stirred at rt for 2 h, at which point no starting material was observed. Toluene and MeCN were added to the reaction mixture before evaporating the solvent under reduced pressure. The crude material was co-evaporated 3 times with MeCN to afford product C-6 (95 mg, 0.2063 mmol, 99% yield) as an orange solid.

LCMS method 1: 98.9% purity at 215 nm, [M+H]+=342.2.

### Example I-10. Synthesis of Common Intermediate C-7

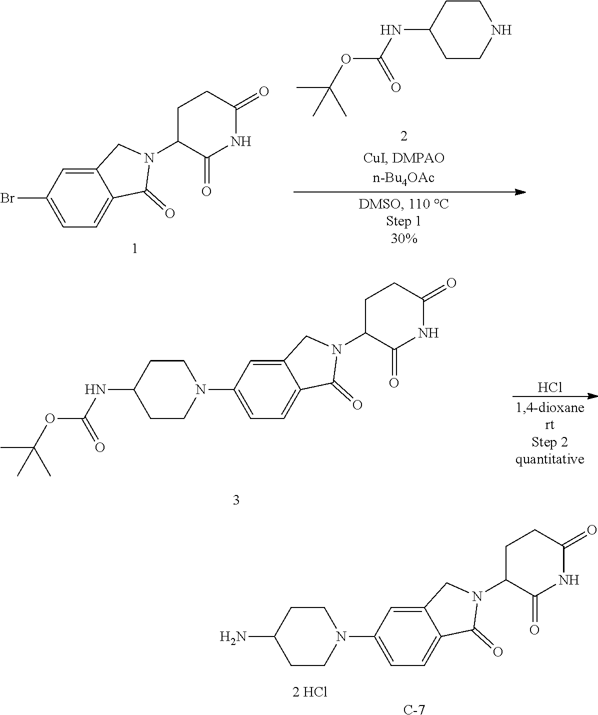

Step 1. Preparation of tert-Butyl N-[1-[2-(2,6-dioxo-3-piperidyl)-1-oxo-isoindolin-5-yl]-4-piperidyl]carbamate (3). In a flame-dried microwave vial, under nitrogen, were introduced 100 mg of freshly activated 3 Å molecular sieves and dry DMSO (9.3 mL, 0.1 M), and the mixture was degassed by sparging with nitrogen for 10 minutes. Reagents were then added in the following order: CuI (88.4 mg, 0.46 mmol, 0.5 eq.), DMPAO (179.4 mg, 0.93 mmol, 1.0 eq.), n-Bu₄OAc (840 mg, 2.79 mmol, 3.0 eq.), 1 (300 mg, 0.93 mmol, 1.0 eq.) and 2 (223 mg, 1.11 mmol, 1.2 eq.). Then the heterogeneous mixture was further degassed by sparging with nitrogen for 10 minutes. The mixture was stirred and heated at 110° C. for 16 h. Complete conversion of 1 was observed by LCMS (method 1). Ethyl acetate and water were added, and the phases were separated. The aqueous phase was extracted 3 times with ethyl acetate. The combined organic phases were washed once with brine, dried over magnesium sulfate, filtered and concentrated. The residue was purified by reverse phase flash chromatography (50 g C18 RediSep Rf Gold column, liquid deposit (DMSO), elution: 5% MeCN/0.1% HCOOH over 4 CV, then 5% to 60% MeCN/0.1% HCOOH over 15 CV). Fractions were combined and concentrated to give 3 (127 mg, 30% yield) as a light orange solid.

LCMS method 1: retention time: 1.555 min, 99.9% purity at 215 nm, [M+H]+=443.2.

¹H NMR (400 MHz, DMSO-d6) δ ppm 1.34-1.49 (m, 11H), 1.75-1.83 (m, 2H), 1.91-2.01 (m, 1H), 2.30-2.43 (m, 1H), 2.53-2.62 (m, 1H), 2.83-2.97 (m, 3H), 3.41-3.55 (m, 1H), 3.82 (br d, J=13.2 Hz, 2H), 4.19 (d, J=16.0 Hz, 1H), 4.31 (d, J=16.0 Hz, 1H), 5.04 (dd, J=13.2, 5.1 Hz, 1H), 6.85 (br. d, J=7.1 Hz, 1H), 7.01-7.09 (m, 2H), 7.49 (d, J=8.3 Hz, 1H), 10.94 (s, 1H).

Step 2. Preparation of 3-[5-(4-Amino-1-piperidyl)-1-oxo-isoindolin-2-yl]piperidine-2,6-dione dihydrochloric acid salt (C-7). Under nitrogen, a mixture of 3 (127 mg, 0.30 mmol, 1.0 eq.) and 4.0 M HCl in 1,4-dioxane (9.43 mL, 120 eq.) was stirred at room temperature for 1.25 h. Complete conversion of 3 was observed by LCMS (method 1). The mixture was concentrated under reduced pressure and co-evaporated 3 times with acetonitrile to give C-7 (156 mg, quantitative yield) as a light orange solid.

LCMS method 1: retention time: 0.751 min, 86.8% purity at 215 nm, [M+H]+=343.2.

¹H NMR (400 MHz, DMSO-d6) δ ppm 1.51-1.64 (m, 2H), 1.90-2.03 (m, 3H), 2.30-2.41 (m, 1H), 2.55-2.64 (m, 1H), 2.84-3.00 (m, 3H), 3.22-3.34 (m, 1H), 3.44-3.74 (m, 1H), 3.94 (br. d, J=13.4 Hz, 2H), 4.21 (d, J=16.0 Hz, 1H), 4.32 (d, J=16.0 Hz, 1H), 5.05 (dd, J=13.4, 5.1 Hz, 1H), 7.04-7.15 (m, 2H), 7.53 (d, J=8.6 Hz, 1H), 7.94-8.07 (m, 3H), 10.94 (s, 1H).

### Example I-11. Synthesis of Common Intermediate C-8

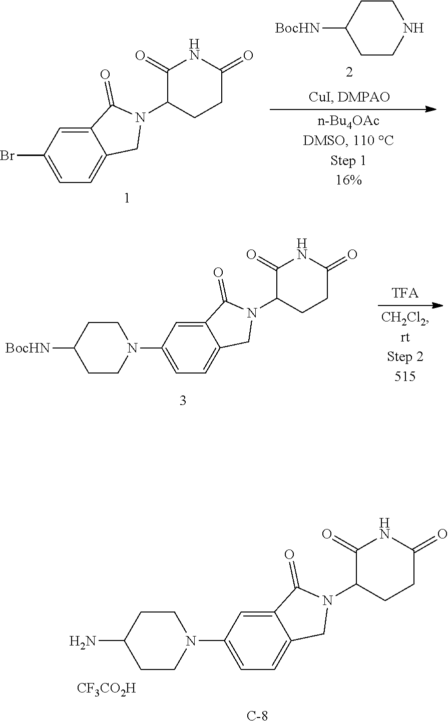

Step 1. Preparation of tert-Butyl N-[1-[2-(2,6-dioxo-3-piperidyl)-3-oxo-isoindolin-5-yl]-4-piperidyl]carbamate (3). To a microwave vial was added DMSO (4.2 mL) and molecular sieves (4 Å, beads), and the solvent was stirred at room temperature with nitrogen bubbling through it for 30 minutes. Next, 3-(6-bromo-1-oxo-isoindolin-2-yl)piperidine-2,6-dione 1 (150 mg, 0.46 mmol), tert-butyl N-(4-piperidyl)carbamate 2 (111.6 mg, 0.56 mmol), DMPAO (89.7 mg, 0.46 mmol), CuI (0.46 mL of a 0.5 M solution in DMSO, 0.23 mmol), and tetrabutylammonium acetate (419.9 mg, 1.39 mmol) were added. Nitrogen was bubbled through the solution for 15 minutes, and the mixture was sonicated. The microwave vial was capped, and the mixture was stirred at 110° C. for 24 hours. After cooling to room temperature, water and EtOAc were added. The aqueous phase was extracted 3 times with EtOAc. The organic phases were combined, washed with water and brine, dried over Na₂SO₄, filtered, and evaporated under reduced pressure. The oily solid was then solubilized in DMSO, and the solution was loaded on a 50 g RediSep Rf Gold C18 chromatography column. Elution was carried out with MeCN/0.1% aqueous formic acid (5% for 4 CVs, then 5 to 60% over 15 CVs). The desired fractions were combined and concentrated to give 3 (43.5 mg, 16% yield) as an orange solid.

LCMS method 1: 76.1% purity at 215 nm, [M+H]+=443.2.

Step 2. Preparation of 3-[6-(4-Amino-1-piperidyl)-1-oxo-isoindolin-2-yl]piperidine-2,6-dione trifluoroacetic acid salt (C-8). To a solution of tert-butyl N-[1-[2-(2,6-dioxo-3-piperidyl)-3-oxo-isoindolin-5-yl]-4-piperidyl]carbamate 3 (99.3 mg, 0.22 mmol) in CH₂Cl₂ (1.6 mL) was added trifluoroacetic acid (0.45 mL, 1.76 mmol). The resulting mixture was stirred at room temperature for 2 hours. MeCN was added and the solvent was removed under reduced pressure. The crude mixture was co-evaporated with MeCN 3 times. The residue was dissolved in water and the solution was loaded on a 15.5 g RediSep Rf Gold C18 chromatography column. Elution was carried out using MeCN/0.05 M aqueous TFA (5% for 5 CVs, then 5 to 20% over 10 CVs). The desired fractions were combined and concentrated to give C-8 (52 mg, 51% yield) as an orange solid.

LCMS method 1: 99.9% purity at 215 nm, [M−TFA+H]=343.2.

¹H NMR (400 MHz, DMSO-d6) δ ppm 1.53-1.66 (m, 2H), 1.89-2.02 (m, 3H), 2.31-2.45 (m, 1H), 2.56-2.69 (m, 1H), 2.79-2.96 (m, 3H), 3.18-3.30 (m, 1H), 3.78-3.86 (m, 2H), 4.15-4.25 (m, 1H), 4.29-4.38 (m, 1H), 5.07 (dd, J=13.3, 5.0 Hz, 1H), 7.20 (d, J=2.0 Hz, 1H), 7.28 (dd, J=8.6, 2.2 Hz, 1H), 7.44 (d, J=8.3 Hz, 1H), 7.87 (br s, 3H), 10.96 (s, 1H).

### Example I-12. Synthesis of Common Intermediate C-9

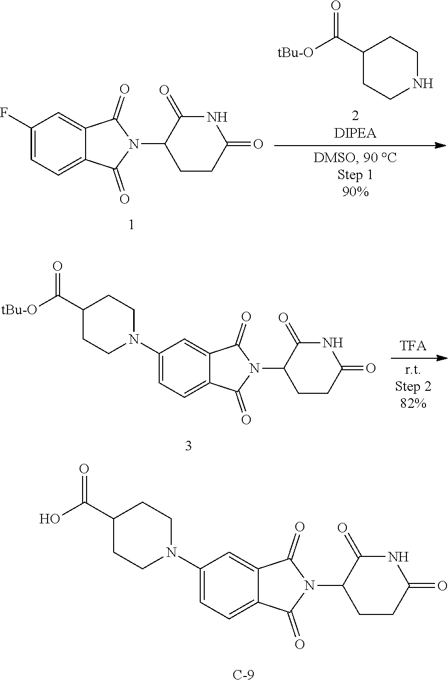

Step 1. Preparation of tert-Butyl 1-[2-2,6-dioxo-3-piperidyl-1,3-dioxo-isoindolin-5-yl]piperidine-4-carboxylate (3). To a solution of 2-(2,6-dioxo-3-piperidyl)-5-fluoro-isoindoline-1,3-dione 1 (200 mg, 0.720 mmol, 1 eq.) and tert-butyl piperidine-4-carboxylate 2 (174.4 mg, 0.940 mmol, 1.3 eq.) in anhydrous DMSO (3.6 mL) was added DIPEA (252 uL, 1.45 mmol, 2 eq.). The resulting mixture was stirred at 90° C. After 18 h, LCMS showed full conversion. The reaction mixture was diluted with water and the aqueous phase was extracted 3 times with EtOAc. The organics were washed 5 times with water, dried over Na₂SO₄ and concentrated to dryness. The residue was purified by reverse phase flash chromatography (50 g C18 RediSep Rf Gold column, liquid deposit (DMSO), elution: 5% MeCN/0.1% HCOOH over 3 CV, then 5 to 40% MeCN/0.1% HCOOH over 12 CV, then 40% MeCN/0.1% HCOOH over 3 CV, then 40 to 55% MeCN/0.1% HCOOH over 4 CV, then 55% MeCN/0.1% HCOOH over 7 CV). Fractions were combined and concentrated to give 3 (297 mg, 90% yield) as a yellow solid.

LCMS method 1: 99.9% purity at 215 nm, [M+H]+=442.2.

¹H NMR (400 MHz, DMSO-d6) δ ppm 1.40 (s, 9H), 1.49-1.63 (m, 2H), 1.82-1.91 (m, 2H), 1.97-2.04 (m, 1H), 2.52-2.63 (m, 3H), 2.81-2.95 (m, 1H), 3.08 (br t, J=11.2 Hz, 2H), 3.96 (br d, J=13.2 Hz, 2H), 5.06 (dd, J=13.0, 5.4 Hz, 1H), 7.24 (dd, J=8.6, 2.0 Hz, 1H), 7.32 (d, J=1.7 Hz, 1H), 7.66 (d, J=8.6 Hz, 1H), 11.07 (s, 1H).

Step 2. Preparation of 1-[2-(2,6-Dioxo-3-piperidyl)-1,3-dioxo-isoindolin-5-yl]piperidine-4-carboxylic acid (C-9). A solution of tert-butyl 1-[2-(2,6-dioxo-3-piperidyl)-1,3-dioxo-isoindolin-5-yl]piperidine-4-carboxylate 3 (297 mg, 0.650 mmol, 1 eq.) in TFA (1.51 mL, 19.52 mmol, 30 eq.) was stirred at room temperature. After 1 h, HPLC showed full conversion. TFA was evaporated under reduced pressure and the residue was purified by reverse phase flash chromatography (50 g C18 RediSep Rf Gold column, liquid deposit (DMSO), elution: 5% MeOH/0.1% HCOOH over 3 CV, then 5 to 40% MeOH/0.1% HCOOH over 11 CV, then 40% MeCN/0.1% HCOOH over 4 CV, then 40 to 60% MeCN/0.1% HCOOH over 9 CV). Fractions were combined and concentrated to give C-9 (206 mg, 82% yield) as a yellow solid.

LCMS method 1: 99.9% purity at 215 nm, [M+H]+=386.2.

¹H NMR (400 MHz, DMSO-d6) δ ppm 1.53-1.67 (m, 2H), 1.84-1.95 (m, 2H), 1.97-2.05 (m, 1H), 2.52-2.63 (m, 3H), 2.81-2.94 (m, 1H), 3.09 (br t, J=11.4 Hz, 2H), 3.97 (br d, J=13.2 Hz, 2H), 5.06 (dd, J=12.8, 5.5 Hz, 1H), 7.25 (dd, J=8.6, 2.0 Hz, 1H), 7.33 (d, J=1.5 Hz, 1H), 7.66 (d, J=8.6 Hz, 1H), 11.07 (s, 1H), 12.29 (dd, J=6.0, 2.1 Hz, 1H).

### Example I-13. Synthesis of Common Intermediate C-10

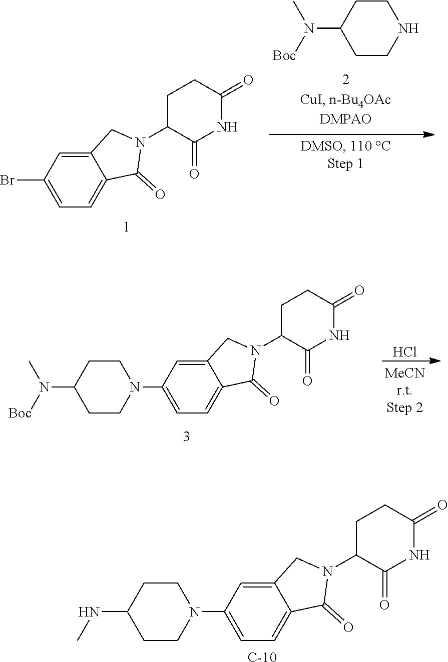

Step 1. Preparation of tert-Butyl N-[1-[2-(2,6-dioxo-3-piperidyl)-1-oxo-isoindolin-5-yl]-4-piperidyl]-N-methyl-carbamate (3). In a flame-dried microwave vial, under nitrogen, were introduced 20 mg of freshly activated 3 Å molecular sieves and dry DMSO (4.6 mL, 0.1 M) and the mixture was degassed by sparging with nitrogen for 20 minutes. Reagents were then added, in the following order: CuI (44.2 mg, 0.23 mmol, 0.5 eq.), 3-(5-bromo-1-oxo-isoindolin-2-yl)piperidine-2,6-dione 1 (150. mg, 0.46 mmol, 1.0 eq.), tert-butyl N-methyl-N-(4-piperidyl)carbamate 2 (119.37 mg, 0.56 mmol, 1.2 eq.), DMPAO (89.68 mg, 0.46 mmol, 1.0 eq.), and tetrabutylammonium acetate (419.88 mg, 1.39 mmol, 3.0 eq.). Next, the heterogeneous mixture was further degassed by sparging with nitrogen for 10 minutes. The mixture was stirred and heated at 110° C. for 16 hours. Complete conversion of 1 was observed by LCMS (method 3). Ethyl acetate and water were added, then the phases were separated. The aqueous phase was extracted 3 times with ethyl acetate. The combined organic phases were washed once with brine, dried over magnesium sulfate, filtered and concentrated. The residue was purified by normal phase flash chromatography (40 g silica column, elution: 0 to 10% CH₂Cl₂/MeOH over 15 CV, product exited at 6.5% MeOH). Fractions were combined and concentrated to give tert-butyl N-[1-[2-(2,6-dioxo-3-piperidyl)-1-oxo-isoindolin-5-yl]-4-piperidyl]-N-methyl-carbamate 3 (127 mg, 47% yield) as a light orange solid.

LCMS method 2: retention time: 1.788 min, 79.1% purity at 215 nm, [M+H]+=457.2.

¹H NMR (400 MHz, CHLOROFORM-d) δ ppm 0.77-0.90 (m, 1H), 1.48 (s, 9H), 1.72-1.84 (m, 4H), 2.19-2.25 (m, 1H), 2.34 (br dd, J=13.0, 5.1 Hz, 1H), 2.75 (s, 3H), 2.82-3.00 (m, 4H), 3.91 (br d, J=13.2 Hz, 2H), 4.20-4.30 (m, 1H), 4.37-4.45 (m, 1H), 5.20 (dd, J=13.3, 5.0 Hz, 1H), 6.91 (br s, 1H), 6.97-7.05 (m, 1H), 7.74 (d, J=8.6 Hz, 1H), 7.96 (br s, 1H).

Step 2. Preparation of 3-[5-[4-(Methylamino)-1-piperidyl]-1-oxo-isoindolin-2-yl]piperidine-2,6-dione hydrochloride (C-10). Under nitrogen, a mixture of tert-butyl N-[1-[2-(2,6-dioxo-3-piperidyl)-1-oxo-isoindolin-5-yl]-4-piperidyl]-N-methyl-carbamate 3 (125. mg, 0.27 mmol, 1.0 eq.) and 4 M HCl in 1,4-dioxane (1.37 mL, 5.48 mmol, 20.0 eq.) in MeCN (2 mL, 0.1 M) was stirred at room temperature for 1 hour. Complete conversion of 3 was observed by LCMS (method 3). The mixture was concentrated under reduced pressure and co-evaporated 3 times with acetonitrile to give 3-[5-[4-(methylamino)-1-piperidyl]-1-oxo-isoindolin-2-yl]piperidine-2,6-dione C-10 (124 mg, quantitative yield) as a light orange solid.

LCMS method 2: retention time: 1.215 min, 85.6% purity at 215 nm, [M−HCl+H]+=357.2.

¹H NMR (400 MHz, DMSO-d6) δ ppm 1.50-1.63 (m, 2H), 1.91-2.07 (m, 3H), 2.31-2.40 (m, 1H), 2.53-2.63 (m, 4H), 2.82-2.97 (m, 3H), 3.16-3.28 (m, 1H), 3.95-4.03 (m, 2H), 4.17-4.24 (m, 1H), 4.29-4.38 (m, 1H), 5.05 (dd, J=13.3, 5.0 Hz, 1H), 7.04-7.15 (m, 2H), 7.53 (d, J=8.3 Hz, 1H), 8.67 (br s, 2H), 10.94 (s, 1H).

### Example I-14. Synthesis of Common Intermediate C-11

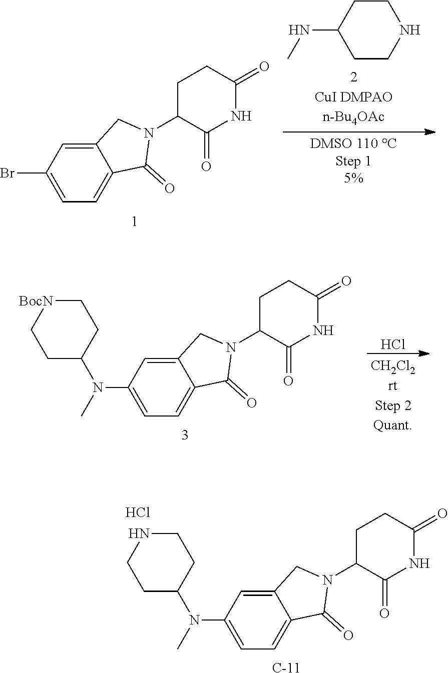

Step 1. Preparation of tert-Butyl 4-[[2-(2,6-Dioxo-3-piperidyl)-1-oxo-isoindolin-5-yl]-methyl-amino]piperidine-1-carboxylate (3). To a sealed tube were added molecular sieves (3 Å beads) previously activated under high vacuum during 1.5 h and heated using a torch, every 10 min. To the tube were then added 3-(5-bromo-1-oxo-isoindolin-2-yl)piperidine-2,6-dione 1 (150 mg, 0.46 mmol, 1 eq.), tert-butyl 4-(methylamino)piperidine-1-carboxylate 2 (119.37 mg, 0.56 mmol, 1.2 eq.), DMPAO (89.68 mg, 0.46 mmol, 1 eq.), CuI (44.2 mg, 0.23 mmol, 0.5 eq.) and tetrabutylammonium acetate (419.88 mg, 1.39 mmol, 3 eq.) in dry DMSO (4.6 mL, 0.1 M). Nitrogen was bubbled through the solution for 15 minutes, the tube was sealed and the resulting mixture was stirred at 110° C. overnight. LCMS after overnight stirring showed 16% conversion into compound 3. Water and EtOAc were added to the reaction, and the aqueous phase was extracted 3× with EtOAc. The organic phases were combined, washed 3× with water and 1× with brine, dried over Na₂SO₄, filtered and concentrated to dryness. The residue was then purified by normal phase FC (40 g column, solid deposit on silica, 0 to 10% DCM/MeOH over 15 CV). Fractions were combined and concentrated to give 3 (12.7 mg, 5% yield) as a yellow oil as a formic acid salt.

LCMS method 2: 86.6% purity at 215 nm, [M-t-Bu+H]+=401.1.

¹H NMR (400 MHz, DMSO-d6) δ ppm 1.40-1.43 (m, 9H), 1.56-1.65 (m, 4H), 2.54 (br s, 2H), 2.77-2.80 (m, 3H), 2.84-2.95 (m, 3H), 3.91-4.11 (m, 4H), 4.15-4.23 (m, 1H), 4.27-4.35 (m, 1H), 5.03 (dd, J=13.3, 5.0 Hz, 1H), 6.90-6.97 (m, 2H), 7.47-7.51 (m, 1H), 8.18 (s, 1H), 10.93 (s, 1H).

Step 2. Preparation of N-[3-(Difluoromethyl)-1-[4-[2-[4-[2-(2,6-dioxo-3-piperidyl)-1-oxo-isoindolin-5-yl]piperazin-1-yl]ethyl]cyclohexyl]pyrazol-4-yl]-5-[(3R,5R)-3-amino-5-fluoro-1-piperidyl]pyrazolo[1,5-a]pyrimidine-3-carboxamide hydrochloride (C-11). To a solution of tert-butyl 4-[[2-(2,6-Dioxo-3-piperidyl)-3-oxo-isoindolin-5-yl]-methyl-amino]piperidine-1-carboxylate 3 (62.8 mg, 0.11 mmol, 1 equiv.) in CH₂Cl₂ (1 mL, 0.11 M) was added 4 M HCl in 1,4-dioxane (0.82 mL, 3.26 mmol, 30 equiv.) and the reaction was stirred at rt. LCMS after 1 h. showed complete conversion into compound C-11. The solvent was evaporated, and the mixture was purified by reverse phase FC (50 g C18 RediSep Rf Gold column, liquid deposit (H₂O), elution: 5% MeCN/H₂O+0.02 M HCl over 4 CV, then 5 to 80% MeCN/H₂O+0.02 M HCl over 20 CV). Fractions were combined and concentrated to give C-11 (48.3 mg, quantitative yield) as a white solid as a hydrochloric salt.

LCMS method 3: 99.9% purity at 215 nm, [M−HCl+H]=357.2.

¹H NMR (400 MHz, DMSO-d6) δ ppm 1.73-1.82 (m, 2H), 1.92-2.06 (m, 3H), 2.33-2.43 (m, 1H), 2.55-2.62 (m, 1H), 2.82 (s, 3H), 2.85-2.95 (m, 1H), 3.00-3.10 (m, 2H), 3.35 (br d, J=13.2 Hz, 2H), 4.13-4.23 (m, 2H), 4.32 (br d, J=16.6 Hz, 1H), 5.04 (dd, J=13.0, 4.9 Hz, 1H), 6.96-7.01 (m, 2H), 7.49-7.54 (m, 1H), 8.68-8.96 (m, 2H), 10.93 (s, 1H).

### Example I-15. Synthesis of Common Intermediate C-12

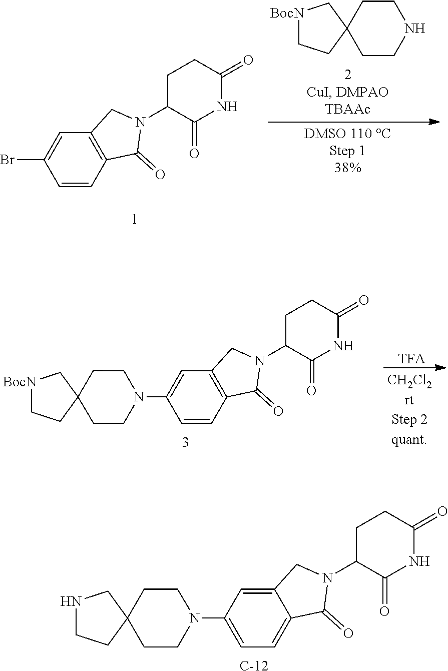

Step 1. Preparation of tert-butyl 8-[2-(2,6-dioxo-3-piperidyl)-1-oxo-isoindolin-5-yl]-2,8-diazaspiro[4.5]decane-2-carboxylate (3). To a flame-dried tube were added DMSO (8.2 mL) and 3 Å molecular sieves. The solvent was sparged with nitrogen for 20 min, then tert-butyl 2,8-diazaspiro[4.5]decane-2-carboxylate 2 (357 mg, 1.49 mmol, 1.2 eq.), 3-(5-bromo-1-oxo-isoindolin-2-yl)piperidine-2,6-dione 1 (400 mg, 1.24 mmol, 1.0 eq.), CuI (118 mg, 0.62 mmol, 0.5 eq.), DMPAO (239 mg, 1.24 mmol, 1.0 eq.), and tetrabutylammonium acetate (1.12 g, 3.71 mmol, 3.0 eq.) were added. Nitrogen was sparged through the reaction mixture once again for 15 min. The tube was sealed and stirred at 110° C. for 16 h. Upon completion, water and EtOAc were added. The aqueous phase was extracted with EtOAc (3×). The organic phases were combined, washed with water (3×), brine (lx), dried over Na₂SO₄, and concentrated under reduced pressure. The residue was purified by reverse phase chromatography (C18 column, MeCN/0.1% HCOOH 5:95, then 5:95 to 50:50) to give the title compound 3 as a pink solid (225 mg, 38%).

LCMS Method 1: 99.9% purity at 215 nm, [M+H]+=483.2.

¹H NMR (400 MHz, DMSO-d6) δ ppm 1.40 (s, 9H), 1.52-1.63 (m, 4H), 1.70-1.79 (m, 2H), 1.89-2.01 (m, 1H), 2.28-2.44 (m, 1H), 2.54-2.63 (m, 1H), 2.82-2.97 (m, 1H), 3.13 (s, 2H), 4.15-4.36 (m, 2H), 5.04 (dd, J=13.3, 5.0 Hz, 1H), 6.97-7.13 (m, 2H), 7.50 (d, J=8.6 Hz, 1H), 10.94 (s, 1H). Note: Six protons signals were partially obscured by the water peak.

Step 2. Preparation of 3-[5-(2,8-diazaspiro[4.5]decan-8-yl)-1-oxo-isoindolin-2-yl]piperidine-2,6-dione (C-12). To a solution of tert-butyl 8-[2-(2,6-dioxo-3-piperidyl)-1-oxo-isoindolin-5-yl]-2,8-diazaspiro[4.5]decane-2-carboxylate 3 (225 mg, 0.47 mmol, 1.0 eq.) in CH₂Cl₂ (4.7 mL) was added TFA (2.9 mL, 11.66 mmol, 25 eq.). The reaction mixture was stirred at room temperature for 1 h. Upon completion, the mixture was concentrated to dryness and residual TFA was chased with toluene (2×) and MeCN (2×) to give the bis-TFA salt of the title compound C-12 as a light-orange semi-solid (320 mg, quantitative). The material was used without purification in the next step.

LCMS Method 1: 99.9% purity at 215 nm, [M+H]=383.2.

### Example I-16. Synthesis of Common Intermediate C-13

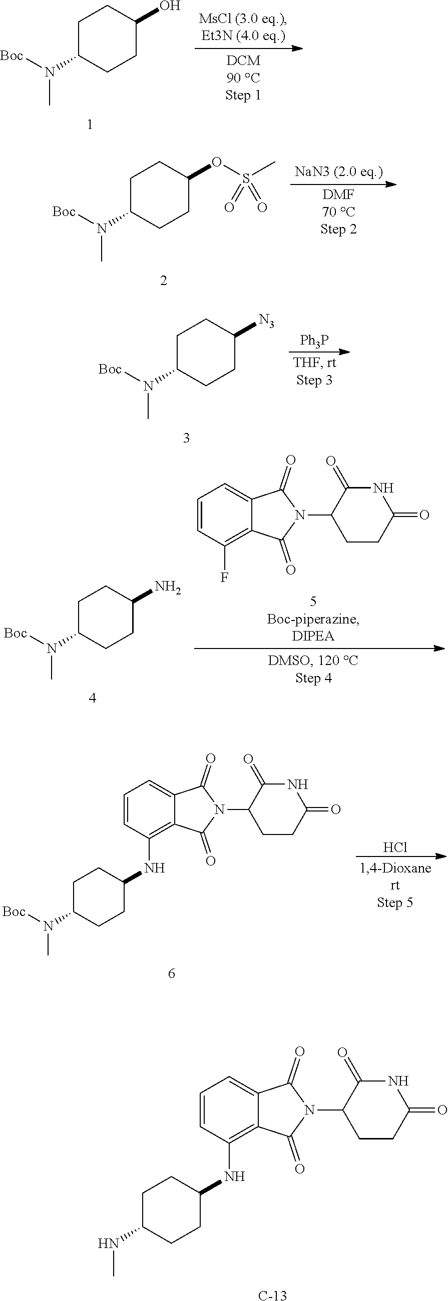

Step 1. Preparation of [4-[tert-butoxycarbonyl(methyl)amino]cyclohexyl]methanesulfonate (2). To a solution of tert-butyl N-(4-hydroxycyclohexyl)-N-methyl-carbamate 1 (220. mg, 0.9600 mmol) in DCM (9.5936 mL) at 0° C. were added triethylamine (0.53 mL, 3.84 mmol) and MsCl (0.22 mL, 2.88 mmol) dropwise. The mixture was stirred at 0° C. for 30 min and then the cold bath was removed. The reaction mixture was stirred at room temperature overnight. TLC analysis showed the formation of one product (SiO₂, 1:1 heptane/EtOAc, Rf SM=0.2, Rf DP=0.3). The reaction was added to 50 mL of sat. NaHCO₃ solution and the organic phase was separated. The aqueous phase was extracted with DCM (4×20 mL), then the combined organic layers were washed with brine (1×100 mL) and evaporated to afford compound 2 (322 mg, 98%) as a light-yellow solid.

Step 2. Preparation of tert-butyl N-(4-azidocyclohexyl)-N-methyl-carbamate (3). The [4-[tert-butoxycarbonyl(methyl)amino]cyclohexyl]methanesulfonate 2 (690 mg, 2.02 mmol) was dissolved in DMF (6.7337 mL), then sodium azide (262.65 mg, 4.04 mmol) was added. The resulting suspension was stirred at 70° C. After this, TLC analysis showed the formation of a new product (SiO₂, 1:1 EtOAc/heptanes, Rf SM=0.3, Rf DP=0.6). The reaction was added to 100 mL of water and extracted with EtOAc (3×20 mL). The combined organic layers were washed with brine (1×50 mL) and dried to give product 3 (481 mg, 70%) as a light-yellow oil, which was used without purification in the next step.

Step 3. Preparation of tert-butyl ((1r,4r)-4-aminocyclohexyl)(methyl)carbamate (4). The tert-butyl N-(4-azidocyclohexyl)-N-methyl-carbamate 3 (117. mg, 0.4600 mmol) was dissolved in THE (5 mL), then triphenylphosphine (241.32 mg, 0.9200 mmol) was added. The resulting solution was stirred at room temperature for 2 h. Afterwards, water (1 mL) was added and the reaction was left stirring overnight. The solvent was removed in vacuo and the residue was purified by reverse phase chromatography (5 to 100% MeOH in buffer solution at pH=10), collected and evaporated before repurification to remove triphenylphosphinoxide (5 to 100% MeOH in 0.1% FA) to afford product 4 (75 mg, 59%) as a colorless semi-solid.

Step 4. Preparation of [4-[tert-butoxycarbonyl(methyl)amino]cyclohexyl]methanesulfonate (6). A grey suspension of tert-butyl N-(4-aminocyclohexyl)-N-methyl-carbamate 4 (92. mg, 0.4000 mmol), 2-(2,6-dioxo-3-piperidyl)-4-fluoro-isoindoline-1,3-dione 5 (122.4 mg, 0.4400 mmol) and DIPEA (0.18 mL, 1.01 mmol) in DMSO (2.7 mL) was stirred at 120° C. for 48 h. Partial conversion was observed by HPLC, but the reaction was stopped because some decomposition products were observed. After this, the resulting black solution was cooled to room temperature and directly injected in a 50 g C18 column and purified by reverse phase chromatography (5 to 100% MeOH in 0.1% FA). Fractions were combined and evaporated to afford product 6 (83 mg, 40%) as a yellow solid.

Step 5. 2-(2,6-dioxopiperidin-3-yl)-4-(((1r,4r)-4-(methylamino)cyclohexyl)amino)isoindoline-1,3-dione (C-13). The tert-butyl N-[4-[[2-(2,6-dioxo-3-piperidyl)-1,3-dioxo-isoindolin-4-yl]amino]cyclohexyl]-N-methyl-carbamate 6 (62 mg, 0.1200 mmol) was dissolved in 1,4-dioxane (1.2 mL), then a 4.0 M solution of hydrogen chloride (0.61 mL, 2.45 mmol) was added and the resulting solution was stirred at rt. After 12 h, the reaction was complete. The solvent was evaporated in vacuo and the residue was co-evaporated with ACN (2×5 mL) to afford product C-13 (62 mg, 99%) as a yellow solid, double HCl salt.

¹H NMR (400 MHz, DMSO-d6) δ ppm 1.40-1.53 (m, 4H), 2.00-2.10 (m, 6H), 2.52-2.56 (m, 4H) 2.83-2.97 (m, 2H), 3.53-3.57 (m, 2H), 5.02-5.07 (dd, 1H), 6.18 (d, 1H), 7.05 (d, 1H), 7.22 (d, 1H), 7.57-7.61 (dd, 1H), 8.75 (s, 2H), 11.09 (s, 1H).

### Final Compound Synthesis

### Final Product General Method 1

### Example S1. Synthesis of P-1

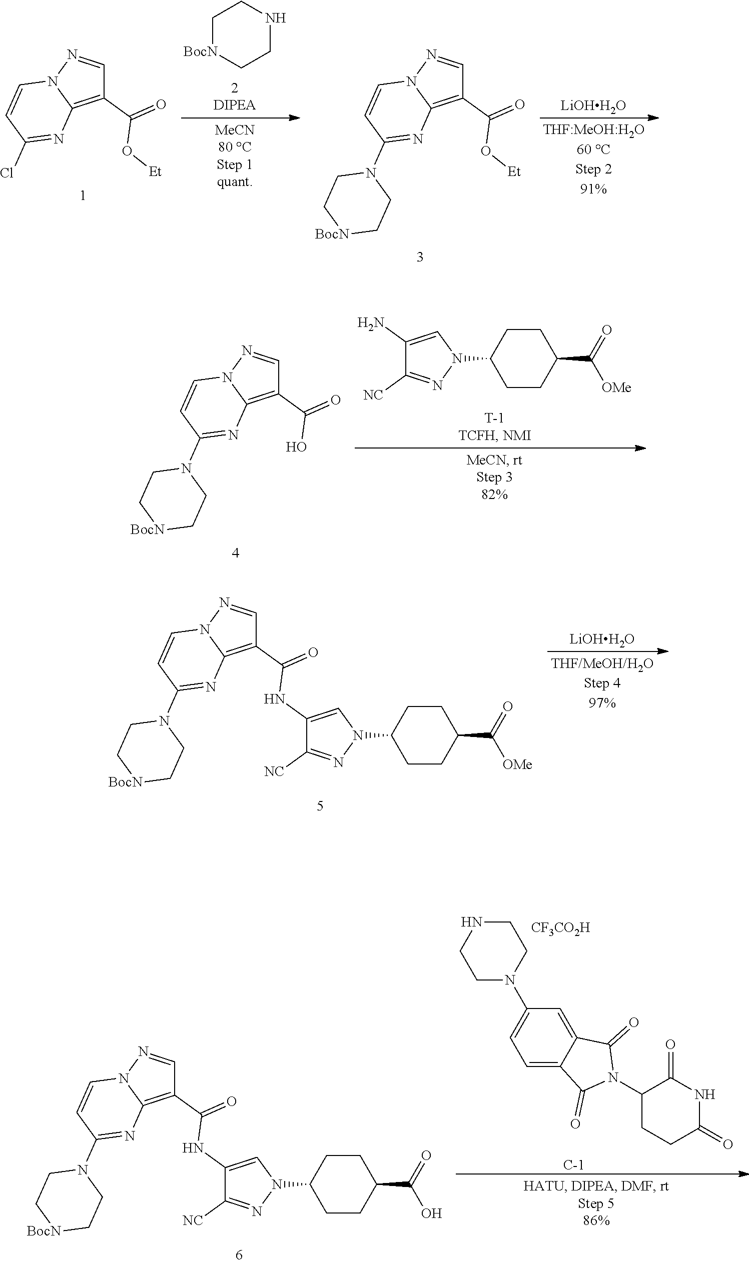

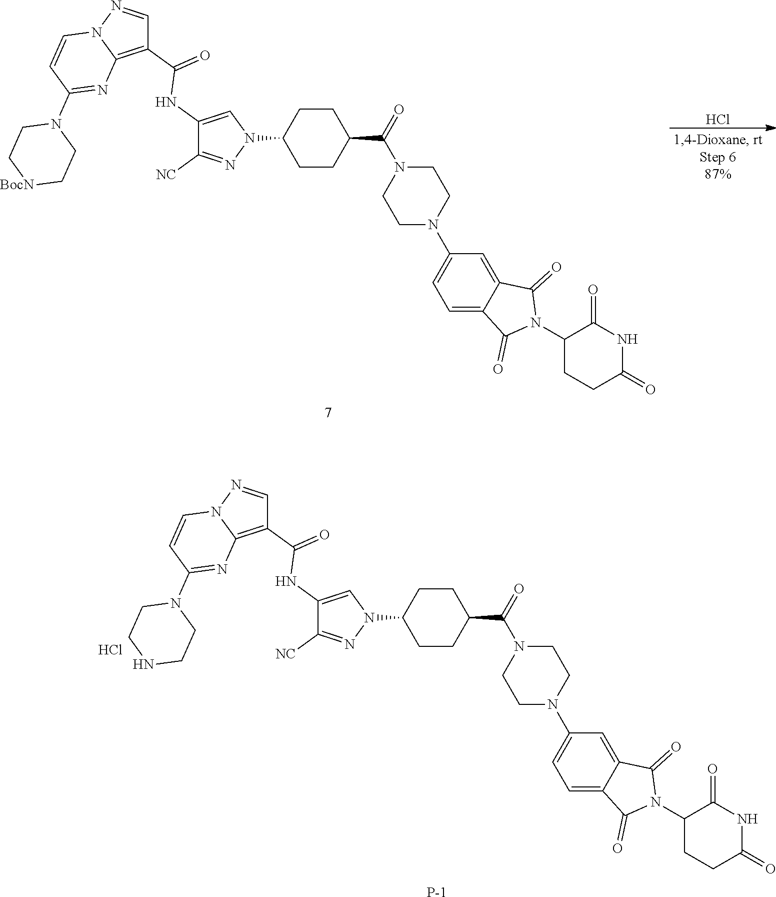

Step 1. Preparation of Ethyl 5-(4-tert-butoxycarbonylpiperazin-1-yl)pyrazolo[1,5-a]pyrimidine-3-carboxylate (3). To a solution of ethyl 5-chloropyrazolo[1,5-a]pyrimidine-3-carboxylate 1 (1.5 g, 6.65 mmol, 1.0 equiv.) and tert-butyl piperazine-1-carboxylate 2 (1.56 g, 8.38 mmol, 1.2 equiv.) in MeCN (30 mL, 0.2 M) was added DIPEA (2.9 mL, 16.60 mmol, 2.5 equiv.). The reaction mixture was stirred at 80° C. overnight. The solvent was removed under reduced pressure to provide the crude oil 3 (2.5 g, quantitative yield), which was used without purification in the next step.

LCMS Method 1: 99.9% purity at 215 nm; [M+H]+=376.2 m/z.

Step 2. Preparation of 5-(4-tert-Butoxycarbonylpiperazin-1-yl)pyrazolo[1,5-a]pyrimidine-3-carboxylic acid (4). To a solution of ethyl 5-(4-tert-butoxycarbonylpiperazin-1-yl)pyrazolo[1,5-a]pyrimidine-3-carboxylate 3 (5.0 g, 13.3 mmol, 1.0 equiv.) in THF (22.2 mL, 0.2 M) and MeOH (22.2 mL, 0.2 M) was added a solution of LiOH H₂O (5.6 g, 133 mmol, 10 equiv.) in water (22.2 mL, 0.2 M). The resulting mixture was stirred at 60° C. for 3 h. Upon completion, THE and MeOH were removed under reduced pressure. The crude mixture was diluted with water. Under vigorous agitation, the mixture was acidified with aqueous 6 M HCl until pH=3 (formation of a precipitate). The suspension was filtered on a Buchner funnel, rinsing the solid with water. The solid was co-evaporated with THE (3×) to remove water to afford 4 (4.2 g, 91% yield).

LCMS Method 1: 99.9% purity at 215 nm; [M+H]+=348.2 m/z.

¹H NMR (400 MHz, CDCl₃) δ ppm 1.50 (s, 9H), 3.60-3.66 (m, 4H), 3.73-3.83 (m, 4H), 6.48 (d, J=8.1 Hz, 1H), 8.34 (s, 1H), 8.37 (d, J=7.8 Hz, 1H).

Step 3. Preparation of tert-butyl 4-[3-[[3-cyano-1-(4-methoxycarbonylcyclohexyl)pyrazol-4-yl]carbamoyl]pyrazolo[1,5-a]pyrimidin-5-yl]piperazine-1-carboxylate (5). To a solution of 5-(4-tert-butoxycarbonylpiperazin-1-yl)pyrazolo[1,5-a]pyrimidine-3-carboxylic acid 4 (945 mg, 2.72 mmol), methyl 4-(4-amino-3-cyano-pyrazol-1-yl)cyclohexanecarboxylate T-1 (675.4 mg, 2.72 mmol) and NMI (754 μL, 9.52 mmol) in MeCN (6.8 mL) at room temperature was added TCFH (916 mg, 3.26 mmol). The resulting mixture was stirred at room temperature for 4 hours. At that point, the reaction mixture was diluted with water. The aqueous layer was extracted 3 times with EtOAc. The combined organic layers were washed with brine, dried over Na₂SO₄, and concentrated to dryness. The residue was suspended in DMSO. The solid was filtered on a Buchner funnel, rinsed with MeCN, and dried under high vacuum to give 5 (1.29 g, 82% yield) as a white solid.

LCMS method 1: 99.9% purity at 215 nm, [M+H]=578.2, [M-t-Bu+H]=522.2.

¹H NMR (400 MHz, acetone-d6) δ ppm 1.47 (s, 9H), 1.57-1.74 (m, 2H), 1.87-2.01 (m, 2H), 2.10-2.27 (m, 4H), 2.48 (br t, J=12.3 Hz, 1H), 3.65 (s, 7H), 3.94 (br s, 4H), 4.37 (br t, J=11.7 Hz, 1H), 6.91 (d, J=8.1 Hz, 1H), 8.28 (s, 1H), 8.46 (s, 1H), 8.63 (d, J=7.8 Hz, 1H), 9.68 (s, 1H).

Step 4. Preparation of (1r,4r)-4-(4-(5-(4-(tert-butoxycarbonyl)piperazin-1-yl)pyrazolo[1,5-a]pyrimidine-3-carboxamido)-3-cyano-1H-pyrazol-1-yl)cyclohexane-1-carboxylic acid (6). To a suspension of tert-butyl 4-[3-[[3-cyano-1-(4-methoxycarbonylcyclohexyl)pyrazol-4-yl]carbamoyl]pyrazolo[1,5-a]pyrimidin-5-yl]piperazine-1-carboxylate 5 (600 mg, 1.04 mmol) in methanol (1.7 mL) and THE (1.7 mL) at 0° C. was added a solution of LiOH monohydrate (174.5 mg, 4.15 mmol) in water (1.7 mL). The resulting mixture was stirred at 0° C. for 10 minutes, and then at room temperature for 1 hour. The solvents were evaporated under reduced pressure, and the residue was suspended in nanopure water and sonicated. Under vigourous agitation, aqueous 6 N HCl was added until pH 3. MeCN was then added. The resulting suspension was sonicated and then filtered on a Buchner funnel. The solid was rinsed with nanopure water and MeCN. It was then suspended in nanopure water, frozen, and lyophilized to give 6 (569 mg, 97% yield) as a light-yellow solid.

LCMS method 5.97.6% purity at 215 nm, [M+Na]=586.2, [M+H]+=564.2, [M-t-Bu+H]+=508.2.

¹H NMR (400 MHz, DMSO-d6) δ ppm 1.43 (s, 9H), 1.47-1.58 (m, 2H), 1.76-1.89 (m, 2H), 1.99-2.12 (m, 4H), 2.27-2.36 (m, 1H), 3.44-3.57 (m, 4H), 3.71-4.02 (m, 4H), 4.31-4.41 (m, 1H), 6.92 (d, J=7.8 Hz, 1H), 8.31 (s, 1H), 8.46 (s, 1H), 8.84 (d, J=8.1 Hz, 1H), 9.61 (s, 1H), 12.17 (s, 1H).

Step 5. Preparation of tert-butyl 4-[3-[[3-cyano-1-[4-[4-[2-(2,6-dioxo-3-piperidyl)-1,3-dioxo-isoindolin-5-yl]piperazine-1-carbonyl]cyclohexyl]pyrazol-4-yl]carbamoyl]pyrazolo[1,5-a]pyrimidin-5-yl]piperazine-1-carboxylate (7). To a solution of (1r,4r)-4-(4-(5-(4-(tert-butoxycarbonyl)piperazin-1-yl)pyrazolo[1,5-a]pyrimidine-3-carboxamido)-3-cyano-1H-pyrazol-1-yl)cyclohexane-1-carboxylic acid 6 (80 mg, 0.14 mmol) and 2-(2,6-dioxo-3-piperidyl)-5-piperazin-1-yl-isoindoline-1,3-dione trifluoroacetic acid salt C-1 (90.7 mg, 0.20 mmol) in dry DMF (2 mL) at room temperature was added DIPEA (0.37 mL, 2.13 mmol). After 5 minutes, HATU (70.2 mg, 0.18 mmol) was added, and the resulting mixture was stirred at room temperature for 20 hours. Then, the solution was directly loaded on a 30 g RediSep Rf Gold C18 Isco chromatography column. Elution was done with MeCN/0.1% aqueous formic acid (5% for 4 CVs, then 5 to 100% over 15 CVs, then 100% for 2 CVs). Fractions were combined and concentrated to give 7 (109 mg, 86% yield) as a yellow solid.

LCMS method 1: 99.9% purity at 215 nm, [M+H]=888.4, [M-t-Bu+H]=832.2.

¹H NMR (400 MHz, DMSO-d6) δ ppm 1.53-1.67 (m, 3H), 1.79-2.15 (m, 9H), 2.64 (s, 2H), 2.73-2.97 (m, 3H), 3.43-3.57 (m, 10H), 3.58-3.66 (m, 3H), 3.68-3.78 (m, 3H), 3.78-3.93 (m, 5H), 4.35-4.47 (m, 1H), 5.08 (dd, J=12.3, 5.0 Hz, 1H), 6.93 (d, J=7.6 Hz, 1H), 7.27 (dd, J=8.7, 1.8 Hz, 1H), 7.37 (d, J=2.2 Hz, 1H), 7.71 (d, J=8.3 Hz, 1H), 8.32 (s, 1H), 8.48 (s, 1H), 8.86 (d, J=7.8 Hz, 1H), 9.64 (s, 1H), 11.08 (s, 1H).

Step 6. Preparation of N-(3-cyano-1-((1r,4r)-4-(4-(2-(2,6-dioxopiperidin-3-yl)-1,3-dioxoisoindolin-5-yl)piperazine-1-carbonyl)cyclohexyl)-1H-pyrazol-4-yl)-5-(piperazin-1-yl)pyrazolo[1,5-a]pyrimidine-3-carboxamide hydrochloride (P-1). In a round-bottom flask, tert-butyl 4-[3-[[3-cyano-1-[4-[4-[2-(2,6-dioxo-3-piperidyl)-1,3-dioxo-isoindolin-5-yl]piperazine-1-carbonyl]cyclohexyl]pyrazol-4-yl]carbamoyl]pyrazolo[1,5-a]pyrimidin-5-yl]piperazine-1-carboxylate 7 (75 mg, 0.08 mmol) was solubilized in a 4 M HCl solution in dioxane (2.1 mL, 8.40 mmol). The solution was stirred at room temperature for 1.5 hour. At this point, the solvent was evaporated under reduced pressure. The remaining dioxane was co-evaporated with water (3 iterations). The residue was solubilized in water, frozen, and lyophilized to give P-1 (60.4 mg, 87% yield) as a yellow solid.

LCMS method 5: 99.9% purity at 215 nm, [M−HCl+Na]=810.2, [M−HCl+2H]²⁺=394.7.

¹H NMR (400 MHz, DMSO-d6) δ ppm 1.53-1.66 (m, 2H), 1.81-2.11 (m, 7H), 2.53-2.63 (m, 2H), 2.76-2.95 (m, 2H), 3.22-3.29 (m, 4H), 3.44-3.57 (m, 4H), 3.59-3.76 (m, 4H), 4.00-4.13 (m, 4H), 4.37-4.46 (m, 1H), 5.08 (dd, J=12.8, 5.5 Hz, 1H), 7.00 (d, J=8.1 Hz, 1H), 7.27 (dd, J=8.7, 1.8 Hz, 1H), 7.37 (d, J=1.5 Hz, 1H), 7.71 (d, J=8.3 Hz, 1H), 8.37 (s, 1H), 8.48 (s, 1H), 8.95 (d, J=8.1 Hz, 1H), 9.04 (br. s, 2H), 9.59 (s, 1H), 11.08 (s, 1H).

### Example S2. Synthesis of P-13

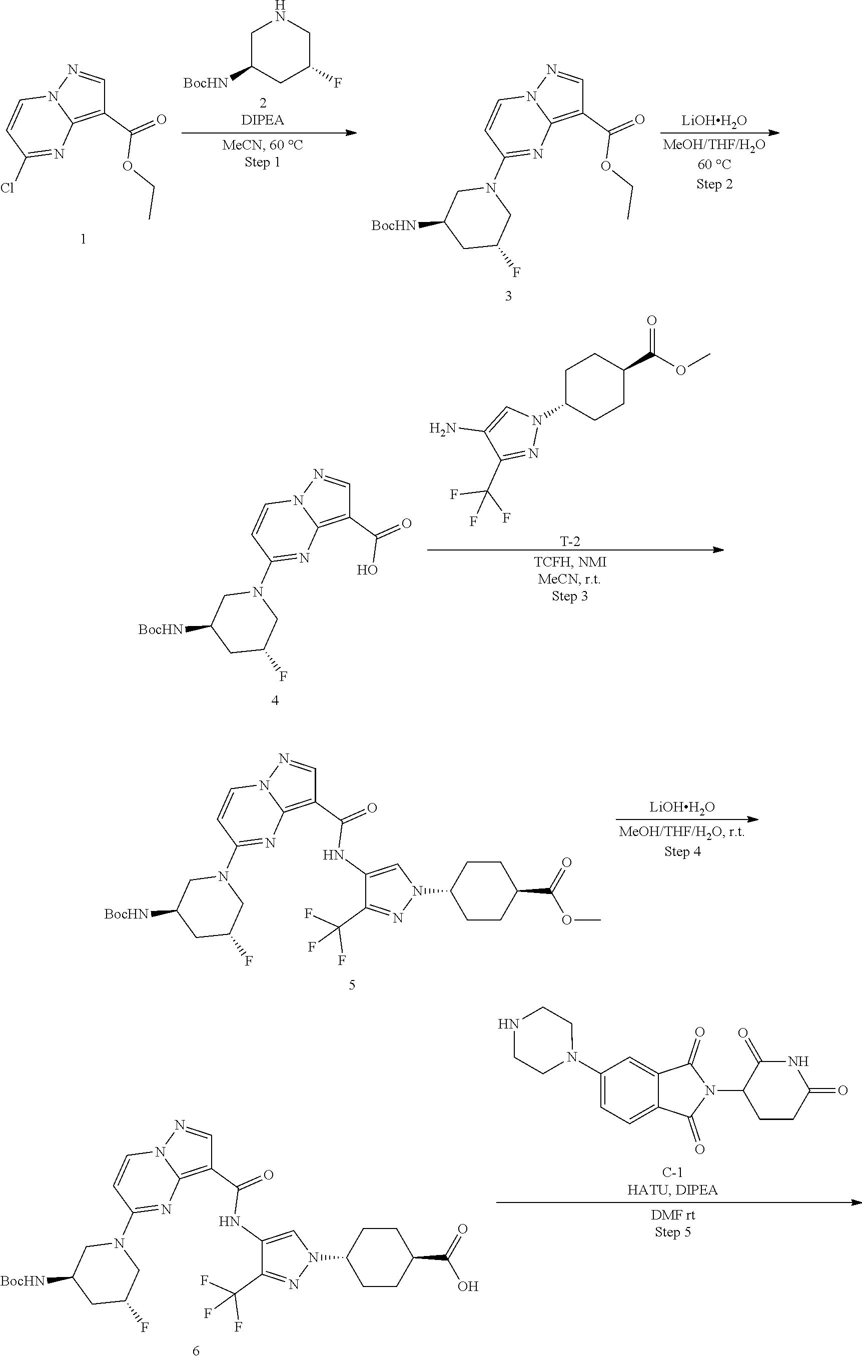

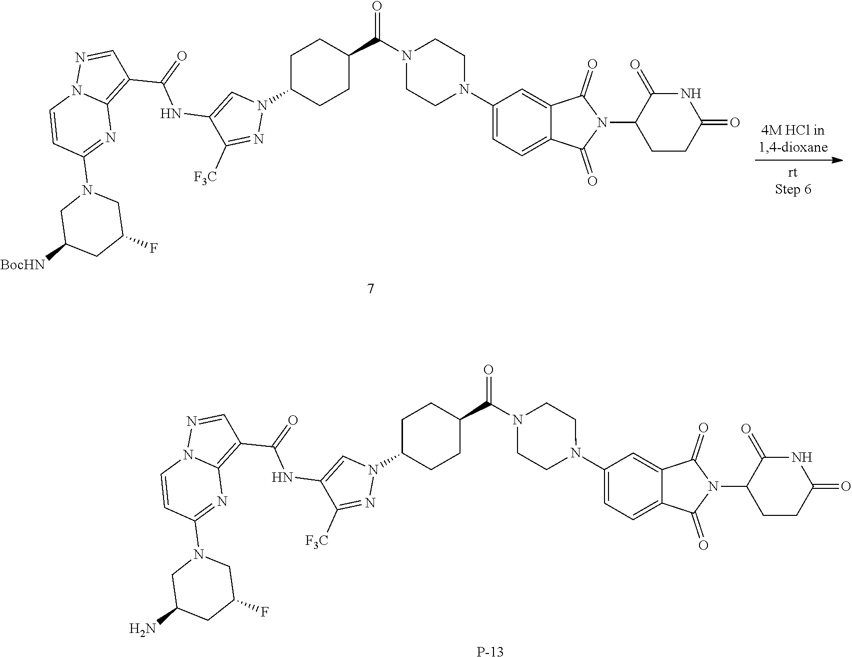

Step 1. Preparation of ethyl 5-[(3R,5R)-3-(tert-butoxycarbonylamino)-5-fluoro-1-piperidyl]pyrazolo[1,5-a]pyrimidine-3-carboxylate (3). To a solution of ethyl 5-chloropyrazolo[1,5-a]pyrimidine-3-carboxylate 1 (800 mg, 3.55 mmol, 1.0 eq.) in MeCN (17.7 mL) was added DIPEA (1.54 mL, 8.86 mmol, 2.5 eq.) and tert-butyl N-[(3R,5R)-5-fluoro-3-piperidyl]carbamate 2 (1.01 g, 4.61 mmol, 1.3 eq.). After stirring at 60° C. over the weekend, LCMS showed complete conversion into 3. The solvent was removed under reduced pressure and the residue was dried under high vacuum to give 3 (1.44 g, quantitative yield) as a white solid. The crude product was used in the next step without further purification.

LCMS method 1: retention time: 1.656 min, 99.9% purity at 215 nm, [M+H]+=408.2.

¹H NMR (400 MHz, DMSO-d6) δ ppm 1.37-1.45 (m, 9H), 1.56-1.92 (m, 1H), 2.09-2.20 (m, 1H), 2.56-2.71 (m, 1H), 2.76-3.03 (m, 1H), 3.08-3.19 (m, 1H), 3.29 (s, 1H), 3.37 (br s, 1H), 3.56-3.73 (m, 2H), 4.11-4.25 (m, 2H), 4.41-4.73 (m, 1H), 4.99 (br s, 1H), 5.11 (br s, 1H), 6.85 (br d, J=8.1 Hz, 1H), 7.10 (br d, J=7.8 Hz, 1H), 8.22 (s, 1H), 8.73 (d, J=7.8 Hz, 1H).

¹⁹F NMR (377 MHz, DMSO-d6) δ ppm-184.32 (s, 1 F).

Step 2. Preparation of 5-[(3R,5R)-3-(tert-Butoxycarbonylamino)-5-fluoro-1-piperidyl]pyrazolo[1,5-a]pyrimidine-3-carboxylic acid (4). To a solution of ethyl 5-[(3R,5R)-3-(tert-butoxycarbonylamino)-5-fluoro-1-piperidyl]pyrazolo[1,5-a]pyrimidine-3-carboxylate 3 (1.44 mg, 3.53 mmol, 1.0 eq) in THE (5.89 mL) and methanol (5.89 mL) was added a solution of LiOH H₂O (1.48 g, 35.34 mmol, 10.0 eq.) in water (5.89 mL). After stirring at 60° C. for 18 h, LCMS showed complete conversion into the desired product 4. The reaction mixture was concentrated under vacuum to remove THF/MeOH and the crude mixture was diluted with water. Under vigorous agitation, the mixture was acidified with a 6N aqueous HCl solution until pH=3 (formation of a precipitate). The suspension was filtered on a Buchner funnel and the solid was rinsed with water. The solid was dried overnight in a stove under vacuum to give 4 (1.40 g, quantitative yield) as a white solid. The crude product was used in the next step without further purification.

LCMS method 1: retention time: 1.487 min, 99.9% purity at 215 nm, [M+H]+=380.1.

¹H NMR (400 MHz, DMSO-d6) δ ppm 1.41 (s, 9H), 1.66-1.95 (m, 1H), 2.05-2.26 (m, 1H), 2.92-3.11 (m, 1H), 3.28-3.30 (m, 1H), 3.34-3.49 (m, 1H), 3.59-3.74 (m, 1H), 4.52-4.75 (m, 1H), 4.99 (br s, 1H), 5.11 (br s, 1H), 6.82 (br d, J=7.6 Hz, 1H), 7.12 (br d, J=7.8 Hz, 1H), 8.19 (s, 1H), 8.73 (d, J=7.8 Hz, 1H).

¹⁹F NMR (377 MHz, DMSO-d6) δ ppm-184.32 (s, 1 F).

Step 3. Preparation of Methyl 4-[4-[[5-[(3R,5R)-3-(tert-butoxycarbonylamino)-5-fluoro-1-piperidyl]pyrazolo[1,5-a]pyrimidine-3-carbonyl]amino]-3-(trifluoromethyl)pyrazol-1-yl]cyclohexanecarboxylate (5). To a solution of 5-[(3R,5R)-3-(tert-butoxycarbonylamino)-5-fluoro-1-piperidyl]pyrazolo[1,5-a]pyrimidine-3-carboxylic acid 4 (330 mg, 0.87 mmol, 1.0 eq.) and methyl 4-[4-amino-3-(trifluoromethyl)pyrazol-1-yl]cyclohexanecarboxylate T-2 (255 mg, 0.87 mmol, 1.0 eq.) in MeCN (4.37 mL, 0.2 M) was added NMI (208 uL, 2.62 mmol, 3.0 eq.) followed by TCFH (368 mg, 1.31 mmol, 1.5 eq.). The resulting mixture was stirred at room temperature. After stirring for 1 hour, LCMS showed complete conversion into 5. Nanopure water was added to the reaction mixture. The suspension was sonicated and filtered on a Buchner funnel. The solid was rinsed with nanopure water and dried under high vacuum for 2 h to give 5 (279 mg, 46% yield) as a white solid. The crude product was used in the next without further purification.

LCMS method 1: retention time: 1.812 min, 99.9% purity at 215 nm, [M+H]+=653.2.

¹H NMR (400 MHz, DMSO-d6) δ ppm 1.30 (br s, 9H), 1.49-1.62 (m, 3H), 1.75-1.92 (m, 4H), 2.01-2.11 (m, 5H), 3.40-3.57 (m, 1H), 3.63 (s, 3H), 4.32 (br t, J=10.9 Hz, 1H), 4.50-4.64 (m, 1H), 4.96-5.18 (m, 1H), 6.92 (br d, J=5.5 Hz, 1H), 7.09 (br s, 1H), 8.30 (s, 1H), 8.41 (br s, 1H), 8.82 (br d, J=7.8 Hz, 1H), 9.22 (br s, 1H). 2H not observed.

¹⁹F NMR (377 MHz, DMSO-d6) δ ppm-185.73-184.64 (m, 1 F), -59.01 (br s, 3 F).

Step 4. Preparation of 4-[4-[[5-[(3R,5R)-3-(tert-Butoxycarbonylamino)-5-fluoro-1-piperidyl]pyrazolo[1,5-a]pyrimidine-3-carbonyl]amino]-3-(trifluoromethyl)pyrazol-1-yl]cyclohexanecarboxylic acid (6). To a solution of methyl 4-[4-[[5-[(3R,5R)-3-(tert-butoxycarbonylamino)-5-fluoro-1-piperidyl]pyrazolo[1,5-a]pyrimidine-3-carbonyl]amino]-3-(trifluoromethyl)pyrazol-1-yl]cyclohexanecarboxylate 5 (279 mg, 0.43 mmol, 1.0 eq.) in THE (2.14 mL, 0.1 M) was added a solution of LiOH H₂O (180 mg, 4.27 mmol, 10.0 eq.) in water (2.14 mL, 0.1 M) and the resulting mixture was stirred at room temperature. After an overnight stirring, LCMS showed complete conversion into 6. The reaction mixture was concentrated under reduced pressure and the residue was suspended in nanopure water. Under vigorous agitation at 0° C., a 6N aqueous solution of HCl was added until pH=3. The solid was filtered on a Buchner funnel, rinsed with nanopure water and dried under high vacuum overnight to give 6 (232 mg, 83% yield) as a white solid. The product was used in the next step without further purification.

LCMS method 1: retention time: 1.667 min, 99.9% purity at 215 nm, [M+H]+=639.2.

¹H NMR (400 MHz, DMSO-d6) δ ppm 1.29 (br s, 9H), 1.43-1.61 (m, 3H), 1.68-1.93 (m, 4H), 1.97-2.14 (m, 5H), 2.25-2.39 (m, 1H), 3.39-3.75 (m, 2H), 4.30 (br t, J=11.7 Hz, 1H), 4.50-4.68 (m, 1H), 4.89-5.19 (m, 1H), 6.91 (br s, 1H), 7.10 (br s, 1H), 8.29 (br s, 1H), 8.41 (br s, 1H), 8.81 (br d, J=6.1 Hz, 1H), 9.21 (br s, 1H), 12.15 (br s, 1H).

¹⁹F NMR (377 MHz, DMSO-d6) δ ppm-186.50-181.97 (m, 1 F), -59.01 (br s, 3 F).

Step 5. Preparation of tert-Butyl N-[(3R,5R)-1-[3-[[1-[4-[4-[2-(2,6-dioxo-3-piperidyl)-1,3-dioxo-isoindolin-5-yl]piperazine-1-carbonyl]cyclohexyl]-3-(trifluoromethyl)pyrazol-4-yl]carbamoyl]pyrazolo[1,5-a]pyrimidin-5-yl]-5-fluoro-3-piperidyl]carbamate (7). To a solution of 4-[4-[[5-[(3R,5R)-3-(tert-butoxycarbonylamino)-5-fluoro-1-piperidyl]pyrazolo[1,5-a]pyrimidine-3-carbonyl]amino]-3-(trifluoromethyl)pyrazol-1-yl]cyclohexanecarboxylic acid 6 (75 mg, 0.12 mmol) and 2-(2,6-dioxo-3-piperidyl)-5-piperazin-1-yl-isoindoline-1,3-dione trifluoroacetate C-1 (75 mg, 0.16 mmol) in anhydrous DMF (1 mL) was added DIPEA (0.1 mL, 0.59 mmol) at room temperature. After 5 minutes, HATU (53.59 mg, 0.14 mmol) was added. The resulting mixture was stirred at room temperature for 18 h. The crude mixture was directly purified by RediSep Rf Gold reverse phase flash chromatography using 10-80% MeCN/0.1% formic acid in water. The fractions containing the desired product were collected and volatiles concentrated in vacuum to afford 7 (86 mg, 90% yield) as a yellow solid.

LCMS method 1: 99.9% purity at 215 nm, [M+H]=963.2 m/z.

¹H NMR (400 MHz, DMSO-d6) δ ppm 1.30 (br. s, 9H), 1.48-1.68 (m, 2H), 1.75-2.10 (m, 8H), 2.53-2.63 (m, 2H), 2.76-2.95 (m, 2H), 3.31 (m, 3H), 3.39-3.83 (m, 10H), 4.29-4.42 (m, 1H), 4.47-4.68 (m, 1H), 4.96-5.16 (m, 2H), 6.92 (d, J=7.8 Hz, 1H), 7.02-7.16 (m, 1H), 7.23-7.31 (m, 1H), 7.37 (s, 1H), 7.71 (d, J=8.6 Hz, 1H), 8.30 (s, 1H), 8.41 (br. s, 1H), 8.82 (d, J=7.8 Hz, 1H), 9.23 (s, 1H), 11.08 (s, 1H).

¹⁹F NMR (377 MHz, DMSO-d6) δ ppm-185.22 (s, 1 F), -58.91 (s, 3 F).

Step 6. Preparation of 5-[(3R,5R)-3-Amino-5-fluoro-1-piperidyl]-N-[1-[4-[4-[2-(2,6-dioxo-3-piperidyl)-1,3-dioxo-isoindolin-5-yl]piperazine-1-carbonyl]cyclohexyl]-3-(trifluoromethyl)pyrazol-4-yl]pyrazolo[1,5-a]pyrimidine-3-carboxamide (P-13). To a round bottom flask containing tert-Butyl N-[(3R,5R)-1-[3-[[1-[4-[4-[2-(2,6-dioxo-3-piperidyl)-1,3-dioxo-isoindolin-5-yl]piperazine-1-carbonyl]cyclohexyl]-3-(trifluoromethyl)pyrazol-4-yl]carbamoyl]pyrazolo[1,5-a]pyrimidin-5-yl]-5-fluoro-3-piperidyl]carbamate 7 (103 mg, 0.11 mmol) was added 4.0 M HCl in 1,4-dioxane (4 mL) and the solution was stirred at rt for 30 min. Upon completion by HPLC, the volatiles were evaporated under reduced pressure. The residue was purified by C18 RediSep Rf Gold reverse phase chromatography eluting 5-50% MeCN/0.02 M HCl in H₂O. The desired fractions were combined, concentrated under reduced pressure and lyophilised to afford P-13 (39.8 mg, 43% yield) as a yellow solid.

LCMS method 5: 99.9% purity at 215 nm, [M−HCl+H]=863.2 m/z, [M−HCl+2H]2+=432.2 m/z.

¹H NMR (400 MHz, DMSO-d6) δ ppm 1.54-1.68 (m, 2H), 1.81-2.12 (m, 8H), 2.35-2.44 (m, 1H), 2.53-2.64 (m, 2H), 2.76-2.96 (m, 2H), 3.22-3.29 (m, 1H), 3.35-3.78 (m, 10H), 4.33-4.43 (m, 1H), 4.44-4.93 (m, 2H), 5.03-5.22 (m, 2H), 6.91 (d, J=7.6 Hz, 1H), 7.28 (dd, J=8.6, 1.5 Hz, 1H), 7.37 (s, 1H), 7.71 (d, J=8.6 Hz, 1H), 8.13 (br s, 3H), 8.34 (s, 1H), 8.46 (s, 1H), 8.94 (d, J=8.1 Hz, 1H), 9.25 (s, 1H), 11.08 (s, 1H).

¹⁹F NMR (377 MHz, DMSO-d6) δ ppm-184.97 (s, 1 F), -58.76 (s, 3 F).

### Example S3. Synthesis of P-31

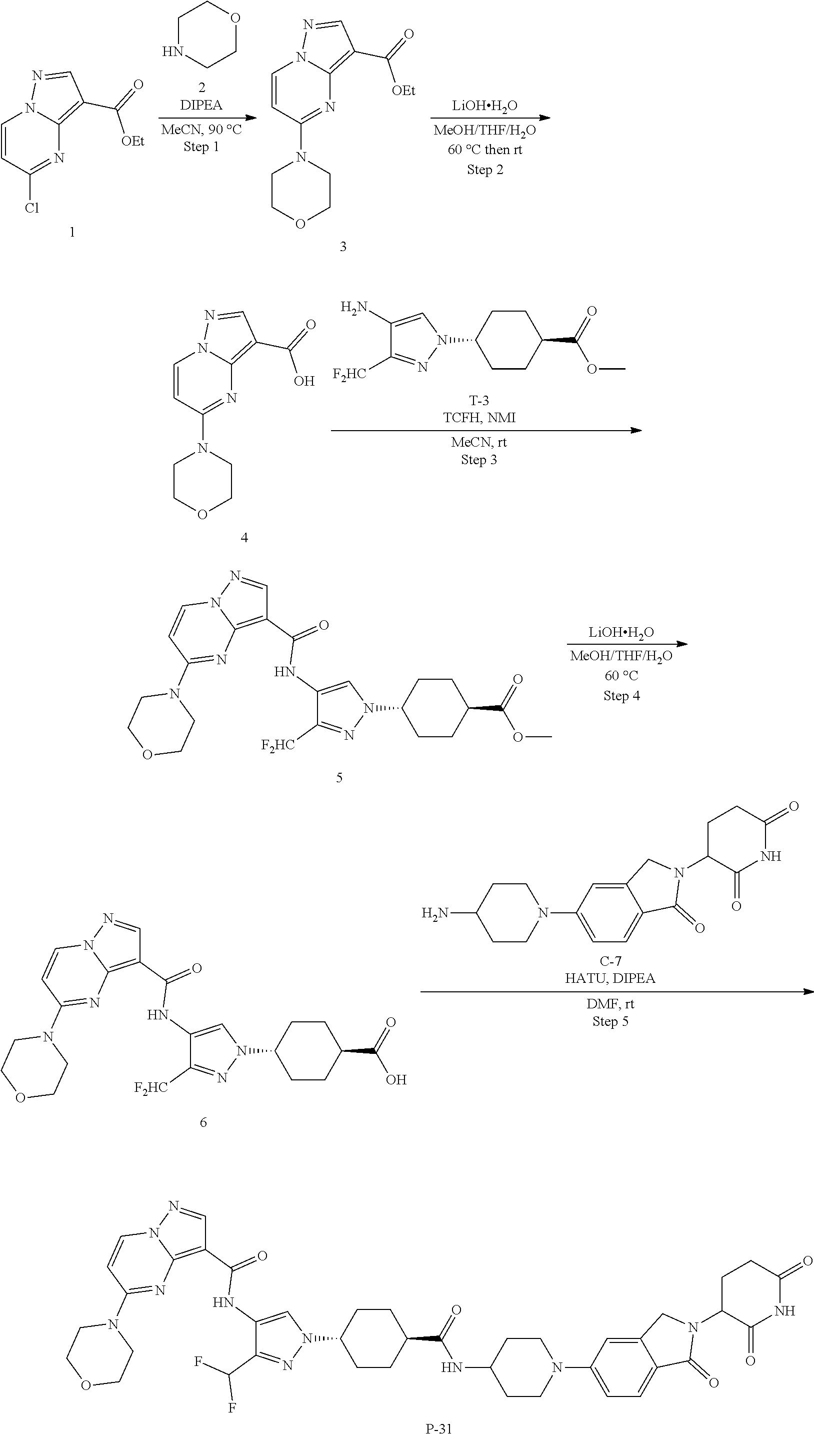

Step 1. Preparation of Ethyl 5-morpholinopyrazolo[1,5-a]pyrimidine-3-carboxylate 3. To a sealed tube were introduced ethyl 5-chloropyrazolo[1,5-a]pyrimidine-3-carboxylate 1 (1.5 g, 6.65 mmol, 1 eq.), MeCN (33.24 mL, 0.2 M) and DIPEA (2.89 mL, 16.62 mmol, 2.5 eq.), followed by morpholine 2 (639.65 uL, 7.31 mmol, 1.1 eq.). The tube was sealed and the mixture was stirred at 90° C. for 16 h. The solvents were evaporated under reduced pressure and dried under high vacuum to give ethyl 5-morpholinopyrazolo[1,5-a]pyrimidine-3-carboxylate 3 (2.96 g, 99% yield) as an orange solid.

LCMS method 1: retention time: 1.432 min, 99% purity at 215 nm, [M+H]=277.2

¹H NMR (400 MHz, CHLOROFORM-d) δ ppm 1.39 (t, J=7.1 Hz, 3H), 3.76-3.86 (m, 8H), 4.35 (q, J=7.1 Hz, 2H), 6.41 (d, J=8.1 Hz, 1H), 8.29-8.34 (m, 2H).

Step 2. Preparation of 5-Morpholinopyrazolo[1,5-a]pyrimidine-3-carboxylic acid 4. To a solution of ethyl 5-morpholinopyrazolo[1,5-a]pyrimidine-3-carboxylate 3 (1.84 g, 6.66 mmol, 1.0 eq.) in THF (17.8 mL, 0.1 M) and methanol (17.8 mL), after 5 minutes of stirring was added a solution of LiOH H₂O (2.8 g, 66.6 mmol, 10 eq.) in water (17.8 mL). The mixture was stirred at 60° C. for 3 h, then the oil bath was removed and the mixture was allowed to stir over the weekend (72 h). The reaction mixture was concentrated under reduced pressure, then the crude mixture was diluted with a small quantity of water and acidified to pH 3 with 6 N HCl. The resulting precipitate was filtered and rinsed with water. The solid was dried under high vacuum to give 5-morpholinopyrazolo[1,5-a]pyrimidine-3-carboxylic acid 4 (1.65 g, 99% yield) as a tan solid.

LCMS method 1: retention time: 1.192 min, 99% purity at 215 nm, [M+H]=249.2

¹H NMR (400 MHz, DMSO-d6) δ ppm 3.72 (br d, J=5.9 Hz, 8H), 6.84 (d, J=8.1 Hz, 1H), 8.19 (s, 1H), 8.74 (d, J=7.8 Hz, 1H), 11.73 (s, 1H).

Step 3. Preparation of Methyl 4-[3-(difluoromethyl)-4-[(5-morpholinopyrazolo[1,5-a]pyrimidine-3-carbonyl)amino]pyrazol-1-yl]cyclohexanecarboxylate 5. To a round bottom flask was introduced methyl 4-[4-amino-3-(difluoromethyl)pyrazol-1-yl]cyclohexanecarboxylate T-3 (201.83 mg, 0.66 mmol, 1.1 eq.), 5-morpholinopyrazolo[1,5-a]pyrimidine-3-carboxylic acid 4 (150.0 mg, 0.60 mmol, 1.0 eq.), MeCN (7.5 mL, 0.08 M) and NMI (174.74 uL, 2.21 mmol, 3.6 eq.), then the mixture was stirred for 5 minutes at 0° C. before TCFH (211.93 mg, 0.76 mmol, 1.25 eq.) was added. The mixture was stirred for 5 min at 0° C., then allowed to warm to room temperature over 30 minutes. The reaction mixture was concentrated in vacuo. Purification by reverse phase chromatography (50 g C18 RediSep Rf Gold column, liquid deposit (DMSO), elution: 5% MeOH/0.1% HCOOH over 4 CV, then 5% to 100% MeOH/0.1% HCOOH over 15 CV). The pure fractions were combined, concentrated under reduced pressure and dried under high vacuum to afford methyl 4-[3-(difluoromethyl)-4-[(5-morpholinopyrazolo[1,5-a]pyrimidine-3-carbonyl)amino]pyrazol-1-yl]cyclohexanecarboxylate 5 (315.8 mg, 95% yield) as a light orange solid.

LCMS method 1: retention time: 1.687 min, 92.4% purity at 215 nm, [M+H]+=504.0.

¹H NMR (400 MHz, DMSO-d6) δ ppm 1.47-1.62 (m, 2H), 1.73-1.88 (m, 2H), 1.98-2.10 (m, 4H), 2.37-2.47 (m, 1H), 3.62 (s, 3H), 3.69-3.74 (m, 4H), 3.76-3.82 (m, 4H), 4.25 (ddt, J=11.7, 7.9, 3.9 Hz, 1H), 6.91 (d, J=7.8 Hz, 1H), 6.95-7.25 (m, 1H), 8.29 (s, 1H), 8.39 (s, 1H), 8.83 (d, J=8.1 Hz, 1H), 9.40 (s, 1H).

Step 4. Preparation of 4-[3-(Difluoromethyl)-4-[(5-morpholinopyrazolo[1,5-a]pyrimidine-3-carbonyl)amino]pyrazol-1-yl]cyclohexanecarboxylic acid 6. To a round bottom flask was introduced methyl 4-[3-(difluoromethyl)-4-[(5-morpholinopyrazolo[1,5-a]pyrimidine-3-carbonyl)amino]pyrazol-1-yl]cyclohexanecarboxylate 5 (291.8 mg, 0.58 mmol, 1.0 eq.), THE (1.9 mL, 0.1 M) and methanol (1.9 mL), then the solution was stirred for 5 minutes before a solution of LiOH H₂O (243.41 mg, 5.8 mmol, 10.0 eq.) in water (1.9 mL) was added. The mixture was stirred at 60° C. for 1 hour. The reaction mixture was concentrated under reduced pressure, then the crude mixture was diluted with a small quantity of water and acidified to pH 3 with 6 N HCl. The precipitate was filtered, washed with acetonitrile and dried under high vacuum to give 4-[3-(difluoromethyl)-4-[(5-morpholinopyrazolo[1,5-a]pyrimidine-3-carbonyl)amino]pyrazol-1-yl]cyclohexanecarboxylic acid 6 (251.0 mg, 81% yield) of as an off-white solid.

LCMS method 1: retention time: 1.518 min, 91.9% purity at 215 nm, [M+H]+=490.2.

¹H NMR (400 MHz, DMSO-d6) δ ppm 1.44-1.59 (m, 2H), 1.71-1.86 (m, 2H), 1.99-2.09 (m, 4H), 2.23-2.35 (m, 1H), 3.68-3.75 (m, 4H), 3.76-3.84 (m, 4H), 4.17-4.31 (m, 1H), 6.91 (d, J=8.1 Hz, 1H), 6.95-7.26 (m, 1H), 8.29 (s, 1H), 8.39 (s, 1H), 8.83 (d, J=8.1 Hz, 1H), 9.40 (s, 1H), 12.15 (br s, 1H).

Step 5. Preparation of N-[3-(difluoromethyl)-1-[4-[[1-[2-(2,6-dioxo-3-piperidyl)-1-oxo-isoindolin-5-yl]-4-piperidyl]carbamoyl]cyclohexyl]pyrazol-4-yl]-5-morpholino-pyrazolo[1,5-a]pyrimidine-3-carboxamide P-31. 4-[3-(difluoromethyl)-4-[(5-morpholinopyrazolo[1,5-a]pyrimidine-3-carbonyl)amino]pyrazol-1-yl]cyclohexanecarboxylic acid 6 (45.0 mg, 0.09 mmol, 1.0 eq.) and 3-[5-(4-amino-1-piperidyl)-1-oxo-isoindolin-2-yl]piperidine-2,6-dione hydrochloride C-7 (41.8 mg, 0.11 mmol, 1.2 eq.) were dissolved in DMF (0.9 mL, 0.1 M), then DIPEA (160.14 uL, 0.92 mmol, 10 eq.) and HATU (52.43 mg, 0.14 mmol, 1.5 eq.) were added in sequence. The reaction mixture was stirred overnight under nitrogen atmosphere. Water (~7 mL) was added to the mixture, then the solid was filtered and washed with water. The solid was collected, solubilised into DMF and purified by reverse phase chromatography (50 g C18 RediSep Rf Gold column, liquid deposit (DMF), elution: 5% MeCN/0.1% HCOOH over 4 CV, then 5% to 50% MeCN/0.1% HCOOH over 15 CV). The pure fractions were combined, concentrated under reduced pressure and lyophilised overnight to afford N-[3-(difluoromethyl)-1-[4-[[1-[2-(2,6-dioxo-3-piperidyl)-1-oxo-isoindolin-5-yl]-4-piperidyl]carbamoyl]cyclohexyl]pyrazol-4-yl]-5-morpholino-pyrazolo[1,5-a]pyrimidine-3-carboxamide P-31 (21.8 mg, 29% yield).

LCMS method 5: retention time: 3.017 min, 99.8% purity at 215 nm, [M+2H]²⁺=407.8, [M+H]+=814.3.

¹H NMR (400 MHz, DMSO-d6) δ ppm 1.42-1.63 (m, 4H), 1.67-1.87 (m, 6H), 1.93-2.00 (m, 1H), 2.04-2.11 (m, 2H), 2.12-2.19 (m, 1H), 2.35-2.44 (m, 1H), 2.55-2.63 (m, 1H), 2.85-3.02 (m, 3H), 3.70-3.75 (m, 4H), 3.77-3.86 (m, 7H), 4.18-4.35 (m, 3H), 5.05 (dd, J=13.2, 5.1 Hz, 1H), 6.91 (d, J=7.8 Hz, 1H), 6.97-7.24 (m, 3H), 7.51 (d, J=8.6 Hz, 1H), 7.77 (d, J=7.8 Hz, 1H), 8.29 (s, 1H), 8.39 (s, 1H), 8.83 (d, J=7.8 Hz, 1H), 9.40 (s, 1H), 10.94 (s, 1H).

¹⁹F NMR (377 MHz, DMSO-d6) δ ppm-111.32 (d, J=54.5 Hz, 2 F).

Example S4. The following compounds were synthesized via the same general routes described above with modifications to the amine 2 in Step 1, Intermediate (T-X) in step 3 and CBM (C—X) in step 5 (Table 2).

TABLE 2

| Compound | | | | | |
|---|---|---|---|---|---|
| No. | Amine | Int | CBM | Structure | Characterization Data |
| P-1 | 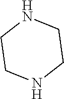 | T-1 | C-1 | 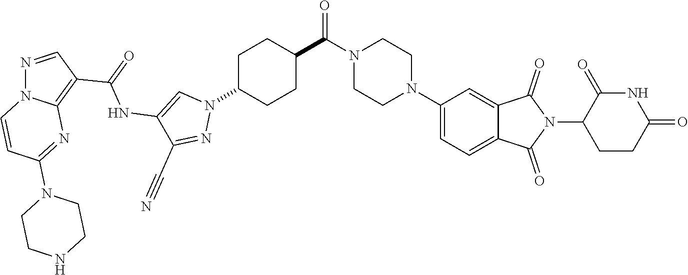 | 91% yield as a yellow solid. LCMS method 5: retention time: 2.309 min, 99% purity at 215 nm, [M − HCl + Na]⁺ = 810.2, [M − HCl + 2H]²⁺ = 394.7. ¹H NMR (400 MHz, DMSO- d6) δ ppm 1.53-1.66 (m, 2 H), 1.81- 2.11 (m, 7 H), 2.53-2.63 (m, 2 H), 2.76-2.95 (m, 2 H), 3.22-3.29 (m, 4 H), 3.44-3.57 (m, 4 H), 3.59-3.76 (m, 4 H), 4.00-4.13 (m, 4 H), 4.37- 4.46 (m, 1 H), 5.08 (dd, J = 12.8, 5.5 Hz, 1 H), 7.00 (d, J = 8.1 Hz, 1 H), 7.27 (dd, J = 8.7, 1.8 Hz, 1 H), 7.37 (d, J = 1.5 Hz, 1 H), 7.71 (d, J = 8.3 Hz, 1 H), 8.37 (s, 1 H), 8.48 (s, 1 H), 8.95 (d, J = 8.1 Hz, 1 H), 9.04 (br. s, 2 H), 9.59 (s, 1 H), |
| | | | | | 11.08 (s, 1 H). |
| P-2 | 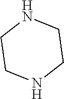 | T-1 | C-2 | 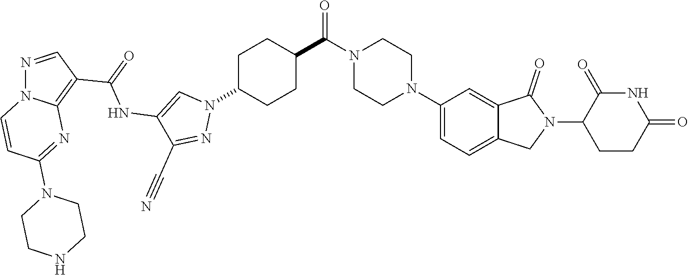 | 73% yield as a white solid. LCMS method 5: retention time: 2.123 min, 98% purity at 215 nm, [M − HCl + H]⁺ = 774.3; [M − HC1 + 2H]²⁺ = 387.8. ¹H NMR (400 MHz, DMSO- d6) δ ppm 1.53-1.67 (m, 2 H), 1.81- 2.10 (m, 7 H), 2.34-2.45 (m, 1 H), 2.55-2.64 (m, 1 H), 2.77-2.97 (m, 2 H), 3.14-3.30 (m, 8 H), 3.60-3.86 (m, 4 H), 3.99-4.16 (m, 4 H), 4.19- 4.26 (m, 1 H), 4.31-4.46 (m, 2 H), 5.10 (dd, J = 13.3, 5.0 Hz, 1 H), 7.00 (d, J = 8.1 Hz, 1 H), 7.22 (d, J = 1.7 Hz, 1 H), 7.31 (dd, J = 8.6, 1.7 Hz, 1 H), 7.47 (d, J = 8.3 Hz, 1 H), 8.37 (s, 1 H), 8.48 (s, 1 H), 8.95 (d, J = 7.8 Hz, 1 H), 9.08-9.23 |
| | | | | | (m, 2 H), 9.59 (s, 1 H), 10.97 (s, 1 |
| | | | | | H). - Four protons are partially |
| | | | | | under the peak of water in the ¹H |
| | | | | | NMR spectrum. |
| P-3 | 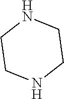 | T-1 | C-3 | 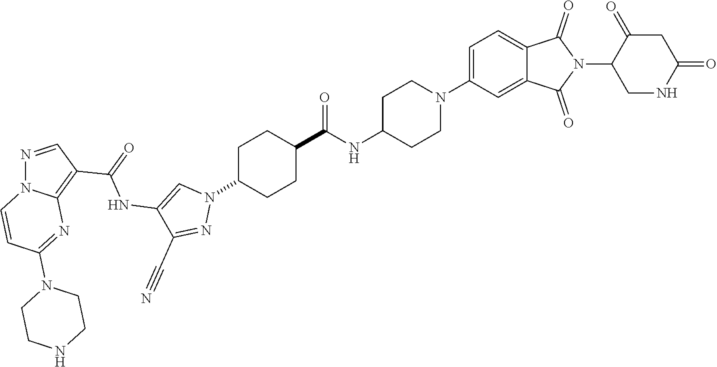 | 52% yield as a yellow solid. LCMS method 5: retention time: 2.236 min, 99% purity at 215 nm, [M + H]⁺ = 802.2 m/z, [M + 2H]2⁺ = 401.6 m/z. ¹H NMR (400 MHz, DMSO-d6) δ ppm 1.36-1.50 (m, 2 H), 1.50-1.64 (m, 2 H), 1.69-1.92 (m, 6 H), 1.97- 2.11 (m, 3 H), 2.13-2.23 (m, 1 H), 2.53-2.63 (m, 2 H), 2.82-2.95 (m, 1 H), 3.07-3.18 (m, 2 H), 3.18-3.33 (m, 4 H), 3.81-3.92 (m, 1 H), 3.92- 4.16 (m, 6 H), 4.22-4.53 (m, 1 H), 5.07 (dd, J = 12.7, 5.4 Hz, 1 H), 6.99 (d, J = 7.8 Hz, 1 H), 7.26 (d, J = 8.6 Hz, 1 H), 7.34 (s, 1 H), 7.67 (d, J = 8.6 Hz, 1 H), 7.80 (d, J = 7.3 Hz, 1 H), 8.36 (s, 1 H), 8.47 (s, 1 H), 8.94 (d, J = 7.8 Hz, 1 H), 9.14- 9.37 (m, 2 H), 9.57 (s, 1 H), 11.08 (s, 1 H). - One proton under water signal and two proton partially under DMSO signal in ¹H NMR. |
| P-4 | 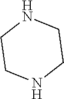 | T-1 | C-4 | 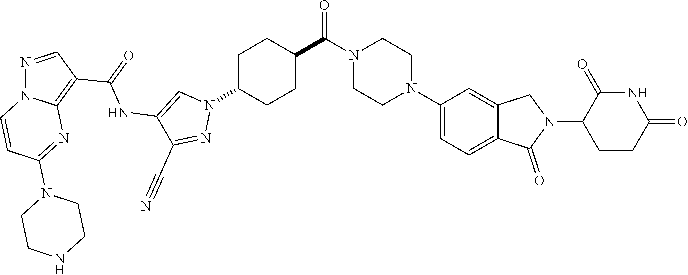 | 46% yield as a white solid. LCMS method 5: retention time: 2.098 min, 9% purity at 215 nm, [M + H]⁺ = 774.2 m/z, [M + 2H]²⁺ = 387.8 m/z. ¹H NMR (400 MHz, DMSO-d6) δ ppm 1.52-1.67 (m, 2 H), 1.80-2.01 (m, 5 H), 2.03-2.12 (m, 2 H), 2.34- 2.42 (m, 1 H), 2.53-2.64 (m, 2 H), 2.76-2.97 (m, 2 H), 3.15-3.45 (m, 9 H), 3.69-3.76 (m, 2 H), 3.94-4.18 (m, 4 H), 4.19-4.27 (m, 1 H), 4.30- 4.46 (m, 2 H), 5.05 (dd, J = 14.5, 4.8 Hz, 1 H), 6.99 (d, J = 8.3 Hz, 1 H), 7.06-7.16 (m, 2 H), 7.55 (d, J = 8.3 Hz, 1 H), 8.37 (s, 1 H), 8.48 (s, 1 H), 8.94 (d, J = 7.8 Hz, 1 H), 9.12-9.32 (m, 2 H), 9.59 (s, 1 H), |
| | | | | | 10.95 (s, 1 H). |
| P-5 | 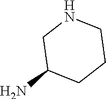 | T-1 | C-1 | 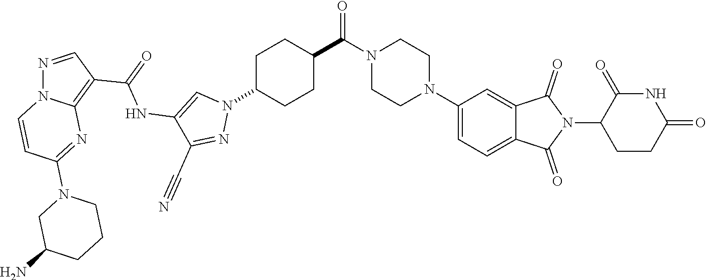 | 63% yield as a yellow solid. LCMS method 5: retention time: 2.275 min, 93% purity at 215 nm, [M + H]⁺ = 802.2 m/z, [M + 2H]²⁺ = 401.7 m/z. ¹H NMR (400 MHz, DMSO-d6) δ ppm 1.53-1.69 (m, 3 H), 1.70-1.80 (m, 1 H), 1.80-1.98 (m, 5 H), 2.00- 2.11 (m, 4 H), 2.53-2.63 (m, 2 H), 2.76-2.95 (m, 2 H), 3.29-3.38 (m, 1 H), 3.69-3.77 (m, 3 H), 3.93-4.07 (m, 1 H), 4.22-4.31 (m, 1 H), 4.37- 4.47 (m, 1 H), 5.08 (dd, J = 12.8, 5.3 Hz, 1 H), 6.93 (d, J = 8.1 Hz, 1 H), 7.27 (dd, J = 8.6, 2.2 Hz, 1 H), 7.37 (d, J = 2.2 Hz, 1 H), 7.72 (d, J = 8.6 Hz, 1 H), 8.10 (s, 3 H), 8.33 (s, 1 H), 8.49 (s, 1 H), 8.88 (d, J = |
| | | | | | 7.8 Hz, 1 H), 9.61 (s, 1 H), 11.08 (s, |
| | | | | | 1 H). - Seven protons are partially |
| | | | | | obscured by the water peak in the |
| | | | | | ¹H NMR. |
| P-7 | 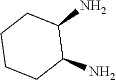 | T-1 | C-1 | 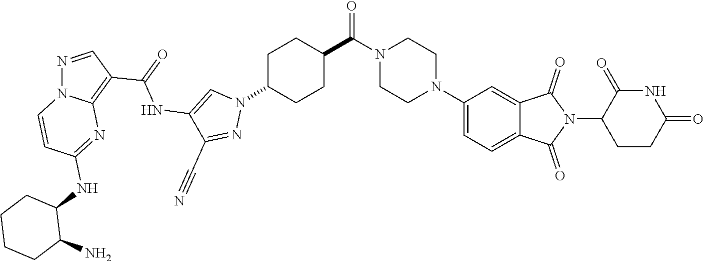 | 38% yield as a yellow solid. LCMS method 5: retention time: 2.424 min, 99% purity at 215 nm, [M − 2HCl + H]⁺ = 816.3, [M − 2HCl + 2H]²⁺ = 408.7. ¹H NMR (400 MHz, DMSO- d6) δ ppm 0.82-0.91 (m, 1 H), 1.16- 1.41 (m, 2 H), 1.47-2.12 (m, 18 H), 2.55-2.65 (m, 2 H), 2.77-2.96 (m, 2 H), 3.27-3.38 (m, 1 H), 3.59-3.78 (m, 5 H), 4.36-4.47 (m, 1 H), 4.96- 5.03 (m, 1 H), 5.08 (dd, J = 12.8, 5.3 Hz, 1 H), 6.73 (d, J = 7.8 Hz, 1 H), 7.28 (d, J = 8.6 Hz, 1 H), 7.37 (s, 1 H), 7.72 (d, J = 8.6 Hz, 1 H), 7.94 (br. s, 3 H), 8.14 (d, J = 9.5 Hz, 1 H), 8.27 (s, 1 H), 8.49 (s, 1 H), 8.68 |
| | | | | | (d, J = 7.6 Hz, 1 H), 9.69 (s, 1 H), |
| | | | | | 11.08 (s, 1 H). |
| P-8 | 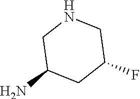 | T-1 | C-1 | 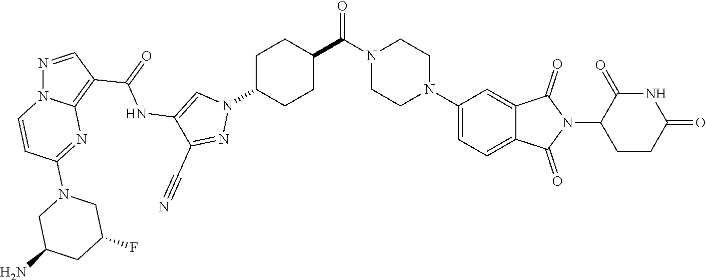 | 16% yield as a yellow solid. LCMS method 5: retention time: 2.260 min, 99% purity at 215 nm, [M − HCl + H]⁺ = 820.2; [M − HCl + 2H]²⁺ = 410.7. ¹H NMR (400 MHz, DMSO- d6) δ ppm 1.55-1.66 (m, 2 H), 1.82- 2.10 (m, 8 H), 2.35-2.41 (m, 1 H), 2.55-2.63 (m, 3 H), 2.78-2.95 (m, 2 H), 3.18-3.25 (m, 1 H), 3.38-3.77 (m, 11 H), 4.38-4.47 (m, 1 H), 5.05- 5.22 (m, 2 H), 6.87-7.04 (m, 1 H), 7.28 (d, J = 8.8 Hz, 1 H), 7.37 (s, 1 H), 7.72 (d, J = 8.8 Hz, 1 H), 8.01 (br s, 3 H), 8.36 (s, 1 H), 8.49 (s, 1 H), 8.92 (d, J = 6.8 Hz, 1 H), 9.58 (s, 1 H), 11.07 (s, 1 H). |
| P-10 | 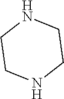 | T-1 | C-5 | 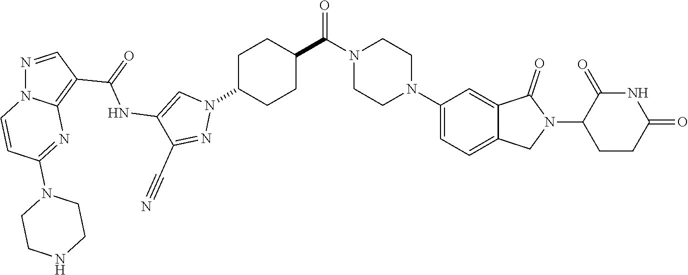 | 51% yield as a white solid. LCMS method 5: retention time: 2.188 min, 98% purity at 215 nm, [M − HC1 + H]⁺ = 773.3; [M − HCl + 2H]²⁺ = 387.3. ¹H NMR (400 MHz, DMSO- d6) δ ppm 1.42-1.72 (m, 5 H), 1.80- 2.10 (m, 10 H), 2.36-2.45 (m, 2 H), 2.56-2.66 (m, 2 H), 2.75-3.00 (m, 4 H), 3.11-3.21 (m, 1 H), 4.00-4.20 (m, 5 H), 4.26-4.32 (m, 1 H), 4.35- 4.46 (m, 2 H), 4.55-4.63 (m, 1 H), 5.11 (dd, J = 12.0, 4.5 Hz, 1 H), 6.99 (d, J = 7.8 Hz, 1 H), 7.54 (s, 2 H), 7.60 (s, 1 H), 8.37 (s, 1 H), 8.48 (s, 1 H), 8.95 (m, 3 H), 9.59 (s, 1 H), 10.98 (s, 1 H). |
| P-11 | 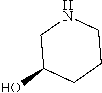 | T-1 | C-1 | 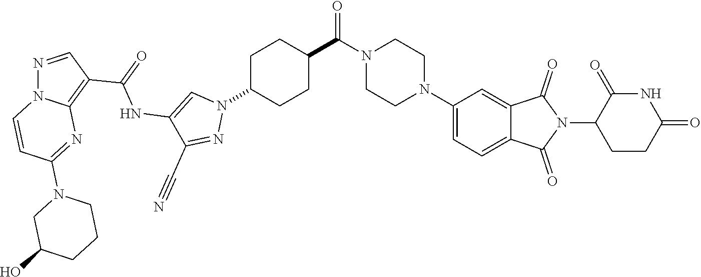 | 16% yield as a yellow solid. LCMS method 5: retention time: 3.209 min, 98% purity at 215 nm, [M + H]⁺ = 803.2 m/z, [M + 2H]²⁺ = 402.3 m/z. ¹H NMR (400 MHz, DMSO-d6) δ ppm 1.52-1.66 (m, 4 H), 1.84-2.13 (m, 9 H), 2.74-2.98 (m, 3 H), 3.47- 3.97 (m, 15 H), 4.40 (m, 1 H), 5.02- 5.14 (m, 1 H), 6.85-6.94 (m, 1 H), 7.22-7.32 (m, 1 H), 7.37 (s, 1 H), 7.66-7.75 (m, 1 H), 8.27 (s, 1 H), 8.46 (s, 1 H), 8.68-8.79 (m, 1 H), 9.65 (s, 1 H), 11.08 (s, 1 H). |
| P-12 | 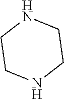 | T-1 | C-6 | 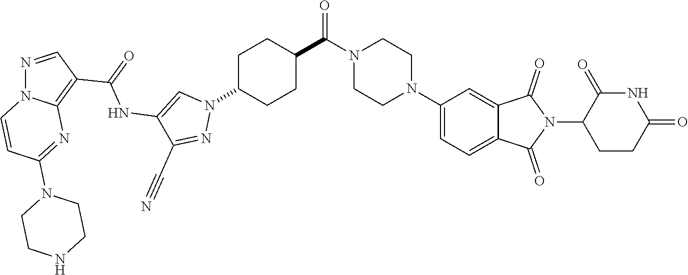 | 81% yield as an off-white solid. LCMS method 5: retention time: 2.250 min, 99% purity at 215 nm, [M − HCl + H]⁺ = 787.2; [M − HCl + 2H]²⁺ = 394.2; [M − HCl + Na]⁺ = 809.0. ¹H NMR (400 MHz, DMSO-d6) δ ppm 1.45-1.99 (m, 11 H), 2.01-2.12 (m, 3 H), 2.53-2.66 (m, 2 H), 2.74-2.97 (m, 2 H), 3.00- 3.21 (m, 2 H), 3.22-3.29 (m, 4 H), 3.98-4.23 (m, 5 H), 4.35-4.48 (m, 1 H), 4.55-4.66 (m, 1 H), 5.14 (dd, J = 13.0, 5.1 Hz, 1 H), 6.99 (d, J = 7.8 Hz, 1 H), 7.73-7.92 (m, 3 H), 8.36 (s, 1 H), 8.48 (s, 1 H), 8.94 (d, J = 7.8 Hz, 1 H), 9.12 (br s, 2 H), 9.59 (s, 1 H), 11.12 (s, 1 H). - Two |
| | | | | | protons partially overlap with |
| | | | | | DMSO in the ¹H NMR spectrum. |
| P-13 | 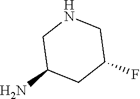 | T-2 | C-1 | 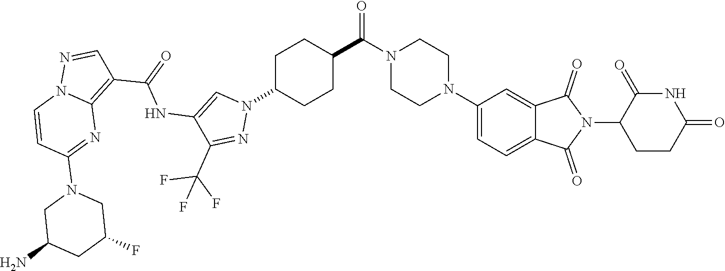 | 43% yield as a yellow solid. LCMS method 5: retention time: 2.303 min, 99% purity at 215 nm, [M − HCl + H]⁺ = 863.2 m/z, [M − HCl + 2H]²⁺ = 432.2 m/z. ¹H NMR (400 MHz, DMSO-d6) δ ppm 1.54-1.68 (m, 2 H), 1.81-2.12 (m, 8 H), 2.35- 2.44 (m, 1 H), 2.53-2.64 (m, 2 H), 2.76-2.96 (m, 2 H), 3.22-3.29 (m, 1 H), 3.35-3.78 (m, 10 H), 4.33-4.43 (m, 1 H), 4.44-4.93 (m, 2 H), 5.03- 5.22 (m, 2 H), 6.91 (d,J = 7.6 Hz, 1 H), 7.28 (dd, J = 8.6, 1.5 Hz, 1 H), 7.37 (s, 1 H), 7.71 (d, J = 8.6 Hz, 1 H), 8.13 (br s, 3 H), 8.34 (s, 1 H), 8.46 (s, 1 H), 8.94 (d, J = 8.1 Hz, 1 H), 9.25 (s, 1 H), 11.08 (s, 1 H). ¹⁹F |
| | | | | | NMR (377 MHz, DMSO-d6) δ ppm |
| | | | | | −184.97 (s, 1 F), −58.76 (s, 3 F). |
| P-14 | 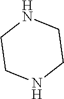 | T-2 | C-1 | 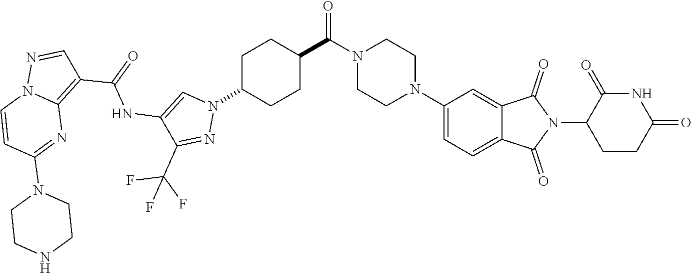 | 84% yield as a yellow solid. LCMS method 5: retention time: 2.253 min, 98% purity at 215 nm, [M − HCl + H]⁺ = 831.2, [M − HCl + 2H]²⁺ = 416.2. ¹H NMR (400 MHz, DMSO- d6) δ ppm 1.54-1.66 (m, 2 H), 1.82- 2.11 (m, 7 H), 2.52-2.64 (m, 2 H), 2.78-2.94 (m, 2 H), 3.21-3.30 (m, 4 H), 3.47-3.79 (m, 8 H), 3.95-4.11 (m, 4 H), 4.31-4.43 (m, 1 H), 5.08 (dd, J = 13.1, 5.3 Hz, 1 H), 6.98 (d, J = 8.1 Hz, 1 H), 7.27 (d, J = 8.6 Hz, 1 H), 7.37 (s, 1 H), 7.71 (d, J = 8.6 Hz, 1 H), 8.35 (s, 1 H), 8.47 (s, 1 H), 8.93 (d, J = 7.8 Hz, 1 H), 9.17-9.38 (m, 3 H), 11.08 (s, 1 H). ¹⁹F NMR (377 MHz, DMSO-d6) δ |
| | | | | | ppm −58.98 (s, 3 F). |
| P-15 | 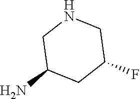 | T-3 | C-1 | 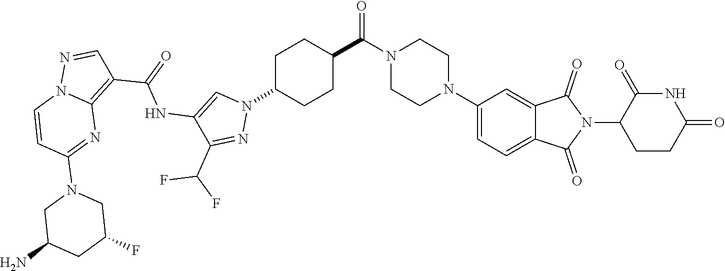 | 52% yield as a yellow solid. LCMS method 5: retention time: 2.223 min, 96% purity at 215 nm, [M − HCl + H]⁺ = 845.5 m/z, [M − HCl + 2H]²⁺ = 423.2 m/z. ¹H NMR (400 MHz, DMSO-d6) δ ppm 1.52-1.69 (m, 2 H), 1.79-2.13 (m, 8 H), 2.34- 2.45 (m, 1 H), 2.53-2.64 (m, 3 H), 2.75-2.94 (m, 2 H), 3.24-3.49 (m, 5 H), 3.61-3.67 (m, 3 H), 3.70-3.76 (m, 2 H), 4.24-4.34 (m, 1 H), 4.52- 4.70 (m, 1 H), 4.75-4.93 (m, 1 H), 5.02-5.19 (m, 2 H), 6.89 (d, J = 8.0 Hz, 1 H), 6.99-7.30 (m, 3 H), 7.36- 7.39 (m, 1 H), 7.71 (d, J = 8.5 Hz, 1 H), 8.16-8.27 (m, 2 H), 8.33 (s, 1 H), 8.39 (s, 1 H), 8.93 (d, J = 7.8 |
| | | | | | Hz, 1 H), 9.34 (s, 1 H), 11.08 (s, 1 |
| | | | | | H). - Three protons partially under |
| | | | | | the peak of DMSO and three |
| | | | | | protons partially under the peak of |
| | | | | | water by ¹H NMR. ¹⁹F NMR (377 |
| | | | | | MHz, DMSO-d6) δ ppm −184.64 (s, |
| | | | | | 1 F), −111.27 (d, J = 42.2 Hz, 2 F). |
| P-16 | 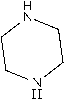 | T-3 | C-1 | 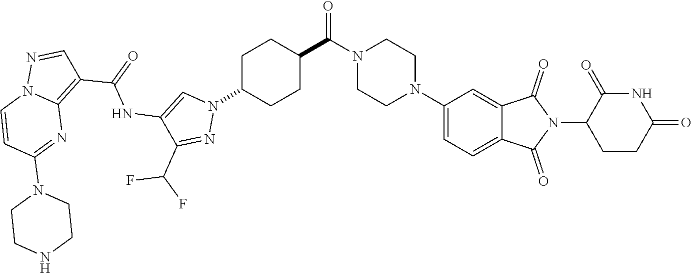 | 65% yield as a yellow solid. LCMS method 5: retention time: 2.193 min, 99% purity at 215 nm, [M − HCl + H]+ = 813.2, [M − HCl + 2H]²⁺ = 407.3. ¹H NMR (400 MHz, DMSO- d6) δ ppm 1.53-1.67 (m, 2 H), 1.81- 2.13 (m, 8 H), 2.53-2.63 (m, 2 H), 2.74-2.95 (m, 2 H), 3.22-3.29 (m, 3 H), 3.45-3.57 (m, 4 H), 3.60-3.67 (m, 2 H), 3.70-3.76 (m, 2 H), 4.01- 4.08 (m, 3 H), 4.24-4.33 (m, 1 H), 5.08 (dd, J = 13.0, 5.5 Hz, 1 H), 6.97 (d, J = 8.0 Hz, 1 H), 7.01-7.27 (m, 2 H), 7.28 (s, 1 H), 7.36-7.38 (m, 1 H), 7.71 (d, J = 8.6 Hz, 1 H), 8.34 (s, 1 H), 8.41 (s, 1 H), 8.93 (d, J = 7.8 Hz, 1 H), 9.08-9.22 (m, 2 |
| | | | | | H), 9.35 (s, 1 H), 11.08 (s, 1 H). |
| | | | | | - Two protons partially under the |
| | | | | | peak of DMSO in ¹H NMR. ¹⁹F |
| | | | | | NMR (377 MHz, DMSO-d6) δ |
| | | | | | ppm −111.18 (d, J = 53.1 Hz, 2 F). |
| P-18 | 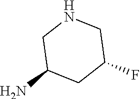 | T-1 | C-2 | 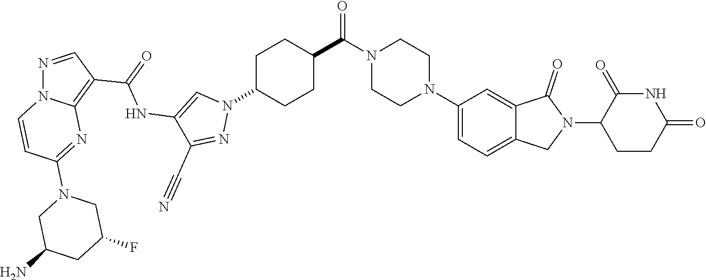 | 54% yield as a white solid. LCMS method 6: retention time: 2.993 min, 98% purity at 215 nm, [M − HCl + H]+ = 806.1; [M − HCl + 2H]²⁺ = 403.6. ¹H NMR (400 MHz, DMSO- d6) δ ppm 1.54-1.68 (m, 2 H), 1.81- 2.11 (m, 8 H), 2.35-2.45 (m, 2 H), 2.55-2.64 (m, 1 H), 2.77-2.98 (m, 2 H), 3.13-3.31 (m, 5 H), 3.34-3.44 (m, 2 H), 3.61-3.76 (m, 4 H), 4.18- 4.27 (m, 1 H), 4.31-4.48 (m, 2 H), 5.06-5.24 (m, 2 H), 6.90-7.02 (m, 1 H), 7.22 (s, 1 H), 7.31 (dd, J = 8.3, 1.7 Hz, 1 H), 7.47 (d, J = 8.3 Hz, 1 H), 8.08-8.28 (m, 3 H), 8.36 (s, 1 H), 8.48 (s, 1 H), 8.92 (d, J = 7.8 Hz, 1 H), 9.58 (s, 1 H), 10.97 (s, 1 |
| | | | | | H). - Two protons are obscured by |
| | | | | | the water and the DMSO-d6 peak |
| | | | | | in the ¹H NMR. |
| P-19 | 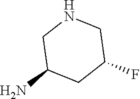 | T-3 | C-3 | 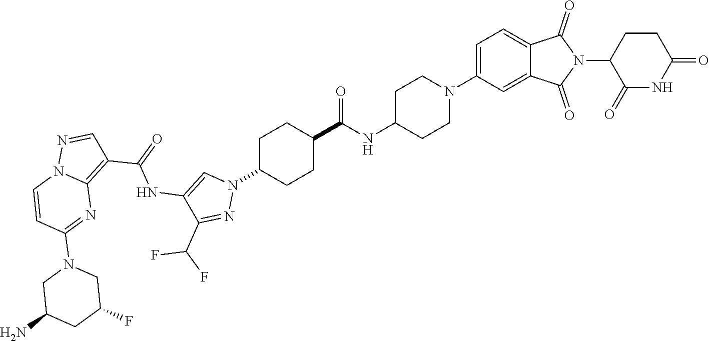 [SMILES: N[C@@H]1C[C@@H](F)CN(c2ccn3ncc(C(=O)Nc4cn([C@H]5CC[C@H](C(=O)NC6CCN(c7ccc8c(c7)C(=O)N(C7CCC(=O)NC7=O)C8=O)CC6)CC5)nc4C(F)F)c3n2)C1] | 79% yield as a yellow solid. LCMS method 5: retention time: 2.238 min, 99% purity at 215 nm, [M − HCl + H]⁺ = 859.2, [M − HCl + 2H]²⁺ = 430.3. ¹H NMR (400 MHz, DMSO- d6) δ ppm 1.35-1.50 (m, 2 H), 1.51- 1.64 (m, 2 H), 1.65-2.23 (m, 11 H), 2.34-2.45 (m, 1 H), 2.53-2.65 (m, 3 H), 2.82-2.96 (m, 1 H), 3.06-3.20 (m, 2 H), 3.36-3.48 (m, 2 H), 3.78- 3.92 (m, 1 H), 3.93-4.05 (m, 2 H), 4.18-4.31 (m, 1 H), 4.46-4.71 (m, 1 H), 4.74-4.97 (m, 1 H), 5.00-5.19 (m, 2 H), 6.82-6.94 (m, 1 H), 6.97- 7.31 (m, 2 H), 7.34 (d, J = 1.7 Hz, 1 H), 7.67 (d, J = 8.6 Hz, 1 H), 7.77 (d, J = 7.8 Hz, 1 H), 8.03-8.27 (m, 3 H), 8.32 (s, 1 H), 8.38 (s, 1 H), 8.93 (d, J = 7.8 Hz, 1 H), 9.33 (s, 1 H), 11.07 (s, 1 H). ¹⁹F NMR (377 MHz, DMSO-d6) δ ppm −184.00 (s, 1F), |
| | | | | | 111.05 (s, 2 F). |
| P-20 | 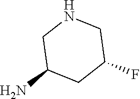 | T-2 | C-3 | 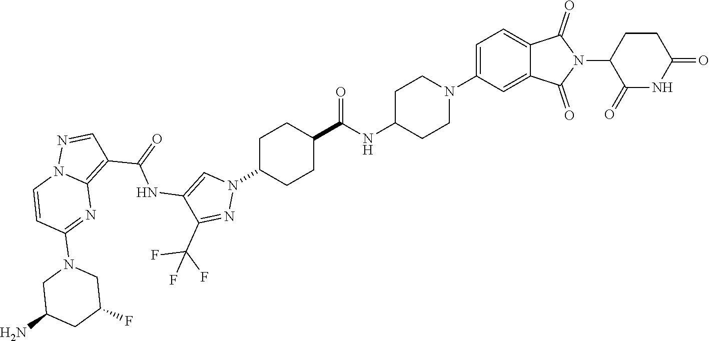 [SMILES: N[C@@H]1C[C@@H](F)CN(c2ccn3ncc(C(=O)Nc4cn([C@H]5CC[C@H](C(=O)NC6CCN(c7ccc8c(c7)C(=O)N(C7CCC(=O)NC7=O)C8=O)CC6)CC5)nc4C(F)(F)F)c3n2)C1] | 35% yield as a yellow solid. LCMS method 5: retention time: 2.300 min, 99% purity at 215 nm, [M − HCl + H]⁺ = 877.2; [M − HCl + 2H]²⁺ = 439.2. ¹H NMR (400 MHz, CDCl₃) δ ppm 1.37-1.50 (m, 2 H), 1.52-1.64 (m, 2 H), 1.70-2.22 (m, 11 H), 2.35- 2.43 (m, 1 H), 2.53-2.65 (m, 3 H), 2.82-2.95 (m, 1 H), 3.06-3.19 (m, 2 H), 3.36-3.58 (m, 2 H), 3.79-3.93 (m, 1 H), 3.94-4.05 (m, 2 H), 4.26- 4.37 (m, 1 H), 4.41-4.83 (m, 2 H), 5.02-5.21 (m, 2 H), 6.90 (d, J = 7.1 Hz, 1 H), 7.26 (dd, J = 8.7, 1.8 Hz, 1 H), 7.35 (d, J = 1.5 Hz, 1 H), 7.67 (d, J = 8.6 Hz, 1 H), 7.77 (d, J = 7.6 Hz, 1 H), 8.14 (br s, 3 H), 8.33 (s, 1 H), 8.45 (s, 1 H), 8.93 (d, J = 7.6 Hz, 1 H), 9.23 (s, 1 H), 11.07 (s, 1 H). ¹⁹F NMR (377 MHz, CDCl₃) δ ppm −184.95 (s, 1 F), −58.73 (s, 3 F). |
| P-21 | 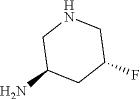 | T-3 | C-2 | 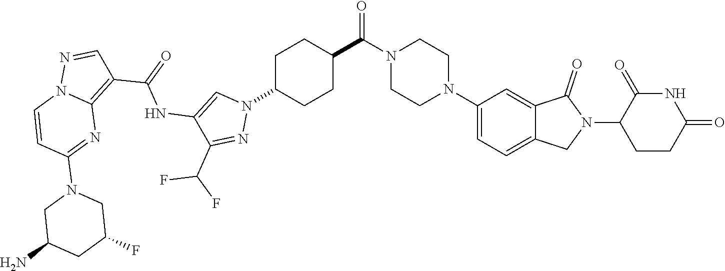 | 79% yield as a off-white solid. LCMS method 5: retention time: 2.135 min, 99.1% purity at 215 nm, [M + H]⁺ = 831.3 m/z, [M + 2H]²⁺ = 416.2 m/z. ¹H NMR (400 MHz, DMSO-d6) δ ppm 1.49-1.70 (m, 2 H), 1.77-2.15 (m, 7 H), 2.33-2.47 (m, 2 H), 2.54-2.64 (m, 1 H), 2.73- 2.98 (m, 2 H), 3.16-3.54 (m, 6 H), 3.56-3.88 (m, 4 H), 4.17-4.26 (m, 1 H), 4.29-4.39 (m, 4 H), 4.57-4.97 (m, 2 H), 5.01-5.20 (m, 2 H), 6.91 (d, J = 7.8 Hz, 1 H), 6.99-7.31 (m, 2 H), 7.33-7.41 (m, 1 H), 7.49 (d, J = 8.6 Hz, 1 H), 8.33 (s, 1 H), 8.35- 8.74 (m, 4 H), 8.93 (d, J = 8.1 Hz, 1 H), 9.34 (s, 1 H), 10.97 (s, 1 |
| | | | | | H). - 7 protons partially or totally |
| | | | | | under water signal in ¹H NMR. ¹⁹F |
| | | | | | NMR (377 MHz, DMSO-d6) δ ppm |
| | | | | | −184.86 (s, 1 F), −111.18 (s, 2 F). |
| P-22 | 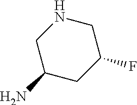 | T-2 | C-2 | 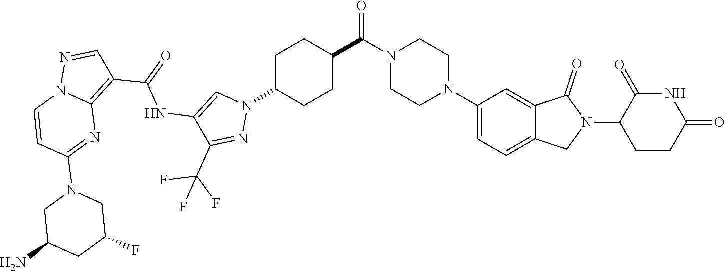 | 69% yield as a white solid. LCMS method 5: retention time: 2.207 min, 99% purity at 215 nm, [M − HCl + H]⁺ = 849.2, [M − HCl + 2H]²⁺ = 425.3. ¹H NMR (400 MHz, DMSO- d6) δ ppm 1.51-1.68 (m, 2 H), 1.78- 2.14 (m, 8 H), 2.34-2.45 (m, 2 H), 2.55-2.64 (m, 1 H), 2.77-2.99 (m, 2 H), 3.10-3.59 (m, 7 H), 3.61-3.79 (m, 4 H), 4.17-4.44 (m, 3 H), 4.47- 4.85 (m, 2 H), 5.02-5.23 (m, 2 H), 6.91 (d, J = 8.1 Hz, 1 H), 7.23 (d, J = 1.5 Hz, 1 H), 7.32 (dd, J = 8.4, 1.8 Hz, 1 H), 7.47 (d, J = 8.3 Hz, 1 H), 8.13-8.40 (m, 4 H), 8.46 (s, 1 H), 8.93 (d, J = 8.1 Hz, 1 H), 9.25 (s, 1 H), 10.97 (s, 1 H). ¹⁹F NMR |
| | | | | | (377 MHz, DMSO-d6) δ ppm |
| | | | | | −184.95 (s, 1F), −58.85 (s, 3 F). |
| P-23 | 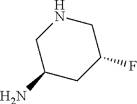 | T-1 | C-3 | 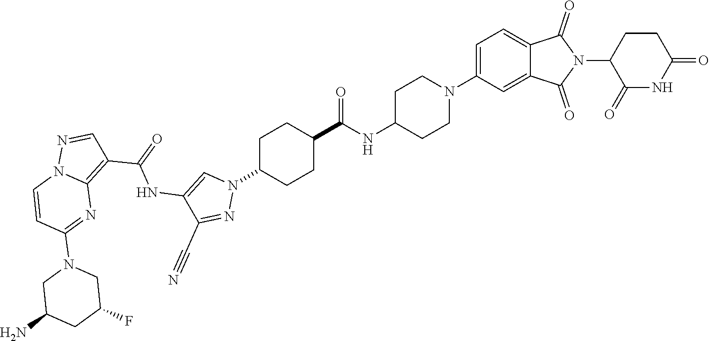 [SMILES: N#Cc1nn([C@H]2CC[C@H](C(=O)NC3CCN(c4ccc5c(c4)C(=O)N(C4CCC(=O)NC4=O)C5=O)CC3)CC2)cc1NC(=O)c1cnn2ccc(N3C[C@H](N)C[C@@H](F)C3)nc12] | 17% yield as a yellow solid. LCMS method 5: retention time: 2.268 min, 95% purity at 215 nm, [M + H]⁺ = 834.2, [M + 2H]²⁺ = 417.7. ¹H NMR (400 MHz, DMSO-d6) δ ppm 1.36- 1.65 (m, 5 H), 1.69-2.22 (m, 13 H), 2.35-2.42 (m, 2 H), 2.53 2.63 (m, 2 H), 2.82-2.94 (m, 1 H), 3.07-3.29 (m, 3 H), 3.80-3.92 (m, 1 H), 3.94- 4.04 (m, 2 H), 4.32-4.43 (m, 1 H), 5.04-5.23 (m, 2 H), 6.90-7.02 (m, 1 H), 7.26 (d, J = 10.0 Hz, 1 H), 7.35 (s, 1 H), 7.67 (d, J = 8.0 Hz, 1 H), 7.79 (d, J = 7.8 Hz, 1 H), 8.06-8.23 (m, 3 H), 8.35 (s, 1 H), 8.48 (s, 1 H), 8.92 (d, J = 7.6 Hz, 1 H), 9.56 (s, 1 H), 11.07 (s, 1 H). - Four protons are partially under the peak of DMSO by ¹H NMR spectrum. |
| P-24 | 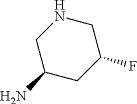 | T-3 | C-7 | 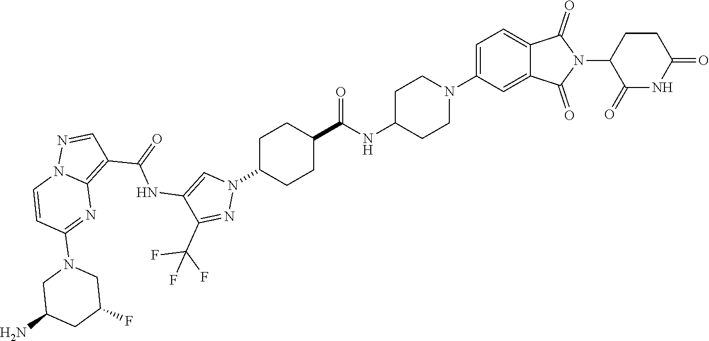 [SMILES: N[C@@H]1C[C@@H](F)CN(c2ccn3ncc(C(=O)Nc4cn([C@H]5CC[C@H](C(=O)NC6CCN(c7ccc8c(c7)C(=O)N(C7CCC(=O)NC7=O)C8=O)CC6)CC5)nc4C(F)(F)F)c3n2)C1] | 36% yield as a off-white solid. LCMS method 5: retention time: 2.097 min, 99% purity at 215 nm, [M − HCl + H]⁺ = 845.5, [M − HCl + 2H]²⁺ = 423.2. ¹H NMR (400 MHz, DMSO-d6) δ ppm 1.41-1.67 (m, 4 H), 1.67-2.27 (m, 11 H), 2.32- 2.46 (m, 2 H), 2.55-2.64 (m, 1 H), 2.85-2.96 (m, 1 H), 2.96-3.10 (m, 2 H), 3.27-3.52 (m, 3 H), 3.76-3.89 (m, 3 H), 4.18-4.37 (m, 3 H), 4.54- 4.97 (m, 2 H), 4.99-5.21 (m, 2 H), 6.90 (d, J = 7.8 Hz, 1 H), 6.97-7.31 (m, 3 H), 7.54 (d, J = 8.6 Hz, 1 H), 7.81 (d, J = 7.6 Hz, 1 H), 8.16-8.52 (m, 5 H), 8.93 (d, J = 7.8 Hz, 1 H), 9.33 (s, 1 H), 10.94 (s, 1 H). - Three protons obscured by water signal in ¹H NMR. ¹⁹F NMR (377 MHz, DMSO-d6) δ ppm −184.59 (s, 1 F), −111.30 (s, 2 F). |
| P-25 | 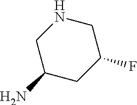 | T-3 | C-8 | 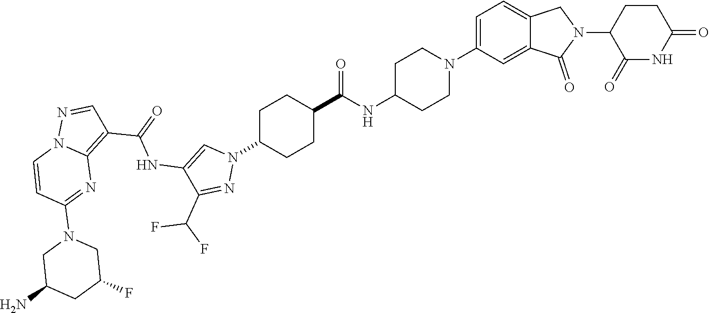 [SMILES: N[C@@H]1C[C@@H](F)CN(c2ccn3ncc(C(=O)Nc4cn([C@H]5CC[C@H](C(=O)NC6CCN(c7ccc8c(c7)C(=O)N(C7CCC(=O)NC7=O)C8)CC6)CC5)nc4C(F)F)c3n2)C1] | 69% yield as a white solid. LCMS method 5: retention time: 2.062 min, 99% purity at 215 nm, [M − HCl + H]⁺ = 845.5, [M − HCl + 2H]²⁺ = 423.3. ¹H NMR (400 MHz, DMSO- d6) δ ppm 1.21-1.31 (m, 1 H), 1.51- 2.13 (m, 15 H), 2.15-2.25 (m, 1 H), 2.34-2.46 (m, 2 H), 2.56-2.63 (m, 1 H), 2.85-3.15 (m, 3 H), 3.22-3.52 (m, 4 H), 4.20-4.30 (m, 2 H), 4.34- 4.42 (m, 1 H), 4.51-4.96 (m, 2 H), 5.02-5.19 (m, 2 H), 6.89 (d, J = 7.1 Hz, 1 H), 6.95-7.68 (m, 4 H), 7.86 (br s, 1 H), 8.18-8.37 (m, 4 H), 8.39 (s, 1 H), 8.93 (d, J = 7.8 Hz, 1 H), 9.33 (s, 1 H), 10.98 (s, 1 H). ¹⁹F NMR (377 MHz, DMSO-d6) δ ppm −184.66 (s, 1 F), −111.25 (s, 2 F) |
| P-31 | 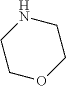 | T-3 | C-7 | 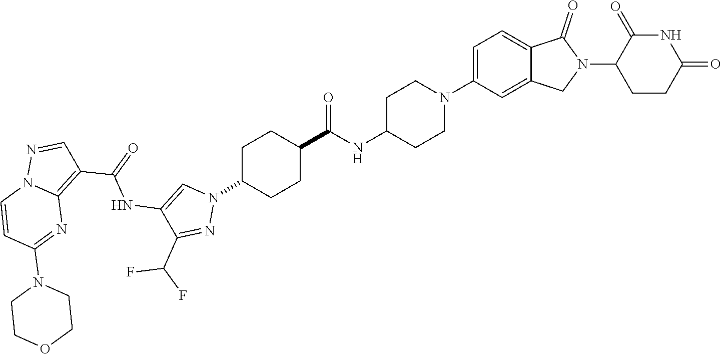 [SMILES: O=C1CCC(N2Cc3cc(N4CCC(NC(=O)[C@H]5CC[C@H](n6cc(NC(=O)c7cnn8ccc(N9CCOCC9)nc78)c(C(F)F)n6)CC5)CC4)ccc3C2=O)C(=O)N1] | 29% yield as a tan solid. LCMS method 5: retention time: 3.017 min, 99% purity at 215 nm, [M + 2H]²⁺ = 407.8, [M + H]⁺ = 814.3. ¹H NMR (400 MHz, DMSO-d6) δ ppm 1.42-1.63 (m, 4 H), 1.67-1.87 (m, 6 H), 1.93-2.00 (m, 1 H), 2.04- 2.11 (m, 2 H), 2.12-2.19 (m, 1 H), 2.35-2.44 (m, 1 H), 2.55-2.63 (m, 1 H), 2.85-3.02 (m, 3 H), 3.70-3.75 (m, 4 H), 3.77-3.86 (m, 7 H), 4.18- 4.35 (m, 3 H), 5.05 (dd, J = 13.2, 5.1 Hz, 1 H), 6.91 (d, J = 7.8 Hz, 1 H), 6.97-7.24 (m, 3 H), 7.51 (d, J = 8.6 Hz, 1 H), 7.77 (d, J = 7.8 Hz, 1 H), 8.29 (s, 1 H), 8.39 (s, 1 H), 8.83 (d, J = 7.8 Hz, 1 H), 9.40 (s, 1 H), 10.94 (s, 1 H). ¹⁹F NMR (377 MHz, DMSO-d6) δ ppm −111.32 (d, J = 54.5 Hz, 2 F). |
| P-33 | 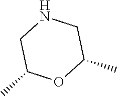 | T-3 | C-7 | 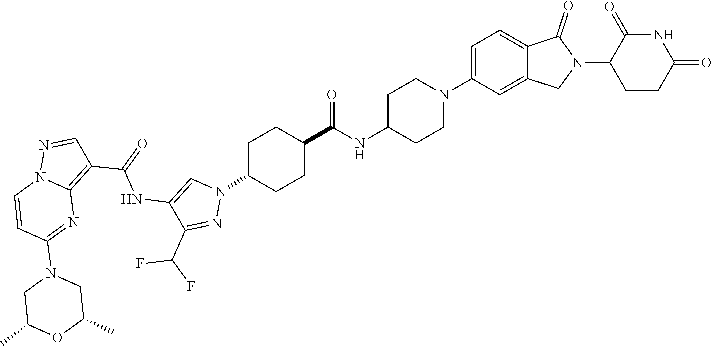 [SMILES: C[C@@H]1CN(c2ccn3ncc(C(=O)Nc4cn([C@H]5CC[C@H](C(=O)NC6CCN(c7ccc8c(c7)CN(C7CCC(=O)NC7=O)C8=O)CC6)CC5)nc4C(F)F)c3n2)C[C@H](C)O1] | 33% yield as a tan solid. LCMS method 5: retention time: 3.330 min, 96% purity at 215 nm, [M + 2H]²⁺ = 421.7; [M + H]⁺ = 842.4. ¹H NMR (400 MHz, DMSO-d6) δ ppm 1.19 (s, 3 H), 1.21 (s, 3 H), 1.38-1.53 (m, 2 H), 1.54-1.65 (m, 2 H), 1.66-1.91 (m, 7 H), 1.93-2.02 (m, 1 H), 2.05-2.12 (m, 2 H), 2.12- 2.22 (m, 1 H), 2.35-2.43 (m, 1 H), 2.56-2.63 (m, 1 H), 2.64-2.74 (m, 2 H), 2.85-3.05 (m, 3 H), 3.59-3.71 (m, 2 H), 3.75-3.89 (m, 3 H), 4.16- 4.39 (m, 4 H), 5.05 (dd, J = 13.3, 5.3 Hz, 1 H), 6.96 (d, J = 8.1 Hz, 1 H), 7.03-7.33 (m, 3 H), 7.52 (d, J = 8.3 Hz, 1 H), 7.77 (d, J = 7.8 Hz, 1 H), 8.29 (s, 1 H), 8.41 (s, 1 H), 8.83 (d, J = 7.8 Hz, 1 H), 9.28 (s, 1 H), 10.95 (s, 1 H). ¹⁹F NMR (377 MHz, DMSO-d6) δ ppm −110.90-−110.24 |
| | | | | | (m, 2 F). |

### Final Product General Method 2

### Example S5. Synthesis of P-6

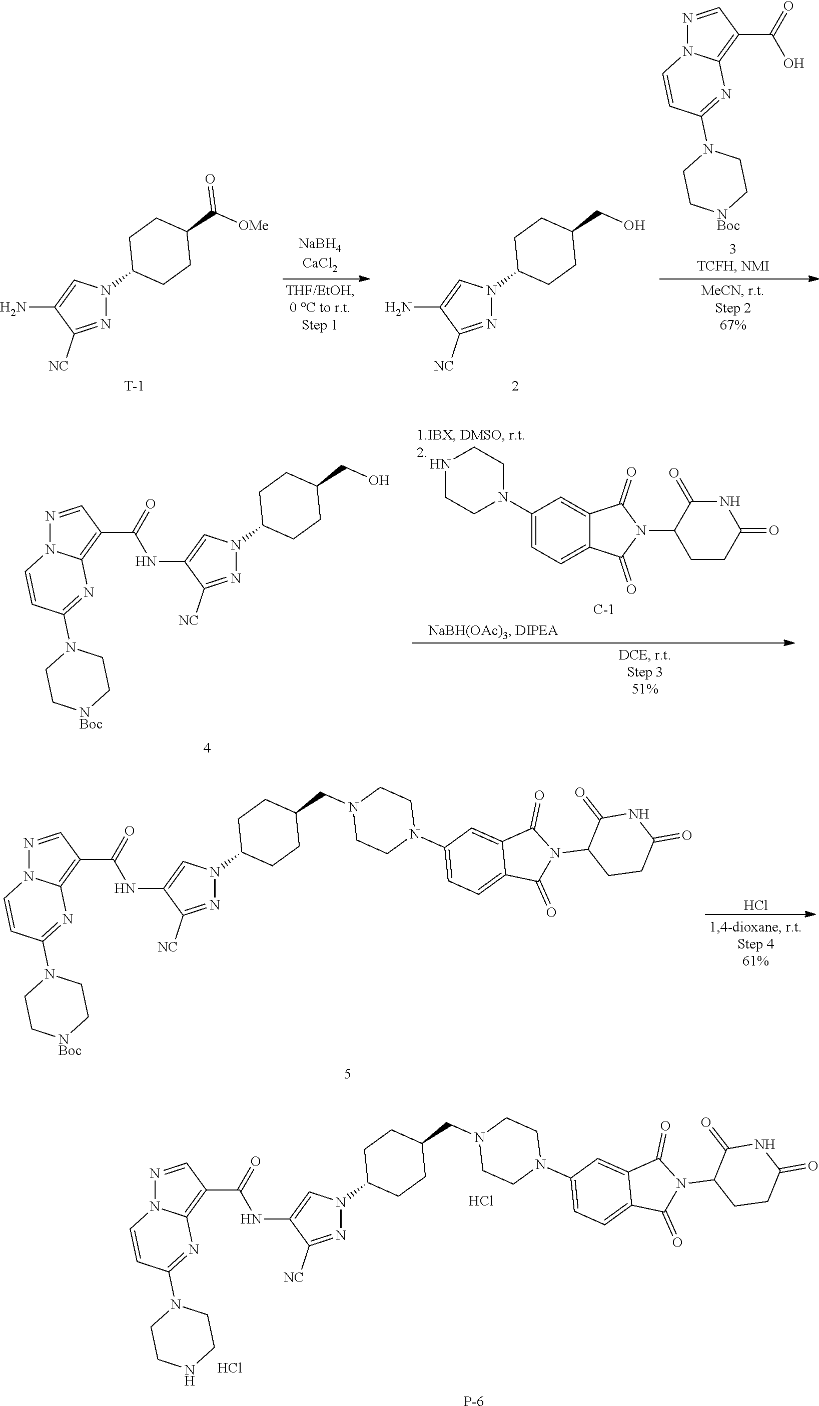

Step 1. Preparation of 4-amino-1-[4-(hydroxymethyl)cyclohexyl]pyrazole-3-carbonitrile (2). To a solution of methyl 4-(4-amino-3-cyano-pyrazol-1-yl)cyclohexanecarboxylate T-1 (25 mg, 0.100 mmol, 1 eq.) in THE (0.3 mL) and ethanol (0.6 mL) at 0° C. was added CaCl₂ (22.4 mg, 0.200 mmol, 2 eq.), followed by NaBH₄ (15.2 mg, 0.400 mmol, 4 eq.). The resulting mixture was stirred at 0° C. for 1 h and then at room temperature. After 17 h, LCMS showed full conversion. Water was added and the reaction mixture was stirred at room temperature. After 1 h, the aqueous layer was extracted 3 times with EtOAc. The organics were washed with brine, dried over Na₂SO₄ and concentrated to dryness. The crude of 2 was used without purification in the next step.

Step 2. Preparation of tert-Butyl 4-[3-[[3-cyano-1-[4-(hydroxymethyl)cyclohexyl]pyrazol-4-yl]carbamoyl]pyrazolo[1,5-a]pyrimidin-5-yl]piperazine-1-carboxylate (4). To a solution of 4-amino-[4-(hydroxymethyl)cyclohexyl]pyrazole-3-carbonitrile 2 (21.6 mg, 0.100 mmol, 1 eq.), 5-(4-tert-butoxycarbonylpiperazin-1-yl)pyrazolo[1,5-a]pyrimidine-3-carboxylic acid 3-[synthesis in Final Product General Method 1 P-1]-(35 mg, 0.100 mmol, 1 eq.) and NMI (28 uL, 0.350 mmol, 3.5 eq.) in MeCN (1 mL) was added TCFH (33.9 mg, 0.120 mmol, 1.2 eq.). The resulting mixture was stirred at room temperature. After 18 h, LCMS showed full conversion. Water was added and the reaction mixture was stirred at 0° C. for 30 min. The solid was filtered on a Buchner funnel, rinsed with water and then dried under high vacuum to give 4 (37.2 mg, 67% yield) as a white solid.

LCMS method 1: 86.9% purity at 215 nm, [M+H]=550.2.

¹H NMR (400 MHz, DMSO-d6) δ ppm 1.03-1.16 (m, 2H), 1.43 (s, 9H), 1.70-1.82 (m, 2H), 1.82-1.92 (m, 2H), 2.00-2.09 (m, 2H), 3.26 (t, J=5.6 Hz, 2H), 3.44 (br dd, J=4.6, 3.4 Hz, 1H), 3.50 (br s, 4H), 3.83 (br s, 4H), 4.30 (tt, J=12.0, 3.9 Hz, 1H), 4.48 (t, J=5.3 Hz, 1H), 6.92 (d, J=8.1 Hz, 1H), 8.31 (s, 1H), 8.46 (s, 1H), 8.85 (d, J=8.1 Hz, 1H), 9.61 (s, 1H).

Step 3. Preparation of tert-Butyl 4-[3-[[3-cyano-1-[4-[[4-[2-(2,6-dioxo-3-piperidyl)-1,3-dioxo-isoindolin-5-yl]piperazin-1-yl]methyl]cyclohexyl]pyrazol-4-yl]carbamoyl]pyrazolo[1,5-a]pyrimidin-5-yl]piperazine-1-carboxylate (5). Solution A: To a solution of tert-butyl 4-[3-[[3-cyano-1-[4-(hydroxymethyl)cyclohexyl]pyrazol-4-yl]carbamoyl]pyrazolo[1,5-a]pyrimidin-5-yl]piperazine-1-carboxylate 4 (37.2 mg, 0.070 mmol, 1 eq.) in anhydrous DMSO (1 mL) was added IBX (24.6 mg, 0.090 mmol, 1.3 eq.). The resulting mixture was stirred at room temperature. After 18 h, LCMS showed full conversion to tert-butyl 4-[3-[[3-cyano-1-(4-formylcyclohexyl)pyrazol-4-yl]carbamoyl]pyrazolo[1,5-a]pyrimidin-5-yl]piperazine-1-carboxylate.

To a solution of 2-(2,6-dioxo-3-piperidyl)-5-piperazin-1-yl-isoindoline-1,3-dione; 2,2,2-trifluoroacetic acid C-1 (30.8 mg, 0.070 mmol, 1 eq.) in anhydrous DCE (1 mL) was added DIPEA (0.024 mL, 0.140 mmol, 2 eq.). The resulting mixture was stirred at room temperature. After 5 min, NaBH(OAc)₃ (18.6 mg, 0.090 mmol, 1.3 eq.) was added, followed by Solution A. The resulting mixture was stirred at room temperature. After 2 h, LCMS showed conversion was almost complete. Volatiles were removed under reduce pressure and the residue was purified by reverse phase flash chromatography (30 g C18 RediSep Rf Gold column, liquid deposit (DMSO), elution: 5% MeOH/0.1% HCOOH over 3 CV, then 5 to 40% MeOH/0.1% HCOOH over 2 CV, then 40 to 60% MeOH/0.1% HCOOH over 11 CV, then 60% MeOH/0.1% HCOOH over 6 CV). Fractions were combined and concentrated to give 5 (30.3 mg, 51% yield) as a yellow solid.

LCMS method 1: 99.9% purity at 215 nm, [M+H]=874.2.

¹H NMR (400 MHz, DMSO-d6) δ ppm 1.01-1.15 (m, 2H), 1.43 (s, 9H), 1.62-1.71 (m, 1H), 1.75-1.87 (m, 2H), 1.90-2.12 (m, 5H), 2.16-2.22 (m, 2H), 2.53-2.63 (m, 2H), 2.82-2.94 (m, 1H), 3.42-3.55 (m, 8H), 3.84 (br d, J=2.7 Hz, 4H), 4.28-4.39 (m, 1H), 5.07 (dd, J=12.8, 5.3 Hz, 1H), 6.93 (d, J=7.8 Hz, 1H), 7.26 (dd, J=8.9, 1.3 Hz, 1H), 7.35 (s, 1H), 7.68 (d, J=8.6 Hz, 1H), 8.32 (s, 1H), 8.47 (s, 1H), 8.86 (d, J=8.1 Hz, 1H), 9.63 (s, 1H), 11.08 (s, 1H).

Step 4. Preparation of N-[3-Cyano-1-[4-[[4-[2-(2,6-dioxo-3-piperidyl)-1,3-dioxo-isoindolin-5-yl]piperazin-1-yl]methyl]cyclohexyl]pyrazol-4-yl]-5-piperazin-1-yl-pyrazolo[1,5-a]pyrimidine-3-carboxamide dihydrochloride (P-6). A solution of tert-butyl 4-[3-[[3-cyano-1-[4-[[4-[2-(2,6-dioxo-3-piperidyl)-1,3-dioxo-isoindolin-5-yl]piperazin-1-yl]methyl]cyclohexyl]pyrazol-4-yl]carbamoyl]pyrazolo[1,5-a]pyrimidin-5-yl]piperazine-1-carboxylate 5 (30 mg, 0.030 mmol, 1 eq.) in 4 M HCl in 1,4-dioxane (1.29 mL, 5.15 mmol, 150 eq.) was stirred at room temperature. After 1 h, LCMS showed full conversion. Solvents were removed under reduced pressure and the residue was purified by reverse phase flash chromatography (30 g C18 RediSep Rf Gold column, liquid deposit (DMSO), elution: 5% MeCN/0.02 M HCl over 3 CV, then 5 to 20% MeCN/0.02 M HCl over 6 CV, then 20 to 30% MeCN/0.02 M HCl over 12 CV). Fractions were combined, concentrated and lyophilized to give P-6 (16.2 mg, 61% yield) as a yellow solid as a full dihydrochloric acid salt.

LCMS method 5: 99.6% purity at 215 nm, [M−2HCl+H]=774.3.

¹H NMR (400 MHz, DMSO-d6) δ ppm 1.16-1.28 (m, 2H), 1.78-1.91 (m, 2H), 1.94-2.12 (m, 6H), 2.53-2.64 (m, 2H), 2.83-2.96 (m, 1H), 3.03-3.20 (m, 4H), 3.21-3.27 (m, 4H), 3.43-3.53 (m, 2H), 3.56-3.68 (m, 2H), 3.99-4.15 (m, 4H), 4.17-4.26 (m, 2H), 4.31-4.44 (m, 1H), 5.10 (dd, J=13.0, 5.1 Hz, 1H), 7.00 (d, J=8.1 Hz, 1H), 7.36 (d, J=9.8 Hz, 1H), 7.50 (s, 1H), 7.77 (d, J=8.6 Hz, 1H), 8.37 (s, 1H), 8.49 (s, 1H), 8.95 (d, J=7.8 Hz, 1H), 9.18 (br s, 2H), 9.59 (s, 1H), 10.18 (br s, 1H), 11.09 (s, 1H).

### Example S6. Synthesis of P-32

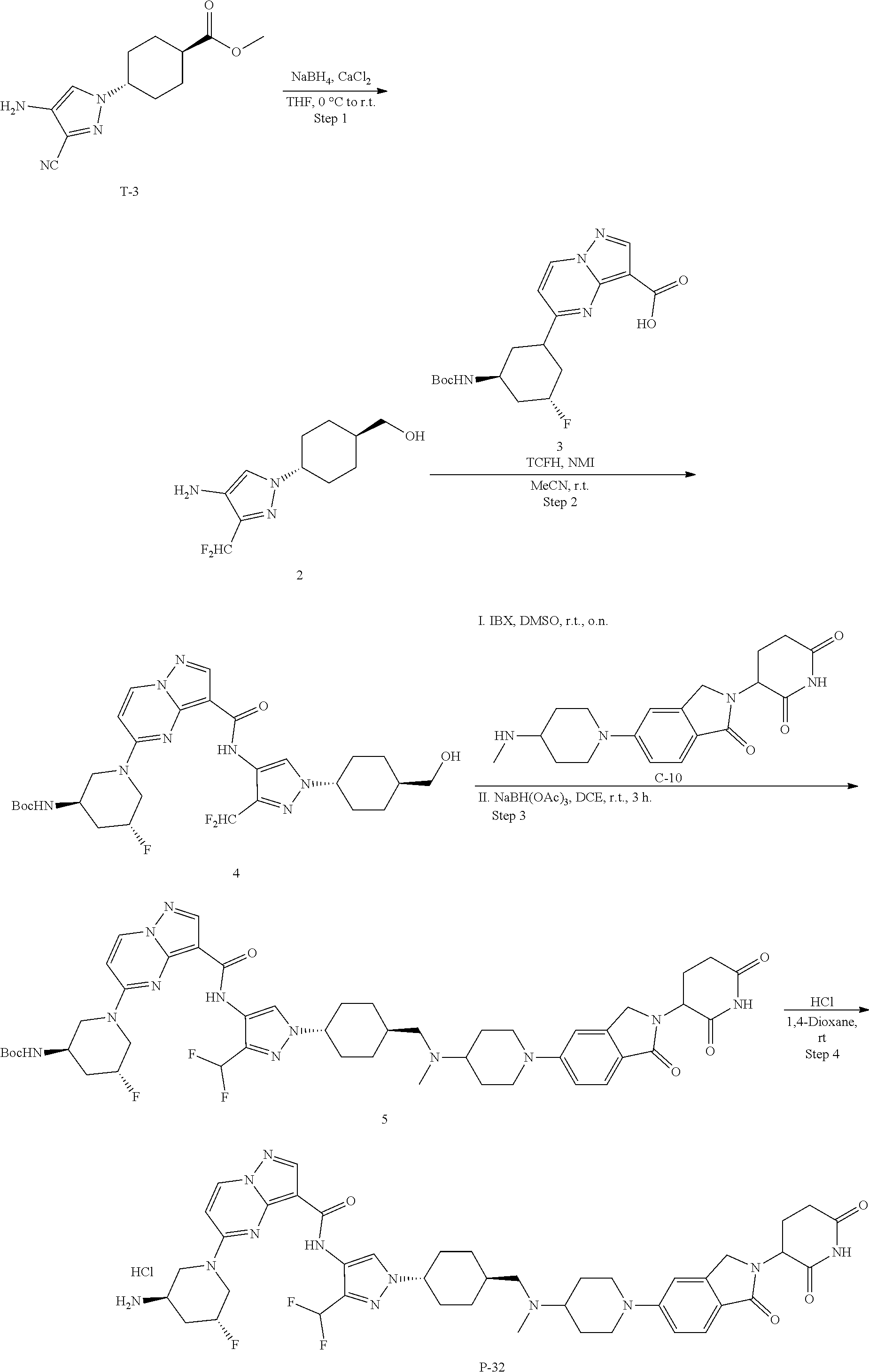

Step 1. Preparation of [4-[4-Amino-3-(difluoromethyl)pyrazol-1-yl]cyclohexyl]methanol (2). To a solution of methyl 4-[4-amino-3-(difluoromethyl)pyrazol-1-yl]cyclohexanecarboxylate T-3 (1.23 g, 4.5 mmol, 1.0 eq.) in THF (15.0 mL, 0.1 M) and ethanol (30.0 mL) at 0° C. was added CaCl₂)(1.0 g, 9.0 mmol, 2.0 eq,), followed by NaBH₄ (0.68 g, 18.0 mmol, 4.0 eq.). The resulting mixture was stirred overnight, allowing it to warm up to room temperature. Water was then added and the reaction mixture was stirred at room temperature for 1 hour. The aqueous phase was extracted with EtOAc (3×). The organic layers were washed with brine, dried over Na₂SO₄ and concentrated to dryness to afford [4-[4-Amino-3-(difluoromethyl)pyrazol-1-yl]cyclohexyl]methanol 2 (1.1 g, 99% yield) as an orange oil.

LCMS method 1: retention time: 0.989 min, 99.0% purity at 215 nm, [M+H]+=246.2.

¹H NMR (400 MHz, DMSO-d6) δ ppm 1.04 (qd, J=12.9, 3.2 Hz, 2H), 1.32-1.43 (m, 1H), 1.62 (qd, J=12.6, 3.3 Hz, 2H), 1.82 (br d, J=11.5 Hz, 2H), 1.93-1.98 (m, 2H), 3.24 (t, J=5.7 Hz, 2H), 3.94 (tt, J=11.9, 3.8 Hz, 1H), 4.00-4.06 (m, 2H), 4.44 (t, J=5.3 Hz, 1H), 6.72-7.02 (m, 1H), 7.14 (s, 1H).

¹⁹F NMR (377 MHz, DMSO-d6) δ ppm-111.21 (s, 2 F).

Step 2. Preparation of tert-Butyl N-[(3r,5r)-1-[3-[[3-(difluoromethyl)-1-[4-(hydroxymethyl)cyclohexyl]pyrazol-4-yl]carbamoyl]pyrazolo[1,5-a]pyrimidin-5-yl]-5-fluoro-3-piperidyl]carbamate (4). To a solution of [4-[4-amino-3-(difluoromethyl)pyrazol-1-yl]cyclohexyl]methanol 2 (511.8 mg, 1.88 mmol, 1.5 eq.), 5-[(3r,5r)-3-(tert-butoxycarbonylamino)-5-fluoro-1-piperidyl]pyrazolo[1,5-a]pyrimidine-3-carboxylic acid 3-[synthesis in Final Product General Method 1 P-13]-(475.0 mg, 1.25 mmol, 1.0 eq.) and NMI (362.72 uL, 4.58 mmol, 3.6 eq.) in MeCN (12.5 mL, 0.1 M) was added TCFH (440.43 mg, 1.57 mmol, 1.2 eq.). The resulting mixture was stirred at room temperature overnight. Water was added and the reaction mixture was stirred at room temperature for 1 hour. The solid was filtered on a Buchner funnel and rinsed with a water/MeCN mixture. Purification by reverse phase chromatography (50 g C18 RediSep Rf Gold column, liquid deposit (DMSO), elution: 5% MeOH/0.1% HCOOH over 4 CV, then 5% to 100% MeOH/0.1% HCOOH over 15 CV). The pure fractions were combined and concentrated under reduced pressure to afford tert-Butyl N-[(3r,5r)-1-[3-[[3-(difluoromethyl)-1-[4-(hydroxymethyl)cyclohexyl]pyrazol-4-yl]carbamoyl]pyrazolo[1,5-a]pyrimidin-5-yl]-5-fluoro-3-piperidyl]carbamate 4 (421 mg, 55% yield) as a tan solid.

LCMS method 1: retention time: 1.629 min, 99.9% purity at 215 nm, [M+H]+=607.2.

¹H NMR (400 MHz, DMSO-d6) δ ppm 1.03-1.15 (m, 2H), 1.34 (br s, 9H), 1.64-1.80 (m, 3H), 1.82-1.93 (m, 3H), 1.99-2.07 (m, 2H), 2.08-2.18 (m, 1H), 2.95-3.14 (m, 1H), 3.26 (t, J=5.7 Hz, 2H), 3.37-3.52 (m, 1H), 3.62-3.71 (m, 1H), 4.12-4.21 (m, 1H), 4.47 (t, J=5.3 Hz, 1H), 4.69 (br s, 1H), 4.95-5.12 (m, 1H), 6.86-6.94 (m, 1H), 7.01-7.22 (m, 2H), 8.28 (s, 1H), 8.34 (br s, 1H), 8.82 (d, J=7.8 Hz, 1H), 9.31 (br s, 1H).

¹⁹F NMR (377 MHz, DMSO-d6) δ ppm-186.02-179.79 (m, 1 F), -113.10-108.69 (m, 2F).

Step 3. Preparation of tert-Butyl N-[(3r,5r)-1-[3-[[3-(difluoromethyl)-1-[4-[[[1-[2-(2,6-dioxo-3-piperidyl)-1-oxo-isoindolin-5-yl]-4-piperidyl]-methyl-amino]methyl]cyclohexyl]pyrazol-4-yl]carbamoyl]pyrazolo[1,5-a]pyrimidin-5-yl]-5-fluoro-3-piperidyl]carbamate (5). To a solution of tert-butyl N-[(3R,5R)-1-[3-[[3-(difluoromethyl)-1-[4-(hydroxymethyl)cyclohexyl]pyrazol-4-yl]carbamoyl]pyrazolo[1,5-a]pyrimidin-5-yl]-5-fluoro-3-piperidyl]carbamate 4 (75.0 mg, 0.12 mmol, 1.0 eq.) in dry DMSO (1 mL, 0.12 M) was added IBX (41.54 mg, 0.15 mmol, 1.2 eq.). The resulting mixture was stirred at room temperature. LCMS (method 3) after overnight stirring showed complete conversion to the desired product tert-butyl N-[(3R,5R)-1-[3-[[3-(difluoromethyl)-1-(4-formylcyclohexyl)pyrazol-4-yl]carbamoyl]pyrazolo[1,5-a]pyrimidin-5-yl]-5-fluoro-3-piperidyl]carbamate. To the reaction mixture was added 3-[5-[4-(methylamino)-1-piperidyl]-1-oxo-isoindolin-2-yl]piperidine-2,6-dione hydrochloride (53.61 mg, 0.14 mmol, 1.2 eq.), DCE (1 mL, 0.06 M) and DIPEA (0.22 mL, 1.24 mmol, 10 eq.). The mixture was stirred at room temperature for 10 minutes. Sodium triacetoxyborohydride (34.18 mg, 0.16 mmol, 1.3 eq.) was added and the mixture stirred at room temperature for 3 hours. The reaction mixture was concentrated in vacuo and purified by reverse phase chromatography (50 g C18 RediSep Rf Gold column, liquid deposit (DMSO), elution: 5% MeCN/0.1% HCOOH over 4 CV, then 5% to 40% MeCN/0.1% HCOOH over 15 CV). The pure fractions were combined and concentrated to give tert-butyl N-[(3R,5R)-1-[3-[[3-(difluoromethyl)-1-[4-[[[1-[2-(2,6-dioxo-3-piperidyl)-1-oxo-isoindolin-5-yl]-4-piperidyl]-methyl-amino]methyl]cyclohexyl]pyrazol-4-yl]carbamoyl]pyrazolo[1,5-a]pyrimidin-5-yl]-5-fluoro-3-piperidyl]carbamate 5 (49 mg, 36% yield) as a tan solid. The desired product contained IBX residue.

LCMS method 2: retention time: 1.685 min, 87.7% purity at 215 nm, [M-Boc+2H]2+=423.2.

¹H NMR (400 MHz, DMSO-d6) δ ppm 0.98-1.06 (m, 2H), 1.31-1.38 (m, 7H), 1.45 (s, 9H), 1.48-1.54 (m, 2H), 1.71-1.80 (m, 4H), 1.86-1.94 (m, 4H), 2.04 (dt, J=15.2, 3.1 Hz, 2H), 2.18-2.27 (m, 5H), 2.79-2.88 (m, 3H), 3.46-3.52 (m, 1H), 3.62-3.72 (m, 2H), 3.94 (br d, J=11.5 Hz, 2H), 4.20 (br d, J=17.1 Hz, 1H), 4.33 (br d, J=16.9 Hz, 1H), 4.96-5.11 (m, 3H), 5.79-5.83 (m, 1H), 7.06 (s, 2H), 7.53 (dd, J=15.9, 8.1 Hz, 2H), 7.97 (br dd, J=8.4, 1.3 Hz, 1H), 8.03 (dd, J=8.6, 1.2 Hz, 1H), 8.29 (s, 1H), 8.33 (br s, 1H), 8.82 (d, J=7.8 Hz, 1H), 9.31 (br s, 1H), 10.94 (s, 1H).

Step 4. Preparation of N-[3-(Difluoromethyl)-1-[4-[[[1-[2-(2,6-dioxo-3-piperidyl)-1-oxo-isoindolin-5-yl]-4-piperidyl]-methyl-amino]methyl]cyclohexyl]pyrazol-4-yl]-5-[(3r,5r)-3-amino-5-fluoro-1-piperidyl]pyrazolo[1,5-a]pyrimidine-3-carboxamide hydrochloride (P-32). HCl 4.0 M in dioxane (1.94 mL, 7.78 mmol, 150.0 eq.) was added to tert-butyl N-[(3r,5r)-1-[3-[[3-(difluoromethyl)-1-[4-[[[1-[2-(2,6-dioxo-3-piperidyl)-1-oxo-isoindolin-5-yl]-4-piperidyl]-methyl-amino]methyl]cyclohexyl]pyrazol-4-yl]carbamoyl]pyrazolo[1,5-a]pyrimidin-5-yl]-5-fluoro-3-piperidyl]carbamate 5 (49. mg, 0.05 mmol, 1.0 eq.). The reaction mixture was stirred for 2 hours at room temperature. The reaction mixture was concentrated in vacuo. Purification by reverse phase chromatography (50 g C18 RediSep Rf Gold column, liquid deposit (H₂O), elution: 5% MeCN/0.02 M HCl over 4 CV, then 5% to 40% MeCN/0.02 M HCl over 15 CV). The pure fractions were combined, concentrated under reduced pressure and lyophilised overnight to afford N-[3-(difluoromethyl)-1-[4-[[[1-[2-(2,6-dioxo-3-piperidyl)-1-oxo-isoindolin-5-yl]-4-piperidyl]-methyl-amino]methyl]cyclohexyl]pyrazol-4-yl]-5-[(3r,5r)-3-amino-5-fluoro-1-piperidyl]pyrazolo[1,5-a]pyrimidine-3-carboxamide P-32 (34.8 mg, 79% yield).

LCMS method 5: retention time: 1.797 min, 99.9% purity at 215 nm, [M−HCl+H]+=845.5; [M−2HCl+2H]2+=423.3.

¹H NMR (400 MHz, DMSO-d6) δ ppm 1.12-1.29 (m, 2H), 1.68-2.00 (m, 8H), 2.03-2.19 (m, 6H), 2.34-2.42 (m, 2H), 2.54-2.63 (m, 2H), 2.72-2.78 (m, 3H), 2.85-2.96 (m, 4H), 3.08-3.15 (m, 1H), 3.28-3.39 (m, 2H), 4.06 (br d, J=12.7 Hz, 2H), 4.20-4.36 (m, 3H), 4.53-4.65 (m, 1H), 4.79-4.91 (m, 1H), 5.02-5.18 (m, 2H), 6.90 (d, J=8.1 Hz, 1H), 7.00-7.28 (m, 3H), 7.55 (d, J=8.6 Hz, 1H), 8.28-8.34 (m, 3H), 8.40 (s, 1H), 8.94 (d, J=7.8 Hz, 1H), 9.34 (s, 1H), 9.56-9.65 (m, 1H), 10.95 (s, 1H).

¹⁹F NMR (377 MHz, DMSO-d6) δ ppm-184.56 (s, 2 F), -111.32 (s, 1 F).

Example S7. The following compounds were synthesized via the same general routes from combination of starting intermediate (T-X) in step 1, acid substrate 3 in step 2, and CBM (C—X) in step 3 (Table 3).

TABLE 3

| Compound | | | | | |
|---|---|---|---|---|---|
| No. | Amine | Int | CBM | Structure | Characterization Data |
| P-6 | 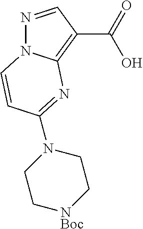 [SMILES: CC(C)(C)OC(=O)N1CCN(c2ccn3ncc(C(=O)O)c3n2)CC1] | T-1 | C-1 | 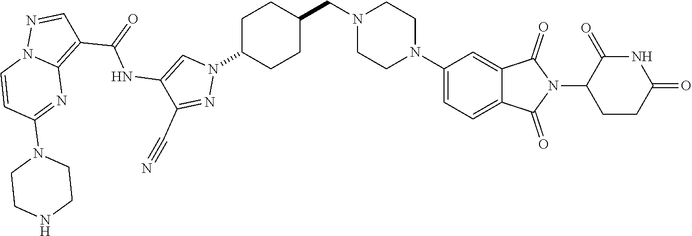 | 61% yield as a yellow solid. LCMS method 5: retention time: 1.808 min, 99% purity at 215 nm, [M − 2HCl + H]⁺ = 774.3; [M − HCl + 2H]²⁺ = 387.8. ¹H NMR (400 MHz, DMSO-d6) δ ppm 1.16-1.28 (m, 2 H), 1.78-1.91 (m, 2 H), 1.94-2.12 (m, 6 H), 2.53-2.64 (m, 2 H), 2.83-2.96 (m, 1 H), 3.03-3.20 (m, 4 H), 3.21-3.27 (m, 4 H), 3.43-3.53 (m, 2 H), 3.56-3.68 (m, 2 H), 3.99-4.15 (m, 4 H), 4.17-4.26 (m, 2 H), 4.31-4.44 (m, 1 H), 5.10 (dd, |
| | | | | | J = 13.0, 5.1 Hz, 1 H), 7.00 |
| | | | | | (d, J = 8.1 Hz, 1 H), 7.36 (d, |
| | | | | | J = 9.8 Hz, 1 H), 7.50 (s, 1 |
| | | | | | H), 7.77 (d, J = 8.6 Hz, 1 H), |
| | | | | | 8.37 (s, 1 H), 8.49 (s, 1 H), |
| | | | | | 8.95 (d, J = 7.8 Hz, 1 H), |
| | | | | | 9.18 (br s, 2 H), 9.59 (s, 1 H), |
| | | | | | 10.18 (br s, 1 H), 11.09 (s, 1 |
| | | | | | H). |
| P-9 | 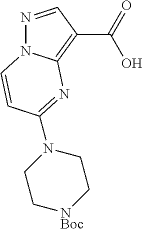 [SMILES: CC(C)(C)OC(=O)N1CCN(c2ccn3ncc(C(=O)O)c3n2)CC1] | T-1 | C-5 | 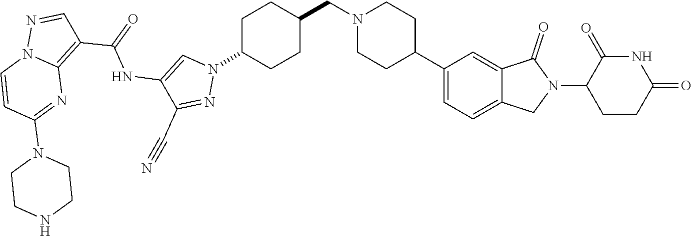 | 71% yield as a white solid. LCMS method 5: retention time: 1.786 min, 99% purity at 215 nm, [M − 2HCl + H]+ = 759.3; [M − 2HCl + 2H]²⁺ = 380.3. ¹H NMR (400 MHz, DMSO-d6) δ ppm 1.15-1.30 (m, 2 H), 1.77-2.30 (m, 12 H), 2.34-2.47 (m, 1 H), 2.56-2.65 (m, 1 H), 2.83-3.17 (m, 6 H), 3.20-3.28 (m, 4 H), 3.55-3.72 (m, 2 H), 3.93-4.23 (m, 4 H), 4.27-4.49 (m, 3 H), 5.11 (dd, J = 12.7, 4.6 Hz, 1 H), 7.00 (d, J = 7.8 Hz, 1 H), |
| | | | | | 7.50-7.66 (m, 3 H), 8.36 (s, 1 |
| | | | | | H), 8.49 (s, 1 H), 8.94 (d, J = |
| | | | | | 7.8 Hz, 1 H), 9.23 (br s, 2 H), |
| | | | | | 9.59 (s, 1 H), 9.72 (br s, 1 H), |
| | | | | | 10.99 (s, 1 H). |
| P-17 | 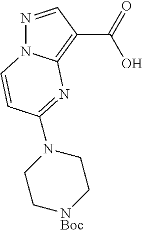 [SMILES: CC(C)(C)OC(=O)N1CCN(c2ccn3ncc(C(=O)O)c3n2)CC1] | T-1 | C-6 | 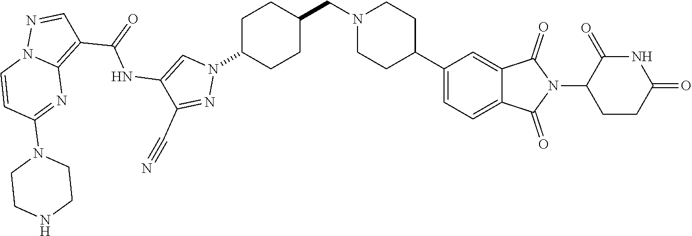 | 65% yield as a white solid. LCMS method 5: retention time: 1.812 min, 98% purity at 215 nm, [M − 2HCl + 2H]²⁺ = 387.2; [M − 2HCl + H]⁺ = 773.2. ¹H NMR (400 MHz, DMSO-d6) δ ppm 1.17-1.30 (m, 2 H), 1.78-1.95 (m, 3 H), 1.95-2.14 (m, 8 H), 2.16-2.30 (m, 2 H), 2.53-2.66 (m, 2 H), 2.84-2.95 (m, 1 H), 2.98-3.15 (m, 4 H), 3.19-3.28 (m, 4 H), 3.60-3.71 (m, 2 H), 3.99-4.15 (m, 4 H), 4.34-4.44 (m, 1 H), 5.15 (dd, J = 12.7, |
| | | | | | 5.1 Hz, 1 H), 7.00 (d, J = 8.1 |
| | | | | | Hz, 1 H), 7.75-7.88 (m, 2 H), |
| | | | | | 7.93 (d, J = 7.8 Hz, 1 H), 8.37 |
| | | | | | (s, 1 H), 8.49 (s, 1 H), 8.95 (d, |
| | | | | | J = 7.8 Hz, 1 H), 9.19 (br s, 2 |
| | | | | | H), 9.59 (s, 1 H), 9.74 (br s, 1 |
| | | | | | H), 11.13 (s, 1 H). |
| P-26 | 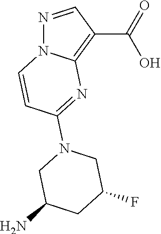 [SMILES: N[C@@H]1C[C@@H](F)CN(c2ccn3ncc(C(=O)O)c3n2)C1] | T-3 | C-1 | 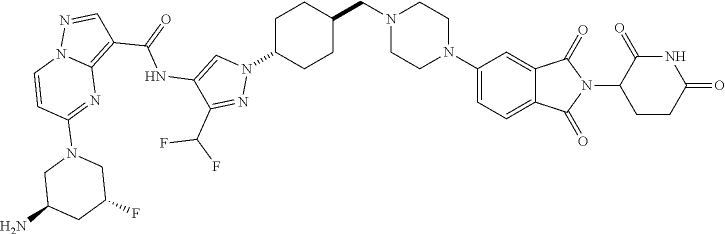 | 75% yield as a yellow solid. LCMS method 5: retention time: 1.832 min, 99% purity at 215 nm, [M − 2HC1+ 2H]²⁺ = 416.2; [M − 2HC1 + H]⁺ = 831.3. ¹H NMR (400 MHz, DMSO-d6) δ ppm 1.15-1.30 (m, 2 H), 1.76-1.88 (m, 2 H), 1.89-2.16 (m, 8 H), 2.35-2.45 (m, 1 H), 2.54-2.65 (m, 2 H), 2.82-2.95 (m, 1 H), 3.03-3.20 (m, 4 H), 3.37-3.44 (m, 1 H), 3.45-3.55 (m, 3 H), 3.58-3.66 (m, 2 H), 4.16-4.30 (m, 3 H), 4.50-4.72 (m, 1 H), |
| | | | | | 4.75-4.98 (m, 1 H), 5.03-5.19 |
| | | | | | (m, 2 H), 6.90 (d, J = 8.1 Hz, |
| | | | | | 1 H), 7.14 (t, J = 54.3 Hz, 1 |
| | | | | | H), 7.37 (dd, J = 8.6, 1.0 Hz, |
| | | | | | 1 H), 7.50 (s, 1 H), 7.77 (d, |
| | | | | | J = 8.3 Hz, 1 H), 8.27-8.48 |
| | | | | | (m, 5 H), 8.94 (d, J = 7.8 Hz, |
| | | | | | 1 H), 9.34 (s, 1 H), 10.27 (br |
| | | | | | s, 1 H), 11.09 (s, 1 H). - One |
| | | | | | proton is obscured by the |
| | | | | | water peak in the ¹H NMR. |
| | | | | | ¹⁹F NMR (377 MHz, DMSO- |
| | | | | | d6) δ ppm −184.64 (s, 1 F), |
| | | | | | −111.28 (s, 2 F). |
| P-32 | 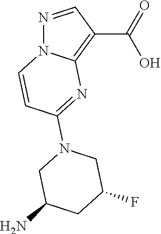 [SMILES: N[C@@H]1C[C@@H](F)CN(c2ccn3ncc(C(=O)O)c3n2)C1] | T-3 | C-10 | 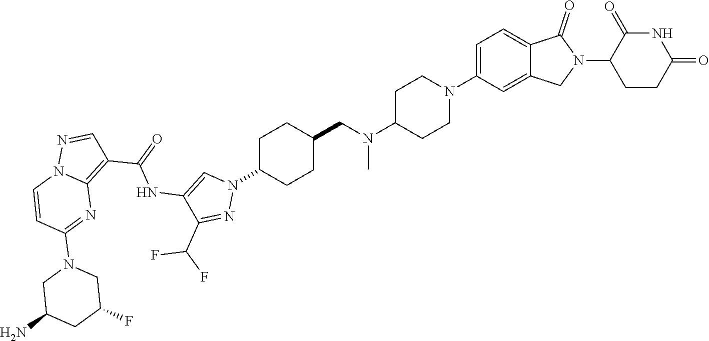 | 79% yield as a light yellow solid. LCMS method 5: retention time: 1.797 min, 99% purity at 215 nm, [M − HCl + H]+ = 845.5; [M − 2HCl + 2H]²⁺ = 423.3. ¹H NMR (400 MHz, DMSO-d6) δ ppm 1.12-1.29 (m, 2 H), 1.68-2.00 (m, 8 H), 2.03-2.19 (m, 6 H), 2.34-2.42 (m, 2 H), 2.54-2.63 (m, 2 H), 2.72-2.78 (m, 3 H), 2.85-2.96 (m, 4 H), 3.08-3.15 (m, 1 H), 3.28-3.39 (m, 2 H), 4.06 (br d, J = 12.7 Hz, 2 H), 4.20-4.36 (m, 3 H), 4.53-4.65 (m, 1 H), 4.79-4.91 (m, 1 H), 5.02-5.18 (m, 2 H), 6.90 (d, J = 8.1 Hz, 1 H), 7.00-7.28 (m, 3 H), 7.55 (d, J = 8.6 Hz, 1 H), 8.28-8.34 (m, 3 H), 8.40 (s, 1 H), 8.94 |
| | | | | | (d, J = 7.8 Hz, 1 H), 9.34 (s, |
| | | | | | 1 H), 9.56-9.65 (m, 1 H), |
| | | | | | 10.95 (s, 1 H). ¹⁹F NMR (377 |
| | | | | | MHz, DMSO-d6) δ ppm |
| | | | | | −184.56 (s, 2 F), −111.32 (s, |
| | | | | | 1 F). |
| P-34 | 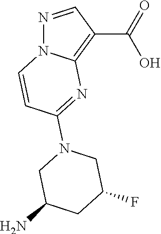 [SMILES: N[C@@H]1C[C@@H](F)CN(c2ccn3ncc(C(=O)O)c3n2)C1] | T-3 | C-11 | 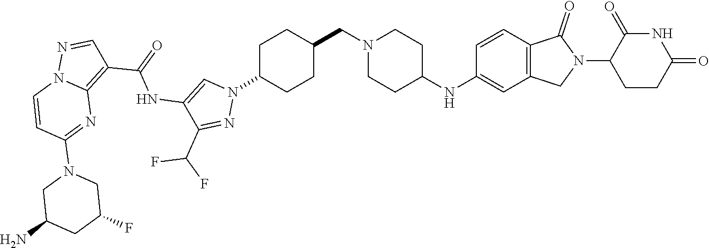 | 56% yield as a tan solid. LCMS method 3: retention time: 1.896 min, 97% purity at 215 nm, [M− 2HCl + H]⁺ = 845.4; [M − 2HCl + 2H]²⁺ = 423.3. ¹H NMR (400 MHz, DMSO-d6) δ ppm 1.16-1.29 (m, 2 H), 1.76-1.89 (m, 4 H), 1.90-2.14 (m, 8 H), 2.32- 2.43 (m, 4 H), 2.54-2.63 (m, 1 H), 2.84 (s, 3 H), 2.88-2.99 (m, 3 H), 3.06-3.19 (m, 2 H), 3.27-3.52 (m, 4 H), 3.54-3.65 (m, 2 H), 4.62 (br s, 1 H), 4.85 (br s, 1 H), 5.00-5.19 |
| | | | | | (m, 2 H), 6.90 (br d, J = 8.1 |
| | | | | | Hz, 1 H), 6.94-7.29 (m, 3 H), |
| | | | | | 7.53 (d, J = 9.0 Hz, 1 H), |
| | | | | | 8.29-8.38 (m, 4 H), 8.40 (s, |
| | | | | | 1 H), 8.93 (d, J = 7.8 Hz, 1 |
| | | | | | H), 9.34 (s, 1 H), 9.83 (s, 1 |
| | | | | | H), 10.94 (s, 1 H). - Two |
| | | | | | protons are not obsereved |
| | | | | | by ¹H NMR. ¹⁹F NMR (377 |
| | | | | | MHz, DMSO-d6) δ ppm |
| | | | | | −185.36-−184.06 (m, 1 F), |
| | | | | | −111.60-−110.99 (m, 2 F). |
| P-35 | 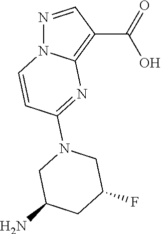 [SMILES: N[C@@H]1C[C@@H](F)CN(c2ccn3ncc(C(=O)O)c3n2)C1] | T-3 | C-12 | 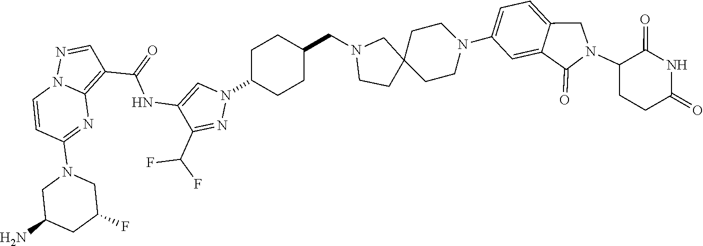 | 27% yield as a yellow solid. LCMS method 5: retention time: 1.835 min, 97% purity at 215 nm, [M − 2HCl + H]⁺ = 871.4, [M − 2HCl + 2H]²⁺ = 436.3, [M − 2HCl + 3H]³⁺ = 291.2. ¹H NMR (400 MHz, DMSO-d6) δ ppm 1.12-1.26 (m, 2 H), 1.71-2.15 (m, 15 H), 2.32-2.46 (m, 2 H), 2.54 (s, 1 H), 2.56-2.63 (m, 1 H), 2.85-2.99 (m, 2 H), 3.02-3.24 (m, 3 H), 3.34-3.48 (m, 6 H), 3.58-3.71 (m, 2 H), 4.17-4.30 (m, 2 H), 4.36 (d, |
| | | | | | J = 1.0 Hz, 1 H), 4.58-4.71 |
| | | | | | (m, 1 H), 4.79-4.94 (m, 1 H), |
| | | | | | 5.02-5.17 (m, 2 H), 6.92 (d, |
| | | | | | J = 7.8 Hz, 1 H), 7.00-7.27 |
| | | | | | (m, 1 H), 7.28-7.42 (m, 2 H), |
| | | | | | 7.60 (d, J = 8.3 Hz, 1 H), |
| | | | | | 8.32 (s, 1 H), 8.39 (s, 1 H), |
| | | | | | 8.53 (s, 3 H), 8.92 (d, J = 7.8 |
| | | | | | Hz, 1 H), 9.33 (s, 1 H), 10.87 |
| | | | | | (s, 1 H), 10.96 (s, 1 H). ¹⁹F |
| | | | | | NMR (377 MHz, DMSO-d6) |
| | | | | | δ ppm 184.84-−184.03 (m, 1 |
| | | | | | F), −111.75-−110.99 (m, 2 F). |
| P-36 | 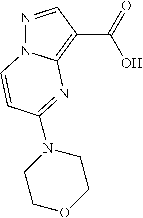 [SMILES: O=C(O)c1cnn2ccc(N3CCOCC3)nc12] | T-3 | C-13 | 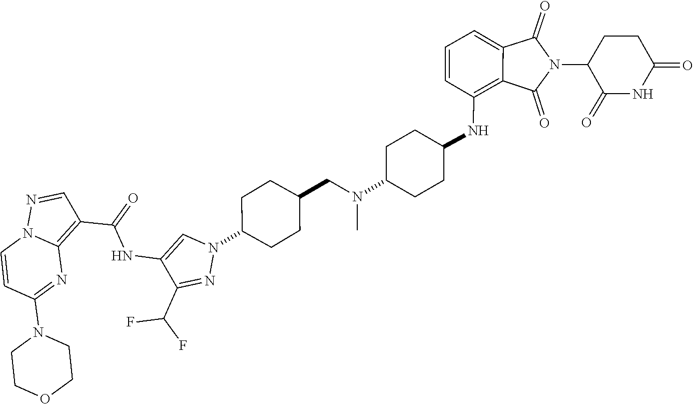 | 29% yield as a yellow solid. LCMS method 5: retention time: 2.447 min, 99% purity at 215 nm, [M + 2H]²⁺ = 421.8, [M + H]⁺ = 842.4. ¹H NMR (400 MHz, DMSO- d6) δ ppm 1.01 (q, J = 11.0 Hz, 2 H), 1.22-1.36 (m, J = 11.7 Hz, 2 H), 1.37-1.54 (m, 3 H), 1.68-1.81 (m, 4 H), 1.91 (br d, J = 11.7 Hz, 2 H), 1.98-2.08 (m, 5 H), 2.20 (s, 3 H), 2.23 (br d, J = 6.8 Hz, 2 H), 2.40 (t, J = 11.5 Hz, 1 H), 2.52-2.64 (m, 2 H), 2.82- 2.93 (m, 1 H), 3.44-3.56 (m, 1 H), 3.69-3.75 (m, 4 H), 3.75-3.85 (m, 4 H), 4.18 (tt, J = 11.5, 3.2 Hz, 1 H), 5.04 (dd, J = 12.7, 5.4 Hz, 1 H), 6.15 (d, J = 8.3 Hz, 1 H), 6.88-7.24 (m, 4 H), 7.58 (dd, J = 8.4, 7.2 Hz, 1 H), 8.29 (s, 1 H), 8.38 (s, 1 H), 8.83 (d, J = 8.1 Hz, 1 H), 9.40 (s, 1 |
| | | | | | H), 11.08 (br s, 1 H). ¹⁹F |
| | | | | | NMR (377 MHz, DMSO-d6) |
| | | | | | δ ppm −111.19 (d, J = 54.5 |
| | | | | | Hz, 2 F). |
| P-37 | 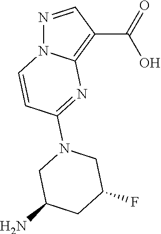 [SMILES: N[C@@H]1C[C@@H](F)CN(c2ccn3ncc(C(=O)O)c3n2)C1] | T-3 | C-13 | 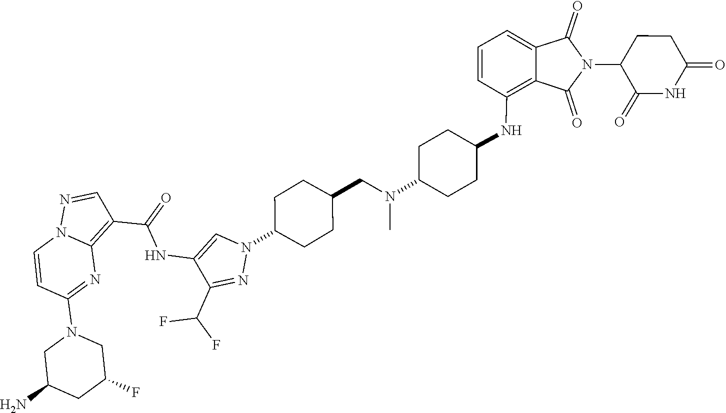 | 32% yield as a yellow solid. LCMS method 5: retention time: 1.978 min, 99% purity at 215 nm, [M − 2HCl + H]⁺ = 873.4, [M − 2HCl + H]²⁺ = 437.3. ¹H NMR (400 MHz, DMSO-d6) δ ppm 1.09-1.24 (m, 2 H), 1.24-1.31 (m, 4 H), 1.37-1.51 (m, 2 H), 1.66-1.97 (m, 6 H), 2.03-2.21 (m, 8 H), 2.36-2.45 (m, 1 H), 2.56-2.63 (m, 1 H), 2.74 (br d, J = 4.6 Hz, 3 H), 2.81-2.95 (m, 2 H), 3.03-3.18 (m, 2 H), 3.22-3.50 (m, 6 H), 4.25 (tt, J = 11.6, 3.7 Hz, 1 H), 4.64 (br s, 1 H), 4.87 (br s, 1 H), 5.00-5.20 (m, 2 H), 6.20 (br d, J = 7.8 Hz, 1 H), 6.91 (br d, J = 8.1 Hz, 1 H), 6.99-7.29 (m, 3 H), 7.62 (dd, J = 8.4, 7.2 Hz, 1 H), 8.32 (s, 1 H), 8.34-8.52 (m, 4 H), 8.93 (d, J = 7.8 Hz, 1 H), 9.34 (s, 1 H), 9.79 (br s, 1 H), 11.10 (s, |
| | | | | | 1 H). ¹⁹F NMR (377 MHz, |
| | | | | | DMSO-d6) δ ppm −184.58 |
| | | | | | (br s, 1 F), −111.32 (br d, J = |
| | | | | | 38.1 Hz, 2 F). |

### Final Product Method 3

### Example S8. Synthesis of P-27

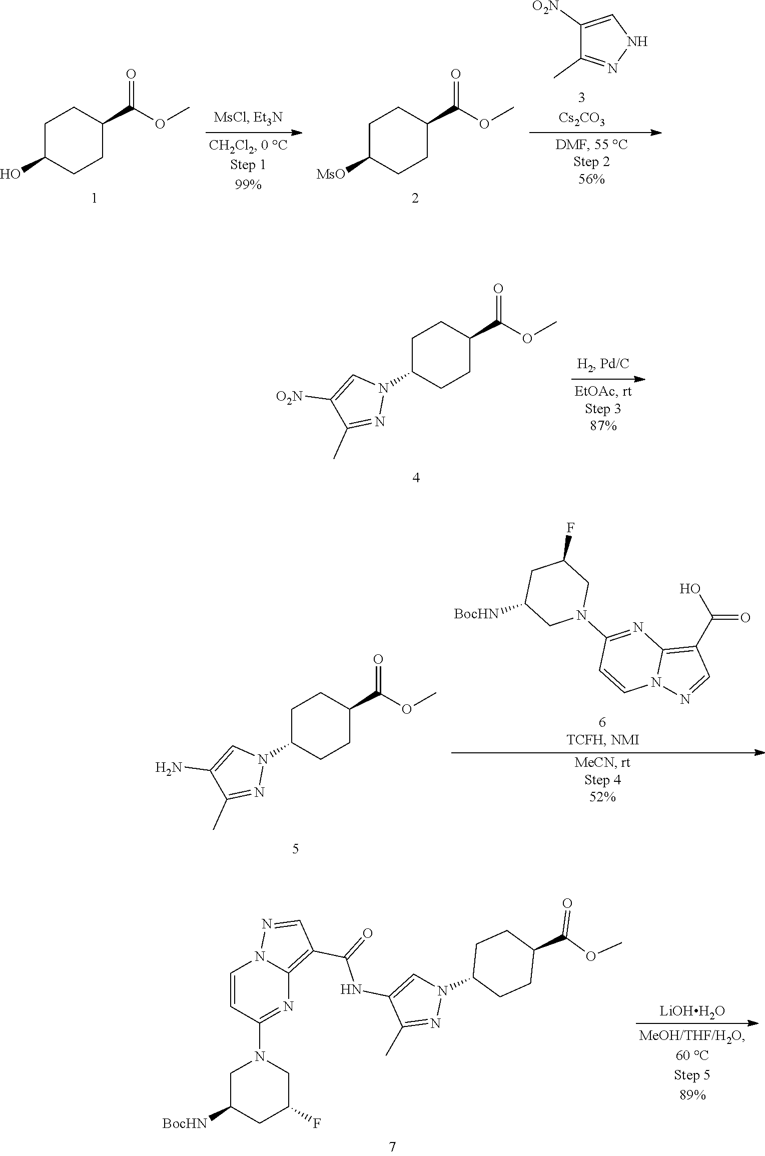

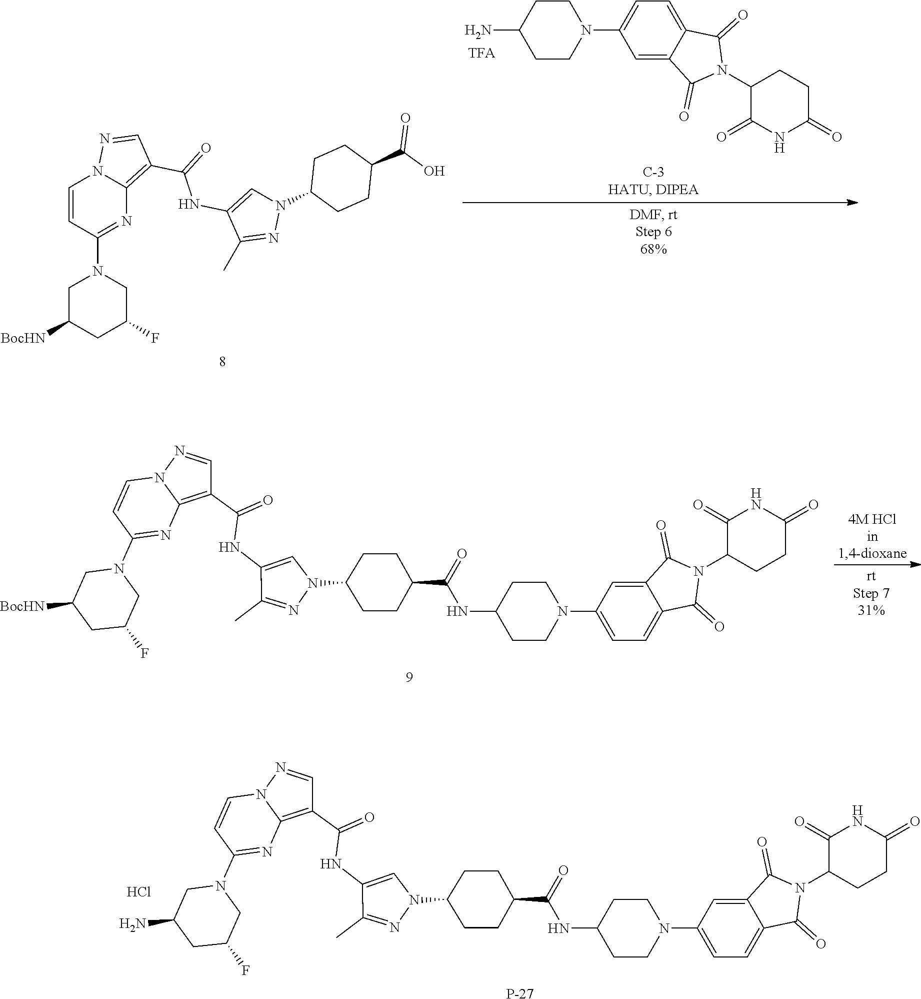

Step 1. Preparation of Methyl 4-methylsulfonyloxycyclohexanecarboxylate (2). Under nitrogen, a solution of methyl 4-hydroxycyclohexanecarboxylate 1 (5.0 g, 31.61 mmol, 1.0 eq.) in CH₂Cl₂ (105 mL, 0.3 M) was cooled to 0° C., then methanesulfonyl chloride (2.69 mL, 34.77 mmol, 1.1 eq.) and triethylamine (5.28 mL, 37.93 mmol, 1.2 eq.) were added, the latter dropwise. After stirring 2 h at 0° C., TLC (CH₂Cl₂/MeOH 5:1, KMnO₄ stain) showed full conversion. The reaction was quenched by addition of water, then phases were separated and the aqueous phase was extracted 3 times with CH₂Cl₂. The combined organic phases were washed twice with brine, dried over magnesium sulfate, filtered and concentrated to give 2 (7.45 g, 99% yield) as a yellow oil.

¹H NMR (400 MHz, CDCl₃) δ ppm 1.67-1.84 (m, 4H), 1.87-1.99 (m, 2H), 2.00-2.09 (m, 2H), 2.36-2.46 (m, 1H), 3.02 (s, 3H), 3.69 (s, 3H), 4.87-4.96 (m, 1H).

Step 2. Preparation of Methyl 4-(3-methyl-4-nitro-pyrazol-1-yl)cyclohexanecarboxylate (4). To a solution of methyl 4-methylsulfonyloxycyclohexanecarboxylate 2 (976.02 mg, 4.13 mmol) and 3-methyl-4-nitro-1H-pyrazole 3 (350. mg, 2.75 mmol) in dry DMF (13.769 mL) was added Cs₂CO₃ (1.79 g, 5.51 mmol). The resulting mixture was stirred overnight at 55° C. After one-night HPLC showed 51% conversion, methyl 4-methylsulfonyloxycyclohexanecarboxylate 2 (976.02 mg, 4.13 mmol) was added and the mixture stirred for one more night. HPLC showed 66% conversion, so methyl 4-methylsulfonyloxycyclohexanecarboxylate 2 (650.68 mg, 2.75 mmol) was again added and stirred for one further night. HPLC showed 95% conversion. Water was added to the mixture, the aqueous phase was extracted with EtOAc three times, and the organic phases were washed with water, dried over sodium sulphate, filtered and concentrated. The residue was purified by normal phase flash column chromatography (elution: 0-30% MTBE in Heptane over 15 CV). Pure fractions were combined and concentrated to give 4 (410 mg, 56% yield) as a white solid.

LCMS method 1: retention time: 1.715 min, 99.9% purity at 215 nm, [M+H]+=268.2.

¹H NMR (400 MHz, DMSO-d6) δ ppm 1.45-1.66 (m, 3H), 1.74-1.95 (m, 6H), 2.41-2.45 (m, 2H), 2.45-2.45 (m, 1H), 2.65 (s, 2H), 4.15-4.26 (m, 1H), 4.30-4.41 (m, 1H), 8.80 (s, 1H).

Step 3. Preparation of Methyl 4-(4-amino-3-methyl-pyrazol-1-yl)cyclohexanecarboxylate (5). Methyl 4-(3-methyl-4-nitro-pyrazol-1-yl)cyclohexanecarboxylate 4 (283 mg, 1.06 mmol) was solubilised in ethyl acetate (7.5 mL), and degassed by purging with N2 for 15 minutes. Then Pd/C (10% w/w) (450.72 mg, 0.42 mmol) was added, and the mixture was degassed by purging with N2 for 15 minutes, followed by purging with H₂ for 15 minutes. The mixture was stirred at rt overnight under H₂ (1 atm). The reaction mixture was filtered on Celite, rinsed with MeOH, and concentrated under reduced pressure to give 5 (220 mg, 87% yield) as a tan solid.

LCMS method 1: 99.9% purity at 215 nm, [M+H]+=238.2.

¹H NMR (400 MHz, DMSO-d6) δ ppm 1.39-1.59 (m, 1H), 1.60-1.71 (m, 1H), 1.73-1.85 (m, 2H), 1.87-2.04 (m, 4H), 2.05-2.18 (m, 1H), 2.28-2.42 (m, 1H), 3.43-3.72 (m, 4H), 3.77-4.02 (m, 1H), 6.81-7.07 (m, 1H).

Step 4. Preparation of Methyl 4-[3-methyl-4-[[5-[(3R,5R)-3-(tert-butoxycarbonylamino)-5-fluoro-1-piperidyl]pyrazolo[1,5-a]pyrimidine-3-carbonyl]amino]pyrazol-1-yl]cyclohexanecarboxylate (7). To a solution of methyl 4-(4-amino-3-methyl-pyrazol-1-yl)cyclohexanecarboxylate 5 (220 mg, 0.93 mmol) and 5-[(3R,5R)-3-(tert-butoxycarbonylamino)-5-fluoro-1-piperidyl]pyrazolo[1,5-a]pyrimidine-3-carboxylic acid 6-[synthesis in Final Product General Method 1 P-13]-(351.73 mg, 0.93 mmol) in MeCN (10.5 mL) was added NMI (0.22 mL, 2.78 mmol) and TCFH (390.19 mg, 1.39 mmol). The mixture was stirred at rt overnight. Then water was added and the precipitate was filtered and dried on the high vacuum pump to give 7 (293 mg, 52% yield) as an off-white solid.

LCMS method 1: 68.4% purity at 215 nm, [M+H]+=599.3.

¹H NMR (400 MHz, DMSO-d6) δ ppm 1.17-1.31 (s, 9H), 1.58 (m, 2H), 1.95-2.05 (m, 4H), 2.01-2.16 (m, 4H), 2.28 (m, 2H), 2.85-3.00 (m, 1H), 3.50 (m, 8H), 3.51-3.55 (m, 1H), 4.16-4.44 (m, 1H), 5.06-5.16 (m, 1H), 7.27-7.30 (m, 1H), 7.30-7.32 (m, 1H), 7.32-7.35 (m, 1H), 7.48-7.53 (m, 1H), 8.64-8.76 (m, 2H), 10.98 (s, 1H).

Step 5. Preparation of 4-[3-Methyl-4-[[5-[(3R,5R)-3-(tert-butoxycarbonylamino)-5-fluoro-1-piperidyl]pyrazolo[1,5-a]pyrimidine-3-carbonyl]amino]pyrazol-1-yl]cyclohexanecarboxylic acid (8). To a suspension of methyl 4-[3-methyl-4-[[5-[(3R,5R)-3-(tert-butoxycarbonylamino)-5-fluoro-1-piperidyl]pyrazolo[1,5-a]pyrimidine-3-carbonyl]amino]pyrazol-1-yl]cyclohexanecarboxylate 7 (290 mg, 0.48 mmol) in THE (2 mL) and methanol (2 mL) was added a solution of LiOH H₂O (81.3 mg, 1.94 mmol) in water (2 mL). The resulting mixture was stirred at 60° C. After 1 h, THE and MeOH were removed under reduced pressure. Nanopure water was added to the residue and pH was adjusted to 3 by adding 6 N HCl. Then MeCN was added and the precipitate was filtered and rinsed with water and MeCN to give 8 (253 mg, 89% yield) as an off-white solid.

LCMS method 1: 99.9% purity at 215 nm, [M+H]=585.4.

¹H NMR (400 MHz, DMSO-d6) δ ppm 1.34 (br s, 4H), 1.47-1.58 (m, 2H), 1.71-1.80 (m, 2H), 1.82-1.94 (m, 2H), 1.97-2.07 (m, 2H), 2.08 (s, 1H), 2.21 (s, 4H), 2.66-2.70 (m, 1H), 3.62-3.79 (m, 2H), 4.01-4.17 (m, 2H), 4.39-4.77 (m, 4H), 5.00-5.21 (m, 3H), 6.88-6.99 (m, 1H), 7.12-7.20 (m, 1H), 7.68-7.73 (m, 1H), 8.00-8.05 (m, 1H), 8.26 (m, 1H), 8.82 (m, 1H), 9.03-9.09 (m, 1H), 12.09-12.24 (m, 1H).

¹⁹F NMR (377 MHz, DMSO-d6) δ ppm-167.26 (s, 1 F).

Step 6. Preparation of tert-butyl N-[(3R,5R)-1-[3-[[1-[4-[[1-[2-(2,6-dioxo-3-piperidyl)-1,3-dioxo-isoindolin-5-yl]-4-piperidyl]carbamoyl]cyclohexyl]-3-methyl-pyrazol-4-yl]carbamoyl]pyrazolo[1,5-a]pyrimidin-5-yl]-5-fluoro-3-piperidyl]carbamate (9). To a solution of 4-[3-methyl-4-[[5-[(3R,5R)-3-(tert-butoxycarbonylamino)-5-fluoro-1-piperidyl]pyrazolo[1,5-a]pyrimidine-3-carbonyl]amino]pyrazol-1-yl]cyclohexanecarboxylic acid 8 (250 mg, 0.43 mmol) and 5-(4-amino-1-piperidyl)-2-(2,6-dioxo-3-piperidyl)isoindoline-1,3-dione 2,2,2-trifluoroacetic acid C-3 (281.61 mg, 0.60 mmol) in dry DMF (5 mL), DIPEA (0.59 mL, 3.42 mmol) was added. After 5 minutes stirring at room temperature, HATU (195.11 mg, 0.51 mmol) was added and the resulting mixture was stirred at room temperature. After 1 h, LCMS showed full conversion. The mixture was purified by reverse phase flash chromatography (150 g C18 RediSep Rf Gold column, liquid deposit (DMF), elution: 5% MeCN/0.1% HCOOH over 5 CV, then 5 to 25% MeCN/0.1% HCOOH over 5 CV, followed by 25 to 60% over 15 CV). Fractions were combined, concentrated and lyophilized to give 9 (267 mg, 68% yield) as a yellow solid.

LCMS method 1: 99.9% purity at 215 nm, [M+H]=923.4.

¹H NMR (400 MHz, DMSO-d6) δ ppm 1.34 (br s, 9H), 1.40-1.49 (m, 2H), 1.52-1.62 (m, 2H), 1.63-1.73 (m, 2H), 1.76-1.93 (m, 6H), 1.98-2.06 (m, 2H), 2.06-2.10 (m, 2H), 2.10-2.18 (m, 2H), 2.21 (s, 2H), 2.25-2.30 (m, 1H), 2.55-2.69 (m, 2H), 2.82-2.97 (m, 2H), 3.13 (br s, 2H), 3.63-3.77 (m, 1H), 3.81-3.93 (m, 1H), 3.97-4.16 (m, 3H), 5.06 (m, 2H), 5.13-5.20 (m, 1H), 6.89-6.98 (m, 1H), 7.09-7.21 (m, 1H), 7.23-7.29 (m, 1H), 7.35 (br d, J=1.5 Hz, 1H), 7.68 (m, 1H), 7.76 (br d, J=8.1 Hz, 1H), 8.03 (s, 1H), 8.15 (s, 1H), 8.23-8.29 (m, 1H), 8.82 (br d, J=8.1 Hz, 1H), 9.05 (br s, 1H), 11.08 (s, 1H).

¹⁹F NMR (377 MHz, DMSO-d6) δ ppm-188.35 (s, 1 F).

Step 7. Preparation of N-[1-[4-[[1-[2-(2,6-dioxo-3-piperidyl)-1,3-dioxo-isoindolin-5-yl]-4-piperidyl]carbamoyl]cyclohexyl]-3-methyl-pyrazol-4-yl]-5-[(3R,5R)-3-amino-5-fluoro-1-piperidyl]pyrazolo[1,5-a]pyrimidine-3-carboxamide hydroxhloride (P-27). tert-Butyl N-[(3R,5R)-1-[3-[[1-[4-[[1-[2-(2,6-dioxo-3-piperidyl)-1,3-dioxo-isoindolin-5-yl]-4-piperidyl]carbamoyl]cyclohexyl]-3-methyl-pyrazol-4-yl]carbamoyl]pyrazolo[1,5-a]pyrimidin-5-yl]-5-fluoro-3-piperidyl]carbamate 9 (300 mg, 0.33 mmol) was solubilized in 4 M HCl in 1,4-dioxane (10 mL, 40 mmol). The solution was stirred at rt for 3 h. The solvent was removed under reduced pressure and the residue was purified by reverse phase flash chromatography (150 g C18 RediSep Rf Gold column, liquid deposit (water), elution: 5% MeCN/0.02 M HCl over 3 CV, then 5 to 30% MeCN/0.02 M HCl over 15 CV). Fractions were combined, concentrated and purified by preparative HPLC to give P-27 (83.97 mg, 31% yield) as a yellow solid as a full HCl salt.

LCMS method 5: 97.3% purity at 215 nm, [M−HCl+2H]2+=412.2; [M−HCl+H]+=823.3; [M−HCl+3H]³+=275.3.

¹H NMR (400 MHz, DMSO-d6) δ ppm 1.37-1.49 (m, 2H), 1.49-1.62 (m, 2H), 1.63-1.76 (m, 2H), 1.76-1.88 (m, 4H), 1.90-2.08 (m, 4H), 2.10-2.19 (m, 1H), 2.23 (s, 3H), 2.35-2.45 (m, 1H), 2.54-2.64 (m, 2H), 2.82-2.95 (m, 1H), 3.12 (t, J=11.5 Hz, 2H), 3.25-3.60 (m, 4H), 3.94-4.12 (m, 4H), 4.64-4.78 (m, 1H), 5.03-5.25 (m, 2H), 6.92 (d, J=7.8 Hz, 1H), 7.26 (dd, J=8.7, 1.8 Hz, 1H), 7.34 (d, J=1.5 Hz, 1H), 7.67 (d, J=8.3 Hz, 1H), 7.77 (d, J=7.6 Hz, 1H), 8.05 (s, 1H), 8.19-8.43 (m, 4H), 8.91 (d, J=8.1 Hz, 1H), 9.05 (s, 1H), 11.08 (s, 1H).

¹⁹F NMR (377 MHz, DMSO-d6) δ ppm-184.87 (s, 1 F).

TABLE 4

| Compound | | | |
|---|---|---|---|
| No. | CBM | Structure | Characterization Data |
| P-27 | C-3 | 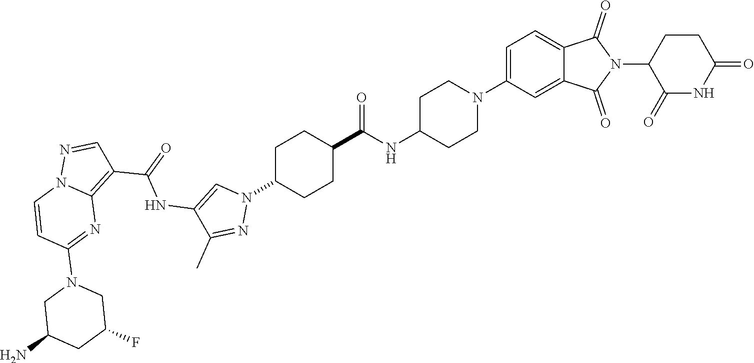 [SMILES: Cc1nn([C@H]2CC[C@H](C(=O)NC3CCN(c4ccc5c(c4)C(=O)N(C4CCC(=O)NC4=O)C5=O)CC3)CC2)cc1NC(=O)c1cnn2ccc(N3C[C@H](N)C[C@@H](F)C3)nc12] | 31% yield as a yellow solid. LCMS method 5: retention time: 2.953 min, 97% purity at 215 nm, [M − HCl + 2H]²⁺ = 412.2; [M − HCl + H]⁺ = 823.3; [M − HCl + 3H]³⁺ = 275.3. ¹H NMR (400 MHz, DMSO-d6) δ ppm 1.37-1.49 (m, 2 H), 1.49-1.62 (m, 2 H), 1.63-1.76 (m, 2 H), 1.76-1.88 (m, 4 H), 1.90-2.08 (m, 4 H), 2.10-2.19 (m, 1 H), 2.23 (s, 3 H), 2.35-2.45 (m, 1 H), 2.54-2.64 (m, 2 H), 2.82-2.95 (m, 1 H), 3.12 (t, J = 11.5 Hz, 2 H), 3.25-3.60 (m, 4 H), 3.94-4.12 (m, 4 H), 4.64-4.78 (m, 1 H), 5.03-5.25 (m, 2 H), 6.92 (d, J = 7.8 Hz, 1 H), 7.26 (dd, J = 8.7, 1.8 Hz, 1 H), 7.34 (d, J = 1.5 Hz, 1 H), 7.67 (d, J = 8.3 Hz, 1 H), 7.77 (d, J = 7.6 Hz, 1 H), 8.05 (s, 1 H), 8.19-8.43 (m, 4 H), 8.91 (d, J = 8.1 Hz, 1 H), 9.05 (s, 1 H), 11.08 (s, 1 H). ¹⁹F NMR (377 MHz, DMSO- d6) δ ppm −184.87 (s, 1 F). |
| P-28 | C-3 | 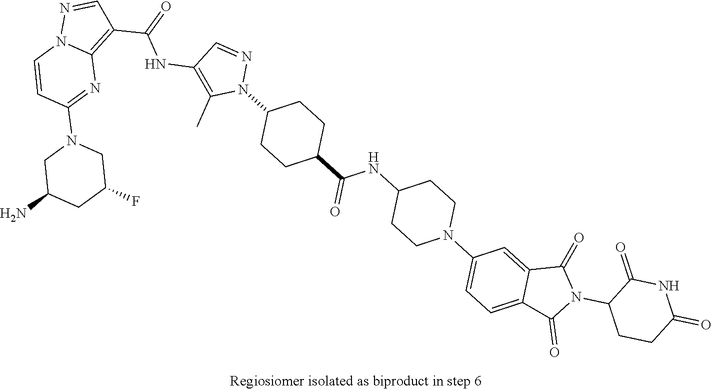 | 12% yield as a yellow solid. LCMS method 5: retention time: 2.882 min, 99% purity at 215 nm, [M − HCl + 2H]²⁺ = 412.2; [M − HCl + H]⁺ = 823.3; [M − HCl + 3H]³⁺ = 275.3. ¹H NMR (400 MHz, DMSO-d6) δ ppm 1.20-1.33 (m, 1 H), 1.36-1.50 (m, 2 H), 1.54-1.68 (m, 2 H), 1.77-2.10 (m, 9 H), 2.11-2.21 (m, 1 H), 2.27 (s, 3 H), 2.35-2.46 (m, 1 H), 2.53-2.64 (m, 2 H), 2.82-2.95 (m, 1 H), 3.13 (t, J = 11.7 Hz, 2 H), 3.23-3.35 (m, 1 H), 3.36- 3.51 (m, 3 H), 3.83-3.89 (m, 1 H), 3.95-4.02 (m, 2 H), 4.08-4.15 (m, 1 H), 4.61-4.76 (m, 1 H), 5.03-5.24 (m, 2 H), 6.92 (d, J = 7.6 Hz, 1 H), 7.27 (d, J = 8.8 Hz, 1 H), 7.34 (s, 1 H), 7.67 (d, J = 8.6 Hz, 1 H), 7.72 (s, 1 H), 7.80 (d, J = 7.6 Hz, 1 H), 8.20-8.48 (m, 4 H), 8.82-9.02 (m, 2 H), 11.08 (s, 1 H). ¹⁹F NMR (377 MHz, DMSO- d6) δ ppm −184.84 (s, 1 F). |

### Final Product Method 4

### Example S-9. Synthesis of P-29

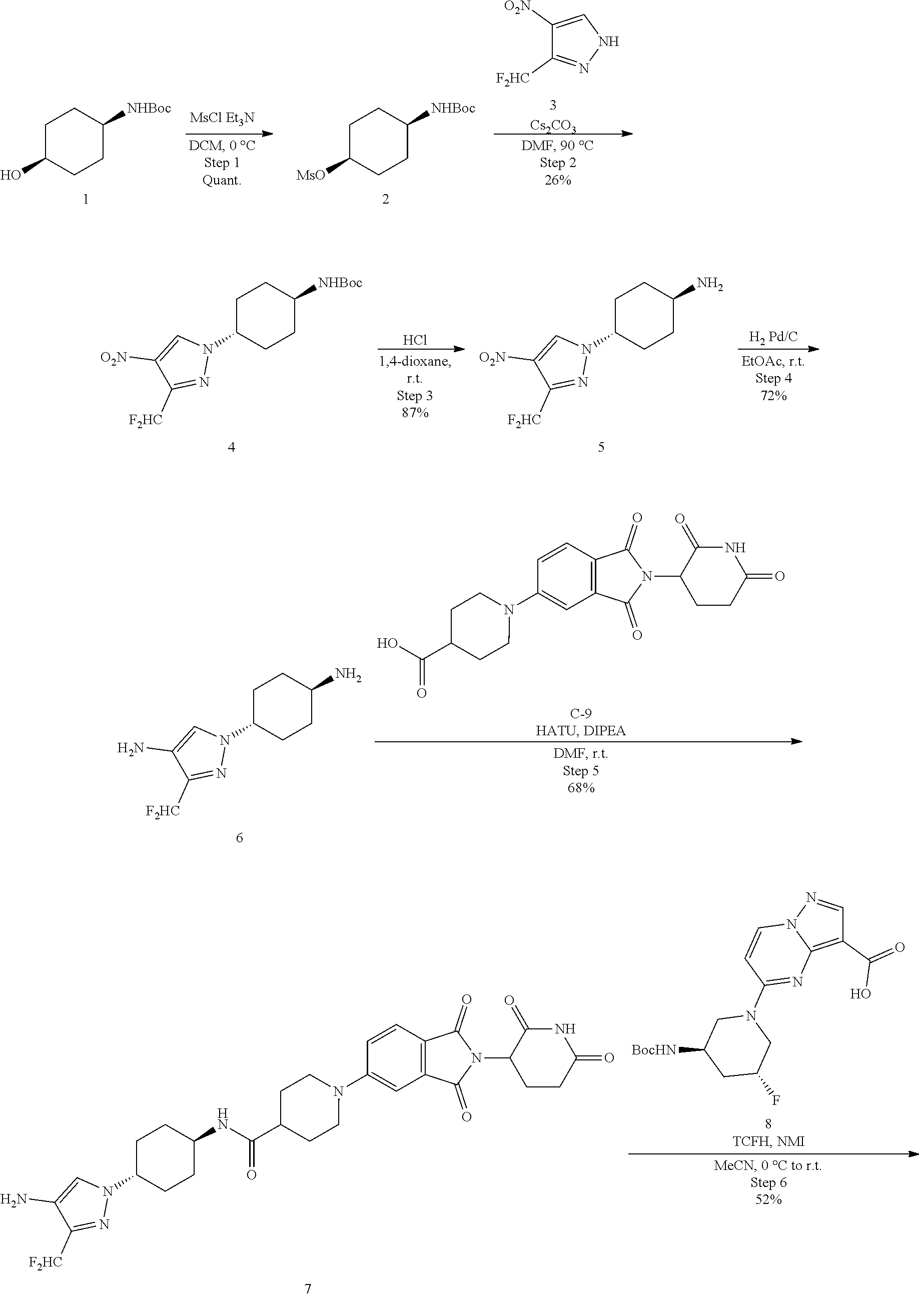

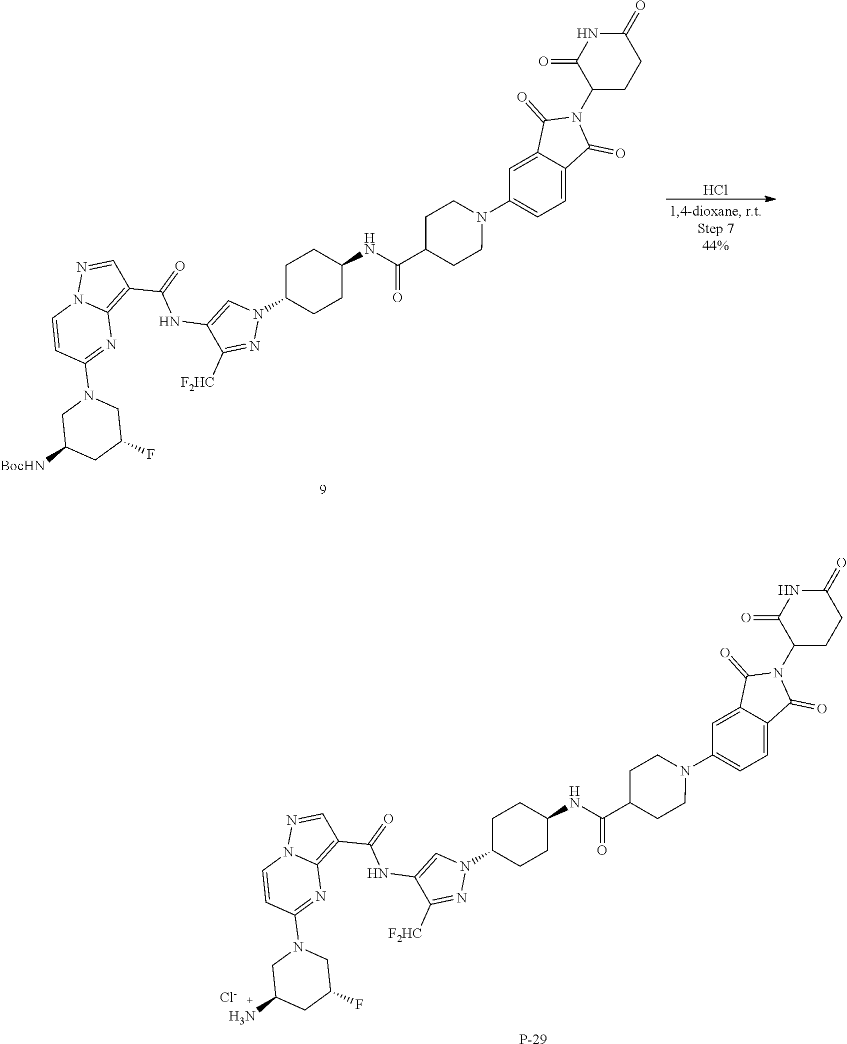

Step 1. Preparation of [4-(tert-Butoxycarbonylamino)cyclohexyl]methanesulfonate (2). To a solution of tert-butyl N-(4-hydroxycyclohexyl)carbamate 1 (4.0 g, 18.6 mmol, 1 eq.) and Et₃N (3.8.8 mL, 27.9 mmol, 1.5 eq.) in anhydrous DCM (93 mL) at 0° C. was added methanesulfonyl chloride (1.87 mL, 24.2 mmol, 1.3 eq.) dropwise. The resulting mixture was stirred at 0° C. After 1 h, TLC (50% EtOAc in heptane, KMnO₄ stain) showed full conversion. Water was added and the aqueous phase was extracted 3 times with DCM. The organics were washed once with aqueous 1N HCl, dried over Na₂SO₄ and concentrated to give 2 (5.45 g, quantitative yield) as a white solid.

LCMS method 1: 99.9% purity at 200 nm, [M+Na]⁺=316.2.

¹H NMR (400 MHz, CDCl₃) δ ppm 1.45 (s, 9H), 1.51-1.64 (m, 2H), 1.68-1.80 (m, 2H), 1.80-1.90 (m, 2H), 2.00-2.11 (m, 2H), 3.02 (s, 3H), 3.53 (br s, 1H), 4.47 (br s, 1H), 4.87-4.92 (m, 1H).

Step 2. Preparation of tert-Butyl N-[4-[3-(difluoromethyl)-4-nitro-pyrazol-1-yl]cyclohexyl]carbamate (4). To a solution of 3-(difluoromethyl)-4-nitro-1H-pyrazole 3 (1.0 g, 6.13 mmol, 1 eq.) and [4-(tert-butoxycarbonylamino)cyclohexyl]methanesulfonate 2 (1.8 g, 6.13 mmol, 1 eq.) in anhydrous DMF (20.4 mL) was added Cs₂CO₃ (4.0 g, 12.3 mmol, 2 eq.). The resulting mixture was stirred at 90° C. After 20 h, LCMS showed that conversion was not complete. Additional [4-(tert-butoxycarbonylamino)cyclohexyl]methanesulfonate 2 (0.9 g, 3.07 mmol, 0.5 eq.) was added to the reaction mixture and the resulting mixture was stirred at 90° C. After 3 days, HPLC showed that conversion was not complete. Additional [4-(tert-butoxycarbonylamino)cyclohexyl]methanesulfonate 2 (1.35 g, 4.60 mmol, 0.75 eq.) was added to the reaction mixture and the resulting mixture was stirred at 90° C. After 24 h, HPLC showed that conversion was almost complete. Water was added to the reaction mixture and the aqueous phase was extracted 3 times with EtOAc. The organics were washed 3 times with water and once with brine, dried over Na₂SO₄ and concentrated to dryness. The residue was then purified by normal phase flash chromatography (120 g silica column, preabsorbed, elution: 0 to 40% MTBE/Heptane over 10 CV, then 40 to 80% MTBE/Heptane over 10 CV, then 80 to 100% MTBE/Heptane over 10 CV). Fractions were combined and concentrated to give an impure product. The crude product was then purified by reverse phase flash chromatography (80 g C18 RediSep Rf Gold column, liquid deposit (DMSO), elution: 5% MeOH/0.1% HCOOH over 3 CV, then 5 to 55% MeOH/0.1% HCOOH over 0.5 CV, then 55 to 90% MeOH/0.1% HCOOH over 15 CV). Fractions were combined and concentrated to give 4 (583 mg, 26% yield) as a white solid.

LCMS method 1: 99.9% purity at 215 nm, [M-tBu+H]=205.2.

¹H NMR (400 MHz, DMSO-d6) δ ppm 1.27-1.38 (m, 3H), 1.38 (s, 9H), 1.75-1.95 (m, 4H), 2.04-2.07 (m, 2H), 4.23-4.33 (m, 1H), 6.83 (br d, J=7.3 Hz, 1H), 7.30 (t, J=53.1 Hz, 1H), 9.05 (s, 1H).

¹⁹F NMR (377 MHz, DMSO-d6) δ ppm-117.36 (s, 2 F).

Step 3. Preparation of 4-[3-(Difluoromethyl)-4-nitro-pyrazol-1-yl]cyclohexanamine (5). To a solution of tert-butyl N-[4-[3-(difluoromethyl)-4-nitro-pyrazol-1-yl]cyclohexyl]carbamate 4 (200 mg, 0.560 mmol, 1 eq.) was added 4 M HCl in 1,4-dioxane (4.16 mL, 16.7 mmol, 30 eq.). The resulting mixture was stirred at room temperature. After 2.5 h, LCMS showed full conversion. The solvents were evaporated under reduced pressure and the crude material was co-evaporated twice with MeCN. The crude mixture was purified by reverse phase flash chromatography (50 g C18 RediSep Rf Gold column, liquid deposit (H₂O), elution: 5% MeOH/buffer solution pH 10 over 10 CV, then 5% MeOH/H₂O over 5 CV, then 5 to 100% MeOH/H₂O over 12 CV). Fractions were combined and concentrated to give 5 (125 mg, 87% yield) as a colorless semi-solid.

LCMS method 1: 99.9% purity at 215 nm, [M+H]=261.4.

¹H NMR (400 MHz, CDCl₃) δ ppm 1.27-1.42 (m, 4H), 1.85 (qd, J=12.7, 3.4 Hz, 2H), 2.03-2.12 (m, 2H), 2.20-2.30 (m, 2H), 2.79-2.89 (m, 1H), 4.19 (tt, J=12.0, 3.9 Hz, 1H), 7.12 (t, J=53.3 Hz, 1H), 8.21 (s, 1H).

¹⁹F NMR (377 MHz, CDCl₃) δ ppm-117.68 (s, 2 F).

Step 4. Preparation of 1-(4-Aminocyclohexyl)-3-(difluoromethyl)pyrazol-4-amine (6). A solution of 4-[3-(difluoromethyl)-4-nitro-pyrazol-1-yl]cyclohexanamine 5 (150 mg, 0.580 mmol, 1 eq.) in ethyl acetate (5.8 mL) was degassed with nitrogen for 15 min. Then, Pd/C 10 wt. % (184 mg, 0.170 mmol, 0.3 eq.) was added and the solution was degassed for a further 15 min. Finally, hydrogen was used to degas the solution for 15 min and the mixture was stirred under hydrogen (1 atm) at room temperature. After 4 h, LCMS showed full conversion. The solution was filtered through a Celite pad, rinsing with EtOAc/MeCN. The solvents were evaporated under reduced pressure to give 6 (95 mg, 72% yield) as a grey oil.

LCMS method 1: 99.9% purity at 254 nm, [M+H]=231.4.

¹H NMR (400 MHz, DMSO-d6) δ ppm 1.10-1.23 (m, 2H), 1.60-1.74 (m, 2H), 1.78-1.95 (m, 4H), 2.58 (ddt, J=11.0, 7.2, 3.7 Hz, 1H), 3.95 (ddt, J=11.7, 7.9, 3.8 Hz, 1H), 4.02 (br s, 2H), 6.86 (t, J=54.0 Hz, 1H), 7.13 (s, 1H).

¹⁹F NMR (377 MHz, CDCl₃) δ ppm-111.24 (d, J=54.5 Hz, 2 F).

Step 5. Preparation of N-[4-[4-Amino-3-(difluoromethyl)pyrazol-1-yl]cyclohexyl]-1-[2-(2,6-dioxo-3-piperidyl)-1,3-dioxo-isoindolin-5-yl]piperidine-4-carboxamide (7). To a solution of 1-[2-(2,6-dioxo-3-piperidyl)-1,3-dioxo-isoindolin-5-yl]piperidine-4-carboxylic acid C-9 (100 mg, 0.260 mmol, 1 eq.) and 1-(4-aminocyclohexyl)-3-(difluoromethyl)pyrazol-4-amine 6 (101.6 mg, 0.44 mmol, 1.7 eq.) in anhydrous DMF (2.5 mL) was added DIPEA (135 uL, 0.780 mmol, 3 eq.) at room temperature. After 5 min, HATU (118.4 mg, 0.310 mmol, 1.2 eq.) was added and the resulting mixture was stirred at room temperature. After 1.5 h, LCMS showed full conversion. The crude mixture was directly purified by reverse phase flash chromatography (100 g C18 gold column, liquid deposit (DMF), elution: 5% MeCN/0.1% HCOOH over 5 CV, then 5 to 25% MeCN/0.1% HCOOH over 15 CV, then 25 to 35% MeCN/0.1% HCOOH over 10 CV). Fractions were combined and concentrated to give 7 (105 mg, 68% yield) as a yellow solid.

LCMS method 1: 99.9% purity at 215 nm, [M+H]+=598.4.

¹H NMR (400 MHz, DMSO-d6) δ ppm 1.26-1.39 (m, 2H), 1.53-1.67 (m, 2H), 1.68-1.78 (m, 3H), 1.81-1.90 (m, 2H), 1.91-2.06 (m, 3H), 2.35-2.44 (m, 1H), 2.53-2.58 (m, 1H), 1.59-2.62 (m, 1H), 2.81-2.93 (m, 1H), 2.93-3.04 (m, 2H), 3.51-3.64 (m, 1H), 3.93-4.13 (m, 4H), 5.06 (dd, J=13.0, 5.4 Hz, 1H), 6.87 (t, J=54.0 Hz, 1H), 7.16 (s, 1H), 7.25 (dd, J=9.0, 2.0 Hz, 1H), 7.33 (d, J=2.0 Hz, 1H), 7.66 (d, J=8.6 Hz, 1H), 7.76 (br d, J=7.8 Hz, 1H), 11.07 (s, 1H).

¹⁹F NMR (377 MHz, DMSO-d6) δ ppm-111.30 (s, 2 F).

Step 6. Preparation of tert-butyl N-[(3R,5R)-1-[3-[[3-(difluoromethyl)-1-[4-[[1-[2-(2,6-dioxo-3-piperidyl)-1,3-dioxo-isoindolin-5-yl]piperidine-4-carbonyl]amino]cyclohexyl]pyrazol-4-yl]carbamoyl]pyrazolo[1,5-a]pyrimidin-5-yl]-5-fluoro-3-piperidyl]carbamate (9). To a solution of N-[4-[4-amino-3-(difluoromethyl)pyrazol-1-yl]cyclohexyl]-1-[2-(2,6-dioxo-3-piperidyl)-1,3-dioxo-isoindolin-5-yl]piperidine-4-carboxamide 7 (100 mg, 0.170 mmol, 1 eq.) and 5-[(3R,5R)-3-(tert-butoxycarbonylamino)-5-fluoro-1-piperidyl]pyrazolo[1,5-a]pyrimidine-3-carboxylic acid 8-[synthesis in Final Product General Method 1 P-13]-(63.5 mg, 0.170 mmol, 1 eq.) in MeCN (1.7 mL) were added NMI (0.04 mL, 0.50 mmol, 3 eq.) and TCFH (70.4 mg, 0.25 mmol, 1.5 eq.) at 0° C. The mixture was stirred at room temperature. After 20 h, LCMS showed full conversion. MeCN was removed under reduced pressure and the residue was purified by reverse phase flash chromatography (50 g C18 RediSep Rf Gold column, liquid deposit (DMSO), elution: 5% MeCN/0.1% HCOOH over 5 CV, then 5 to 40% MeCN/0.1% HCOOH over 1 CV, then 40 to 70% MeCN/0.1% HCOOH over 15 CV). Fractions were combined and concentrated to give 9 (84.3 mg, 52% yield) as a yellow solid.

LCMS method 1: 99.9% purity at 215 nm, [M+H]=959.4.

¹H NMR (400 MHz, DMSO-d6) δ ppm 1.31-1.44 (m, 2H), 1.54-1.69 (m, 2H), 1.70-1.95 (m, 7H), 1.97-2.06 (m, 3H), 2.09-2.19 (m, 1H), 2.35-2.46 (m, 2H), 2.55-2.62 (m, 1H), 2.81-2.94 (m, 2H), 2.97-3.03 (m, 3H), 3.35-3.54 (m, 2H), 3.55-3.74 (m, 3H), 4.07 (br d, J=12.7 Hz, 2H), 4.17-4.29 (m, 1H), 4.96-5.11 (m, 2H), 6.84-6.95 (m, 1H), 7.05-7.15 (m, 1H), 7.25 (br d, J=8.8 Hz, 1H), 7.33 (br s, 1H), 7.66 (d, J=8.6 Hz, 1H), 7.79 (d, J=7.8 Hz, 1H), 8.29 (s, 1H), 8.36 (br s, 1H), 8.82 (d, J=7.8 Hz, 1H), 9.31 (br s, 1H), 11.07 (s, 1H).

¹⁹F NMR (377 MHz, DMSO-d6) δ ppm-111.80-110.41 (m, 2 F).

Step 7. Preparation of 5-[(3R,5R)-3-Amino-5-fluoro-1-piperidyl]-N-[3-(difluoromethyl)-1-[4-[[1-[2-(2,6-dioxo-3-piperidyl)-1,3-dioxo-isoindolin-5-yl]piperidine-4-carbonyl]amino]cyclohexyl]pyrazol-4-yl]pyrazolo[1,5-a]pyrimidine-3-carboxamide hydrochloride (P-29). To tert-butyl N-[(3R,5R)-1-[3-[[3-(difluoromethyl)-1-[4-[[1-[2-(2,6-dioxo-3-piperidyl)-1,3-dioxo-isoindolin-5-yl]piperidine-4-carbonyl]amino]cyclohexyl]pyrazol-4-yl]carbamoyl]pyrazolo[1,5-a]pyrimidin-5-yl]-5-fluoro-3-piperidyl]carbamate 9 (80 mg, 0.080 mmol, 1 eq.) was added 4 M HCl in 1,4-dioxane (3.13 mL, 12.5 mmol, 150 eq.). The mixture was sonicated and stirred at room temperature. After 20 h, HPLC showed full conversion. The solvents were removed under reduced pressure and the residue was purified by reverse phase flash chromatography (30 g C18 RediSep Rf Gold column, liquid deposit (DMSO), elution: 5% MeCN/0.02 M HCl over 5 CV, then 5 to 100% MeCN/0.02 M HCl over 10 CV). Fractions were combined, concentrated and lyophilized to give P-29 (30.60 mg, 44% yield) as a yellow solid as a full hydrochloric acid salt.

LCMS method 5: 99.4% purity at 215 nm, [M−HCl+H]+=859.4.

¹H NMR (400 MHz, DMSO-d6) δ ppm 1.31-1.44 (m, 2H), 1.55-1.68 (m, 2H), 1.69-1.94 (m, 7H), 1.94-2.10 (m, 4H), 2.37-2.44 (m, 1H), 2.53-2.63 (m, 2H), 2.83-2.94 (m, 1H), 2.94-3.05 (m, 2H), 3.22-3.28 (m, 1H), 3.37-3.53 (m, 2H), 3.55-3.67 (m, 1H), 4.02-4.12 (m, 2H), 4.20-4.30 (m, 1H), 4.52-4.93 (m, 2H), 5.02-5.19 (m, 2H), 6.88 (d, J=7.8 Hz, 1H), 6.99-7.28 (m, 2H), 7.33 (d, J=2.0 Hz, 1H), 7.67 (d, J=8.6 Hz, 1H), 7.80 (d, J=7.3 Hz, 1H), 8.14 (br s, 3H), 8.33 (s, 1H), 8.40 (s, 1H), 8.93 (d, J=7.8 Hz, 1H), 9.33 (s, 1H), 11.07 (s, 1H).

¹⁹F NMR (377 MHz, DMSO-d6) δ ppm-184.79 (s, 1 F), -111.25 (d, J=16.3 Hz, 2 F).

TABLE 5

| Compound | | | |
|---|---|---|---|
| No. | CBM | Structure | Characterization Data |
| P-29 | C-9 | 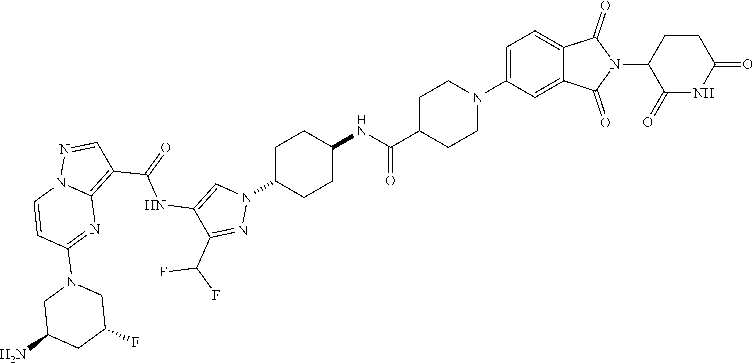 | 44% yield as a yellow solid. LCMS method 5: retention time: 2.249 min, 99% purity at 215 nm, [M − HCl + 2H]²⁺ = 430.3; [M − HCl + H]⁺ = 859.4. ¹H NMR (400 MHz, DMSO-d6) d ppm 1.31-1.44 (m, 2 H), 1.55-1.68 (m, 2 H), 1.69-1.94 (m, 7 H), 1.94-2.10 (m, 4 H), 2.37- 2.44 (m, 1 H), 2.53-2.63 (m, 2 H), 2.83-2.94 (m, 1 H), 2.94-3.05 (m, 2 H), 3.22-3.28 (m, 1 H), 3.37-3.53 (m, 2 H), 3.55-3.67 (m, 1 H), 4.02-4.12 (m, 2 H), 4.20-4.30 (m, 1 H), 4.52-4.93 (m, 2 H), 5.02-5.19 (m, 2 H), 6.88 (d, J = 7.8 Hz, 1 H), 6.99-7.28 (m, 2 H), 7.33 (d, J = 2.0 Hz, 1 H), 7.67 (d, J = 8.6 Hz, 1 H), 7.80 (d, J = 7.3 Hz, 1 H), 8.14 (br s, 3 H), 8.33 (s, 1 H), 8.40 (s, 1 H), 8.93 (d, J = 7.8 Hz, 1 H), 9.33 (s, 1 H), 11.07 (s, 1 H). ¹⁹F NMR (377 MHz, DMSO-d6) d ppm −184.79 (s, 1 F), −111.25 (d, J = 16.3 Hz, 2 F). |

### Final Product Method 5

### Example S-10. Synthesis of P-30

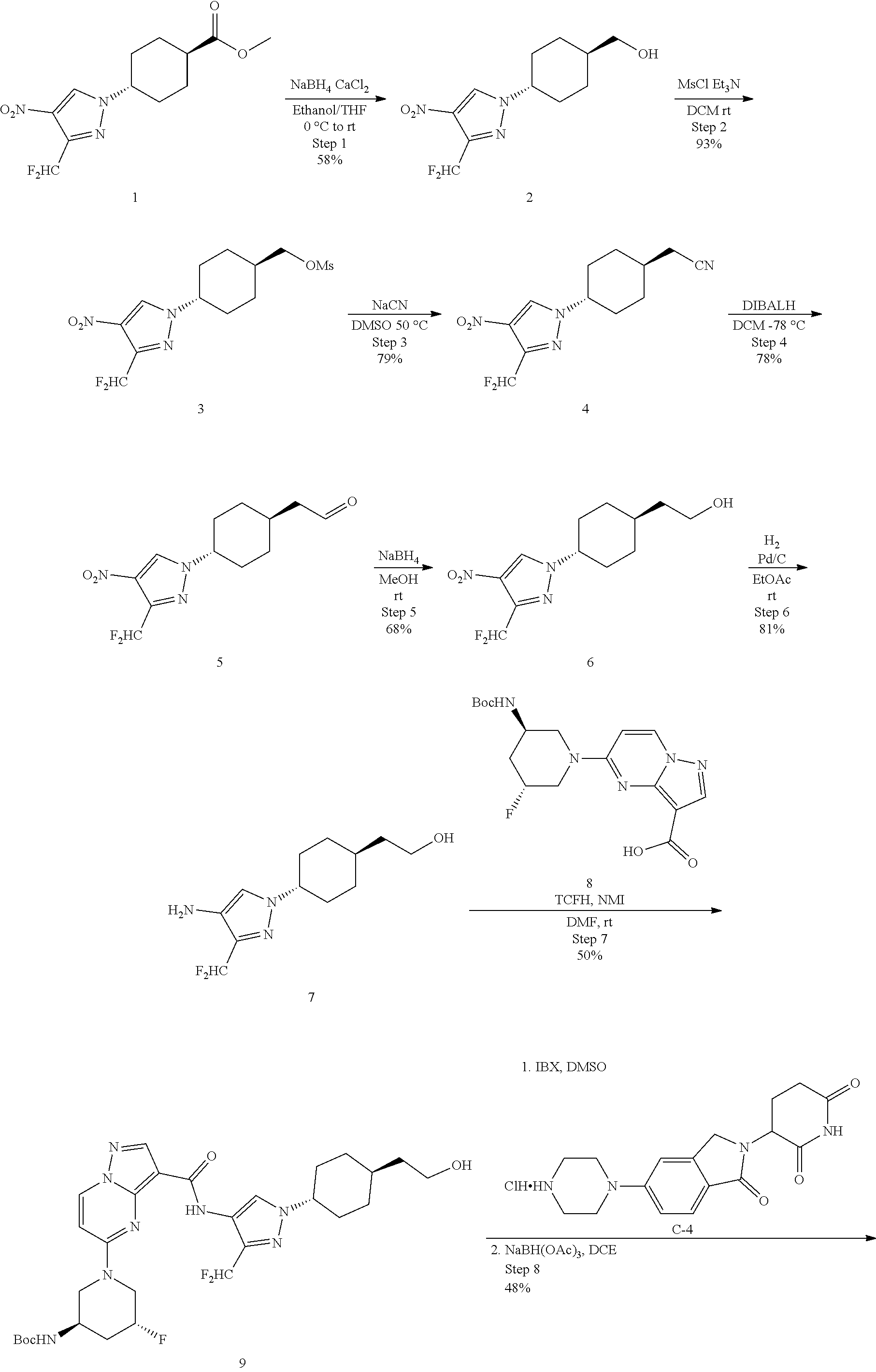

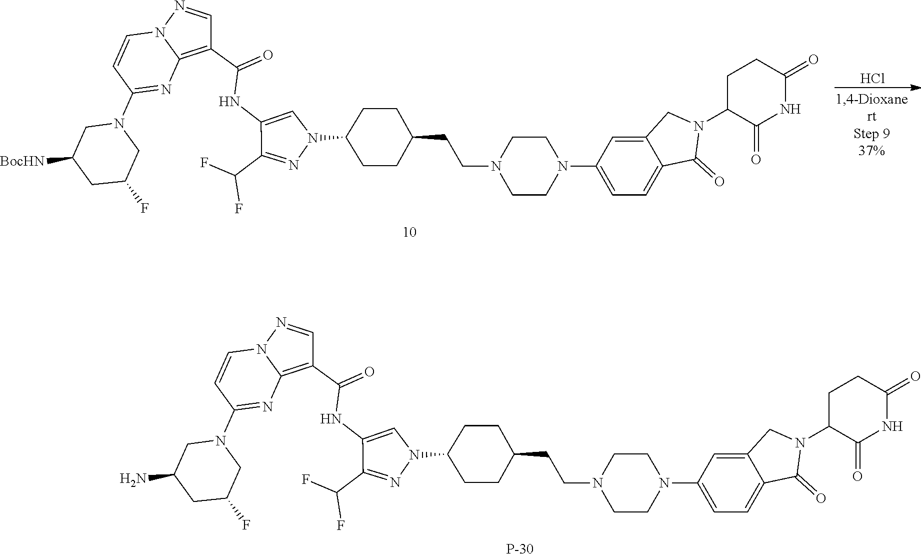

Step 1. Preparation of 14-[3-(Difluoromethyl)-4-nitro-pyrazol-1-yl]cyclohexyl]methanol (2). To a solution of methyl 4-[3-(difluoromethyl)-4-nitro-pyrazol-1-yl]cyclohexanecarboxylate 1 (730.0 mg, 2.26 mmol, 1 eq.) in 1:2 THF (3.8 mL)/ethanol (7.5 mL) at 0° C. was added CaCl₂) (501.2 mg, 4.52 mmol, 2 eq.) followed by the addition of NaBH₄ (341.6 mg, 9.03 mmol, 4 eq.). The resulting mixture was stirred at rt. After 16 h, at that temperature, LCMS showed total conversion. The reaction was quenched dropwise with water, and the resulting mixture was stirred at rt for 1 h. Next, the product was extracted three times with EtOAc, and the organic layers were combined and washed once with water and once with brine, and finally dried over MgSO₄. EtOAc was removed under reduced pressure and the residue was purified by normal phase flash chromatography (80 g silica column, liquid deposit (DCM), elution: 00 EtOAc/Heptane over 3 CV, then 0 to 100% EtOAc/Heptane over 10 CV, then 100% EtOAc/Heptane over 3 CV (desired product was released around 50% EtOAc). Fractions were combined and concentrated to give 2(371.9 mg, 58% yield) as a colorless oil.

LCMS method 1: 97.5 3 purity at 215 nm, [M+H]n=276.2.

¹H NMR (400 MHz, CDCl₃-d) n ppm 1.14-1.30 (mi, 3H), 1.70-1.87 (mi, 2H), 1.97-2.09 (i, 2H), 2.25-2.34 (m, 2H), 3.54 (d, J=6.1 Hz, 2H), 4.18 (tt, J=12.1, 3.9 Hz, 1H), 7.10 (t, J=53.3 Hz, 1H), 8.22 (s, 1H).

¹⁹F NMR (377 MHz, CDCl₃-d) δ ppm-117.64 (d, J=53.13, 2 F).

Step 2. Preparation of [4-[3-(Difluoromethyl)-4-nitro-pyrazol-1-yl]cyclohexyl]methyl methanesulfonate (3). To a solution of [4-[3-(Difluoromethyl)-4-nitro-pyrazol-1-yl]cyclohexyl]methanol 2 (428.5 mg, 1.52 mmol) and Et₃N (0.28 mL, 1.98 mmol) in DCM (7.62 mL) at 0° C. was added dropwise MsCl (0.13 mL, 1.68 mmol). Next, the reaction mixture was slowly warmed to rt and stirred. After 3 h, TLC (3:7 Heptane/EtOAc) showed completion. Then, the reaction was partitioned between water and EtOAc. The phases were separated and the organic phase was washed three times with water, once with 1 N HCl and once with brine, and then dried over MgSO₄. EtOAc was removed under reduced pressure to give 3 (529 mg, 93% yield) as a light-yellow oil. The product was used without further purification in the next step.

LCMS method 1: 94.8% purity at 215 nm, [M+H]=354.0.

¹H NMR (400 MHz, CDCl₃-d) δ ppm 1.22-1.37 (m, 3H), 1.80-1.90 (m, 2H), 2.05-2.13 (m, 2H), 2.27-2.36 (m, 2H), 3.04 (s, 3H), 4.08-4.14 (m, 2H), 4.15-4.24 (m, 1H), 7.11 (t, J=53.4 Hz, 1H), 8.22 (s, 1H).

¹⁹F NMR (377 MHz, CDCl₃-d) δ ppm-117.64 (d, J=53.13, 2 F).

Step 3. Preparation of 2-[4-[3-(Difluoromethyl)-4-nitro-pyrazol-1-yl]cyclohexyl]acetaldehyde (4). A solution of [4-[3-(Difluoromethyl)-4-nitro-pyrazol-1-yl]cyclohexyl]methyl methanesulfonate 3 (529.0 mg, 1.5 mmol, 1 eq.) and NaCN (183.4 mg, 3.74 mmol, 2.5 eq.) in DMSO (7.49 mL) was heated to 50° C. and stirred. After 16 h at 50° C., LCMS showed total completion. The reaction was cooled down to rt, and was quenched with water under vigorous agitation. Then, EtOAc was added and the product was extracted two times with EtOAc. The combined organic layers were then washed once with concentrated NaHCO₃ aqueous solution and once with brine. Finally, the latter was dried over MgSO₄ and EtOAc was removed under reduced pressure to give 4 (342 mg, 79% yield) as a yellow oil.

LCMS method 1: 98.8% purity at 215 nm, [M+H]=285.2.

¹H NMR (400 MHz, CDCl₃-d) δ ppm 1.30-1.46 (m, 2H), 1.76-1.96 (m, 3H), 2.06-2.16 (m, 2H), 2.29-2.37 (m, 2H), 2.37-2.40 (m, 2H), 4.20 (tt, J=12.0, 3.9 Hz, 1H), 7.12 (t, J=54.2 Hz, 1H), 8.22 (s, 1H).

¹⁹F NMR (377 MHz, CDCl₃-d) δ ppm-117.68 (s, 2 F).

Step 4. Preparation of 2-[4-[3-(Difluoromethyl)-4-nitro-pyrazol-1-yl]cyclohexyl]acetaldehyde (5). To a solution of 2-[4-[3-(Difluoromethyl)-4-nitro-pyrazol-1-yl]cyclohexyl]acetonitrile 4 (342 mg, 1.18 mmol, 1 eq.) in DCM (5.88 mL) was added a 1 M solution of DIBAL-H in DCM (3.53 mL, 3.53 mmol, 3 eq.) at −78° C. The reaction was then stirred at that temperature. After 2 h, LCMS showed total completion. The reaction was slowly quenched with a solution of Rochelle salt at −78° C., and this was stirred for 1 h at rt. Then, the product was extracted three times with EtOAc, and the combined organic layers were washed two times with 1 M HCl and once with brine. Finally, the latter was dried over MgSO₄ and EtOAc was removed under reduced pressure to give 5 (264 mg, 78% yield) as a yellow oil.

LCMS method 1: 99.9% purity at 215 nm, [M+H]=288.2.

¹H NMR (400 MHz, DMSO-d6) δ ppm 1.36 (q, J=6.5 Hz, 2H), 1.75 (qd, J=12.5, 3.2 Hz, 2H), 1.85 (d, J=12.5 Hz, 2H), 2.02-2.15 (m, 2H), 3.40-3.50 (m, 2H), 4.19-4.32 (m, 1H), 4.32-4.40 (m, 1H), 7.30 (t, J=52.3 Hz, 1H), 9.06 (s, 1H).

¹⁹F NMR (377 MHz, DMSO-d6) δ ppm-117.36 (s, 2 F).

Step 5. Preparation of 2-[4-[3-(Difluoromethyl)-4-nitro-pyrazol-1-yl]cyclohexyl]ethanol (6). To a solution of 2-[4-[3-(Difluoromethyl)-4-nitro-pyrazol-1-yl]cyclohexyl]acetaldehyde 5 (264 mg, 0.92 mmol, 1 eq.) in methanol (4.59 mL) at 0° C. was added NaBH₄ (69.52 mg, 1.84 mmol). The resulting mixture was stirred at rt. After 16 h, LCMS showed total conversion. Water was added and the resulting mixture was stirred at rt for 1 h. The product was extracted three times with EtOAc and the combined organic layers were washed twice with brine. Finally, the latter was dried over MgSO₄ and EtOAc was removed under reduced pressure to give 6 (216 mg, 68% yield) as a yellow oil.

LCMS method 1: 83.3% purity at 215 nm, [M+H]=290.2.

¹H NMR (400 MHz, DMSO-d6) δ ppm 1.03-1.15 (m, 2H), 1.36 (q, J=6.5 Hz, 2H), 1.41-1.52 (m, 1H), 1.69-1.81 (m, 2H), 1.85 (br d, J=12.5 Hz, 2H), 2.02-2.12 (m, 2H), 3.37-3.52 (m, 2H), 4.22-4.32 (m, 1H), 4.33-4.39 (m, 1H), 7.30 (t, J=53.2 Hz, 1H), 9.06 (s, 1H).

¹⁹F NMR (377 MHz, DMSO-d6) δ ppm-117.36 (s, 2 F).

Step 6. Preparation of 2-[4-[4-Amino-3-(difluoromethyl)pyrazol-1-yl]cyclohexyl]ethanol (7). N2 was bubbled through a solution of 2-[4-[3-(Difluoromethyl)-4-nitro-pyrazol-1-yl]cyclohexyl]ethanol 6 (216.1 mg, 0.75 mmol, 1 eq.) in EtOAc (3.74 mL) for 5 min. Then 10% Pd/C (238.5 mg, 0.22 mmol, 0.3 eq.) was added and N2 was bubbled through the solution for another 5 min. Then H₂ was bubbled through the solution for 5 min and the resulting mixture was stirred at rt under 1 atm H₂. After 3 h, LCMS showed total conversion. The solution was filtered through a celite pad and washed thoroughly with EtOAc. Finally, the filtrate was concentrated under reduced pressure to give 7 (169 mg, 81% yield) as a yellow oil. The product was used without purification in the next step.

LCMS method 1: 92.9% purity at 215 nm, [M+H]=260.2.

Step 7. Preparation of tert-Butyl N-[(3R,5R)-1-[3-[[3-(difluoromethyl)-1-[4-(2-hydroxyethyl)cyclohexyl]pyrazol-4-yl]carbamoyl]pyrazolo[1,5-a]pyrimidin-5-yl]-5-fluoro-3-piperidyl]carbamate (9). To a solution of 2-[4-[4-Amino-3-(difluoromethyl)pyrazol-1-yl]cyclohexyl]ethanol 7 (168.8 mg, 0.65 mmol, 1.3 eq.), 5-[(3R,5R)-3-(tert-butoxycarbonylamino)-5-fluoro-1-piperidyl]pyrazolo[1,5-a]pyrimidine-3-carboxylic acid 8-[synthesis in Final Product General Method 1 P-13]-(190 mg, 0.50 mmol, 1 eq.) and NMI (0.14 mL, 1.75 mmol, 3.5 eq.) in MeCN (2.50 mL) was added TCFH (168.6 mg, 0.60 mmol, 1.2 eq.) at 0° C. The reaction was then stirred at rt. After 16 h, LCMS showed total completion. MeCN was removed under reduced pressure and the residue was purified by reverse phase flash chromatography (30 g C18 column, liquid deposit (DMSO), elution: 5% MeOH/0.1% HCOOH over 3 CV, then 5 to 100% MeOH/0.1% HCOOH over 15 CV, then 100% MeOH/0.1% HCOOH over 3 CV (desired product was released around 80% MeCN). Fractions were combined and concentrated to give 9 (155.6 mg, 50% yield) as an off-white solid.

LCMS method 1: 99.9% purity at 215 nm, [M+H]=621.2.

¹H NMR (400 MHz, DMSO-d6) δ ppm 1.04-1.17 (m, 3H), 1.29-1.44 (m, 12H), 1.65-1.77 (m, 3H), 1.80-1.93 (m, 3H), 1.97-2.07 (m, 2H), 2.10-2.18 (m, 1H), 2.94-3.14 (m, 1H), 3.36-3.53 (m, 3H), 3.61-3.72 (m, 1H), 4.11-4.23 (m, 1H), 4.31-4.40 (m, 1H), 4.94-5.13 (m, 1H), 6.83-6.90 (m, 1H), 6.91-7.23 (m, 2H), 8.28 (s, 1H), 8.33 (br s, 1H), 8.82 (d, J=8.1 Hz, 1H), 9.31 (br s, 1H).

¹⁹F NMR (377 MHz, DMSO-d6) δ ppm-184.27-183.87 (m, 1 F), -112.01-110.10 (m, 2 F).

Step 8. Preparation of tert-Butyl N-[(3R,5R)-1-[3-[[3-(difluoromethyl)-1-[4-[2-[4-[2-(2,6-dioxo-3-piperidyl)-1-oxo-isoindolin-5-yl]piperazin-1-yl]ethyl]cyclohexyl]pyrazol-4-yl]carbamoyl]pyrazolo[1,5-a]pyrimidin-5-yl]-5-fluoro-3-piperidyl]carbamate (10). To a solution of tert-butyl N-[(3R,5R)-1-[3-[[3-(difluoromethyl)-1-[4-(2-hydroxyethyl)cyclohexyl]pyrazol-4-yl]carbamoyl]pyrazolo[1,5-a]pyrimidin-5-yl]-5-fluoro-3-piperidyl]carbamate 9 (81 mg, 0.13 mmol, 1 eq.) in dry DMSO (1 mL, 0.13 M) was added IBX (44.9 mg, 0.16 mmol, 1.2 eq.). The resulting mixture was stirred at room temperature overnight. LCMS after overnight stirring showed complete conversion to the desired aldehyde. To the reaction mixture was then added 3-(1-oxo-5-piperazin-1-yl-isoindolin-2-yl)piperidine-2,6-dione hydrochloride C-4 (52.5 mg, 0.14 mmol, 1.1 eq.), DCE (1 mL, 0.13 M) and DIPEA (0.23 mL, 1.31 mmol, 10 eq.). The mixture was stirred at rt for 10 minutes, then sodium triacetoxyborohydride (36.1 mg, 0.17 mmol, 1.3 eq.) was added. After 90 min., LCMS showed full conversion into compound 10. The DCE was evaporated and the reaction mixture was purified by reverse phase FC (50 g C18 RediSep Rf Gold column, liquid deposit (DMSO), 5% MeCN/0.1% HCOOH over 4 CV, then 5 to 95% MeCN/0.1% HCOOH over 20 CV). Pure fractions were combined and concentrated to give 10 (73 mg, 48% yield) as a tan solid. The desired product contained IBX residue.

LCMS method 2: 79.9% purity at 215 nm, [M-t-Bu+2H]2+=438.2.

¹H NMR (400 MHz, DMSO-d6) δ ppm 1.07-1.22 (m, 3H), 1.33 (s, 9H), 1.39-1.47 (m, 3H), 1.67-1.82 (m, 3H), 1.84-1.93 (m, 3H), 1.93-1.99 (m, 1H), 2.03 (br d, J=11.2 Hz, 2H), 2.09-2.18 (m, 1H), 2.34-2.43 (m, 3H), 2.53 (br s, 2H), 2.55-2.63 (m, 1H), 2.84-2.96 (m, 1H), 2.99-3.14 (m, 1H), 3.37-3.52 (m, 4H), 3.66 (br s, 1H), 4.14-4.24 (m, 2H), 4.29-4.36 (m, 1H), 4.50-4.78 (m, 1H), 4.93-5.12 (m, 2H), 6.86-6.93 (m, 1H), 7.07 (s, 2H), 7.18-7.48 (m, J=1.2 Hz, 1H), 7.52 (d, J=8.8 Hz, 1H), 8.14 (s, 2H), 8.28 (s, 1H), 8.34 (br s, 1H), 8.82 (d, J=7.8 Hz, 1H), 9.31 (s, 1H), 10.94 (s, 1H).

¹⁹F NMR (377 MHz, DMSO-d6) δ ppm-184.78 (br s, 1 F), -111.22 (br dd, J=53.1, 19.1 Hz, 2 F).

Step 9. Preparation of N-[3-(Difluoromethyl)-1-[4-[2-[4-[2-(2,6-dioxo-3-piperidyl)-1-oxo-isoindolin-5-yl]piperazin-1-yl]ethyl]cyclohexyl]pyrazol-4-yl]-5-[(3R,5R)-3-amino-5-fluoro-1-piperidyl]pyrazolo[1,5-a]pyrimidine-3-carboxamide dihydrochloride (P-30). To a round bottom flask was added tert-butyl N-[(3R,5R)-1-[3-[[3-(difluoromethyl)-1-[4-[2-[4-[2-(2,6-dioxo-3-piperidyl)-1-oxo-isoindolin-5-yl]piperazin-1-yl]ethyl]cyclohexyl]pyrazol-4-yl]carbamoyl]pyrazolo[1,5-a]pyrimidin-5-yl]-5-fluoro-3 piperidyl]carbamate 10 (73 mg, 0.08 mmol, 1.0 eq.) and HCl 4.0 M in dioxane (2.43 mL, 9.72 mmol, 124 eq.). The reaction was stirred at rt. LCMS after 18 h showed complete conversion. The reaction mixture was concentrated in vacuo and the residue was purified by reverse phase FC (50 g C18 RediSep Rf Gold column, liquid deposit (H₂O), elution: 5% MeCN/0.02 M HCl over 4 CV, then 5% to 30% MeCN/0.02 M HCl over 20 CV). Pure fractions were combined, concentrated and lyophilised to give P-30 (24.2 mg, 37% yield) as a white solid as a dihydrochloride salt.

LCMS method 3: 99.1% purity at 215 nm, [M−2HCl+H]+=831.3.

¹H NMR (400 MHz, DMSO-d6) δ ppm 1.11-1.27 (m, 2H), 1.42 (br s, 1H), 1.62-1.81 (m, 4H), 1.84-1.92 (m, 2H), 1.93-2.10 (m, 4H), 2.35-2.43 (m, 2H), 2.59 (br d, J=16.6 Hz, 1H), 2.84-2.98 (m, 1H), 3.04-3.48 (m, 11H), 4.02 (br d, J=11.7 Hz, 2H), 4.19-4.28 (m, 2H), 4.36 (d, J=15.4 Hz, 1H), 4.58 (br s, 1H), 4.87 (br s, 1H), 5.02-5.18 (m, 2H), 6.88 (br d, J=7.6 Hz, 1H), 6.98-7.28 (m, 3H), 7.60 (d, J=8.6 Hz, 1H), 8.20 (br s, 3H), 8.32 (s, 1H), 8.39 (s, 1H), 8.94 (d, J=7.8 Hz, 1H), 9.33 (s, 1H), 10.27 (br s, 1H), 10.96 (s, 1H).

¹⁹F NMR (377 MHz, DMSO-d6) δ ppm-184.51 (br s, 1F), -111.23 (br dd, J=52.5, 18.4 Hz, 2 F).

TABLE 6

| Compound | | | |
|---|---|---|---|
| No. | CBM | Structure | Characterization Data |
| P-30 | C-4 | 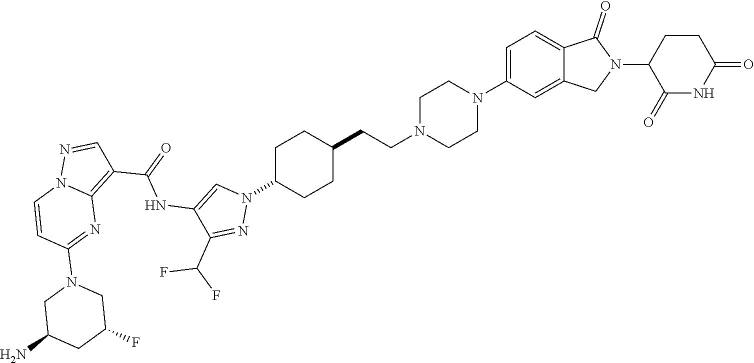 | 37% yield as a white solid. LCMS method 3: retention time: 1.897 min, 99% purity at 215 nm, [M − 2HCl + H]⁺ = 831.3; [M − 2HCl + 2H]²⁺ = 416.3. ¹H NMR (400 MHz, DMSO-d6) δ ppm 1.11-1.27 (m, 2 H), 1.42 (br s, 1 H), 1.62-1.81 (m, 4 H), 1.84-1.92 (m, 2 H), 1.93-2.10 (m, 4 H), 2.35-2.43 (m, 2 H), 2.59 (br d, J = 16.6 Hz, 1 H), 2.84-2.98 (m, 1 H), 3.04-3.48 (m, 11 H), 4.02 (br d, J = 11.7 Hz, 2 H), 4.19-4.28 (m, 2 H), 4.36 (d, J = 15.4 Hz, 1 H), 4.58 (br s, 1 H), 4.87 (br s, 1 H), 5.02-5.18 (m, 2 H), 6.88 (br d, J = 7.6 Hz, 1 H), 6.98- 7.28 (m, 3 H), 7.60 (d, J = 8.6 Hz, 1 H), 8.20 (br s, 3 H), 8.32 (s, 1 H), 8.39 (s, 1 H), 8.94 (d, J = 7.8 Hz, 1 H), 9.33 (s, 1 H), 10.27 (br s, 1 H), 10.96 (s, 1 H). ¹⁹F NMR (377 MHz, DMSO-d6) δ ppm −184.51 (br s, 1 F), −111.23 (br dd, J = 52.5, 18.4 Hz, 2 F). |

### Final Product Method 6

### Example S-11. Synthesis of P-38

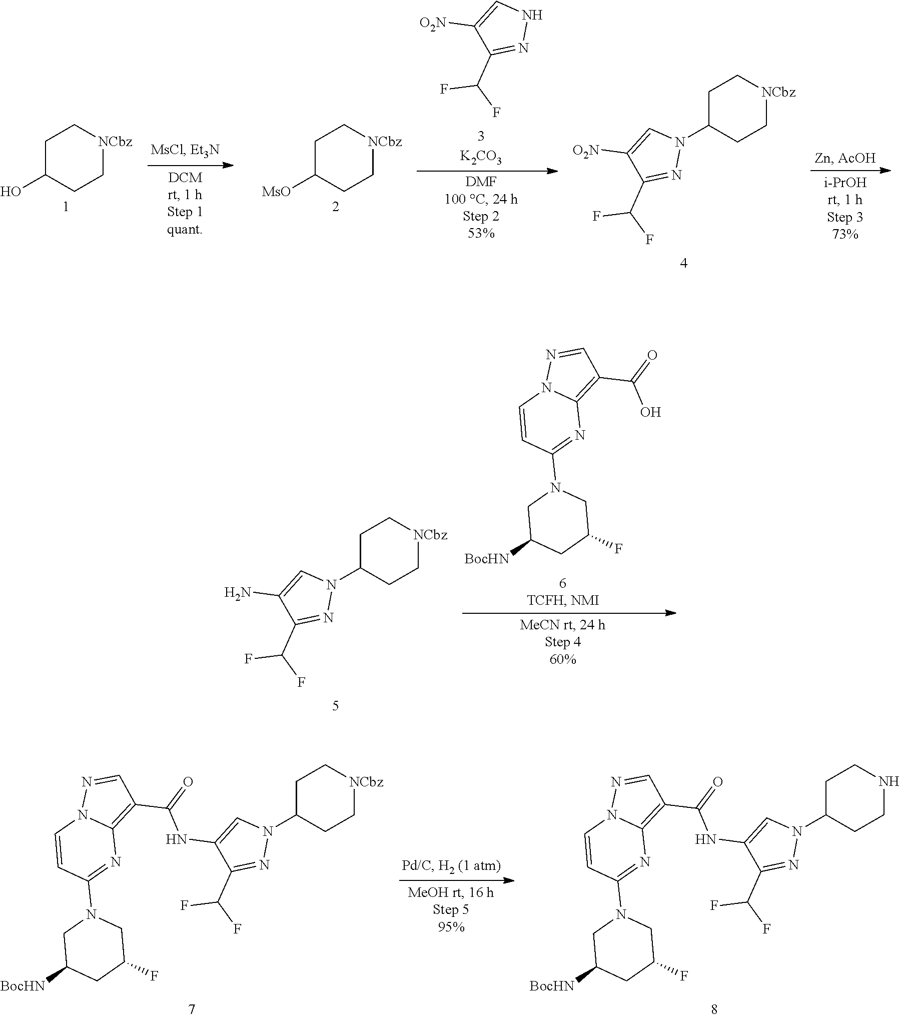

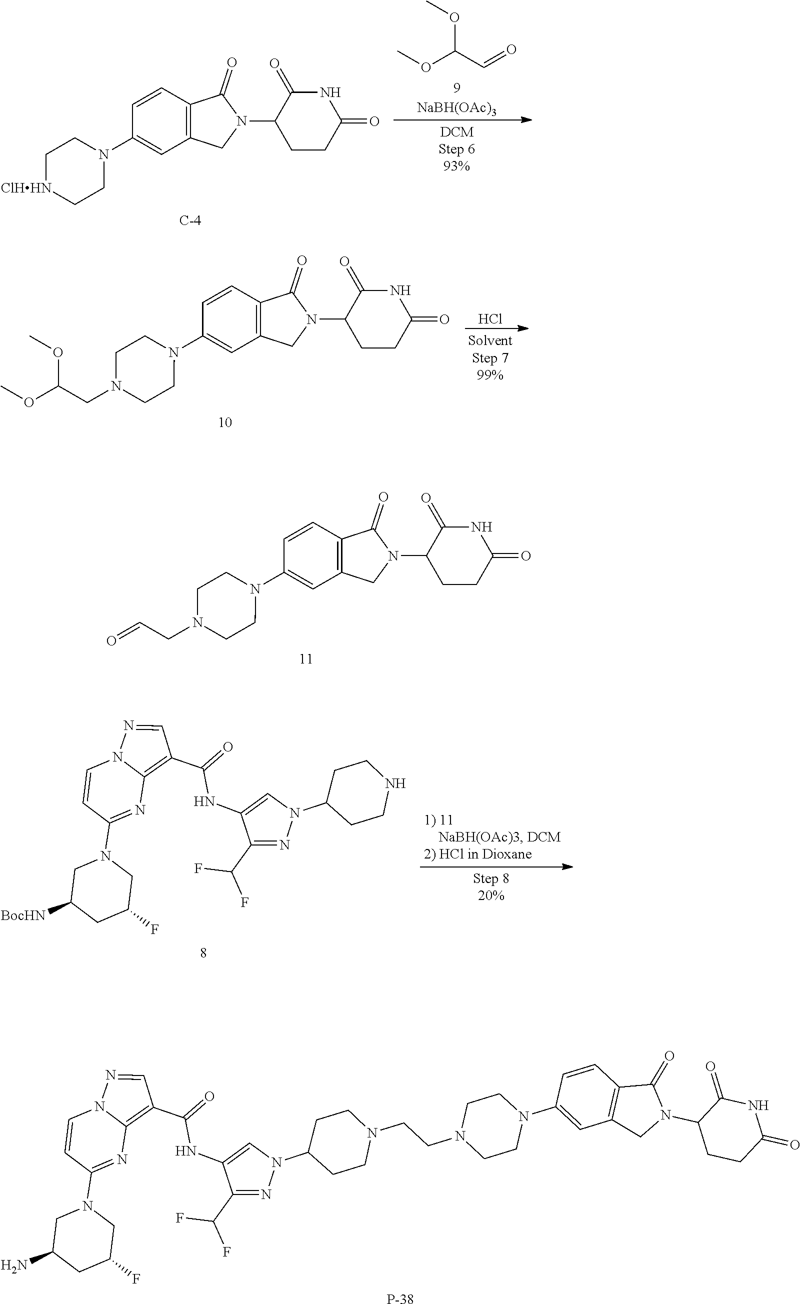

Step 1. Preparation of benzyl 4-methylsulfonyloxypiperidine-1-carboxylate (2). Methanesulfonyl chloride (0.821 mL, 10.6 mmol, 1.25 eq.) was added dropwise to an ice-cooled solution of 4-hydroxypiperidine-1-carboxylate 1 (2.00 g, 8.50 mmol, 1.0 eq.) and triethylamine (1.78 mL, 12.8 mmol, 1.5 eq.) in DCM (100 mL). The reaction mixture was stirred at room temperature. After 1 h, LCMS showed full conversion. Water (50 mL) and saturated NaHCO₃(aq) (50 mL) were added to the reaction mixture, the phases were separated, then the aqueous layer was extracted with DCM (3×50 mL). The combined organic layers were washed with brine (50 mL), then dried over MgSO₄, filtered and evaporated under reduced pressure to afford 2 (2.80 g, 8.50 mmol, quantitative yield) as a light-orange oil.

LCMS method 1: 95.8% purity at 215 nm, [M+H]+=314.1.

¹H NMR (400 MHz, CDCl₃) δ ppm 1.77-1.92 (m, 2H), 1.93-2.08 (m, 2H), 3.05 (s, 3H), 3.43 (ddd, J=13.7, 7.9, 3.9 Hz, 2H), 3.70-3.85 (m, 2H), 4.91 (tt, J=7.5, 3.8 Hz, 1 H), 5.14 (s, 2H), 7.27-7.43 (m, 5H).

Step 2. Preparation of benzyl 4-[3-(difluoromethyl)-4-nitro-pyrazol-1-yl]piperidine-1-carboxylate (4). A mixture of benzyl 4-methylsulfonyloxypiperidine-1-carboxylate 2 (1.00 g, 3.19 mmol, 1.2 eq.), 3-(difluoromethyl)-4-nitro-1H-pyrazole 3 (450 mg, 2.76 mmol, 1.0 eq.), and potassium carbonate (800 mg, 5.80 mmol, 2.1 eq.) in DMF (22 mL) was stirred at 100° C. under nitrogen atmosphere. After 24 h, LCMS showed full conversion. Water (100 mL) was added to the reaction mixture and the aqueous phase was extracted with EtOAc (3×50 mL). The combined organics were washed with 1:1 water/brine (3×50 mL) and brine (50 mL), dried over MgSO₄ and evaporated to dryness. The crude was purified by normal phase flash chromatography (80 g silica column, preabsorbed, elution: Heptanes/EtOAc, 95:5 to 70:30, 12 CV) to afford 4 (644 mg, 1.46 mmol, 53% yield) as a light-yellow oil.

LCMS method 1: 86.0% purity at 215 nm, [M+H]=381.1.

¹H NMR (400 MHz, CDCl₃) δ ppm 1.90-2.01 (m, 2H), 2.22 (br d, J=12.1 Hz, 2H), 2.88-3.04 (m, 2H), 4.37 (tt, J=11.7, 4.0 Hz, 3H), 5.16 (s, 2H), 7.12 (t, J=53.3 Hz, 1H), 7.32-7.43 (m, 5H), 8.21 (s, 1H).

¹⁹F NMR (377 MHz, CDCl₃) δ ppm-117.79 (s, 2 F).

Step 3. Preparation of benzyl 4-[4-amino-3-(difluoromethyl)pyrazol-1-yl]piperidine-1-carboxylate (5). Zinc (2.21 g, 33.9 mmol, 20.0 eq.; activated with 1.0 M HCl(aq)) was added to a solution of benzyl 4-[3-(difluoromethyl)-4-nitro-pyrazol-1-yl]piperidine-1-carboxylate 4 (644 mg, 1.46 mmol, 1.0 eq.) in i-PrOH (17 mL). Acetic acid (0.970 mL, 16.9 mmol, 10.0 eq.) was then added to the solution and the reaction mixture was stirred at room temperature. After 1 h, LCMS showed full conversion. The reaction mixture was filtered through a celite pad and evaporated to dryness. The residue was taken up in EtOAc (50 mL), washed with saturated NaHCO₃(aq) (10 mL) and brine (10 mL), dried over MgSO₄ and evaporated to dryness to afford 5 (500 mg, 1.24 mmol, 73% yield) as a brown oil.

LCMS method 1: 86.9% purity at 215 nm, [M+H]=351.1.

¹H NMR (400 MHz, DMSO-d6) δ ppm 1.73 (qd, J=12.2, 4.5 Hz, 2H), 1.91-2.01 (m, 2H), 2.86-3.10 (m, 2H), 4.08 (br d, J=12.8 Hz, 4H), 4.17-4.30 (m, 1H), 5.09 (s, 2H), 6.88 (t, J=54.2 Hz, 1H), 7.18 (s, 1H), 7.31-7.39 (m, 5H).

¹⁹F NMR (377 MHz, DMSO-d6) δ ppm-111.51 (s, 2 F).

Step 4. Preparation of benzyl 4-[3-(difluoromethyl)-4-[[5-[(3R,5R)-3-(tert-butoxycarbonylamino)-5-fluoro-1-piperidyl]pyrazolo[1,5-a]pyrimidine-3-carbonyl]amino]pyrazol-1-yl]piperidine-1-carboxylate (7). TCFH (614 mg, 2.19 mmol, 1.2 eq.) was added in one portion to a solution of 5-[(3R,5R)-3-(tert-butoxycarbonylamino)-5-fluoro-1-piperidyl]pyrazolo[1,5-a]pyrimidine-3-carboxylic acid 6-[synthesis in Final Product General Method 1 P-13]-(692 mg, 1.82 mmol, 1.0 eq.), benzyl 4-[4-amino-3-(difluoromethyl)pyrazol-1-yl]piperidine-1-carboxylate 5 (708 mg, 1.92 mmol, 1.05 eq.) and NMI (506 μL, 6.38 mmol, 3.5 eq.) in MeCN (12.1 mL), then the reaction mixture was stirred at room temperature. After 24 h, LCMS showed full conversion. The volatiles were evaporated under reduced pressure. The residue was purified by reverse phase flash chromatography purification (150 g C18 RediSep Rf Gold column, liquid deposit (DMSO), MeOH in 0.1% HCOOH(aq), 40%-90%, 15 CV). Fractions were combined and concentrated to afford 7 (823 mg, 1.10 mmol, 60% yield) as an off-white solid.

LCMS method 1: 95.0% purity at 215 nm, [M+H]=712.3.

¹H NMR (400 MHz, DMSO-d6) δ ppm 1.33 (br s, 9H), 1.68-1.97 (m, 4H), 2.03 (br d, J=10.6 Hz, 2H), 2.09-2.22 (m, 1H), 2.88-3.18 (m, 3H), 3.36-3.56 (m, 1H), 3.67 (br d, J=7.0 Hz, 1H), 4.12 (br d, J=13.2 Hz, 2H), 4.42-4.56 (m, 1H), 4.57-4.92 (m, 1H), 4.97 (d, J=45.4 Hz, 1H), 5.10 (s, 2H), 6.89 (br d, J=8.1 Hz, 1H), 6.92-7.26 (m, 2H), 7.29-7.36 (m, 1H), 7.38 (s, 2H), 7.39 (s, 2H), 8.29 (s, 1H), 8.38 (br s, 1H), 8.82 (d, J=7.8 Hz, 1H), 9.31 (br s, 1H).

¹⁹F NMR (377 MHz, DMSO-d6) δ ppm-186.27-182.64 (m, 1 F), -114.40-109.21 (m, 2 F).

Step 5. Preparation of tert-butyl N-[(3R,5R)-1-[3-[[3-(difluoromethyl)-1-(4-piperidyl)pyrazol-4-yl]carbamoyl]pyrazolo[1,5-a]pyrimidin-5-yl]-5-fluoro-3-piperidyl]carbamate (8). Benzyl 4-[3-(difluoromethyl)-4-[[5-[(3R,5R)-3-(tert-butoxycarbonylamino)-5-fluoro-1-piperidyl]pyrazolo[1,5-a]pyrimidine-3-carbonyl]amino]pyrazol-1-yl]piperidine-1-carboxylate 7 (739 mg, 1.04 mmol, 1.0 eq.) was dissolved in methanol (4.0 mL), then the solution was sparged with nitrogen under sonication. After 20 min, Pd/C 10% w/w (200 mg, 0.181 mmol, 0.18 eq.) was added, then the mixture was further sparged with nitrogen under sonication. After 20 min, the nitrogen balloon was switched with one filled with hydrogen and the reaction was sparged. After 10 min, the reaction mixture was stirred under hydrogen atmosphere. After 18 h, LCMS showed full conversion. The reaction mixture was filtered through celite, then the celite was washed thoroughly with MeOH. The filtrate was evaporated under reduced pressure and the residue was taken up in EtOAc. Heptanes were added until the formation of a precipitate. The suspension was evaporated to dryness to give 8 (620 mg, 0.987 mmol, 95% yield) as a pink solid.

LCMS method 1: 92.0% purity at 215 nm, [M+H]=578.3.

¹H NMR (400 MHz, DMSO-d6) δ ppm 0.81-0.87 (m, 1H), 1.20-1.27 (m, 1H), 1.34 (br s, 9H), 1.69-1.82 (m, 3H), 1.88-1.95 (m, 2H), 2.07-2.23 (m, 1H), 2.53-2.61 (m, 2H), 3.03 (br d, J=12.8 Hz, 3H), 3.37-3.51 (m, 1H), 3.67 (br d, J=2.1 Hz, 1H), 4.19-4.33 (m, 1H), 4.45-4.87 (m, 1H), 5.04 (d, J=46.3 Hz, 1H), 6.89 (br d, J=8.1 Hz, 1H), 6.92-7.33 (m, 2H), 8.29 (s, 1H), 8.33 (br s, 1H), 8.82 (d, J=7.8 Hz, 1H), 9.32 (br s, 1H).

¹⁹F NMR (377 MHz, DMSO-d6) δ ppm-187.05-180.57 (m, 1 F), -113.62-108.43 (m, 2 F).

Step 6. Preparation of 3-[5-[4-(2,2-dimethoxyethyl)piperazin-1-yl]-1-oxo-isoindolin-2-yl]piperidine-2,6-dione (10). To a round bottom flask was added 3-(1-oxo-5-piperazin-1-yl-isoindolin-2-yl)piperidine-2,6-dione; hydrochloride C-4 (175 mg, 0.48 mmol, 1 eq.) in CH₂Cl₂ (6 mL, 0.08 M) followed by 2,2-dimethoxyacetaldehyde 9 (0.12 mL, 0.69 mmol, 1.44 eq.), DIPEA (0.5 mL, 3.25 mmol, 6.8 eq.) and NaBH(OAc)₃ (180 mg, 0.85 mmol, 1.8 eq.). The reaction was stirred at rt. After 2 h, LCMS showed complete conversion into compound 10. The mixture was evaporated to dryness and taken up in DCM and water. The aqueous phase was extracted with DCM (2×), the organic layers were combined and washed with brine, dried over sodium sulfate, filtered and concentrated to dryness, to afford 10 (200 mg, 94% yield) as a yellow solid.

LCMS method 2: 93.7% purity at 215 nm, [M+H]=417.2.

¹H NMR (400 MHz, CHLOROFORM-d) δ ppm 1.52-1.71 (m, 6H), 2.18-2.28 (m, 1H), 2.29-2.43 (m, 1H), 2.79-2.96 (m, 3H), 3.41-3.53 (m, 7H), 3.61-3.82 (m, 3H), 4.24-4.30 (m, 1H), 4.40-4.46 (m, 1H), 5.20 (dd, J=13.1, 5.0 Hz, 1H), 6.90 (s, 1H), 7.00 (dd, J=8.6, 2.2 Hz, 1H), 7.76 (br d, J=8.1 Hz, 1H), 7.91 (s, 1H).

Step 7. Preparation of 3-[5-[4-(2,2-dihydroxyethyl)piperazin-1-yl]-1-oxo-isoindolin-2-yl]piperidine-2,6-dione (11). To 3-[5-[4-(2,2-dimethoxyethyl)piperazin-1-yl]-1-oxo-isoindolin-2-yl]piperidine-2,6-dione 10 (200 mg, 0.45 mmol, 1 eq.) was added 4 M HCl in dioxane (2 mL, 8 mmol, 18 eq.) and few drops of water. The reaction was stirred at rt. After an overnight period, LCMS showed complete conversion into compound 11. The solvent was evaporated and co-evaporated with MeCN (3×) to give 11 (200 mg, quantitative yield) as a brown solid.

LCMS method 2: 96.7% purity at 254 nm, [M+H₂O+H]+=389.1.

¹H NMR (400 MHz, DMSO-d6) δ ppm 1.88-2.01 (m, 1H), 2.32-2.47 (m, 1H), 2.51-2.68 (m, 2H), 2.84-2.99 (m, 1H), 3.03-3.43 (m, 5H), 3.45-3.54 (m, 1H), 3.56-3.75 (m, 2H), 3.92-4.06 (m, 1H), 4.18-4.28 (m, 1H), 4.31-4.41 (m, 1H), 5.06 (dd, J=13.3, 5.0 Hz, 1H), 7.07-7.22 (m, 2H), 7.58 (dd, J=8.4, 1.8 Hz, 1H), 7.51-7.51 (m, 1H), 10.09-10.60 (m, 1H), 10.95 (s, 1H).

Step 8. Preparation of N-[3-(difluoromethyl)-1-[1-[2-[4-[2-(2,6-dioxo-3-piperidyl)-1-oxo-isoindolin-5-yl]piperazin-1-yl]ethyl]-4-piperidyl]pyrazol-4-yl]-5-[(3R,5R)-3-amino-5-fluoro-1-piperidyl]pyrazolo[1,5-a]pyrimidine-3-carboxamide (P-38).

To a solution of 3-[5-[4-(2,2-dihydroxyethyl)piperazin-1-yl]-1-oxo-isoindolin-2-yl]piperidine-2,6-dione 11 (81.48 mg, 0.21 mmol, 1.2 eq.) in MeCN (1.75 mL, 0.1 M) was added DIPEA (0.2 mL, 1.3 mmol, 7.4 eq.) and tert-butyl N-[(3R,5R)-1-[3-[[3-(difluoromethyl)-1-(4-piperidyl)pyrazol-4-yl]carbamoyl]pyrazolo[1,5-a]pyrimidin-5-yl]-5-fluoro-3-piperidyl]carbamate 8 (100.97 mg, 0.17 mmol, 1 eq.), followed by NaBH(OAc)₃ (74.1 mg, 0.35 mmol, 2 eq.). The reaction was stirred at rt. LCMS after 4 h showed complete conversion into the desired product. The solvent was evaporated and the residue was purified by reverse-phase FC (50 g C18 gold column, liquid deposit (DMSO), elution: 30 to 60% MeOH/0.1% HCOOH over 12 CV), affording the desired product with good purity. The resulting product was then solubilized in HCl 4 M in dioxane (1 mL, 4 mmol, 23 eq.). After 2 h, LCMS showed complete conversion into P-38. The solution was concentrated and purified by reverse phase FC purification (50 g C18 column, liquid deposit (DMSO), 5% MeCN/0.02 M HCl over 4 CV, then 5 to 20% MeCN/0.02 M HCl over 15 CV, desired product came out around 20% MeCN), to afford P-38 (28.8 mg, 20% yield) as a white solid as a dihydrochloride salt.

LCMS method 3: 99.4% purity at 215 nm, [M+H]=832.4.

¹H NMR (400 MHz, DMSO-d6) δ ppm 1.90-2.10 (m, 2H), 2.27-2.46 (m, 6H), 2.56-2.64 (m, 3H), 2.86-2.98 (m, 2H), 3.19-3.33 (m, 6H), 3.62-3.95 (m, 8H), 4.05-4.17 (m, 2H), 4.20-4.29 (m, 1H), 4.32-4.41 (m, 1H), 4.53-4.70 (m, 2H), 4.80-4.94 (m, 1H), 5.03-5.19 (m, 2H), 6.90 (br d, J=8.3 Hz, 1H), 7.04-7.34 (m, 3H), 7.59 (br d, J=8.1 Hz, 1H), 8.25-8.38 (m, 4H), 8.47 (s, 1H), 8.94 (d, J=7.8 Hz, 1H), 9.37 (s, 1H), 10.62-11.07 (m, 2H).

¹⁹F NMR (377 MHz, DMSO-d6) δ ppm-185.16-184.13 (m, 2 F), -111.92-111.26 (m, 1 F).

TABLE 7

| Compound | | | |
|---|---|---|---|
| No. | CBM | Structure | Characterization Data |
| P-38 | C-4 | 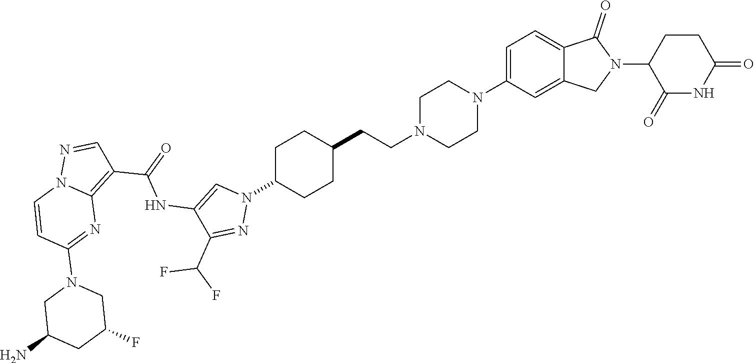 | 20% yield as a white solid. LCMS method 3: retention time: 1.794 min, 99% purity at 215 nm, [M − 2HCl + H]⁺ = 832.4, [M − 2HCl + 2H]²⁺ = 416.8 ¹H NMR (400 MHz, DMSO-d6) δ ppm 1.90-2.10 (m, 2 H), 2.27-2.46 (m, 6 H), 2.56-2.64 (m, 3 H), 2.86-2.98 (m, 2 H), 3.19- 3.33 (m, 6 H), 3.62-3.95 (m, 8 H), 4.05-4.17 (m, 2 H), 4.20-4.29 (m, 1 H), 4.32-4.41 (m, 1 H), 4.53-4.70 (m, 2 H), 4.80-4.94 (m, 1 H), 5.03-5.19 (m, 2 H), 6.90 (br d, J = 8.3 Hz, 1 H), 7.04-7.34 (m, 3 H), 7.59 (br d, J = 8.1 Hz, 1 H), 8.25-8.38 (m, 4 H), 8.47 (s, 1 H), 8.94 (d, J = 7.8 Hz, 1 H), 9.37 (s, 1 H), 10.62-11.07 (m, 2 H). ¹⁹F NMR (377 MHz, DMSO-d6) δ ppm −185.16- −184.13 (m, 2 F), −111.92-−111.26 (m, 1 F). |

### Biological Examples

### Example B1. IRAK4 PhosphoSens Biochemical Assay (BIOCHEM) Procedure

The PhosphoSens® biochemical assay was performed as described by the vendor (AssayQuant Technologies Inc. Marlborough, MA). A 1.25× stock solution of IRAK4 (PV4002, ThermoFisher Scientific, Waltham, MA) and a 5× stock solution of ATP and Sox conjugated peptide substrate, AQT0326 (CSKS-AQT0326B, AssayQuant Technologies), were prepared in 1× kinase reaction buffer consisting of 50 mM HEPES, pH 7.5, 0.01% Brij-35, 0.5 mM EGTA, 20 mM MgCl₂ and 1 mM DTT. 10 μL of the ATP and substrate solution mix, for a final concentration of 200 M ATP and 10 M peptide substrate, was added to a Corning 3574 384-well, white, non-binding surface microtiter plate containing 0.5 μL of serially diluted test compounds prepared in DMSO. The reactions were started with the addition of 40 μL of the enzyme solution, for a final IRAK4 concentration of 1 nM, and monitored every 71 seconds for 240 minutes at λ_{EX} 360/λ_{EM} 485 in a BioTek Synergy H₄ plate reader (Agilent Technologies, Santa Clara, CA) at room temperature. The initial linear portions of the progress curves were fit according to a linear equation to yield the slopes and converted to % inhibition based on a value of 100% activity for the no inhibitor treated control. IC₅₀ values of each compound were obtained by fitting the % inhibition-compound concentration curves using Dotmatics software (Dotmatics, Bishops Stortford, Hertfordshire, England),

### Example B2. Reagent Preparations

Cell culture media was prepared in a tissue culture hood in a sterile environment by adding 10% FBS and 1% Penicillin Streptomycin to 500 mL no phenol red RPMI 1640 media. The media was filtered through a Nalgene Bottle Top Filter and stored at 4° C.

The Cell titer Glo (CTG) buffer and substrate (CellTiter-Glo Luminescent Cell Viability Assay, Promega Ref. #G7573) were stored at −20° C. The CTG buffer (100 mL) was warmed in a bead bath and added to the CTG substrate bottle in a tissue culture hood. The solution was mixed with a pipette until it became homogenous. CTG reagent was aliquoted into 15 mL falcon tubes and stored at −20° C.

For Homogenous Time Resolved Fluorescence (HTRF) assays, a Cisbio HTRF kit was used, which included: Lysis Buffer #1 4×, Blocking Reagent #3 100×, 20× Antibody 1 (Anti-IRAK4 d2), 20× Antibody 2 (Anti-IRAK4 k), and Detection Buffer.

4× Lysis Buffer was stored at 4° C. For use as 1× Lysis buffer, the 4× solution was diluted with de-ionized water (distilled water, Gibco Cat. #15230279) and 100× Blocking Reagent in a 1:3:0.04 volume ratio.

20× Antibody Solution aliquots were stored at −80° C. and the Detection Buffer was stored at 4° C. For use as a 1× Antibody Solution, the 20× Antibody Solution aliquot was diluted with Detection Buffer in a 1:19 volume ratio.

### Example B3. Advanced Lipoxidation End Product THP1 Homogeneous Time Resolved Fluorescence (ALE THP1 HTRF) Procedure

Cells were lysed at room temperature for 45 min with shaking. A BCA protein assay was performed and normalization was conducted to the desired total protein concentration with 1× lysis buffer. Next, 1× Antibody Solutions were prepared by adding 380 μL Detection Buffer to 20 μL 20× Antibody Solution aliquots and mixing well. 1× Antibody Solutions were combined 1:1 and briefly vortexed. For control wells, 20 μL 1× anti IRAK4-k Antibody Solution was saved. The 384-well plate (ProxiPlate-384 Plus, Perkin Elmer Cat. #6008289) was loaded by adding 4 μL of the mixed Antibody Solution to empty wells using a single channel repeater. Using a multi-channel repeater, 16 μL lysate was added per well, and any bubbles that were formed were popped with 20 μL pipette tips and Kimwipe edges. In column 10, triplicates of each control were prepared. In wells A10, B10, and C₁₀, buffer control was prepared by adding 16 μL Lysis Buffer and 4 μL Detection Buffer. In wells D10, E10, and F10, cryptate control was prepared by adding 16 μL Lysis Buffer, 2 μL Detection Buffer, and 2 μL 1× anti-IRAK4-k Antibody Solution. In wells G10, H₁₀, and 110, a negative control was prepared by adding 16 μL Lysis Buffer and 4 μL mixed Antibody Solution. The plate was sealed with a clear seal and covered with an aluminum lid. The plate was spun down at 800 g for 5 min and incubated in the dark at room temperature overnight. The next day, the plate was spun down at 800 g for 5 min. The samples were analyzed by a plate reader (Envision, PerkinElmer) using the Desnor 384 HTRF program.

A summary of the ALE THP1 HTRF data for the tested compounds is provided in Table 8 below.

TABLE 8
ALE THP1 HTRF and LVL THP1 HTRF
Results of the Compounds.

| Compound | BIOCHEM | ALE THP1 HTRF | ALE THP1 HTRF |
|---|---|---|---|
| No. | IC₅₀ (nM) | EC₅₀ (μM) | Y_{MIN} (%) |
| P-1 | 8.6 | 0.067 | 30 |
| P-2 | 15.3 | >10 | 63 |
| P-3 | 4.3 | >10 | 54 |
| P-4 | 3.2 | >10 | 94 |
| P-5 | 0.5 | >10 | 60 |
| P-6 | 8.1 | 2.816 | 26 |
| P-7 | <0.5 | >10 | 74 |
| P-8 | <0.5 | 0.0089 | 33 |
| P-9 | 1.4 | >10 | 78 |
| P-10 | 1.2 | >10 | 73 |
| P-11 | 0.5 | >10 | 74 |
| P-12 | 0.5 | 4.275 | 66 |
| P-13 | <0.5 | 0.008 | 24 |
| P-14 | 1.6 | 10 | 73 |
| P-15 | <0.5 | 0.008 | 18 |
| P-16 | 0.5 | 0.013 | 41 |
| P-17 | 1.2 | >10 | >95 |
| P-18 | <0.5 | >10 | 69 |
| P-19 | <0.5 | 0.007 | 14 |
| P-20 | <0.5 | 0.011 | 22 |
| P-21 | <0.5 | 0.005 | 18 |
| P-22 | <0.5 | 0.003 | 27 |
| P-23 | <0.5 | 0.008 | 20 |
| P-24 | <0.5 | 0.004 | 11 |
| P-25 | <0.5 | 0.007 | 11 |
| P-26 | <0.5 | 0.006 | 29 |
| P-27 | <0.5 | 0.026 | 24 |
| P-28 | >1 | >10 | >95 |
| P-29 | <0.5 | 0.004 | 10 |
| P-30 | <0.5 | 0.008 | 10 |
| P-31 | <0.5 | 0.007 | 27 |
| P-32 | <0.5 | 0.006 | 12 |
| P-33 | 9.4 | 0.024 | 68 |
| P-34 | <0.5 | 0.006 | 9 |
| P-35 | <0.5 | 0.004 | 9 |
| P-36 | 0.9 | 0.009 | 29 |
| P-37 | <0.5 | 0.020 | 12 |
| P-38 | <0.5 | 0.006 | 10 |

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A compound of Formula (I): 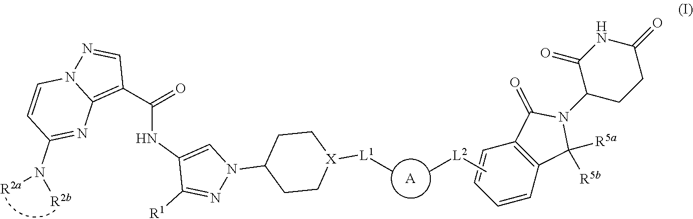
or a pharmaceutically acceptable salt thereof, wherein:
R¹ is C₁-C₆ haloalkyl, C₁-C₆ alkyl, or —CN;
R^{2a} is H or C₁-C₆ alkyl;
R^{2b} is C₅-C₆ cycloalkyl optionally substituted with 1-5 R³ groups;
or the dashed line between R^{2a} and R^{2b} represents a ring structure where R^{2a} and R^{2b} are taken together with the nitrogen atom to which they are attached to form a 6-membered heterocyclyl optionally containing one additional heteroatom selected from N and O, wherein the heterocyclyl is optionally substituted by 1-5 R³ groups;
each R³ is independently —NH₂, —OH, halo, C₁-C₆ alkyl, or C₁-C₆ haloalkyl;
X is CH or N;
L¹ is —C(O)N(H)—, —N(H)C(O)—, —C(O)—, —(C₁-C₆ alkylene)N(R⁴)—, or C₁-C₆ alkylene;
R⁴ is H or C₁-C₆ alkyl;
R^{5a} and R^{5b} are each H or are taken together to form an oxo group;
Ring A is 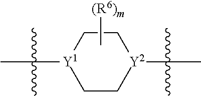
or an 8- to 10-membered spiro heterocyclylene containing 1-3 nitrogen atoms, wherein the heterocyclylene is substituted by m R⁶ groups;
Y¹ and Y² are independently CH or N;
each R⁶ is independently halo, C₁-C₆ alkyl, or C₁-C₆ haloalkyl;
m is 0-5;
L² is a bond or —N(R⁷)—; and
R⁷ is H or C₁-C₆ alkyl.

2. The compound of claim 1, or a pharmaceutically acceptable salt thereof, wherein:
R¹ is C₁-C₃ haloalkyl, C₁-C₃ alkyl, or —CN.

3. The compound of claim 2, or a pharmaceutically acceptable salt thereof, wherein:
R¹ is —CHF₂, —CF₃, —CH₃, or —CN.

4. The compound of any one of claims 1-3, or a pharmaceutically acceptable salt thereof, wherein:
R^{2a} is H or C₁-C₃ alkyl; and
R^{2b} is cyclohexyl optionally substituted with 1-3 R³ groups.

5. The compound of any one of claims 1-3, or a pharmaceutically acceptable salt thereof, wherein:
R^{2a} and R^{2b} are taken together with the nitrogen atom to which they are attached to form a 6-membered heterocyclyl optionally containing one additional heteroatom selected from N and O, wherein the heterocyclyl is optionally substituted by 1-2 R³ groups.

6. The compound of any one of claims 1-5, or a pharmaceutically acceptable salt thereof, wherein:
each R³ is independently —NH₂, —OH, halo, C₁-C₃ alkyl, or C₁-C₃ haloalkyl.

7. The compound of claim 6, or a pharmaceutically acceptable salt thereof, wherein:
each R³ is independently —NH₂, —OH, F, or —CH₃.

8. The compound of any one of claims 1-7, or a pharmaceutically acceptable salt thereof, wherein: 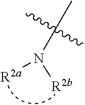
is

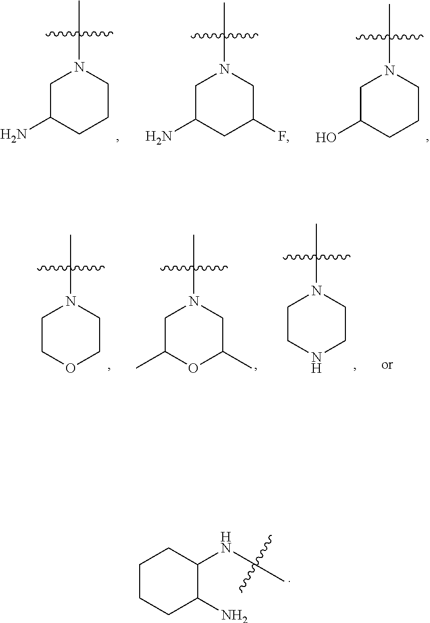

9. The compound of any one of claims 1-8, or a pharmaceutically acceptable salt thereof, wherein:
L¹ is —C(O)N(H)—, —N(H)C(O)—, —C(O)—, —(C₁-C₃ alkylene)N(R⁴)—, or C₁-C₃ alkylene; and
R⁴ is H or C₁-C₃ alkyl.

10. The compound of claim 9, or a pharmaceutically acceptable salt thereof, wherein:
L¹ is —C(O)N(H)—, —N(H)C(O)—, —C(O)—, —CH₂N(CH₃)—, —CH₂—, or —CH₂CH₂—.

11. The compound of any one of claims 1-10, or a pharmaceutically acceptable salt thereof, wherein:
Ring A is 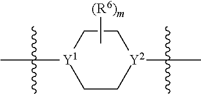
or a 10-membered spiro heterocyclylene containing 2 nitrogen atoms,
wherein the spiro heterocyclylene is substituted by m R⁶ groups.

12. The compound of any one of claims 1-11, or a pharmaceutically acceptable salt thereof, wherein:
each R⁶ is independently halo, C₁-C₃ alkyl, or C₁-C₃ haloalkyl.

13. The compound of any one of claims 1-12, or a pharmaceutically acceptable salt thereof, wherein:
m is 0, 1, or 2.

14. The compound of any one of claims 1-13, or a pharmaceutically acceptable salt thereof, wherein:
Ring A is

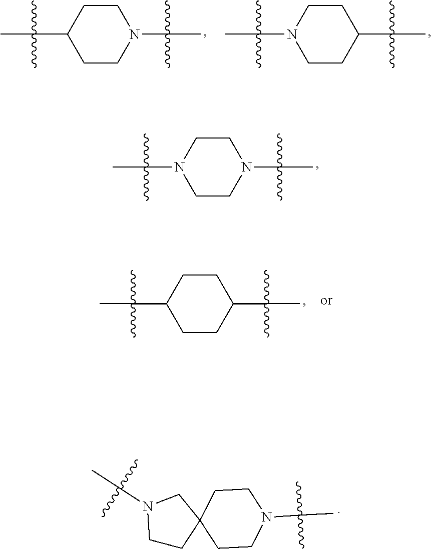 [SMILES: *C1CCN(*)CC1 ; *C1CCN(*)CC1 ; *N1CCN(*)CC1 ; *C1CCC(*)CC1 ; *N1CCC2(CC1)CCN(*)C2]

15. The compound of any one of claims 1-14, or a pharmaceutically acceptable salt thereof, wherein:
(i) L² is a bond; or
(ii) L² is —N(R⁷)—; and
R⁷ is H or C₁-C₃ alkyl.

16. The compound of any one of claims 1-15, or a pharmaceutically acceptable salt thereof, wherein: 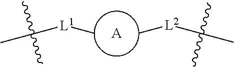
is

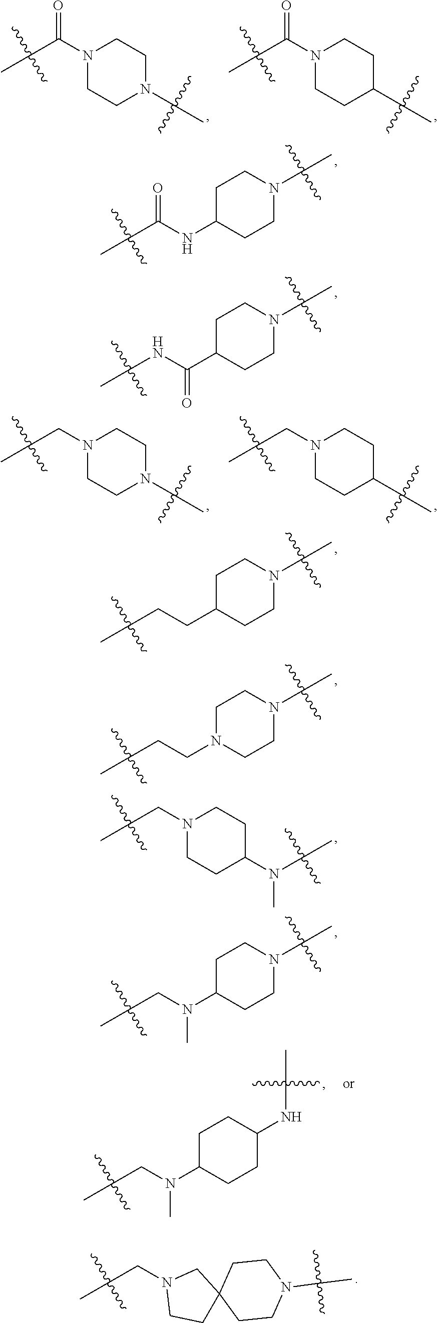

17. The compound of any one of claims 1-16, or a pharmaceutically acceptable salt thereof, wherein the compound is of Formula (IIa), (IIb), (IIIa), or (IIb):

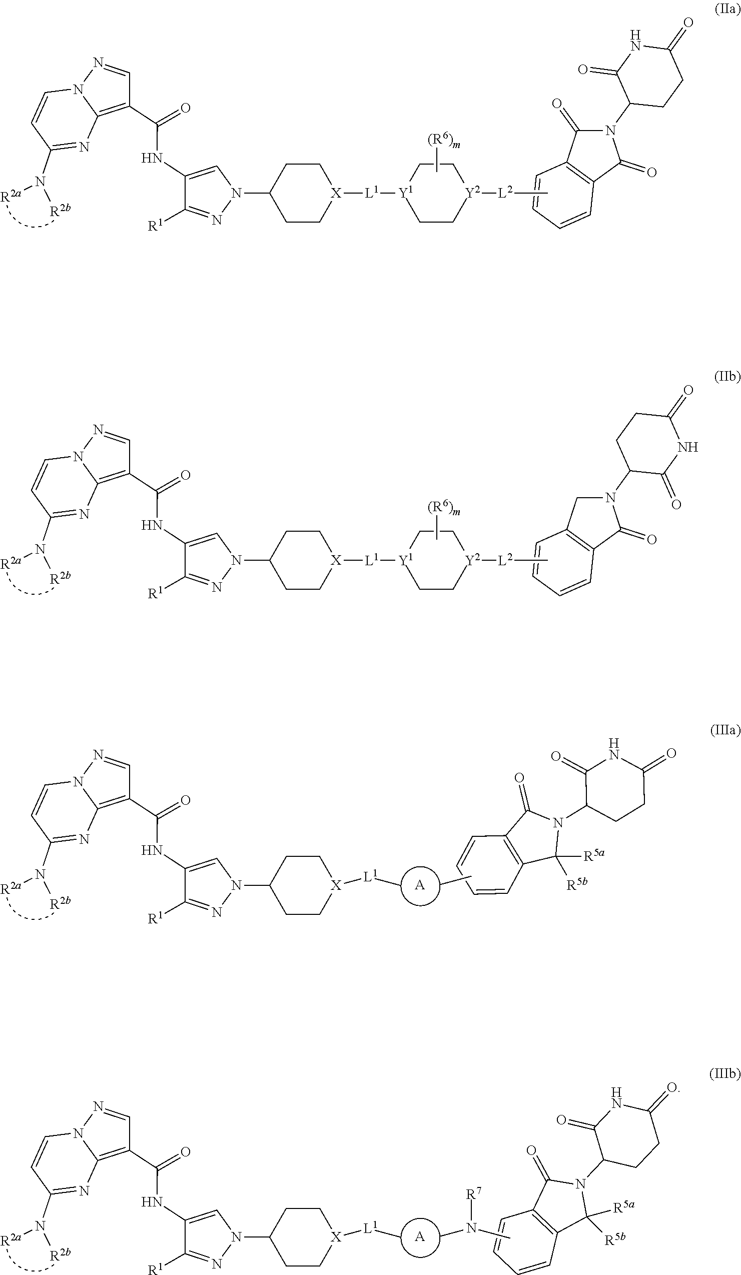

18. A compound selected from the compounds of Table 1 or a pharmaceutically acceptable salt thereof.

19. A pharmaceutical composition comprising the compound of any one of claims 1-18, or a pharmaceutically acceptable salt thereof, and a pharmaceutically acceptable excipient.

20. A method of (i) modulating interleukin-1 (IL1) receptor-associated kinase 4 (IRAK4) comprising contacting IRAK4 with an effective amount of the compound of any one of claims 1-18, or a pharmaceutically acceptable salt thereof, or the pharmaceutical composition of claim 19; or (ii) treating an inflammatory or autoimmune disease in a subject in need thereof, comprising administering to the subject an effective amount of the compound of any one of claims 1-18, or a pharmaceutically acceptable salt thereof, or the pharmaceutical composition of claim 19, optionally wherein the inflammatory or autoimmune disease is atopic dermatitis, asthma, lupus, rheumatoid arthritis, familial mediterranean fever, psoriasis, generalized pustular psoriasis, cryoprin-associated periodic syndrome, hidradenitis suppurativa, Bechet's syndrome, or familial cold autoinflammatory syndrome.
